(19) United States
(12) Patent Application Publication
(10) Pub. No.: US 2024/0336924 A1
(43) Pub. Date: Oct. 10, 2024

(54) MODULATION OF GENE TRANSCRIPTION USING ANTISENSE OLIGONUCLEOTIDES TARGETING REGULATORY RNAS

(71) Applicant: Camp4 Therapeutics Corporation, Cambridge, MA (US)

(72) Inventors: Alfica Sehgal, Belmont, MA (US); Bryan J. Matthews, Somerville, MA (US); David A. Bumcrot, Belmont, MA (US); Justin A. Caravella, Cambridge, MA (US); Mario Esteban Contreras Gamboa, Cambridge, MA (US); Rachana S. Kelkar, Malden, MA (US); Yun Joon Jung, Lexington, MA (US); Yuting Liu, Lexington, MA (US); Rutuja Sudhakar Pai, Charlestown, MA (US); Henry Munir Moukheiber, Cambridge, MA (US); Subhadeep Roy, Lafayette, CO (US); Yuchun Guo, Framingham, MA (US)

(21) Appl. No.: 18/752,290

(22) Filed: Jun. 24, 2024

Related U.S. Application Data
(63) Continuation of application No. PCT/US2022/082295, filed on Dec. 22, 2022.
(60) Provisional application No. 63/308,373, filed on Feb. 9, 2022.
(60) Provisional application No. 63/292,920, filed on Dec. 22, 2021.

(51) Int. Cl. C12N 15/113; A61P 13/00

(52) U.S. Cl. CPC C12N 15/1137; A61P 13/00; C12Y 603/04016; C12N 2310/113; C12N 2310/315; C12N 2310/3231; C12N 2310/3341; C12N 2310/341; C12N 2310/351; C12N 2310/3525

(57) ABSTRACT

Described herein are methods of modulating gene transcription using antisense oligonucleotides (ASOs) targeting regulatory RNAs, such as promoter-associated RNAs and enhancer RNAs. These methods are useful for modulating the levels of gene products, for example, increasing expression of Carbamoyl-Phosphatase Synthetase 1 (CPS1), thereby treating diseases associated with aberrant gene expression.

### CROSS REFERENCE TO RELATED APPLICATIONS

This application is a continuation of International Application No. PCT/US2022/082295, filed Dec. 22, 2022, which claims the benefit of U.S. Provisional Application No. 63/292,920, filed Dec. 22, 2021, and U.S. Provisional Application No. 63/308,373, filed Feb. 9, 2022, each of which are hereby incorporated in their entirety by reference.

### SEQUENCE LISTING

The instant application contains a Sequence Listing which has been submitted via EFS-Web and is hereby incorporated by reference in its entirety. Said XML copy, created on Dec. 21, 2022, is named CTC-028WO_140628340009.xml, and is 925,586 bytes in size.

### FIELD OF THE INVENTION

The disclosure relates to methods of upregulating or downregulating gene transcription using antisense oligonucleotides (ASOs) targeting regulatory RNAs (e.g. CPS1 regulatory RNAs), such as promoter-associated RNAs and enhancer RNAs.

### BACKGROUND

Transcription factors bind specific sequences in promoter and enhancer DNA elements to regulate gene transcription. It was recently reported that active promoters and enhancer elements are themselves transcribed, generating noncoding regulatory RNAs (regRNAs) such as promoter-associated RNAs (paRNAs) and enhancer RNAs (eRNAs) (see Sartorelli and Lauberth, Nat. Struct. Mol. Biol. (2020) 27, 521-28). Unlike coding RNAs, regRNAs are transcribed bi-directionally. Various models have been proposed for the functions of regRNAs, including nucleosome remodeling (see Mousavi et al., Mol. Cell (2013) 51(5):606-17), modulation of enhancer-promoter looping (see Lai et al., Nature (2013) 494(7438):497-501), and direct interaction with transcription regulators (see Sigova et al., Science (2015) 350, 978-81).

Gene expression has been generally known as an undruggable biological process. Despite on-going efforts into understanding the biology of gene transcription and regRNAs, clinically suitable methods of modulating gene expression are limited. There remains a need for new and useful methods for treating diseases associated with aberrant gene expression.

### SUMMARY

In one aspect, provided herein are antisense oligonucleotides (ASO) complementary to at least 8 contiguous nucleotides of a regulatory RNA (regRNA) of Carbamoyl-Phosphate Synthetase 1 (CPS1), wherein the regRNA has a nucleotide sequence selected from the group consisting of SEQ ID NO: 49, 50, 69-79 and 89-90.

In some embodiments, the ASO is complementary to a sequence in the regRNA that is no more than 200 nucleotides from the 3′ end of the regRNA.

In some embodiments, the ASO is complementary to a sequence in the regRNA that is no more than 200 nucleotides from the 5′ end of the regRNA.

In some embodiments, the regulatory RNA has a nucleotide sequence of SEQ ID NO: 49, and the ASO comprises a nucleotide sequence of SEQ ID NO: 1.

In some embodiments, the regulatory RNA has a nucleotide sequence of SEQ ID NO: 49, and the ASO comprises a nucleotide sequence of SEQ ID NO: 2.

In some embodiments, the regulatory RNA has a nucleotide sequence of SEQ ID NO: 49, and the ASO comprises a nucleotide sequence selected from the group consisting of SEQ ID NOs: 1-3.

In some embodiments, the regulatory RNA has a nucleotide sequence of SEQ ID NO: 50, and the ASO comprises a nucleotide sequence selected from the group consisting of SEQ ID NOs: 4-15.

In some embodiments, the regulatory RNA has a nucleotide sequence of SEQ ID NO: 89, and the ASO comprises a nucleotide sequence selected from the group consisting of SEQ ID NOs: 487-574.

In some embodiments, the regulatory RNA has a nucleotide sequence of SEQ ID NO: 90, and the ASO comprises a nucleotide sequence selected from the group consisting of SEQ ID NOs: 575-662.

In some embodiments, the ASO is no more than 50, 40, 30, or 25 nucleotides in length.

In some embodiments, the ASO comprises an polynucleotide comprising one or more chemical modifications. In some embodiments, the ASO comprises an RNA polynucleotide comprising one or more chemical modifications. In some embodiments, the ASO comprises an RNA and DNA polynucleotide comprising one or more chemical modifications.

In some embodiments, at least 3, 4, or 5 nucleotides at the 5′ end and at least 3, 4, or 5 nucleotides at the 3′ end of the ASO comprise ribonucleotides with one or more chemical modifications.

In some embodiments, the one or more chemical modifications comprise a nucleotide sugar modification comprising one or more of 2′-O—C1-4alkyl such as 2′-O-methyl (2′-OMe), 2′-deoxy (2′-H), 2′-O—C1-3alkyl-O—C1-3alkyl such as 2′-methoxyethyl (“2′-MOE”), 2′-fluoro (“2′-F”), 2′-amino (“2′-NH2”), 2′-arabinosyl (“2′-arabino”) nucleotide, 2′-F-arabinosyl (“2′-F-arabino”) nucleotide, 2′-locked nucleic acid (“LNA”) nucleotide, 2′-amido bridge nucleic acid (AmNA), 2′-unlocked nucleic acid (“ULNA”) nucleotide, a sugar in L form (“L-sugar”), 4′-thioribosyl nucleotide, constrained ethyl (cET), 2′-fluoro-arabino (FANA), or thiomorpholino.

In some embodiments, the one or more chemical modifications comprise an internucleotide linkage modification comprising one or more of phosphorothioate (“PS” or (P(S))), phosphoramidate (P(NR1R2) such as dimethylaminophosphoramidate (P(N(CH3)2)), phosphonocarboxylate (P(CH2)nCOOR) such as phosphonoacetate “PACE” (P(CH2COO—)), thiophosphonocarboxylate ((S)P(CH2)nCOOR) such as thiophosphonoacetate “thioPACE” ((S)P(CH2COO—)), alkylphosphonate (P(C1-3alkyl) such as methylphosphonate —P(CH3), boranophosphonate (P(BH3)), or phosphorodithioate (P(S)2).

In some embodiments, the one or more chemical modifications comprise a nucleobase modification comprising one or more of 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, Unstructured Nucleic Acid (“UNA”), isoguanine (“isoG”), isocytosine (“isoC”) a glycerol nucleic acid (GNA), glycerol nucleic acid (GNA), or thiophosphoramidate morpholinos (TMOs).

In some embodiments, the one or more chemical modifications comprise 2′-O-methoxyethyl, 5-methyl cytidine, locked nucleic acid (LNA), and phosphorothioate internucleotide bond.

In some embodiments, the ASO comprises 8 or more contiguous nucleotides of unmodified DNA flanked by at least 3 nucleotides of modified ribonucleotides at each of the 5′ end and the 3′ end.

In some embodiments, the ASO comprises the nucleotide sequence and/or chemical modification of any one of SEQ ID NO: 16, 22-27, 31-39, 403-412, 414-415, 417-432, 435-480, or 482-486.

In some embodiments, the ASO comprises the nucleotide sequence and/or chemical modification of SEQ ID NO: 409.

In some embodiments, the ASO further comprises at least one phosphorothioate internucleotide bond.

In some embodiments, the ASO comprises the nucleotide sequence of SEQ ID NOs: 403-406, 409-415, 417-424, 470, 473, 477, 480, or 486.

In some embodiments, the chemical modification is cET.

In some embodiments, the ASO comprises the nucleotide sequence of SEQ ID NOs: 425-432 or 435-442.

In some embodiments, the chemical modification is LNA.

In some embodiments, the ASO comprises the nucleotide sequence of SEQ ID NOs: 443-458.

In some embodiments, the chemical modification is LNA and 2′-O-methoxyethyl.

In some embodiments, the ASO comprises the nucleotide sequence of SEQ ID NOs: 400, 408, 460, 462, 464-466, 468, 469, 471, 472, 475, 476, or 479.

In some embodiments, the ASO does not comprise 8 or more contiguous nucleotides of unmodified DNA.

In some embodiments, the ASO does not comprise an unmodified ribonucleotide.

In some embodiments, the ASO does not comprise a deoxyribonucleotide.

In some embodiments, the length of the ASO is 2×n+4 nucleotides (n is an integer of 8 or greater), wherein the nucleotides at positions 2×m are ribonucleotides modified by LNA (m is an integer from 1 to n) and the remaining nucleotides are ribonucleotides modified by 2′-O-methoxyethyl.

In some embodiments, the ASO comprises the nucleotide sequence of SEQ ID NOs: 393 or 394.

In some embodiments, the length of the ASO is 3×n+2 nucleotides (n is an integer of 4 or greater), wherein the nucleotides at positions 3×m are ribonucleotides modified by LNA (m is an integer from 1 to n) and the remaining nucleotides are ribonucleotides modified by 2′-O-methoxyethyl.

In some embodiments, the ASO comprises the nucleotide sequence of SEQ ID NOs: 392 or 395.

In some embodiments, the length of the ASO is 3×n+2 nucleotides (n is an integer of 6 or greater), wherein the nucleotides at positions 3×m are ribonucleotides modified by LNA (m is an integer from 1 to n) and the five nucleotides at the 3′ and 5′ positions are ribonucleotides modified by 2′-O-methoxyethyl.

In some embodiments, the ASO comprises the nucleotide sequence of SEQ ID NO: 19 or 20.

In some embodiments, the length of the ASO is 4×n nucleotides (n is an integer of 3 or greater), wherein the nucleotides at positions 4×m are ribonucleotides modified by LNA (m is an integer from 1 to n) and the five nucleotides at the 3′ and 5′ positions are ribonucleotides modified by 2′-O-methoxyethyl.

In some embodiments, the ASO comprises the nucleotide sequence of SEQ ID NO: 21.

In some embodiments, the length of the ASO is 4×n+4 nucleotides (n is an integer of 3 or greater), wherein the nucleotides at positions 4×m are ribonucleotides modified by LNA (m is an integer from 1 to n) and the remaining nucleotides are ribonucleotides modified by 2′-O-methoxyethyl.

In some embodiments, the ASO comprises the nucleotide sequence of SEQ ID NOs: 396 or 397

In some embodiments, each ribonucleotide of the ASO is modified by 2′-O-methoxyethyl.

In some embodiments, the ASO comprises the nucleotide sequence of any one of SEQ ID NOs: 17, 28-30, and 40-48.

In some embodiments, each nucleotide of the ASO is a ribonucleotide modified by 2′-O-methoxyethyl.

In some embodiments, the ASO comprises the nucleotide sequence of any one of SEQ ID NOs: 17, 28-30, and 40-48.

In some embodiments, each cytidine in the ASO is modified by 5-methyl.

In some embodiments, the length of the ASO is 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl.

In some embodiments, the ASO comprises the nucleotide sequence of SEQ ID NOs: 398 or 399.

In some embodiments, the ASO further comprises a GalNAc moiety, optionally a GalNAc3 moiety.

In some embodiments, the ASO further comprises a biotin or cholesterol moiety.

In some embodiments, each cytidine in the ASO is modified by 5-methyl.

In some embodiments, the regRNA is an enhancer RNA (eRNA).

In another aspect, provided herein are pharmaceutical compositions comprising the ASO disclosed herein and a pharmaceutically acceptable carrier or excipient carrier.

In another aspect, provided herein are methods of increasing transcription of CPS1 in a human cell, the method comprising contacting the cell with the ASO disclosed herein or the pharmaceutical composition disclosed herein.

In some embodiments, the cell is a hepatocyte.

In some embodiments, the ASO increases the amount of the regulatory RNA in the cell as compared to a cell that has not been contacted with the ASO or the pharmaceutical composition.

In some embodiments, the ASO increases the stability of the regulatory RNA in the cell as compared to a cell that has not been contacted with the ASO or the pharmaceutical composition.

In another aspect, provided herein are methods of treating a urea cycle disorder, the method comprising administering to a subject in need thereof an effective amount of the ASO disclosed herein or the pharmaceutical composition disclosed herein.

In some embodiments, the urea cycle disorder is CPS1-deficiency.

In some embodiments, the urea cycle disorder is hyperammonemia.

In some embodiments, the ASO increases the amount of the regulatory RNA in a cell of the subject (e.g., as compared to a cell (e.g., a similar cell from the subject) that has not been contacted with the ASO or the pharmaceutical composition).

In some embodiments, the ASO increases the stability of the regulatory RNA in a cell of the subject (e.g., as compared to a cell (e.g., a similar cell from the subject) that has not been contacted with the ASO or the pharmaceutical composition).

In some embodiments, the cell is a hepatocyte.

### BRIEF DESCRIPTION OF THE DRAWINGS

FIG. 1 shows an illustrative schematic of eRNA, paRNA, mRNA, and natural antisense transcript (NAT) of a gene on the chromosome. The eRNA, paRNA, and NAT are all non-coding RNAs. The eRNA is transcribed bidirectionally from an enhancer of the gene. The paRNA is transcribed from the promoter of the gene, same as the mRNA, but in the antisense direction. The NAT is transcribed from a downstream promoter of its own in the antisense direction, such that the transcript overlaps at least partially with the mRNA. Generally, eRNAs and paRNAs upregulate gene expression whereas NATs downregulate gene expression.

FIG. 2 shows analysis of mouse and human CPS1 ATAC-seq and H3K27Ac ChIP-SEQ for identification of regRNAs.

FIG. 3 shows relative CPS1 mRNA expression in hepatocytes treated with an ASO and non-targeting control (NTC).

FIG. 4A shows a schematic of various human CPS1 ASOs with chemical modifications. Light gray indicates a 2′-O-(2-methoxyethyl) (2′-MOE) modification. Dark gray indicates a locked nucleic acid (LNA) modification. *C indicates a 5-methyl on the cytidine. FIG. 4B shows relative CPS1 mRNA expression in hepatocytes treated with the ASOs.

FIG. 5A shows a schematic of various human CPS1 ASOs with chemical modifications. Light gray indicates a 2′-O-(2-methoxyethyl) (2′-MOE) modification. Dark gray indicates a locked nucleic acid (LNA) modification. Line brackets indicate a phosphodiester (PO) linkage. *C indicates a 5-methyl on the cytidine. Unique sequence identifiers are assigned to nucleotide sequences having the specific chemical modifications shown in this figure. FIG. 5B shows a schematic of various mouse CPS1 ASOs with chemical modifications.

FIG. 6A shows CPS1 mRNA levels in wildtype and OTC-deficient hepatocytes treated with CPS1 ASOs. Heatmaps of CPS1 and OTC mRNA levels are shown in FIG. 6B and FIG. 6C.

FIG. 7A shows OTC mRNA levels in wildtype and OTC-deficient hepatocytes treated with CPS1 ASOs. Heatmaps of CPS1 and OTC mRNA levels are shown in FIG. 7B and FIG. 7C.

FIG. 8 is a schematic depiction of the in vivo ammonia challenge for mice treated with CPS1 ASOs.

FIG. 9A shows relative CPS1 mRNA expression in the mouse liver. FIG. 9B shows plasma ammonia levels of treated mice. FIG. 9C shows relative mRNA expression of other urea cycle genes in the treated mice. FIG. 9D shows mRNA expression of genes neighboring the CPS1 locus following ASO treatment.

FIG. 10 shows ammonia levels in OTC deficient mice treated with a CPS1 ASO.

FIG. 11A shows a dose dependent effect on plasma ammonia in vivo in male OTC-D mice (Otc^{spf/ash}) after the ASO treatment. FIG. 11B, there was a dose dependent effect on urea in vivo in male OTC-D mice (Otc^{spf/ash}) after the ASO treatment. FIG. 11C shows that the reduction in ammonia correlated with the total ASO administered.

FIG. 12A shows that CPS1 ASO treatment increased mouse OTC and CPS1 mRNA expression in the OTC deficient mice (1.26 FC of CPS1 and 1.43 FC of OTC). FIG. 12B shows that ASO treatment increased additional urea cycle genes, such as Nags, Ass1, Asl, and Arg1. In both figures WT C57 mice mRNA is shown on the left bar, Otc^{spf-ash/J} treated with PBS is shown in middle bar, and Otc^{spf-ash/J} treated with ASO is shown on the right.

FIG. 13 shows that mouse Otc deficient livers showed hyperammonia 30 min after ammonia treatment. Treatment with mouse CPS1 ASO decreased ammonia to WT levels.

FIG. 14 provides shows a schematic of various human CPS1 ASOs with chemical modifications. Unique sequence identifiers are assigned to nucleotide sequences having the specific chemical modifications shown in this figure.

FIG. 15A shows that hCPS1-ASO-1x decreased in vivo plasma ammonia in the OTC^{def} mice with humanized liver. FIG. 15B shows that hCPS1-ASO-1x increased in vivo urea in the OTC^{def} mice with humanized liver. FIG. 15C shows that hCPS1-ASO-1g also increased OTC protein levels after treatment with 5 mg/kg and 20 mg/kg ASO.

FIG. 16 shows that the indicated ASOs increased CPS1 mRNA and ureagenesis in both healthy and OTC^{def} primary human hepatocytes

FIG. 17 shows that hCPS1-ASO-1x decreased ammonia production in NHPs by a statistically significant amount as compared to PBS only treatment by Day 27. FIG. 17 also provides the ammonia AUC both over time and per dose in the NHP experiment.

FIG. 18 shows that hCPS1-ASO-1x decreased ammonia in NHPs after a single dose of 5 mg/kg for up to 5 weeks

FIG. 19 provides the NHP study ammonia AUC quantification on Days 22, 29 and 36.

FIG. 20 shows that combination of hCPS1-ASO-1x with ¹³C-sodium acetate treatment enhanced ureagenesis in NHPs at day 43 as compared to hCPS1-ASO-1x alone.

### DETAILED DESCRIPTION

The present disclosure provides antisense oligonucleotides (ASOs) targeting regulatory RNAs, such as promoter-associated RNAs and enhancer RNAs, and methods using these ASOs to regulate gene expression. These methods are useful for modulating the levels of gene products, for example, modulating expression levels of disease-causing genes such as Carbamoyl-Phosphate Synthetase 1 (CPS1), thereby to treat diseases associated with aberrant gene expression such as a urea cycle disorder. Urea cycle disorders are reviewed in Haberle et al., 2012, Orphanet J. Rare Dis. 7:32, hereby incorporated by reference in its entirety. Urea cycle disorders result in the accumulation of ammonia and other precursor metabolites, causing development of hyperammonia and related symptoms, such as cerebral edema and the related signs of lethargy, anorexia, hyper- or hypoventilation, hypothermia, seizures, neurologic posturing, and coma. Milder or partial urea cycle enzyme deficiencies cause ammonia accumulation and elevations of plasma ammonia concentration. CPS1 deficiency is the most severe of the urea cycle disorders. Individuals with complete CPS1 deficiency rapidly develop hyperammonemia and are at risk for additional hyperammonemia events.

### I. Definitions

To facilitate an understanding of the present application, a number of terms and phrases are defined below.

The terms “a” and “an” as used herein mean “one or more” and include the plural unless the context is inappropriate.

As used herein, the term “Carbamoyl-Phosphate Synthetase 1” or “CPS1” refers to the protein of UniProt Accession No. P31327 as set forth in the applicable database as of the priority date of the instant application and related isoforms and orthologs, the gene encoding the protein (e.g., NCBI Entrez Gene: 1373), or the mRNA encoding the protein (e.g., Accession Nos. NM_001122633.3, NM_001369256.1, NM_001369257.1, and NM_001875.5).

As used herein, the terms “regulatory RNA” and “regRNA” are used interchangeably to refer to a noncoding RNA transcribed from a regulatory element of a gene (e.g., a protein-coding gene), wherein the gene is not the noncoding RNA itself. Exemplary regulatory elements include but are not limited to promoters, enhancers, and super-enhancers. A noncoding RNA transcribed from a promoter, in the antisense direction, is also called “promoter RNA” or “paRNA.” A noncoding RNA transcribed from an enhancer or super-enhancer, in either the sense direction or the anti-sense direction, is also called “enhancer RNA” or “eRNA.” It is understood that a natural antisense transcript (NAT) complementary with at least a portion of the transcript of the gene is not a regulatory RNA as used herein.

As used herein, the term “nascent RNA” refers to an RNA that is still being transcribed or has just been transcribed by RNA polymerase and remains tethered to the DNA from which it is transcribed. An RNA that has dissociated from the DNA from which it is transcribed is also called an “untethered RNA.”

As used herein, the term “antisense oligonucleotide” or “ASO” refers to a single-stranded oligonucleotide having a nucleotide sequence that hybridizes with a target nucleic acid under suitable conditions or a conjugate comprising such single-stranded oligonucleotide.

As used herein, the stability of a regRNA is reversely correlated with the degradation rate of the regRNA. Where an ASO increases the stability of a regRNA, it reduces the degradation rate of the regRNA. Where an ASO decreases the stability of a regRNA, it increases the degradation rate of the regRNA. The degradation rate of a regRNA can be measured by blocking synthesis of new regRNA and assessing the half-life of the existing regRNA.

As used herein, the terms “subject” and “patient” refer to an organism to be treated by the methods and compositions described herein. Such organisms preferably include, but are not limited to, mammals (e.g., rodents, primates, simians, equines, bovines, porcines, canines, felines, and the like), and more preferably include humans.

As used herein, the term “effective amount” refers to the amount of a compound (e.g., a compound of the present application) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA (1975).

Throughout the description, where compositions are described as having, including, or comprising specific components, or where processes and methods are described as having, including, or comprising specific steps, it is contemplated that, additionally, there are compositions described in the present application that consist essentially of, or consist of, the recited components, and that there are processes and methods according to the present application that consist essentially of, or consist of, the recited processing steps.

As a general matter, compositions specifying a percentage are by weight unless otherwise specified. Further, if a variable is not accompanied by a definition, then the previous definition of the variable controls.

### II. Antisense Oligonucleotides

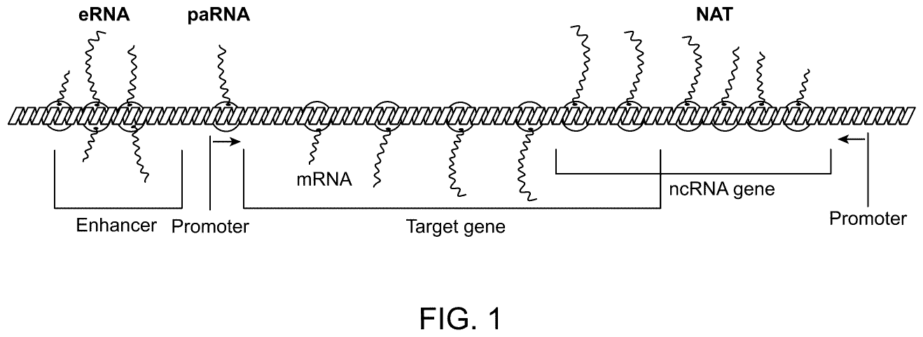

The antisense oligonucleotides (ASOs) disclosed herein hybridize with a regRNA transcribed from a regulatory element of a target gene. It is understood that both eRNAs and paRNAs are regRNAs facilitating or upregulating gene expression (FIG. 1). In certain embodiments, the target regRNA is an eRNA. In certain embodiments, the target regRNA is a paRNA. eRNAs can be identified using methods known in the art, such as Assay for Transposase-Accessible Chromatin using sequencing (ATAC-seq), global run-on sequencing, precision run-on sequencing, cap analysis gene expression, and histone modification analysis (see, e.g., Sartorelli & Lauberth, Nat. Struct. Mol. Biol. (2020) 27:521-28; PCT Application Publication No. WO2013/177248). paRNAs are RNAs transcribed from promoters of target genes in the antisense direction (transcripts in the sense direction are mRNAs of the target genes). They can be identified by similar methods, taking into account their specific location and orientation. In the human CPS1 gene, multiple, distinct eRNAs have been identified to transcribe from the same enhancer region. The nucleotide sequences of exemplary regRNAs are provided in Table 1 below. Any of these regRNAs are contemplated as a target regRNA of an ASO disclosed herein.

TABLE 1
Exemplary regRNAs

| regRNA | Nucleotide Sequence |
|---|---|
| Human CPS1 | GCCTGATGTGTGTGCCTGCATTCATGCCTGTCACTCTGCTCAGCTGCGTGCAGACAG |
| RR44_v1 | CTTATATGAAGGAGGACTGTGTACATGTGGTCAGAGAAGAAAAAAATGCTTATTTT |
| SEQ ID | TTAAAATGTTTTGAAAAATAATCTGTCCTAAAAGTGTAACATTTCCACTAAATTTTT |
| NO: 49 | TCATGTCTGACTTTAACATTCCCTACCAACCAACTCTTATAGCCTCAACTCCTTTGA |
| | TCTATCCTAATGTAAACTCCCTCACTAATGTGTAATATATTTTTTTCCAGTTTGGTTA |
| | TAGATTTCAGTCTAGCCTGAACACTCAAGTATGAAACTAGCCACTATTTTATTTATT |
| | GGCTGTTCCAACCTCCATTGTGGAATTATAATTGATGGATTTTCCAAGTCTCTAATT |
| | CAGAATATTTAATCAAATTTTCCAAAATTTCTGCCTTCTCCTCCTTCATTCTGATGA |
| | CTAAAGACATGGACCATAAAATATATATACATATATACATACATATGTATATGTAT |
| | GTATATACACATATATATATGTATGTATATATACATACATATATATATGTATATATA |
| | CACACACATATACATATGTATGTATATATACACACACACATATATATATGTATGTA |
| | TATATATACACACACATATATATATGTATGTATATATATACACACACATATATATAT |
| | GTATGTATATATATACACACACACACCGTAGTTTGCAAGTCTGCATCTGGGACCCT |
| | TTACTGATAAAAATGAAAGCCCATTAAAACTCTCATGCCCACATTTCTCTCTAGACT |
| | TTAGAGTAGGAGAATTCTATCCTAGAATTGTTGATTGTTGACATTTCTATGGAAAA |
| | ACATGTTTAATTCATCATGCTTTGTAGTCAACTGCTATAAATGGTGATGAAGTCTTT |
| | TCCTTGGTCAAGCTCTGGACTCCTCAGTACTTCCACTGTCATATTTGATTAAAACAT |
| | AAGTGATGAAGGGAGGAAATATATTATAGTATTCATTGCATGGGTACCTTAGGGTA |
| | ATGGGACTATGACTGATTTAAATTTATTTTGCATCCTAAAAAGTTTCCATTAGTGGG |
| | TGCAGCACACCAGCATGGCACATGTATACATATGTAACTAACCTGCACAATGTGCA |
| | CATGTACCCTAAAACTTAAAGTATAATAATAAAAGAAAAAAAAACTTAAAAAGAA |
| | AAGTTTCCATAATGAGGATTGAGTTTATATCGTTTTCTTAATTAAGGAAAGACAAC |
| | CCTCACTACACACACACACACACACACACACACACACATAAAAAGACAAGAGGTG |
| | AGATCAAGGCGTAAACTAGTATTGGACTAAGCCAGTAAGTACTGCTTCTCAGTCCT |
| | CAGTTCTCTAGGTTGGGAGTTTTCCTTTTGCTATTTTCATTCTCCTCCAATTCATTTT |
| | TCAATGATCTTCAACACAAATTTCGCCTTTATTTCTTGTTCAAAATACTTCCATGAC |
| | TATTATGAATACTATTTATGGATGAAGGCTGAACATTTTAAGGCATACTAAGTCTC |
| | CCATGATATGGTACCTTTTCATTTTCCCATTCTATACCCTGCATCTAGGCTTTTAAA |
| | AAATATACCACATGACTTCATGCCTCTAGGCTATTGCTCATGCCACGGCATCCCTTC |
| | TGGGTTTTTTCCCCTCTTTATCTACTTGGTGAATTTGCATCCTTCAGTATAACACAGC |
| | TCAACATAATTCTATGCTTAAACTTTACTGAAAATGCACAAGGAACTTGATCTTTCT |
| | TTCTTATTTTCCCCATTTTATTTATTTTCCCATTGCATTTGGGCAACTTTCATGGCAC |
| | CAGTTATTTTATTAATTATTTGTATAAACTAGTATGAGCCTATTTTGTTTAGCTTTCT |
| | ATCACCAGGAGCCATGAAGCACTTTGAAATCAAATACTGTTTGTACTTGAATATAT |
| | TAAGTAAGCGTGAGGACTTAAATTAAACTTTGTTCCTCTTTAAAAAAATCCTTACCC |
| | CAACTCTAGCACTGA |
| Human CPS1 | ATGAAAATGGAATATTTTGTAATCATTTAAAGTAATGTTTTAAAAGAACTTTGTAA |
| RR44_v2 | TAACATCAAAATACATTTAACATAATATTGCCTCCAAAAAGCATTATGCAAGTCAA |
| SEQ ID | TGGCAATTTTCAAAACTATGTTCTGAGCATTGACATAAATGCTAGAAGATAATACT |
| NO: 50 | TTAGTGTTAACAGAGATTATGTTTGTGTGGTGGGTTATTATCTATTATTCTGTATTTC |
| | CACATTTCTTTAAATATCAAATGAAAGTGAATACAACCTAGTACTTGACATTTTAA |
| | CATTTTCAATACCAGATATGTCAATAAGGAATTCATAAAATAAAGTGCCCTGAGGG |
| | ATTTCACAGGCTAAATGCTTCTATTTTTTAAAATGTATTAATAGAAAAGAAGACTA |
| | AATCTAAAAAAAAAAATTCATTTTATCAGAGAGTGACTCATAAGTTTAGAAAACCT |
| | TTGATGACCTGGCTCACAAAGAATGTACTTCCAAGTACACAGGGTCCCCAGCCTGA |
| | TGTGTGTGCCTGCATTCATGCCTGTCACTCTGCTCAGCTGCGTGCAGACAGCTTA |
| Human CPS1 | TAGATAGAAAGGAGCAGCCAGAGATGTGCCTCAGGCTGAAGCCAGTGCTGCAAAT |
| RR7_v1 | GGGACAGCGCTCTCAAGCTACATTATATGCATTTCATTATCCAACAATTTGTTCTGG |
| SEQ ID | GAGGACAGTTTGATTGGCCAGGCAAGAAATTCAAAATGAGAGACTTTTAAATATC |
| NO: 69 | ACAACTCAGCTAAAAGAGCTCTTCTGTTTTCTGACCTGAGAAATTCACTTCTATCAG |
| | TCTCAATAAAGATGCTTGTAATATTAGGTGATGCAGACTCTGAACATTTGTCCCTCT |
| | GGACTGAGTGCACTCCCGATGATCTGAGTCTTGGGGCTATGCCTCAGGCTTCACAA |
| | TTCCTAGTGTGAAAGCTACTTGCATATCCACCCCTCCATCCAGCTGGGTTTTGTTCT |
| | GCTTTTCTTGTGTAACTAATGACTGTGAGTCAAAAAGATGGGAGGTCAACTACTTG |
| | CCTTTGACCATTAAATAGGTGTTTAAACTAAGCTAACTAAGGAATAACATTGTTTTT |
| | CCATTTCGTGATATCGTTTTCCATTTTATCCTAGATTCTTAACTAGGATGTGAAGAA |
| | AATAATAGTAGTCAATATTATAAAGTTTAAAATAATACAAAGTAGTCGATATTATA |
| | AACACAGACATATTTCTAGAATGGAACAATAAGATAGTAGGCTTCTATGTTTTATT |
| | TTAAAAGGTTCCGAAATTACCCACTTACCATTTGTGTGACCTTGAACAAGTTGCTTT |
| | AACTCCGAACCTCAGTTTCTTCATATACAAAATGGGAATACACCTACTATGGAGGT |
| | TTGCTGATAGAATTTAAACAGAATAAGTTACGCAAAATATCTGGTGTTAGTAAAGA |
| | ATTCTCTCAGTGACTCATAGCAAGAGTATAAATTGGTCTAGTCACTTTGTAGCATG |
| | ATTTGTCATTATACAGTGAAATGTAAAATGTGCATACCTTACAGCAATTACATTTTT |
| | TCAGGATGAAACTCAGAAAAATACTAAAACATGTGCACAAAATGATAGATACATA |
| | CATAGATATATAGATATTCATTGCAGCATAAATGAACCAGTGGGAATAATATTTAA |
| | ATGCATGAATGTTCTTTTCCCTTTTGAAATTATTTTACTGACCTTCTCCACAAACTCA |
| | CCTATTTCTTTTGGGTGGTTTGGAATTATTACAACAGGAAGGCAAAGAGAAAGAAA |
| | AAGTTAATATTTATCTTCTGGCAAGGCAACGGGATTTGCAGTAAGAACACAGTGCT |
| | AACAAGTAGATTTAATCAGGGAGAGGGGAGTTCGTTAAATTCTTAAACTGCTTTAA |
| | CTTATCATAACTCAAACTAGAGGATTCAGATTATATGAATTTTTCACATCTGGTGAA |
| | GTGGTAGGAAGAAGAATGGAAACACAGACGTCACATCAGCGTCCTTTATTTCCACT |
| | TCCACTTTCAACCCCTGATCCAGGACCACTCTTTAAGGCAGTAAAGGAGCAATGTA |
| | GTAGAAATGACAGCAGCAGGCCAGCAGATACCTATTCCTGAGACAGAGCCCATGG |
| | CATTCAGGGCCTGGAAACTGGGGACCCAAAATAACCCAAGTCTGGTTGTGACAAC |
| | ACAGAGGTTCTGAGAGTACAGGAAGAATTAAGAAGCCTGGGGACAAGGTAGAAA |
| | ACCACTGCAGGTAGATCATAACAGACAAGGGACAAATGACCAGACCCCCTAACGC |
| | CCCAAAGAGCAGATGTCAACAGCACAGAGAGCAGACCAGCTCAGCTGCTCTGCAG |
| | ATATCAAGGAGGACTCAGAGGATACATCAATCTGATTACCCTTTCCTTCTGCACCA |
| | CTAAATCATGTAAACTAAACAACCAGATGCCCTCTTGAAGAGGAATAGGGTAAGC |
| | AGGTCAAACTTGGAAAAGTGGAACAAATTGACCAAGCAAGGACAGGGTTACCTAA |
| | ATGGAGTATTGGACTGCATTACTGGACTCAAGACAAAACTTACCAGATTAGACTCG |
| | TAGATCAGATCAGTTATTTAAAAAATAAATAAAATAAGGTA |
| Human CPS1 | GTCCATCCTGACTCCTGGCAGGGACCAGGGCCATCCAGTTATACTACGGTGTTCTTT |
| RR8_v1 | CACCACCTCAGTGATGGCAGCTGGCTGGAGTCCAAAGCGTTTCAGTACTCCCAAGC |
| SEQ ID | TCTGACTACTTTTTTCTCTTCTTTTCTTCATCTTGCCCTACACATTTATCCAAAGAAA |
| NO: 70 | ATATTTAGGATGTGAAAGAAGGGGTGTAGATAAGAAATGAATCAAAGGAGAAATG |
| | TGTGATTATAAAACAATAAAATACGTAACTGAACCTTACTGTGGCTTAACACACAG |
| | CCAATAGTGCCAAAGATCATATAACTTCTGTCTGCTGAAGTTTTCTCTTTATTAAAT |
| | GGCATTGACAGAGAAAAACTATAAAGAATAATTAATTGAAATAGACTGCAAATCA |
| | CTTAGCCAACATAAAATGATGCAATATTTTATCTGATGAAGAAACCTAAAGTTAAA |
| | TATTGGTTAGATCAATTTTATCAATTTGTCTATATCCTATCAATCCTTCAAAGCCAT |
| | ATGAAATCATACTCTTTTAATGAACTTTATGTATGTATATCTTGTTTGTATCATACA |
| | ATTAATATACTGTCCACGTACTCTTATTTCACACATTAATATCTTCCTGGTTGAAAT |
| | CATTAAATAGCTCCACACTGCTTAACTACAGTGCGAAGTTAAAAATATAATATATT |
| | CATTTTTCTAAAGCTTTAGGCTTTTGTTTGCTTCATTCACGTTGTGTGATCTAAATTA |
| | GTATCTTCACTGTCTCTGTTTAATATCGACAAATTAGAAAGAGTAACAACCATGAC |
| | CTCTCATAGTCCATAAGTAATAATTAATTCTTCAATTCCTTCTATAAATACTTATTA |
| | AAGCCCTTTTACATGCCAAGCATGGTGCTAGATCCGAGAATAAAAATAAGTCTGGA |
| | ACACAGACTTGTCCCTCAAAAGCTCCCTCTTGAGAAGCCTTTCAGTTTTATTCTTAG |
| | AAGTGGACAAAAATGTGTATTTTCTACTCTCAAGATTGAA |
| Human CPS1 | AAATTAAACATATTTGAGGATCACAGAGAAGCATGTGGTGCAACATGTCTTGAAGT |
| RR9_v1 | TACCGACTTCGTGAAGTGTAAGGGGCAGTAGTTTACACTGGAATGACGTAGAGAA |
| SEQ ID | CAGGTCACCTACGCACATTCCTACTCCACCCCCTTTTGTAGAAAATTTGTTATACTG |
| NO: 71 | AACCTGATTTCTTCCATGGCCTCCTATTTTCAGCGGAATGTGATTCAGGAAGATACA |
| | TTCCACCCTCCTGGAAATCGGAAATAGGACCCGTGCTTTCACAAACATTAAGTTCC |
| | ATCTTCCCTAAAACTTTATTGCAATTCCCCAGGAATGGATCACAGAGCTATTTCTTC |
| | CTCCAGGTTTGTAGATTTGTAAGTTGAGTCATAGGGCAGGAGTACCTTTTTTTATTT |
| | TATATAGGCTAAAAATTTGTTGGAGAAGATTAGTGATCTGGAGAGGGGAAAAAAA |
| | AGAAGAACAAGAAGGAGATGAAGAAGGGGAGGAAGAAGAGAAAGAAAGACAAA |
| | AGGAAGCCAACCCTAATCCTTGAATTCTACAAACTACTAACAATTAGTCAAAAGTG |
| | CCTACTTGGATTTCAATACCTGACATGCCTTTGAGTGTTGAAGAATTAAATGTTTTA |
| | TTCTGGTATTGTTTTTTGATGTCATTTATTTTTAACCTATACTTGCCCCTATCAAGAT |
| | CATTCTCAGTTGATAGTAAGAAAAATATGTAATGTTGTTGCTAATTTATCTTGCATT |
| | AGGTAATTGATTATTTCAAAAGAGCATTCTTGAACATCTTGTAAACTAAAAAAGCA |
| | AAGAACTCGGTATGAGGAAGTTAAAAACTCTGTCAAGATTTCAAAAATTTGTGTAT |
| | AAAAATTAATTGCCATGTTAGAAGTCGAGGTCAAGCAGTAATGATAGAAATTTTGT |
| | GAAATGATGATTAGACATAAAATGTAAGACAAATGAGAGAAACAAAGGTACACAA |
| | TAGAAAAAGTATTATTAGTACTATATTAGTGACTCTGAATGGCATAA |
| Human CPS1 | GTGCAGAAGGAAAGGGTAATCAGATTGATGTATCCTCTGAGTCCTCCTTGATATCT |
| RR21_v1 | GCAGAGCAGCTGAGCTGGTCTGCTCTCTGTGCTGTTGACATCTGCTCTTTGGGGCGT |
| SEQ ID | TAGGGGGTCTGGTCATTTGTCCCTTGTCTGTTATGATCTACCTGCAGTGGTTTTCTA |
| NO: 72 | CCTTGTCCCCAGGCTTCTTAATTCTTCCTGTACTCTCAGAACCTCTGTGTTGTCACA |
| | ACCAGACTTGGGTTATTTTGGGTCCCCAGTTTCCAGGCCCTGAATGCCATGGGCTCT |
| | GTCTCAGGAATAGGTATCTGCTGGCCTGCTGCTGTCATTTCTACTACATTGCTCCTT |
| | TACTGCCTTAAAGAGTGGTCCTGGATCAGGGGTTGAAAGTGGAAGTGGAAATAAA |
| | GGACGCTGATGTGACGTCTGTGTTTCCATTCTTCTTCCTACCACTTCACCAGATGTG |
| | AAAAATTCATATAATCTGAATCCTCTAGTTTGAGTTATGATAAGTTAAAGCAGTTT |
| | AAGAATTTAACGAACTCCCCTCTCCCTGATTAAATCTACTTGTTAGCACTGTGTTCT |
| | TACTGCAAATCCCGTTGCCTTGCCAGAAGATAAATATTAACTTTTTCTTTCTCTTTG |
| | CCTTCCTGTTGTAATAATTCCAAACCACCCAAAAGAAATAGGTGAGTTTGTGGAGA |
| | AGGTCAGTAAAATAATTTCAAAAGGGAAAAGAACATTCATGCATTTAAATATTATT |
| | CCCACTGGTTCATTTATGCTGCAATGAATATCTATATATCTATGTATGTATCTATCA |
| | TTTTGTGCACATGTTTTAGTATTTTTCTGAGTTTCATCCTGAAAAAATGTAATTGCT |
| | GTAAGGTATGCACATTTTACATTTCACTGTATAATGACAAATCATGCTACAAAGTG |
| | ACTAGACCAATTTATACTCTTGCTATGAGTCACTGAGAGAATTCTTTACTAACACCA |
| | GATATTTTGCGTAACTTATTCTGTTTAAATTCTATCAGCAAACCTCCATAGTAGGTG |
| | TATTCCCATTTTGTATATGAAGAAACTGAGGTTCGGAGTTAAAGCAACTTGTTCAA |
| | GGTCACACAAATGGTAAGTGGGTAATTTCGGAACCTTTTAAAATAAAACATAGAA |
| | GCCTACTATCTTATTGTTCCATTCTAGAAATATGTCTGTGTTTATAATATCGACTAC |
| | TTTGTATTATTTTAAACTTTATAATATTGACTACTATTATTTTCTTCACATCCTAGTT |
| | AAGAATCTAGGATAAAATGGAAAACGATATCACGAAATGGAAAAACAATGTTATT |
| | CCTTAGTTAGCTTAGTTTAAACACCTATTTAATGGTCAAAGGCAAGTAGTTGACCTC |
| | CCATCTTTTTGACTCACAGTCATTAGTTACACAAGAAAAGCAGAACAAAACCCAGC |
| | TGGATGGAGGGGTGGATATGCAAGTAGCTTTCACACTAGGAATTGTGAAGCCTGA |
| | GGCATAGCCCCAAGACTCAGATCATCGGGAGTGCACTCAGTCCAGAGGGACAAAT |
| | GTTCAGAGTCTGCATCACCTAATATTACAAGCATCTTTATTGAGACTGATAGAAGT |
| | GAATTTCTCAGGTCAGAAAACAGAAGAGCTCTTTTAGCTGAGTTGTGATATTTAAA |
| | AGTCTCTCATTTTGAATTTCTTGCCTGGCCAATCAAACTGTCCTCCCAGAACAAATT |
| | GTTGGATAATGAAATGCATATAATGTAGCTTGAGAGCGCTGTCCCATTTGCAGCAC |
| | TGGCTTCAGCCTGAGGCACATCTCTGGCTGCTCCTTTCTATCTATAAGTTAAAGCAA |
| | TGGCCTCAGCAAGCAGTACACTCTGTACAGCAGCTGCAACTCGTTATCGGCTTTGT |
| | CCAGAATAACAAACAGAACAAACACATTTTGCAAGGGCTTTTACCCCTCTTATGTA |
| | GAGAAGGAGGAATTAAGCAATCAGTGAAATGCTGATTGATCAGTTGTGCTGACTA |
| | AGAGAAGTCCAAGGCCACATTAGTACTATTAAGCCCTAAAAAACAAAGCAAAAAG |
| | AAAATGTTCTTAGAACTTTATTTAAACCAGTATTTCTGTGATGTGAAGTACATTTCC |
| | CCCAAAGGGAAAAATGGGAAAATATTTGCATTTCACTTAGTACACTTTACTCATCC |
| | AGACTGCTTTGTAATGTAATGATGGTTAAGTATTTTTACAAAGATAATTCATGTTTT |
| | TTGGCAGATTTTGGTTGATCATGGAGTATCAGGGTTGTGTGAACCAAAGGTGTGAT |
| | GCAGAGCAGCTTTAGTAAACCATCCCATTCCTATGCCACCCGTTTGATTTCAGTACC |
| | AACTGCTGTGGCCACATCTAGACCACTCTTTACCAGAAACTGTGCCACCTTCAAAA |
| | TTCCAGCATCCTTTTCCCCTCCTCCCTTATTTTTTGATCTCATATACTCAAGTATTCT |
| | TAGTGTAACAATTCTTTGAGCATTCTTTCTTATCTAGCTTAAAATCTAGGGTCTACC |
| | ATTATAATCATTCCTTTCAAGATAACCTTAACTTCCACACCCCAATTTCTAACTGGC |
| | CTGGCAAAACTCAGACCTTTGGTGCAAAATCCTCCATCTCTATGCCTGAGCCCTTGT |
| | GTTTGAGAATGGCTGAAGAAAACCATGTGCATGGACATTCTGATGTCACTATAAAT |
| | TCATGGTCACGGATCTCAAATGGGAGCCCAAGACTACTGGCTTTCCTGCTGTGGTT |
| | GTCTAATGACAGCATGATCTTACTCTTCACAGATATTAGTTTAAACTCTCCAACCTT |
| | CAATTGAAAAGTCTACATCTTACTTTATTAGAAAGTAGAAACAATCAAGGGTTATG |
| | CTCCCATTTTCACACTACCAAGTAAACAAACCTTCAGTAAGAGAAGATATATTTCC |
| | TATATCTCCTCCTACCCTCCTGTTAAAAGGGTGCAAACGTCCCCCCTCCTAACAAAA |
| | ACCAATCCTTCTTCATGCTCTTTAGATGCCGTGTTGTCTCTCTTTCTCAAAGTCTTCA |
| | TTTTTTGGTTATACTCTTTCTCTCATGCAAATTTTTCTCTCTTTTTCAAGTAGAGCAG |
| | GCTCAGCAGCCTACAAACGTGTCTTGGGCACCTTTCATCAAAAAATAAAAAACATA |
| | AAAACAACAAACGAAACCCTTCTTGGCTCCACATTCTCATCCAGTTTCTATTCTTTC |
| | ACTTAGCTGCTTTTCTTCTCATGTTTTGTCTATTTTTTAACATCCTAGTCACTCTCTG |
| | ATCTACTTCAGTTTGACTTCTACCTCCATCACTTGAGTAAATTCAATACATGGTTTC |
| | AAGTATATTCTAGCCCTAGCCTGATCCACAGGGAAGGTTCTGGACCACAAGCAACA |
| | TGGCAGAGTTGTTCACTCTTGAGGTAAAGGAGTTTGGCACCTCATGCCCCTATAAT |
| | GGATTAAAGAAACACTCCTTAATCGGGGTTAGTCCCTGGGAAAGGTTTTAGGTGTG |
| | AGCAATTAGCAACCATCACCCACAGTGGCTGGGAGAGGGGGGCACTAGCCCAATA |
| | AAGAGGATCTAAGTAGTGCACCCAAGCAGGTCTACCACAGTTAGCTTCCTATGTGG |
| | GCTCACGTTGCATGCAGTGCATCTCTATACAGCAATTAATCTATTTCATAATATTAA |
| | TAAAATAATTTCACTTTCCATGTTAAAATCCTTCAATGACTTCCTATTGCACTATAA |
| | ATGGCAAATCAATATGTTAACATGCCCTGCCAACACCTACAGGGTCTGGGGTCTGA |
| | TTGCCTTTCAGATCTCTC |
| Human CPS1 | GAAAGAACACCGTAGTATAACTGGATGGCCCTGGTCCCTGCCAGGAGTCAGGATG |
| RR22_v1 | GACTTTCAGTGTTGAAGAGCCATCTTGGCCTGTTACTAGCGCTGCCCCACACCATTG |
| SEQ ID | TGTTTGCCTAAGAGCTGAGCCAAAGAATTTACATATGTATGAGCCAAGGTCTGAAA |
| NO: 73 | ATAGAAGTTAAAAAAATGGTGCTAAATTGACATATTTGCTATTTTATTTTTTTGGTC |
| | AGTTAATATGATAAACTTTTATTTAAATGTAAACATTATTTTAAAAAGCACTTTGTT |
| | TTAAATAACTGATTTTCATTGAAATTAATTCTGGCCTCTAAGCAGAGTTAGCTACAA |
| | AGACAATTTGTAAAAATGCAGAAAATTCAGAGTTTAACTTGGAGTTGTTTAAAGTT |
| | TATTTTCATATTTGAGTCTGATATTTCAGTTCACTGTAATATTTCAATTTATTTAAAA |
| | TATTATCAAGACAAATATTGGGTTACCTTCACAATATTTTTTTCATTTTTCTTACCCC |
| | ATATATTAAAAATGAACAAAAATGAAAGAATTATATAGTCTAGTGGATGGCAATTC |
| | TCTTGGTACAGTACTCACAGGATGTCAGAAGTGCTTTGATTTGCATGCAAATAGAA |
| | ACTGCATTCCTAAAAGACCCTTAATTGAAGATATTAATAATTTTCATTAGGTTTTAA |
| | GTCTTTTAACCTTATCATATGGATAGAAAAGTAAAAGATGTGTTTAATAAAATGTC |
| | TTTTACTTATTTTTATTTGTCATTTATTTAAAGGTTTATTAGTGAATCTGTTTACCTG |
| | GAAAATGTGCAATTCTATTTGCAAATTTGGAGCACTACATATATTTAAGAGAATCC |
| | TTAGTTTCTGTTGGGAGTGGCAATATTTTATAACTGGGCCTTCATATTCCTATGTTA |
| | CAGCCCTGGTCCCTGAAAGTCCTACAGCTATCTTTGTTTCAAGTGTGTATGTGTTAT |
| | AATAAGATAAGTGTTAATTAGTTAATCAAGTACTAATTAACCTTTTTAATTTATAGA |
| | AAAACAGACATTTATGGACAAGTAAATTTTTGGGGTCAAAGTCTTTAATAATTTGA |
| | AGAATGCAGTTATTTTTAGCACAAATATTCTAATGTGAAAAGAATTTCTTTTTCTGA |
| | GTTATATATTTTTATGCCATTCAGAGTCACTAATATAGTACTAATAATACTTTTTCT |
| | ATTGTGTACCTTTGTTTCTCTCATTTGTCTTACATTTTATGTCTAATCATCATTTCAC |
| | AAAATTTCTATCATTACTGCTTGACCTCGACTTCTAACATGGCAATTAATTTTTATA |
| | CACAAATTTTTGAAATCTTGACAGAGTTTTTAACTTCCTCATACCGAGTTCTTTGCT |
| | TTTTTAGTTTACAAGATGTTCAAGAATGCTCTTTTGAAATAATCAATTACCTAATGC |
| | AAGATAAATTAGCAACAACATTACATATTTTTCTTACTATCAACTGAGAATGATCTT |
| | GATAGGGGCAAGTATAGGTTAAAAATAAATGACATCAAAAAACAATACCAGAATA |
| | AAACATTTAATTCTTCAACACTCAAAGGCATGTCAGGTATTGAAATCCAAGTAGGC |
| | ACTTTTGACTAATTGTTAGTAGTTTGTAGAATTCAAGGATTAGGGTTGGCTTCCTTT |
| | TGTCTTTCTTTCTCTTCTTCCTCCCCTTCTTCATCTCCTTCTTGTTCTTCTTTTTTTTCC |
| | CCTCTCCAGATCACTAATCTTCTCCAACAAATTTTTAGCCTATATAAAATAAAAAA |
| | AGGTACTCCTGCCCTATGACTCAACTTACAAATCTACAAACCTGGAGGAAGAAATA |
| | GCTCTGTGATCCATTCCTGGGGAATTGCAATAAAGTTTTAGGGAAGATGGAACTTA |
| | ATGTTTGTGAAAGCACGGGTCCTATTTCCGATTTCCAGGAGGGTGGAATGTATCTT |
| | CCTGAATCACATTCCGCTG |
| Human CPS1 | CATTCCGCTGAAAATAGGAGGCCATGGAAGAAATCAGGTTCAGTATAACAAATTTT |
| RR23_v1 | CTACAAAAGGGGGTGGAGTAGGAATGTGCGTAGGTGACCTGTTCTCTACGTCATTC |
| SEQ ID | CAGTGTAAACTACTGCCCCTTACACTTCACGAAGTCGGTAACTTCAAGACATGTTG |
| NO: 74 | CACCACATGCTTCTCTGTGATCCTCAAATATGTTTAATTTAAAGAGGGTCCAGTAGT |
| | GTCCTGGCACATGATCTGGATTGCCATAGATAACCATCTACCTCACAGCTAGGGTT |
| | GCTCTTTAGAATCTTGCAAAATCATTTGTTTACTCTTGACAAAAGTTAAGAAAACA |
| | AGCCCATCAGAGTTGTTTGTTCTGTCAGCATGTTAGAAGATGGTTTTGTTGCAATGA |
| | TAATCGTTGTGCAAAGAAGACTGATGATGATTTTTTTTTACATTTTCTTAACAGTAT |
| | TTGCTATTTAGAATGAATGTTGTCTAATTATTTAGCCATTTTATTTTGTAAAATTTAT |
| | GTTGTAGGCATATTTAGACCAAGTTATAAGAAAATGCTTCAGCCAAAATTAAGTGT |
| | TGAGTTTGATTTGTGTAATTGTTAGTTTCTTTACTAGTTGTTCCATCATTTACACAAT |
| | TATTTCTATTTGAAATGCAGTAATTGTTCAGAACTTATATTTCTATACTGATGTCTA |
| | CTAACAGCTTTAGATCAAATATTAAATAACTCAAGAATAATGAGATGATCTTGGCT |
| | TACTTAGATATTTGGTTTTTATATCTATAGAACAAAGGAATTAGAAAATAATTTTGA |
| | AGATTTCATCCAGCTATGTAAAACTATCTAGGGAATACATTTACTAGGTTTTCAATT |
| | TTCTACAAAACATCTTTCAGCAGAAAGCAATCCTGTTTCCTGATATACAATGTCTGA |
| | TACATAGAAACTACTCAGTACATAATTCCTGAATTGATTATTCTTTTGGAAATCCTA |
| | GATTTGATTTCTGAACAATCATAAACATTTAATGGCATGAAATTACCCAGATTCCA |
| | TGGTTCTGGAATACATAACTTCAAGCAATAAGATGCAAGATAGAAACATATAAGA |
| | CATTCTTTGCTATTTTAGGTAAGTCCAGCTGAATCAGTTAATCAGCTAGAAACGTG |
| | GCTCACAGATGAATTAGTTTTATTATTAGGTGGATTACTGAAAAATTAATAGCTTTA |
| | TTTCCGTATTACCTTATCATTTATTTAATATAAAACATAATAAACCAGAGAAGTTGT |
| | GGTTACTTTCTTCTGTCTCATTGTTCAGTTTTTACTGAAGTTATGTTTTACTGATTTT |
| | CACGAAATGCAAATATTTCTGAGCATCAGAAATCCAGCTTTTGCTACCTCCCACTC |
| | ATGTATTCTTCCCATCAAATTGAATCTTAACCAGGTCACTCTTTCCTGTTCATTCCAT |
| | GCTGTTGCCCCTGTTTTCTCCAGATATAATTGACATGCTCCAAGAGGATGAACATTG |
| | CCTAAGACTTGATTTCTGGTCTTGGCTTTGCAACTTACTAGCTGTGTGACCTTGAAC |
| | AAGCTACTTAATTCTCTGAGACTCATCATTATTGTTTATAAAATAGGGATAGCATTA |
| | GCTTATTTCAAGAATTGTTTTTAAAATTAGCAAGGAAGGCATACCAATGCCTAGCT |
| | CAGGGGATCAACAAGTGGCAAGTACCTCAACTTTCAGGCAAGATCATACTTATCAT |
| | CAGTGGAGCCAATAGAACAGATTTATTTAAATACCCTATTTTCACTTACTGGCATA |
| | ACAGCAAGTGGTGTACATATTTATATATCTCTCTATATAGATGCAGATAATATACT |
| | AATATATTGTGGTTATATCTTCCAATGTTTTAGTTTCTATTGAGGG |
| Human CPS1 | TCATATAAGCTGTCTGCACGCAGCTGAGCAGAGTGACAGGCATGAATGCAGGCAC |
| RR43_v1 | ACACATCAGGCTGGGGACCCTGTGTACTTGGAAGTACATTCTTTGTGAGCCAGGTC |
| SEQ ID | ATCAAAGGTTTTCTAAACTTATGAGTCACTCTCTGATAAAATGAATTTTTTTTTTTA |
| NO: 75 | GATTTAGTCTTCTTTTCTATTAATACATTTTAAAAAATAGAAGCATTTAGCCTGTGA |
| | AATCCCTCAGGGCACTTTATTTTATGAATTCCTTATTGACATATCTGGTATTGAAAA |
| | TGTTAAAATGTCAAGTACTAGGTTGTATTCACTTTCATTTGATATTTAAAGAAATGT |
| | GGAAATACAGAATAATAGATAATAACCCACCACACAAACATAATCTCTGTTAACA |
| | CTAAAGTATTATCTTCTAGCATTTATGTCAATGCTCAGAACATAGTTTTGAAAATTG |
| | CCATTGACTTGCATAATGCTTTTTGGAGGCAATATTATGTTAAATGTATTTTGATGT |
| | TATTACAAAGTTCTTTTAAAACATTACTTTAAATGATTACAAAATATTCCATTTTCA |
| | TTGCTGCTACAATGCATGTCAATATTGCCATATTTGATTACTATTTACAAATTTCTC |
| | TCCAAAGAGAGTGTACTCACCTACATACCAGTGCAATATATGGGTGTCCCTTTAAA |
| | AATGTCCTCACCAACATTATGTATTATCTTTCATTTTATTTGCTCCGCTGTAGAAAA |
| | TATTATTTTGCTTTCATTTCTATTTTAAATCACTGGTGAAGTTGAATACTTCACAGTA |
| | TGTTTTTAATTGATACTATTCTTTTATCAACTACTGTTTACATTTTTGTTTCCATTAA |
| | CTCATTTATATTAATCATTACGAAAATGTCTTTAAAGGAAACATAAAGAAACACTG |
| | ACAGCCTGTGTCTATACTGATTTAAACAGTCCACTTCTCAAACAGTCCACTCTTCAT |
| | TTAAACAGTCCACTGACATAAGCAAGGGAAGACCTGCTACAGATTGAAAAAAAAA |
| | ATGAGAACAGGATGTCTTAGAGAATAAAGGTCTTAGGTTTTGGAATGAAGAGAAA |
| | ATAAGGGTGTTAGTAATTCCTTTGAAGGTACAGAGACTTCCAACTGCCCTCTTGAA |
| | ATGTTCCTAAGTAATGAATCAGAAACAACTCTAAGAAGTTGGTGCATCTATCAAAA |
| | AGTCTCTAACATAGCAAGCATGCAAGAGATGCTTCTGGAAAAATGACTGAATGAA |
| | TGGATTGCTGTGTGCTTAATTCCTGCCTGATCAGTGATGATTATTTAAGCAGGAACA |
| | AATAAAATTACATCAGTTAAGCATGAGAGGAAATAAGGATGCTTAGGAGTCATGA |
| | AAAAAAAAACCTGGCAATCAATAATTCTATAAAGTCTATTTTTGAAGAGATACATT |
| | AAAGCAAGGAGTAAGGTATGACTGGAGAGCAAACAAATTCAGAGTAAAAGGAGG |
| | TGCAATAACTCCCTGAAGAAAGTTCTACTGTTTTGTGTTTGACCAGAAATGTGTGA |
| | AAATAGAATATAAAAGGTAGAAGTAATTGGGTTCAGTTCTATGATTTTTATTAGCC |
| | TTAGTATCATAACAAGGAACACGGTCACAATTAAAGATATGGATGAAGAAGGCAT |
| | CTCTGAGGGAAAGTGGTGTGGATTGTACCCATGTAAAAGGCACACATTTTCTCCTA |
| | TCACAGAAACTAGGACAAGCTGGTAGGAGGATTCCTCAAAGTTTGGCCAGGAAGA |
| | AGACTTAAAGTTGTTTGAAAACCTTCCCAATATGTCAAGGATTGAAAATGACAGAC |
| | AGATATCATGTTGTTAGATGATGAGGGTTCAAACTAACCAGATATCCTTGGAAATC |
| | CAATCAAGTAAAACCTATTCATTAAGATCCAATTGGCATTTTGAGGAATAGAAAGT |
| | GAGGATCAAATTATGATGAATATGCTTATCAATCTAACATTTGATAACATAAAGTT |
| | TGGAGTGATACAAAAGAAGGTGAAAAATGAAGCTG |
| Human CPS1 | GCCTGATGTGTGTGCCTGCATTCATGCCTGTCACTCTGCTCAGCTGCGTGCAGACAG |
| RR21_v2 | CTTATATGAAGGAGGACTGTGTACATGTGGTCAGAGAAGAAAAAAATGCTTATTTT |
| SEQ ID | TTAAAATGTTTTGAAAAATAATCTGTCCTAAAAGTGTAACATTTCCACTAAATTTTT |
| NO: 76 | TCATGTCTGACTTTAACATTCCCTACCAACCAACTCTTATAGCCTCAACTCCTTTGA |
| | TCTATCCTAATGTAAACTCCCTCACTAATGTGTAATATATTTTTTTCCAGTTTGGTTA |
| | TAGATTTCAGTCTAGCCTGAACACTCAAGTATGAAACTAGCCACTATTTTATTTATT |
| | GGCTGTTCCAACCTCCATTGTGGAATTATAATTGATGGATTTTCCAAGTCTCTAATT |
| | CAGAATATTTAATCAAATTTTCCAAAATTTCTGCCTTCTCCTCCTTCATTCTGATGA |
| | CTAAAGACATGGACCATAAAATATATATACATATATACATACATATGTATATGTAT |
| | GTATATACACATATATATATGTATGTATATATACATACATATATATATGTATATATA |
| | CACACACATATACATATGTATGTATATATACACACACACATATATATATGTATGTA |
| | TATATATACACACACATATATATATGTATGTATATATATACACACACATATATATAT |
| | GTATGTATATATATACACACACACACCGTAGTTTGCAAGTCTGCATCTGGGACCCT |
| | TTACTGATAAAAATGAAAGCCCATTAAAACTCTCATGCCCACATTTCTCTCTAGACT |
| | TTAGAGTAGGAGAATTCTATCCTAGAATTGTTGATTGTTGACATTTCTATGGAAAA |
| | ACATGTTTAATTCATCATGCTTTGTAGTCAACTGCTATAAATGGTGATGAAGTCTTT |
| | TCCTTGGTCAAGCTCTGGACTCCTCAGTACTTCCACTGTCATATTTGATTAAAACAT |
| | AAGTGATGAAGGGAGGAAATATATTATAGTATTCATTGCATGGGTACCTTAGGGTA |
| | ATGGGACTATGACTGATTTAAATTTATTTTGCATCCTAAAAAGTTTCCATTAGTGGG |
| | TGCAGCACACCAGCATGGCACATGTATACATATGTAACTAACCTGCACAATGTGCA |
| | CATGTACCCTAAAACTTAAAGTATAATAATAAAAGAAAAAAAAACTTAAAAAGAA |
| | AAGTTTCCATAATGAGGATTGAGTTTATATCGTTTTCTTAATTAAGGAAAGACAAC |
| | CCTCACTACACACACACACACACACACACACACACACATAAAAAGACAAGAGGTG |
| | AGATCAAGGCGTAAACTAGTATTGGACTAAGCCAGTAAGTACTGCTTCTCAGTCCT |
| | CAGTTCTCTAGGTTGGGAGTTTTCCTTTTGCTATTTTCATTCTCCTCCAATTCATTTT |
| | TCAATGATCTTCAACACAAATTTCGCCTTTATTTCTTGTTCAAAATACTTCCATGAC |
| | TATTATGAATACTATTTATGGATGAAGGCTGAACATTTTAAGGCATACTAAGTCTC |
| | CCATGATATGGTACCTTTTCATTTTCCCATTCTATACCCTGCATCTAGGCTTTTAAA |
| | AAATATACCACATGACTTCATGCCTCTAGGCTATTGCTCATGCCACGGCATCCCTTC |
| | TGGGTTTTTTCCCCTCTTTATCTACTTGGTGAATTTGCATCCTTCAGTATAACACAGC |
| | TCAACATAATTCTATGCTTAAACTTTACTGAAAATGCACAAGGAACTTGATCTTTCT |
| | TTCTTATTTTCCCCATTTTATTTATTTTCCCATTGCATTTGGGCAACTTTCATGGCAC |
| | CAGTTATTTTATTAATTATTTGTATAAACTAGTATGAGCCTATTTTGTTTAGCTTTCT |
| | ATCACCAGGAGCCATGAAGCACTTTGAAATCAAATACTGTTTGTACTTGAATATAT |
| | TAAGTAAGCGTGAGGACTTAAATTAAACTTTGTTCCTCTTTAAAAAAATCCTTACCC |
| | CAACTCTAGCACTGA |
| Human CPS1 | TCTTCCTATGGGAAAAAATAATTTCCTCTATCAGAGGAAAAATTTATTACTATTATG |
| RR7_v2 | ATAATACCCCTTCAGACCTTTTTCTAGTCACTTACAGGTAAAAATTGTAGTACTGGT |
| SEQ ID | TGGTACATAGTGTATTTTATTAATGTTTTACTGCGTATATTGTTGTATAAATTGCTTT |
| NO: 77 | CCGCACTCATTGGCATGCCTTAGAATTCTTTTTATTTAGGCTTCATGACTAATTTGT |
| | AGTGGAACCTCAAGAAGATCACTTATGTGCTTCACTTTCCACATATGTAAAATGGG |
| | AATAACAACAGAATCTGCCTCATAGTACTGCTGAAGGGTGACATGGGGAACACCT |
| | GTGAGGCATTCAGAACAGAGCCTGTCATACAATAAATGCCATGTCAGTGTTTGCTC |
| | TTATTCTCAGTATACAGAGATCTGCTTTGATCTTTTCATCTTCTGCAAAATATTCCA |
| | CAAATTGGCATATTGTGTTTTGTAATAATCTCTATTGATTATTTTCAAGCAAGATTT |
| | GGAATTATCCGCAGTAGTTTCAAATGTACAAAAAAGTACCTTATTTTATTTATTTTT |
| | TAAATAACTGATCTGATCTACGAGTCTAATCTGGTAAGTTTTGTCTTGAGTCCAGTA |
| | ATGCAGTCCAATACTCCATTTAGGTAACCCTGTCCTTGCTTGGTCAATTTGTTCCAC |
| | TTTTCCAAGTTTGACCTGCTTACCCTATTCCTCTTCAAGAGGGCATCTGGTTGTTTA |
| | GTTTACATGATTTAGTGGTGCAGAAGGAAAGGGTAATCAGATTGATGTATCCTCTG |
| | AGTCCTCCTTGATATCTGCAGAGCAGCTGAGCTGGTCTGCTCTCTGTGCTGTTGACA |
| | TCTGCTCTTTGGGGCGTTAGGGGGTCTGGTCATTTGTCCCTTGTCTGTTATGATCTA |
| | CCTGCAGTGGTTTTCTACCTTGTCCCCAGGCTTCTTAATTCTTCCTGTACTCTCAGAA |
| | CCTCTGTGTTGTCACAACCAGACTTGGGTTATTTTGGGTCCCCAGTTTCCAGGCCCT |
| | GAATGCCATGGGCTCTGTCTCAGGAATAGGTATCTGCTGGCCTGCTGCTGTCATTTC |
| | TACTACATTGCTCCTTTACTGCCTTAAAGAGTGGTCCTGGATCAGGGGTTGAAAGT |
| | GGAAGTGGAAATAAAGGACGCTGATGTGACGTCTGTGTTTCCATTCTTCTTCCTAC |
| | CACTTCACCAGATGTGAAAAATTCATATAATCTGAATCCTCTAGTTTGAGTTATGAT |
| | AAGTTAAAGCAGTTTAAGAATTTAACGAACTCCCCTCTCCCTGATTAAATCTACTTG |
| | TTAGCACTGTGTTCTTACTGCAAATCCCGTTGCCTTGCCAGAAGATAAATATTAACT |
| | TTTTCTTTCTCTTTGCCTTCCTGTTGTAATAATTCCAAACCACCCAAAAGAAATAGG |
| | TGAGTTTGTGGAGAAGGTCAGTAAAATAATTTCAAAAGGGAAAAGAACATTCATG |
| | CATTTAAATATTATTCCCACTGGTTCATTTATGCTGCAATGAATATCTATATATCTA |
| | TGTATGTATCTATCATTTTGTGCACATGTTTTAGTATTTTTCTGAGTTTCATCCTGAA |
| | AAAATGTAATTGCTGTAAGGTATGCACATTTTACATTTCACTGTATAATGACAAAT |
| | CATGCTACAAAGTGACTAGACCAATTTATACTCTTGCTATGAGTCACTGAGAGAAT |
| | TCTTTACTAACACCAGATATTTTGCGTAACTTATTCTGTTTAAATTCTATCAGCAAA |
| | CCTCCATAGTAGGTGTATTCCCATTTTGTATATGAAGAAACTGAGGTTCGGAGTTA |
| | AAGCAACTTGTTCAAGGTCACACAAATGGTAAGTGGGTAATTTCGGAACCTTTTAA |
| | AATAAAACATAGAAGCCTACTATCTTATTGTTCCATTCTAGAAATATGTCTGTGTTT |
| | ATAATATCGACTACTTTGTATTATTTTAAACTTTATAATATTGACTACTATTATTTTC |
| | TTCACATCCTAGTTAAGAATCTAGGATAAAATGGAAAACGATATCACGAAATGGA |
| | AAAACAATGTTATTCCTTAGTTAGCTTAGTTTAAACACCTATTTAATGGTCAAAGGC |
| | AAGTAGTTGACCTCCCATCTTTTTGACTCACAGTCATTAGTTACACAAGAAAAGCA |
| | GAACAAAACCCAGCTGGATGGAGGGGTGGATATGCAAGTAGCTTTCACACTAGGA |
| | ATTGTGAAGCCTGAGGCATAGCCCCAAGACTCAGATCATCGGGAGTGCACTCAGTC |
| | CAGAGGGACAAATGTTCAGAGTCTGCATCACCTAATATTACAAGCATCTTTATTGA |
| | GACTGATAGAAGTGAATTTCTCAGGTCAGAAAACAGAAGAGCTCTTTTAGCTGAGT |
| | TGTGATATTTAAAAGTCTCTCATTTTGAATTTCTTGCCTGGCCAATCAAACTGTCCT |
| | CCCAGAACAAATTGTTGGATAATGAAATGCATATAATGTAGCTTGAGAGCGCTGTC |
| | CCATTTGCAGCACTGGCTTCAGCCTGAGGCACATCTCTGGCTGCTCCTTTCTATCTA |
| | TAAGTTAAAGCAATGGCCTCAGCAAGCAGTACACTCTGTACAGCAGCTGCAACTCG |
| | TTATCGGCTTTGTCCAGAATAACAAACAGAACAAACACATTTTGCAAGGGCTTTTA |
| | CCCCTCTTATGTAGAGAAGGAGGAATTAAGCAATCAGTGAAATGCTGATTGATCAG |
| | TTGTGCTGACTAAGAGAAGTCCAAGGCCACATTAGTACTATTAAGCCCTAAAAAAC |
| | AAAGCAAAAAGAAAATGTTCTTAGAACTTTATTTAAACCAGTATTTCTGTGATGTG |
| | AAGTACATTTCCCCCAAAGGGAAAAATGGGAAAATATTTGCATTTCACTTAGTACA |
| | CTTTACTCATCCAGACTGCTTTGTAATGTAATGATGGTTAAGTATTTTTACAAAGAT |
| | AATTCATGTTTTTTGGCAGATTTTGGTTGATCATGGAGTATCAGGGTTGTGTGAACC |
| | AAAGGTGTGATGCAGAGCAGCTTTAGTAAACCATCCCATTCCTATGCCACCCGTTT |
| | GATTTCAGTACCAACTGCTGTGGCCACATCTAGACCACTCTTTACCAGAAACTGTG |
| | CCACCTTCAAAATTCCAGCATCCTTTTCCCCTCCTCCCTTATTTTTTGATCTCATATA |
| | CTCAAGTATTCTTAGTGTAACAATTCTTTGAGCATTCTTTCTTATCTAGCTTAAAAT |
| | CTAGGGTCTACCATTATAATCATTCCTTTCAAGATAACCTTAACTTCCACACCCCAA |
| | TTTCTAACTGGCCTGGCAAAACTCAGACCTTTGGTGCAAAATCCTCCATCTCTATGC |
| | CTGAGCCCTTGTGTTTGAGAATGGCTGAAGAAAACCATGTGCATGGACATTCTGAT |
| | GTCACTATAAATTCATGGTCACGGATCTCAAATGGGAGCCCAAGACTACTGGCTTT |
| | CCTGCTGTGGTTGTCTAATGACAGCATGATCTTACTCTTCACAGATATTAGTTTAAA |
| | CTCTCCAACCTTCAATTGAAAAGTCTACATCTTACTTTATTAGAAAGTAGAAACAA |
| | TCAAGGGTTATGCTCCCATTTTCACACTACCAAGTAAACAAACCTTCAGTAAGAGA |
| | AGATATATTTCCTATATCTCCTCCTACCCTCCTGTTAAAAGGGTGCAAACGTCCCCC |
| | CTCCTAACAAAAACCAATCCTTCTTCATGCTCTTTAGATGCCGTGTTGTCTCTCTTTC |
| | TCAAAGTCTTCATTTTTTGGTTATACTCTTTCTCTCATGCAAATTTTTCTCTCTTTTTC |
| | AAGTAGAGCAGGCTCAGCAGCCTACAAACGTGTCTTGGGCACCTTTCATCAAAAAA |
| | TAAAAAACATAAAAA |
| Human CPS1 | TGTTTGTTCTGTTTGTTATTCTGGACAAAGCCGATAACGAGTTGCAGCTGCTGTACA |
| RR23_v2 | GAGTGTACTGCTTGCTGAGGCCATTGCTTTAACTTATAGATAGAAAGGAGCAGCCA |
| SEQ ID | GAGATGTGCCTCAGGCTGAAGCCAGTGCTGCAAATGGGACAGCGCTCTCAAGCTA |
| NO: 78 | CATTATATGCATTTCATTATCCAACAATTTGTTCTGGGAGGACAGTTTGATTGGCCA |
| | GGCAAGAAATTCAAAATGAGAGACTTTTAAATATCACAACTCAGCTAAAAGAGCT |
| | CTTCTGTTTTCTGACCTGAGAAATTCACTTCTATCAGTCTCAATAAAGATGCTTGTA |
| | ATATTAGGTGATGCAGACTCTGAACATTTGTCCCTCTGGACTGAGTGCACTCCCGA |
| | TGATCTGAGTCTTGGGGCTATGCCTCAGGCTTCACAATTCCTAGTGTGAAAGCTACT |
| | TGCATATCCACCCCTCCATCCAGCTGGGTTTTGTTCTGCTTTTCTTGTGTAACTAATG |
| | ACTGTGAGTCAAAAAGATGGGAGGTCAACTACTTGCCTTTGACCATTAAATAGGTG |
| | TTTAAACTAAGCTAACTAAGGAATAACATTGTTTTTCCATTTCGTGATATCGTTTTC |
| | CATTTTATCCTAGATTCTTAACTAGGATGTGAAGAAAATAATAGTAGTCAATATTA |
| | TAAAGTTTAAAATAATACAAAGTAGTCGATATTATAAACACAGACATATTTCTAGA |
| | ATGGAACAATAAGATAGTAGGCTTCTATGTTTTATTTTAAAAGGTTCCGAAATTAC |
| | CCACTTACCATTTGTGTGACCTTGAACAAGTTGCTTTAACTCCGAACCTCAGTTTCT |
| | TCATATACAAAATGGGAATACACCTACTATGGAGGTTTGCTGATAGAATTTAAACA |
| | GAATAAGTTACGCAAAATATCTGGTGTTAGTAAAGAATTCTCTCAGTGACTCATAG |
| | CAAGAGTATAAATTGGTCTAGTCACTTTGTAGCATGATTTGTCATTATACAGTGAA |
| | ATGTAAAATGTGCATACCTTACAGCAATTACATTTTTTCAGGATGAAACTCAGAAA |
| | AATACTAAAACATGTGCACAAAATGATAGATACATACATAGATATATAGATATTCA |
| | TTGCAGCATAAATGAACCAGTGGGAATAATATTTAAATGCATGAATGTTCTTTTCC |
| | CTTTTGAAATTATTTTACTGACCTTCTCCACAAACTCACCTATTTCTTTTGGGTGGTT |
| | TGGAATTATTACAACAGGAAGGCAAAGAGAAAGAAAAAGTTAATATTTATCTTCT |
| | GGCAAGGCAACGGGATTTGCAGTAAGAACACAGTGCTAACAAGTAGATTTAATCA |
| | GGGAGAGGGGAGTTCGTTAAATTCTTAAACTGCTTTAACTTATCATAACTCAAACT |
| | AGAGGATTCAGATTATATGAATTTTTCACATCTGGTGAAGTGGTAGGAAGAAGAAT |
| | GGAAACACAGACGTCACATCAGCGTCCTTTATTTCCACTTCCACTTTCAACCCCTGA |
| | TCCAGGACCACTCTTTAAGGCAGTAAAGGAGCAATGTAGTAGAAATGACAGCAGC |
| | AGGCCAGCAGATACCTATTCCTGAGACAGAGCCCATGGCATTCAGGGCCTGGAAA |
| | CTGGGGACCCAAAATAACCCAAGTCTGGTTGTGACAACACAGAGGTTCTGAGAGT |
| | ACAGGAAGAATTAAGAAGCCTGGGGACAAGGTAGAAAACCACTGCAGGTAGATCA |
| | TAACAGACAAGGGACAAATGACCAGACCCCCTAACGCCCCAAAGAGCAGATGTCA |
| | ACAGCACAGAGAGCAGACCAGCTCAGCTGCTCTGCAGATATCAAGGAGGACTCAG |
| | AGGATACATCAATCTGATTACCCTTTCCTTCTGCACCACTAAATCATGTAAACTAAA |
| | CAACCAGATGCCCTCTTGAAGAGGAATAGGGTAAGCAGGTCAAACTTGGAAAAGT |
| | GGAACAAATTGACCAAGCAAGGACAGGGTTACCTAAATGGAGTATTGGACTGCAT |
| | TACTGGACTCAAGACAAAACTTACCAGATTAGACTCGTAGATCAGATCAGTTATTT |
| | AAAAAATAAATAAAATAAGGTACTTTTTTGTACATTTGAAACTACTGCGGATAATT |
| | CCAAATCTTGCTTGAAAATAATCAATAGAGATTATTACAAAACACAATATGCCAAT |
| | TTGTGGAATATTTTGCAGAAGATGAAAAGATCAAAGCAGATCTCTGTATACTGAGA |
| | ATAAGAGCAAACACTGACATGGCATTTATTGTATGACAGGCTCTGTTCTGAATGCC |
| | TCACAGGTGTTCCCCATGTCACCCTTCAGCAGTACTATGAGGCAGATTCTGTTGTTA |
| | TTCCCATTTTACATATGTGGAAAGTGAAGCACATAAGTGATCTTCTTGAGGTTCCAC |
| | TACAAATTAGTCATGAAGCCTAAATAAAAAGAATTCTAAGGCATGCCAATGAGTG |
| | CGGAAAGCAATTTATACAACAATATACGCAGTAAAACATTAATAAAATACACTAT |
| | GTACCAACCAGTACTACAATTTTTACCTGTAAGTGACTAGAAAAAGGTCTGAAGGG |
| | GTATTATCATAATAGTAATAAATTTTTCCTCTGATAGAGGAAATTATTTTTTCCCAT |
| | AGGAAGATCTTAAACTTATTTGTAATGTTTCAATTTTTTCACATGATGAACAAAGCA |
| | ATACACTGCTCATGTTACTGAAACTCGATAAAATATATGAAGCTAAAATTGGTCAT |
| | CTAAAAGTATGATAATATATAATATTTATTTTCTACTTTATCTCCAATATGCTTATC |
| | ATACAGAAATTATAACAAAATGCAAGCAAATGTTTTGGATAATTTGAAAGTTAATA |
| | AGTTGTACACTGTAGCCCTACCATCCCCACTGATGTCAAAGGGCTGATTTTTAATTA |
| | TGCAATAGTAATTAAATAGGGATCAATGTCATTACCTGAGAAAACAC |
| Human CPS1 | AATTCTAAGTCAGGTGGTCCAACAAGGCCTCTCATCCTATTGGTATTACAGTATGA |
| RR9_v2 | CCTGTCTCCTGAACTTTGAAGCCATTTCTTTCCTTCAGAGGAGATAAGAAAATGTGT |
| SEQ ID | CTTATCTTAAGTCTGGAAGAAACACGGAGCCCAGTATCAAGGGAGTGGAGTAGTTC |
| NO: 79 | TTTTCCCAGACGTAGCACAGGGTTTTTCAAGCCTGAAGAAAAAGATTAATCTCCTT |
| | AAAACAAGGCTTTGTTGACTTGGGTCTGGTCATGGCAATCCTGTTTCCAGCCATCCT |
| | GTGTATAAGCTCTAACGGTCAGAAGTTTGTTTGCTTTGATTTTGATGTGGCTCATCT |
| | TTTCTTAGCTGCTCATATTCAAGATTTGGAAAAGAAGGAACGGTGGTGAGCTGAGC |
| | AATTAATTGGATTCTGATAAGGAGTTCTGGAGAAATTTTATTATTCCTGAGTTTTAA |
| | ACCCGTGTAACTTTAATATAACTTTGGTATGAGCCTTTACATTTTTTCTCTGTGTTTT |
| | TGTAATTATATTGTGGCATTCTTATTGGGTGTGGTAGTAGCAATAAATTATAAATGT |
| | ATGAATTTATACATTATGGATTTATAATTTCATAAGTTTTTGAGATTTTAAGACTTA |
| | CTATTTTTTACACGATGCCTTATGTATTTATAGCATTTAATATTAATCTAAATTCATT |
| | TTGAGATATATTTTATCATGCTTTTATCTTTTATATGTTATTAGAAAGCACTTGGTAT |
| | TTTAAAAATATATATTAATGTGAGATTTCACAATTTATACCTTGTCTTTGTAGTCAC |
| | ATGCGTAGAGGATATTTACAATTTTACTCAAGTTAATTTGCTCAGTGTTGATCATCA |
| | GACCTTTTAGCCAGTATTGGCTGGGAAAAAAAGTGTAATTCTTTTTGTTCTCTTGAG |
| | AATTTTTTGGATCTAAAGTGATTCCTAGGGTGTGGGTCAACTTCTATGATATGAAA |
| | GATGACATGGAGGTTTTTTATTTTGTGAAGTGCATTAATAGGTGATCACATTTCATC |
| | CTGGGAGAGCTGCTGGAGAGGAATATTGATTTAGCCTGTTATGGTTGAGTCTTTGG |
| | GGTCTTGAGCTTCTTAGATTTTTAGTTCTTTTTGTTATTCTTTATTGTTCAATCTTGA |
| | GAGTAGAAAATACACATTTTTGTCCACTTCTAAGAATAAAACTGAAAGGCTTCTCA |
| | AGAGGGAGCTTTTGAGGGACAAGTCTGTGTTCCAGACTTATTTTTATTCTCGGATCT |
| | AGCACCATGCTTGGCATGTAAAAGGGCTTTAATAAGTATTTATAGAAGGAATTGAA |
| | GAATTAATTATTACTTATGGACTATGAGAGGTCATGGTTGTTACTCTTTCTAATTTG |
| | TCGATATTAAACAGAGACAGTGAAGATACTAATTTAGATCACACAACGTGAATGA |
| | AGCAAACAAAAGCCTAAAGCTTTAGAAAAATGAATATATTATATTTTTAACTTCGC |
| | ACTGTAGTTAAGCAGTGTGGAGCTATTTAATGATTTCAACCAGGAAGATATTAATG |
| | TGTGAAATAAGAGTACGTGGACAGTATATTAATTGTATGATACAAACAAGATATAC |
| | ATACATAAAGTTCATTAAAAGAGTATGATTTCATATGGCTTTGAAGGATTGATAGG |
| | ATATAGACAAATTGATAAAATTGATCTAACCAATATTTAACTTTAGGTTTCTTCATC |
| | AGATAAAATATTGCATCATTTTATGTTGGCTAAGTGATTTGCAGTCTATTTCAATTA |
| | ATTATTCTTTATAGTTTTTCTCTGTCAATGCCATTTAATAAAGAGAAAACTTCAGCA |
| | GACAGAAGTTATATGATCTTTGGCACTATTGGCTGTGTGTTAAGCCACAGTAAGGT |
| | TCAGTTACGTATTTTATTGTTTTATAATCACACATTTCTCCTTTGATTCATTTCTTAT |
| | CTACACCCCTTCTTTCACATCCTAAATATTTTCTTTGGATAAATGTGTAGGGCAAGA |
| | TGAAGAAAAGAAGAGAAAAAAGTAGTCAGAGCTTGGGAGTACTGAAACGCTTTGG |
| | ACTCCAGCCAGCTGCCATCACTGAGGTGGTGAAAGAACACCGTAGTATAACTGGAT |
| | GGCCCTGGTCCCTGCCAGGAGTCAGGATGGACTTTCAGTGTTGAAGAGCCATCTTG |
| | GCCTGTTACTAGCGCTGCCCCACACCATTGTGTTTGCCTAAGAGCTGAGCCAAAGA |
| | ATTTACATATGTATGAGCCAAGGTCTGAAAATAGAAGTTAAAAAAATGGTGCTAA |
| | ATTGACATATTTGCTATTTTATTTTTTTGGTCAGTTAATATGATAAACTTTTATTTAA |
| | ATGTAAACATTATTTTAAAAAGCACTTTGTTTTAAATAACTGATTTTCATTGAAATT |
| | AATTCTGGCCTCTAAGCAGAGTTAGCTACAAAGACAATTTGTAAAAATGCAGAAA |
| | ATTCAGAGTTTAACTTGGAGTTGTTTAAAGTTTATTTTCATATTTGAGTCTGATATTT |
| | CAGTTCACTGTAATATTTCAATTTATTTAAAATATTATCAAGACAAATATTGGGTTA |
| | CCTTCACAATATTTTTTTCATTTTTCTTACCCCATATATTAAAAATGAACAAAAATG |
| | AAAGAATTATATAGTCTAGTGGATGGCAATTCTCTTGGTACAGTACTCACAGGATG |
| | TCAGAAGTGCTTTGATTTGCATGCAAATAGAAACTGCATTCCTAAAAGACCCTTAA |
| | TTGAAGATATTAATAATTTTCATTAGGTTTTAAGTCTTTTAACCTTATCATATGGAT |
| | AGAAAAGTAAAAGATGTGTTTAATAAAATGTCTTTTACTTATTTTTATTTGTCATTT |
| | ATTTAAAGGTTTATTAGTGAATCTGTTTACCTGGAAAATGTGCAATTCTATTTGCAA |
| | ATTTGGAGCACTACATATATTTAAGAGAATCCTTAGTTTCTGTTGGGAGTGGCAAT |
| | ATTTTATAACTGGGCCTTCATATTCCTATGTTACAGCCCTGGTCCCTGAAAGTCCTA |
| | CAGCTATCTTTGTTTCAAGTGTGTATGTGTTATAATAAGATAAGTGTTAATTAGTTA |
| | ATCAAGTACTAATTAACCTTTTTAATTTATAGAAAAACAGACATTTATGGACAAGT |
| | AAATTTTTGGGGTCAAAGTCTTTAATAATTTGAAGAATGCAGTTATTTTTAGCACAA |
| | ATATTCTAATGTGAAAAGAATTTCTTTTTCTGAGTTATATATTTTTATGCCATTCAG |
| | AGTCACTAATATAGTACTAATAATACTTTTTCTATTGTGTACCTTTGTTTCTCTCATT |
| | TGTCTTACATTTTATGTCTAATCATCATTTCACAAAATTTCTATCATTACTGCTTGAC |
| | CTCGACTTCTAACATGGCAATTAATTTTTATACACAAATTTTTGAAATCTTGACAGA |
| | GTTTTTAACTTCCTCATACCGAGTTCTTTGCTTTTTTAGTTTACAAGATGTTCAAGAA |
| | TGCTCTTTTGAAATAATCAATTACCTAATGCAAGATAAATTAGCAACAACATTACA |
| | TATTTTTCTTACTATCAACTGAGAATGATCTTGATAGGGGCAAGTATAGGTTAAAA |
| | ATAAATGACATCAAAAAACAATACCAGAATAAAACATTTAATTCTTCAACACTCAA |
| | AGGCATGTCAGGTATTGAAATCCAAGTAGGCACTTTTGACTAATTGTTAGTAGTTT |
| | GTAGAATTCAAGGATTAGGGTTGGCTTCCTTTTGTCTTTCTTTCTCTTCTTCCTCCCC |
| | TTCTTCATCTCCTTCTTGTTCTTCTTTTTTTTCCCCTCTCCAGATCACTAATCTTCTCC |
| | AACAAATTTTTAGCCTATATAAAATAAAAAAAGGTACTCCTGCCCTATGACTCAAC |
| | TTACAAATCTACAAACCTGGAGGAAGAAATAGCTCTGTGATCCATTCCTGGGGAAT |
| | TGCAATAAAGTTTTAGGGAAGATGGAACTTAATGTTTGTGAAAGCACGGGTCCTAT |
| | TTCCGATTTCCAGGAGGGTGGAATGTATCTTCCTGAATCACATTCCGCTGAAAATA |
| | GGAGGCCATGGAAGAAATCAGGTTCAGTATAACAAATTTTCTACAAAAGGGGGTG |
| | GAGTAGGAATGTGCGTAGGTGACCTGTTCTCTACGTCATTCCAGTGTAAACTACTG |
| | CCCCTTACACTTCACGAAGTCGGTAACTTCAAGACATGTTGCACCACATGCTTCTCT |
| | GTGATCCTCAAATATGTTTAATTTAAAGAGGGTCCAGTAGTGTCCTGGCACATGAT |
| | CTGGATTGCCATAGATAACCATCTACCTCACAGCTAGGGTTGCTCTTTAGAATCTTG |
| | CAAAATCATTTGTTTACTCTTGACAAAAGTTAAGAAAACAAGCCCATCAGAGTTGT |
| | TTGTTCTGTCAGCATGTTAGAAGATGGTTTTGTTGCAATGATAATCGTTGTGCAAAG |
| | AAGACTGATGATGATTTTTTTTTACATTTTCTTAACAGTATTTGCTATTTAGAATGA |
| | ATGTTGTCTAATTATTTAGCCATTTTATTTTGTAAAATTTATGTTGTAGGCATATTTA |
| | GACCAAGTTATAAGAAAATGCTTCAGCCAAAATTAAGTGTTGAGTTTGATTTGTGT |
| | AATTGTTAGTTTCTTTACTAGTTGTTCCATCATTTACACAATTATTTCTATTTGAAAT |
| | GCAGTAATTGTTCAGAACTTATATTTCTATACTGATGTCTACTAACAGCTTTAGATC |
| | AAATATTAAATAACTCAAGAATAATGAGATGATCTTGGCTTACTTAGATATTTGGT |
| | TTTTATATCTATAGAACAAAGGAATTAGAAAATAATTTTGAAGATTTCATCCAGCT |
| | ATGTAAAACTATCTAGGGAATACATTTACTAGGTTTTCAATTTTCTACAAAACATCT |
| | TTCAGCAGAAAGCAATCCTGTTTCCTGATATACAATGTCTGATACATAGAAACTAC |
| | TCAGTACATAATTCCTGAATTGATTATTCTTTTGGAAATCCTAGATTTGATTTCTGA |
| | ACAATCATAAACATTTAATGGCATGAAATTACCCAGATTCCATGGTTCTGGAATAC |
| | ATAACTTCAAGCAATAAGATGCAAGATAGAAACATATAAGACATTCTTTGCTATTT |
| | TAGGTAAGTCCAGCTGAATCAGTTAATCAGCTAGAAACGTGGCTCACAGATGAATT |
| | AGTTTTATTATTAGGTGGATTACTGAAAAATTAATAGCTTTATTTCCGTATTACCTT |
| | ATCATTTATTTAATATAAAACATAATAAACCAGAGAAGTIGTGGTTACTTTC |
| Mouse CPS1 | CACTGCTGGTTCCACCTCCTTCTCTCACCATGGGGAGAAAAAGGTGACCTCTTCTG |
| RR52_v1 | GCATTGCAAGCACAACCTGACTCACAGGGCAATGCTTTTGTATCTGAGTGTCTTTC |
| SEQ ID | ACAATCCATTTACTATCTGTAACTAGCTGTTCTGTTACTAACCTTTAAAAACTGCTC |
| NO: 80 | CCATGCCCTTCAATGACAATTATGTAAATATACTCTAATGACTTTTAACTATCTTTA |
| | AAATACTGTGCCCACATCAAAAAGAATTAATTTGGATGTGTATTTCTTAGAAAATT |
| | GAGTTTTAAAAGAGAAGGCAAAAAAAAAATCATACATAAAGGACTATTTCAAAAT |
| | AAGTGACTAAGAACACAGGTCCCCCTTGGACTCTGTGGGACTTTAATTCACTACCA |
| | TGAAGCTTAAGAACTTCCATTTTTCATGCCTGGCATTCCTCCAAAGTTCTGGCCAGT |
| | AATGACTGTATGTGAGTAATGGCTTTTGCACCTTGAAATGATGGCTTATTCCTCCTG |
| | AAAATTTGTCATTCTTAGCTATGAACTATGGAAGACCCTTGAAACAGATCTACCAC |
| | CATGAGCTAGAACTGCTTTCCCAGGGGTCGGTCCACATAATTAGCTCATGTTGTTGT |
| | GTGGTAATTGCTGTGTAGAGTGCTTGCTGAGTCTAGGGATCTGATTTATAATTATTG |
| | TGGCCTCCACTATTCATTGCTCTGTTTTTGGTATTTAACAGAGACCTGCTCATCCTT |
| | AGCTACCCACTTCTGTGAGAATAAAGTGGTCCAGGTGACACAGTTCAGAAATATGA |
| | CAAAGCACTTAAACTGTAGATGGTACTGTTGTCCTTACAGTTTCTTCTCTTCAAGAT |
| | GGCATGTGATGTCTTCAAGGCATTGAAACACTTAACTAAAGCTAAACCACCATTCT |
| | CTGTATATCAAGGTACTATGTCCTTATTGATGCTTTAGAATATCCATTCTCCTTTCTG |
| | GCTTCCATCTTATTATCAAAATGAATCCAACATGGCTTTCTTAGGCTTTGTAATAAA |
| | TTCTCAGGGTAGCAGGCTCAAGTTGGTATCATGTCATTGATGTAAGCATAAGTGTC |
| | TCTCTCCCTTTCCCTCCCACTCCTCCTCCCCTTCCCCATCCTCCTCTTCCTCTCCCTAC |
| | CCTCCATGCCACCATCCTTTCCTTCCACCCTCTTTTTCTCTCCCACCAGTCTTCTTAT |
| | GTCTCTCTCCTGTCCCTGGAATCTATAAGAACCTATAGAAATTGCCTGTTCCCACAC |
| | ATGCCAGAATCCAAAGCCCCTGAAATCCTGACCAAGGCATTATAAGCAAAAGAAT |
| | CTCTTTTGGTATAGCATTGGAAATGTAAATGAGCTAAATACCTAATAAAAAATGGA |
| | AAAAAAAAAAGAGTCAAATGTCAAAAAAAAAAAAAAAGGAATCTAAGGAGCACC |
| Mouse CPS1 | ACTTCTTTTGCTTCATAATCATGTTACAAGAGGCCCTTGACCTGAGGTACCCTCCAC |
| RR34_v1 | TAAATACACATTTGATTTGCACAGTGGGGTCATTGGCTCTGGCAAACAGCAGTGTC |
| SEQ ID | AACATTGCTAATCTGAAACAACAATCTCTCATGCCCCTCTCTGCCCCACCTTCAAAC |
| NO: 81 | ACACCCACAGCAAGTTCTGACAAACACTTTATATGTGCCGAACTGAGCATTCTTTA |
| | TCACTTGAGTAGACATGACAACCAGCTAACCTTGCAGGAACGTCCCTCTCTCCTGG |
| | CTTCTTTCTAGTGAGAATGGAATCCAGCTCACCTTATCCTAAAATACTTGGTGTAGA |
| | ACATGAAATTGTTTTGGAGTTTTCTTCATATATATTTAGATCTAGATAGAGCCCATG |
| | CTTATCTTAAGTTAACGAAAGAGAAATTCAAATTAAAACAAAAAAAAAAACAAAC |
| | AAACAAACAATTGCTCAATCCCTCACACTTGTTAGCTCTGGGCCTGGTCTTTAACCC |
| | CGGTTTTCCATTTCTACTTTAGTTCTTTTCTCAGTCAGCAGGGTTTGCTAAGATTCAT |
| | GCTTGCTTTACATTTTCTACAAAAGCCCAGAGACTGAAACCATGTGTTTTAAGTGGT |
| | TTTAACTCAAAATGTTCTTTTTTGCTTGTTGAGAACTCATAACCCAACTTGATAGAA |
| | TGTTCTTTCTAATTATAAAGTATTGTAATGAGGTCCCTGTATCCAAGAAGCACCATT |
| | TAATGTCCGCTCTGATCTCTGAGGGTTTTATTTTTACATCAATCCAGTTCTGACCAC |
| | CCAGACATGAAAGTGCCTTGGTCACTGAGCCAGCAGCACCCTCCAGTTGGCAAAA |
| | GCAAGGTCATGAGAAATGGTGACAAATTTCAGAGCTTCTTACTCCTCATTTGGATT |
| | CAAAGTCCCTAAAATGGAAATATAATTCTTTTACCACTTCTTGAACTACTCTATCAG |
| | GCAGGGTTCAAAAAATAAAATCCACTTTGAGAGTTTCTTTAAATAGTAACACCCTT |
| | AATAATATCAAAACATGATTTGCATAATTGCACTCTATTAACTAATATGAGTGTATT |
| | TCTTGTGAATAGTTATGTTGAAAAGACGTTCTTGGAAGCATCAAAAATATTAACTA |
| | AAATATTTAAAGAATGATTTAAGCTCCATCAAAATTTTAAAATGTTTACTCAAAAG |
| | ATACTGTTAAGAAAGTGAAAAATAAGGCATAGACTTAAAAGCAAATATTCACAAA |
| | TGATATTATTGGTAAAAAAAAAAAAAATTGTATCCAGGCCAGATCAAGAGCTTTTA |
| | GAAGTAGATAGCAAGATGAATGAGCCAACTTAAAATAAGCAGAATAGGAGCTGGA |
| | AAATGCTCAGTGGGAAAATGCTTGCTATGCAAACATGAGGATCAGATCTCAGATAC |
| | CCAACACCCCTATAGCTCACAGCCAACAATTGGACTGAGCTCCAGGGGACCTGGAT |
| | GGAGGAGATGGAGAAGGGATTGAAGGAGCTAGGGGTTTGCAGCCCCATGGAGGG |
| | AGCAAGAAGTGTCAACAGGCCAGATACCCTAGAGCTTCCAGGGACTGGATCAACC |
| | ACCAAAGAATACACATGGAGCAGCCCATGGCGCTGGCCACATATGTGGCAGAAGA |
| | TGGCCTGGTTGAACATCAGTGAGAGGAGAGGCCCTTGGGCCTGAAGGTGTTCAATG |
| | TCCCAGTGTAGGAGAATGCCAGGGAGGGAGAATGGGAGTGAGGGAGTCGGGGAG |
| | CACCCTCATAGAGGCATGGGAGGGGGAATGGGATAGGG |
| Mouse CPS1 | AGTTTTGGCACACCATTTTAATGAGACAGCAGATGCAAAAGTGCTTACAACAGCTG |
| RR35_v1 | CCCTAAGTAGACATTTTATCATCCTCCCTCACAGCAGCCATTCTGTGCCTTCTTCTG |
| SEQ ID | CATGGCTATTTAGCAATTGTAAGTCTCTAGAACCATTCAATGGTATTCTGTACAGTT |
| NO: 82 | AATGTTTTTAATTATATTCTGCTTCAGACTTTGATTCTTTTGTAAGCTTTTTCTTTAC |
| | ATAAAATTTACCAACTGCTTCTTTTCCTTCTTGGACTTACACATGTCTATCATAACC |
| | ACACTGACTTTATTTGGAGACAAAGTAATAAGGTAACTAAACTTTACTTACTCATT |
| | ATAGTGTTAAGGAATCAACCTCATGTCTCACACAGACTAGGTAACTGTTCTGTCAC |
| | TGAGCTGATTCCTAGCCTTTCTAATGTTCTATGAGTGATTATTATAATCTACGAAAG |
| | ACTAAGGGCACAGTAAGGAACAACACAAAATTTTCTGCCTCCACTGAACTTGTATT |
| | GTAAATGTTATAATGAAATGTAGGGACCATGAATGAATAATGGATATGGTATCTCC |
| | AAATCTTATGCATCGTCATTTTCTAATTCCTGTATCCTGATATTTTGTGGGAAACTT |
| | GCTACCCTGTTTGACTGATGTCTTGGTGGGACAGTCTTTGATCTTACTCCAATCTCG |
| | TTCTCTTTGCCAACTTGTAGACACCTGATCATGCTTGTCCAATCAGACTCTCAGGAA |
| | AACTGAATATAATTGGACTATGTTCAACTCAAAGATGGCTCTTTGACAAAGGCCAT |
| | TGACTCCTGCCACCTAGTCCTCAGGGACTCATTTGTCCTTATTTGTTCCAAAGGCCT |
| | CTTTCCTTGGCTACTCCTTGTATGCAAAATCTTCACTGTTTCTTGCAAGTTATTTTAA |
| | GTGTAATTTTGTGTTGGCTGGAATCCAGTTATTCTAATACATTAAATTTGGCAAAGG |
| | AAAGAGCATAGCATTTCTTTATTTCTCCAAGTCAACTGTCTTATCCTTTTATCCACT |
| | GGGCTTGTAAGAAATTAAATTGAAACTTTAGTGGGGATTCCCCTCTATGATTCCGT |
| | GGCTGAGAACCAGTAGCCAGTTTCTGAGGTGTCCACTGTGGCCGCCTTTGTTCTAT |
| | GCCAGGGATAAGAAGATCGTTAGAGAGACTGGTTATCAGGGAGCTAAGGTTCGCT |
| | TCTACAGGTGCAACGTGCAAAGCCTACTGTATGTAAGTCTGTATTATGCCTGGGAG |
| | ACTTTGTACCATCGGGCTGATTACTCTGATGCAAAGCCTCCCTCAGACAGTTAACTT |
| | ATCTTAAAGCCTCTGTTATCGTTCATGAACAGAGGGCAAACTCATGTCCATTCTGTC |
| | TTGTTCTCCCACCTGTCTCTTAAGTTGCTCTGGATTGCTACATTAAACTCTGAGGAA |
| | CAATGAACCCATTCTTCCATTTAGTATGAATTTAGTAAGTGTCTGATCCATTCCAAA |
| | TTCTCTATGAGACTCTGAAAAGACAAAGGGAAACTCCATTTAGTTATTGTTCTACA |
| | GGAGTACATTTTGGGTTGGAGAAAGGGAAGATTAAGTAAGCTAGTGGCTAAGCAA |
| | CAGGGCAGTGATGAACTCGACAAGAACCATGACAACATAACAATAGAAAAGATCT |
| | GAGTCTATTTGGAGAATGTCATGTCAGACATGGACTACAAAAGGAGAGAACGGCT |
| | GGGTTGGACCTTTGGGAGGATGTGGGAATTTGCTTACTATACAGGTGACCAGTAAT |
| | GGCACATACCAAATCATAGAGGGAGGAGGAAAGAAAGGAAGGAAAAGAGCAGGT |
| | TATAAAGAAGGAGGAAGAAACAACATGGGAAAAAAACAGGGTCAGAGAAGAGGC |
| | AAGAATAAGAACAAGAGAGAAGGAAGGAGGGAGAGGAAAAGGAAAAGGAAGAA |
| | AGAAAAGAAAAAAAAAACAGAAGAAGGCTGGGGGGGGGGGGTTATGTTGTCCT |
| | GTAACAAGTAGCACACTATAGCTGACAGGATTAGGGTGAAGTCCACTAAGAGGCT |
| | TGGGTGAGACAGACATAGGAAACAGGTAGACAGAAAAACCCAGAAGGAACATTG |
| | TCATCTTGCTCTCCTGTGTGAAAACGGAAGCTTTGCCTCTGTGAATCCAAGAAAGA |
| | GACGTACCTCTGTTCTTATCTACACAAAAGAGAAGGTGGCAGAAACCTCATCTTAC |
| | TGTCACTGAGAGGACTAAATGGACTTTTGAATCAAAAGCACTTAGCACAGTACCTA |
| | CCACACAGCAGAAATCAAGGGCTTTTACCTGTCCTCCTTCTTCCAAACCTCACACCC |
| | TGAAAATCCATCACAAACCCTGTGGAACCATAAAAAGATATCCACCCCT |
| Mouse CPS1 | CCTCTATCTCCACTCTCATTAATATGGGTGTCCTATTGCTCTTATTTTCTTATTAAAA |
| RR36_v1 | GAAAAGGGGGACACAGACACACACATAAATATATACAAACACATGCACACACACA |
| SEQ ID | TACATATATACACACATGTACATACACTCCAAGATGTTTTGATGAAAAAGGCAGTT |
| NO: 83 | ATAAATTAGAATCTGGATGAGAATCAAGCCAGGCAGGTTTTATTTCTTGCTATCTG |
| | TTGGTTTGGTTTTACTTGGGTTTGGAGACTAGGGCTGTGTTTTGAGATGGGAGACA |
| | ATGAACAAATATCTGGATATTTACCTGGAAAGTGGAGTTGTAAATTTTTATGATAG |
| | AAAGGGTATAACCAAGGAGTAAAGACTCAAAGAGTGGCCAAAGGGGGGATATGA |
| | AGAAGATGGAAAGTAAGTCATTTCTGATCTGCCAGACATACTTCCACTAGTATAAC |
| | TGGAAGGCAGAAGGACTAGACAACCTTTCCTTCCTGCAAGTTGATAAATTCAGTAG |
| | TGTAAAGAGTAGAGCACAGCCATCTCATGACTTCCATTTTTCAGTGATGTGGGGTG |
| | CAGTTTTTCACTGAAGGTTGCAAGGATGCAAACCCTTTAAAGGATTTGACCTGTGA |
| | TAGGCTGCCCATGACCCAGTAGATGCCCTGTACCTATGCTCATACTGGCAGCACTA |
| | AGTAAACTCAGTGAGTTTAAAAAACAAAACGAAATATAAAGCTTTGGGAGGGAGG |
| | GGATGGTGGTGGGCAGGAAGGAGGAGTTGGAGAGGGGAGAATGGAAGGAGACTA |
| | GATCAAAACATGTTATGTGCCTGAATGAATTCTCAAACAGTAACAAAAGAAAACTC |
| | CTTAGGAGTAAACTAGATCAAAACATGTTATGTGCATTCATGAATTCCCAAACAGT |
| | AACAAAAGAAAAGTCTCAGGAAACTAGGGCCATGTTCTCCTTACAGTAAGATCCTC |
| | AGTAGTATCAGATTTTCACCCGCGATAGCAATGGTGAATTCATATGACATCAGAAT |
| | GTACATGAATATGGCTTTCTCCAGCTATTTTCAAAAACAAGTGTGCAGCCTAGCAG |
| | GTGGCATATTACTCACCGAGAGTTCAAGCATTACCTAGCAATATAGGAAGAAATGG |
| | GGTGTGAAATATGAGACTCTAGAAAGAAATAACTACAATAGTAGACCCTAAATTCT |
| | AAGACAAATGAAAAGATTGCTCAAAAGTGAGGAGATGTCTCAGTCAGTGGTGGCA |
| | CATACCTTTAATCCCAGCACCCTAGAGGCAGGGGAATCTCTGAGTTCGAGGACAGA |
| | CAAGTAAGTCTCCAGAGCAAGTTCTGGGACAGCCAAGGCTACACAGAGAATCCCT |
| | GACACATGCATGTGCACATATGTACCTACGTGTACACACCACACACACACACACAC |
| | ACACACACACACACACACAAAAATCACAGCTCTAAAAACTACTGAGGCAGATTCT |
| | GAAAGATCCATACAGTAAATATATGAATTCAAAAAATAGGAAAAAAGATGAGAAA |
| | ACTAAGACATTTGGATTTTAAAGGTGGCTCTGTTTCTGATAAAGATCATCTGGGAG |
| | TTGGGGGGGGCATAATTAAGCTGTTACGCTGACAATCTTTTTTCATTTCACACAATT |
| | CTACCTCTCTGTGGTCAACCAACACAGTCCAAATACTATGAATCCTGTTTATAAGG |
| | CATAACCACCATCAATATGAGAAAATCATAATAAACAAAGCAAATTTCTTTCCTTT |
| | CTTTAGGCAAAATTGAACATCACAGAAATATTAGTATAAATAAAGTTCTAAGAGTA |
| | ATCTTTGTTGAGATTTTCTTTTTTCATTTTACAGGGTTAATAGCACTCATGTGGCCTT |
| | GAACCTCTCTATGCCAGTACAATTGATCAATCAGCATTTCACCGATTGCTTCACTCC |
| | TCCTTCTCCCCATAAGAGGAGAGAAACCCTTGCAAAACGTGTTTGTTCTGTTTGTTA |
| | TTCTGGATAAAGCCGATAACGAGTTGCAGCTGCTGAACAGGGTGTACTGCTTGCCG |
| | AGGCCACGGCTGTAACTTATAGATAGAACAGAGCAGCCAAAGATGTGCCTCGGGC |
| | TGGAGCCAGCTCTGCAAATAGGCCACACTCTGGGCTATGATACATGCATTTCATTA |
| | GCTGACAATTTACTCCTGGAGAAGCTTGATTGGCGACAGACTCAAAAAGAGAGAA |
| | TTTGAAATATCACTCCTCAGCTCAAAGTGTTTTTCTCTGTTTCTGACCTGAAGGATT |
| | GGCTTATGTCAGTCTCAATCAAGATGCCTTTAGTAGTACTTGAGGCTGACTTGGAA |
| | CAATATGGGTCCCTCTGGACAGAGTGTACACCTGATGATTCGTGTCTTGGGGCAAT |
| | GCCTGAGGCCGCACAGTCCCTAGTGCTGAAACTGAGCTCACGCCCAACTCAACTGA |
| | GTTTTGTGCCTCTTCCCTGTGTACTTAAGGGCTGTGGGCCAAAAAGACATGCAGCC |
| | AACAATCTGCCCTGAACCATTAAACAGCTCTCTAAACCGAGCTGATGGGGAACAG |
| | AGTATCATTGTCCATTATTTATAATGTGATCTTCTACTTTTATCCTCAATTATTAAGA |
| | CACAAGGAAAACAAGAACAGTCAACATTATAAGGATTGGGTCAAAATGAATACAT |
| | GCTTTGAGCATAAACCAGAATCAAGATAACTCTTGTGCTTGCTTGAAAAAAGTTTT |
| | CAAGCCTTCATCCTTTAGCAAGTTGCTCTGACTCCTCAGACCTCTGTTTGTTCACAG |
| | GTAGAATGGGAATGATCCTCACAGCTGTGGAGGGTTATCCAGAGACTTCTGATAAA |
| | GTAAGTTATATGAAATGTCTACCATTAGCAAGGAATACTCTAAAGTTGTCTAGCAG |
| | AAATGACGAGCAGGTTCAGCCAGTCAAGGTTCCTGTCACCAAGCCTGAAGACTAA |
| | CTTCATCCCCGGGATCCACATAATGGAAGGAGAGTGAGAACCCCACAGTTGTCCTC |
| | TACATACACACACGCACATACACCACAAAATAAGATGTAATAAAATATGTTCTAAT |
| | GCAATTTTTCATTATATAGTGGAATATCAATTGTGTATATCCTATAGTAATTGTACT |
| | TTTCAATATGTAACTTAAAGAGATACAAAAATGTGTGCAAAAATTATATATGTTTT |
| | TACAATAAGAATATATATATATACACACTGTGCCATTAATGAATCAAGCCAAATAA |
| | CATTTTAATGTATTAGTGTTCTTTTCTGTTTTGAAAGTCATCTTACTGGCCTTGCCCG |
| | CTTATATGTTTGAACTCCTTTTGGATTATTGTAACTGGAAAACAATGTGAGGTCTGG |
| | GATCCAGATAAAAAGACTCCCTGAGGCAGATTACTCCACACACCTAAGTATCACAT |
| | GGCTGAACCAGTATCCAGTACCACACTCAGTGAACAGACTGAGCAGCAAACAAGG |
| | CGCTGAGGGCCAATACAGGGCCCCCAACAATTCATCCCTCTGCAGAAATCATGGAT |
| | GGTCCAGAGCACACCACTGGTCCCTACCACCTTTCCCCTCTCTACCACCCAATCAAT |
| | CACATGAGCTAAACAACTGTCTTGAATTGGAATGGCGAGCCAGTTCCCTAAATGGA |
| | GGGCTTGGACCGCAGAGCTGCACCTAAGAAGAGAATTTAGCTCGTTAAATGAAAG |
| | ATGTGTTTCCAGTCAATGAAATAGATCAGGTTAATTATTTAAAAATCTCCAGCTTCA |
| | TTGCACATGTGAGTCGACTGCAGGGAATTCGAAACCTTGCTGCAAAGTAATCAATA |
| | GAGATATTTCTAGGCAAAAAAAAAAAAAGTATGTTGGTGAGCATGGTGGCACACG |
| | CCTTTAATCCCAGCAGTCAGGAGGCAAGTGGAGCCATGAGTGCGAGGCCAGCCTG |
| | ATCTACAGAGTGAGTTCTGGGACAGCCAGGACTACACAGAGACACCATCTCAAAA |
| | AATAATAATAATAACAACAATAATAATAAAAGTATTTGGACCACAGGCTGCACGT |
| | CTCTCTCACAAGATCAGAGTGACCTGGCACAGTGTCACTGCATAACAGCTCTGGTC |
| | TGAGGGTTTCAAAGACATTAACTGGTATCATGCATAATGCAAGATAGATGCTGTTG |
| | TTCTTTCCATTACACATATAAGGGAACTGAAGCAGAGACTCCCTTAAGTTGTCTTCT |
| | CAAGGTTTCCCTATTTATTTATGATGGGGCTTAAATTGAAAAAGTCCTAAGGCACA |
| | CAGATGTGTCTTTTTTTTAAGCAATTATACAGAGCAATGCATCTCTTCAAACGTTAG |
| | TAATGCAGACACATGCCGGCATTTCATAGTTTCATTAGCAAATGACTAGGACAAAA |
| | TCTAAAGAAATATTGTAAAGTTCATCATGATGCTTCCCTCTGCACTTAAAAATATTA |
| | TCACTTCATAGGGAGGTTTGTTTTTAAGTTTATCTCTGATAATTTAATTATCTCACTT |
| | TGGAAATATGGCAATGTACTGCTCATTTGACTAAAATTGGATAAAATAAATGAAGC |
| | TAAGAAGGATGGTATGGTAATATTAGCAATAGTTTGTACTTTATTTGAAATACGTTT |
| | GTATTATAGAAATAAAAGTAAACATGGGGAAGTATGTTAGATGCTGTTGAAAAAC |
| | AGTGCCACATTGTTTCCCTAACATTCCCAGTGATTGTCAAAGAGCTGCTTTCTAATT |
| | ATAGAATAGTATTTAAATAGGGACCAATGACATTCTAAAGAACACTAATAGAAAG |
| | TAGTTATTATTCTCCTGTATTTCTTTAATAATAATAGCTACTTTCTATTAGTGTTCTT |
| | TAGAATAGAACCTTGGTTGAGTGGTTACTCTGGTCAGTCTACCCTTGATTTTCTGTC |
| | TTGGATGAGTTTGCGTTGTGTGTCTACAAAAAAATCTCACAAACAGCAGCATATGA |
| | GGAATCACATTAAAACTTCTTTGAAAAAAGAAAGTATTTATTATAAGCAACATAAT |
| | TTCCATTGAAAAGTAAAAGAATGGAAAGTCAATTTCTAAAATTAAATACATAAGG |
| | ATAACTCACTTCAATAAATTGAGTAAGTTTTGCAATTATAGAATTATATTTTCCTAA |
| | ATTCTCATGAAGAAGTAAACATCTGTAATTCCAGAACTTGGGAAGCTAAACCAAGA |
| | AAATCATGAGTTTGGAGCTAACCTGAACTTCATAGTCACCCTGACTCAAAACAAAA |
| | GTTCTGATTCCTGATAAAGAGCAATTTGATACTCATTCTATATGGGTTCTATTATGC |
| | AATCTTTAATATACAAAACAGAATACTTTAAAATGACATTATTATTGTGAATTGAT |
| | GGAAAAAATAGACAAAGCTTTATTATAAAATTAAATATAAGTTTGTATGAAATTCA |
| | TAATGTCCTTTAAAATGTGAATGACATATTAGAAAAAAAGTCACCTTATCAACTGT |
| | GAATCTAATTTTATATATAGTTACATTATCTTTATAACTGTTTTAAGTCCTGTCAGA |
| | AAAAAATGTATAATTAATGAAAGATGATGACTGACAGCACCATCTCTCAGATCAG |
| | GAAAACCAGCTCAGATGAAATGAGAAAAGAATACAATGTTCACTACCAAAAAATC |
| | TATATCAGGTAGTTTATTTATTTAGATTTTTAACATTGTTCCTGGTCTATATATCAAG |
| | GTGATGCAGATAGATAGATAGATAGATAGATA |
| Mouse CPS1 | TTTAAATAATTAACCTGATCTATTTCATTGACTGGAAACACATCTTTCATTTAACGA |
| RR37_v1 | GCTAAATTCTCTTCTTAGGTGCAGCTCTGCGGTCCAAGCCCTCCATTTAGGGAACTG |
| SEQ ID | GCTCGCCATTCCAATTCAAGACAGTTGTTTAGCTCATGTGATTGATTGGGTGGTAG |
| NO: 84 | AGAGGGGAAAGGTGGTAGGGACCAGTGGTGTGCTCTGGACCATCCATGATTTCTGC |
| | AGAGGGATGAATTGTTGGGGGCCCTGTATTGGCCCTCAGCGCCTTGTTTGCTGCTC |
| | AGTCTGTTCACTGAGTGTGGTACTGGATACTGGTTCAGCCATGTGATACTTAGGTGT |
| | GTGGAGTAATCTGCCTCAGGGAGTCTTTTTATCTGGATCCCAGACCTCACATTGTTT |
| | TCCAGTTACAATAATCCAAAAGGAGTTCAAACATATAAGCGGGCAAGGCCAGTAA |
| | GATGACTTTCAAAACAGAAAAGAACACTAATACATTAAAATGTTATTTGGCTTGAT |
| | TCATTAATGGCACAGTGTGTATATATATATATTCTTATTGTAAAAACATATATAATT |
| | TTTGCACACATTTTTGTATCTCTTTAAGTTACATATTGAAAAGTACAATTACTATAG |
| | GATATACACAATTGATATTCCACTATATAATGAAAAATTGCATTAGAACATATTTT |
| | ATTACATCTTATTTTGTGGTGTATGTGCGTGTGTGTATGTAGAGGACAACTGTGGGG |
| | TTCTCACTCTCCTTCCATTATGTGGATCCCGGGGATGAAGTTAGTCTTCAGGCTTGG |
| | TGACAGGAACCTTGACTGGCTGAACCTGCTCGTCATTTCTGCTAGACAACTTTAGA |
| | GTATTCCTTGCTAATGGTAGACATTTCATATAACTTACTTTATCAGAAGTCTCTGGA |
| | TAACCCTCCACAGCTGTGAGGATCATTCCCATTCTACCTGTGAACAAACAGAGGTC |
| | TGAGGAGTCAGAGCAACTTGCTAAAGGATGAAGGCTTGAAAACTTTTTTCAAGCAA |
| | GCACAAGAGTTATCTTGATTCTGGTTTATGCTCAAAGCATGTATTCATTTTGACCCA |
| | ATCCTTATAATGTTGACTGTTCTTGTTTTCCTTGTGTCTTAATAATTGAGGATAAAA |
| | GTAGAAGATCACATTATAAATAATGGACAATGATACTCTGTTCCCCATCAGCTCGG |
| | TTTAGAGAGCTGTTTAATGGTTCAGGGCAGATTGTTGGCTGCATGTCTTTTTGGCCC |
| | ACAGCCCTTAAGTACACAGGGAAGAGGCACAAAACTCAGTTGAGTTGGGCGTGAG |
| | CTCAGTTTCAGCACTAGGGACTGTGCGGCCTCAGGCATTGCCCCAAGACACGAATC |
| | ATCAGGTGTACACTCTGTCCAGAGGGACCCATATTGTTCCAAGTCAGCCTCAAGTA |
| | CTACTAAAGGCATCTTGATTGAGACTGACATAAGCCAATCCTTCAGGTCAGAAACA |
| | GAGAAAAACACTTTGAGCTGAGGAGTGATATTTCAAATTCTCTCTTTTTGAGTCTGT |
| | CGCCAATCAAGCTTCTCCAGGAGTAAATTGTCAGCTAATGAAATGCATGTATCATA |
| | GCCCAGAGTGTGGCCTATTTGCAGAGCTGGCTCCAGCCCGAGGCACATCTTTGGCT |
| | GCTCTGTTCTATCTATAAGTTACAGCCGTGGCCTCGGCAAGCAGTACACCCTGTTCA |
| | GCAGCTGCAACTCGTTATCGGCTTTATCCAGAATAACAAACAGAACAAACACGTTT |
| | TGCAAGGGTTTCTCTCCTCTTATGGGGAGAAGGAGGAGTGAAGCAATCGGTGAAAT |
| | GCTGATTGATCAATTGTACTGGCATAGAGAGGTTCAAGGCCACATGAGTGCTATTA |
| | ACCCTGTAAAATGAAAAAAGAAAATCTCAACAAAGATTACTCTTAGAACTTTATTT |
| | ATACTAATATTTCTGTGATGTTCAATTTTGCCTAAAGAAAGGAAAGAAATTTGCTTT |
| | GTTTATTATGATTTTCTCATATTGATGGTGGTTATGCCTTATAAACAGGATTCATAG |
| | TATTTGGACTGT |
| Mouse CPS1 | AGAAAGTTCAGTGTTATTCAGCGGCAGATTTCCTCACAGGGCAAAGCATTGCATTA |
| RR38_v1 | ATTTATGTTGGCTGAGTGAGTCACAGTCTGCTCTATTTATTTTTCCCCTGTCAACTTC |
| SEQ ID | ATTTTGTACAGAGAAAACTTTAGTAAACACAAGGCGGATGGTTTTTGACACAGGGA |
| NO: 85 | GTGTCAAGCCACTGTAAAGAATCAGTTATGTATTTTAGCTGTATTATTTTACAATCA |
| | TGTGGTCCCCCTCCCCCCCCGGGATTTTTTTTTTTCTTCTTCTTTGCCCCCCACTCTA |
| | TCATGTCCTTAGTAATTTTTTCTGGTGAAATTTGTATTGCTAGAGGAGAAAAGTATC |
| | ATGTGGGATTGTGAGTACTAGAACAGGCTGGAGTCCAGCCAGCCGTCCTAGTGAA |
| | GAATTCACAGAACTGGATCTTCCTCTCCCTAGTCTCTGCCAGAAATCTCTGTTCACA |
| | GTAATGCTCAAAAGCCATTTTGGCCTATCACCGTCCCTGTCACACCTAGAATATGA |
| | TCTCAAAATTCTACTTTCAAACTTTAAGGGCGGAAAAAGGACCATGTAAATGAAGC |
| | AATGATGCTAAATTAATATATTTTCTACTTTTGTCTTATAGAAGCATTTACTTCAAT |
| | GTAAATCTTATTTAAGAAAGTGCTTCCTCGTTAAAACATAGACTTTCATGGTACTTC |
| | TCCTGATGTCGACGTGAAGTGAAGTAGCTACAAAGGCAGTTTGTATGTGCAGACAA |
| | CTCAGAACTTAGTTTTGAGATATTAAAACTTCTTATTACCATAGTTGCATTCCTACT |
| | CCTCAGGTCACTATAACATTTTAATTTTCTATAATCAAGACAAGTTGGACTATATCT |
| | TCACAATAAGTACATTTATTTCTCTTACCTTGTCTACCAAAAAGAAAATTAAAATG |
| | GAAGTTTTTTCTTTTTTCGCCATCCTAATGGACAACAAATCTTTTGTGCTGTGTTCA |
| | AAGAACAACCAAGGTGTTTT |
| Mouse CPS1 | GAGCAGTTTGTTCTGTTCAGCACATTATAAGAGGGGGAGGGTTTTGGCAATAATAA |
| RR39_v1 | TCACTGTGCAAAGAAGGGCTGCTGATGAATTTGCTGTTTTCAAAAAACATTTTCGT |
| SEQ ID | AGAAATAATTTACTAGTTAGAATGAGCACATATCTAATGATTTAGTCATCACACTC |
| NO: 86 | TATGAAATTTACACTGTAAGCATGTTTTGGTCAAGTTTTAAGAAACTTCTTTGACCA |
| | GGATTAGCTAAACTGACACAGTTAGCATCTTTCTAATTGTTTTAATATTCATCAAGT |
| | TATTTTTTTAAAGGTATCAATTATGTGGAACTCAGGTCTAGATTGACCCTTTTTTCC |
| | TTTTTAAAGAAAAGCTAAACTAAAAACTTTTACTAAAGGCTATTTAGTACTGAATA |
| | ACTCATAAATAACGAGATGACTCTATATTTATCCAGATGCCAGATTTTTATATATAC |
| | AAAACAAAAGAACCAGAAAACTATTTTAAAGGTTTATTTTTTAATCTATCTAAAAC |
| | TATCTATATAATTTATACTATTTGCCTTCTAAAAATGTAGGTAAGGCACTTAGATGT |
| | TGTGAATTTTCTTGAAATCATTTTTAGTTAAAGCCAATACTGTTTCCTGATATATAT |
| | TGATTGATATCCTAGCATTTCTTAGTAAGCATTTCCTGAATTCTTTTAGTTACTCTAC |
| | ATCTTATTTCCAAACAAATATAAGAATTCAGTGAGACTAAAGTACTCAGATCCTGT |
| | GGCTTTGGAATGAAAAATTATAAACAATAAGGTATAAGATAGAAAACAGTGAGAA |
| | CATGATTTGCTATTGTAGTTAAGTCTGGTCAAGTCTCAATCCAATTTACCTGATCTG |
| | AATTACAAAAACAAGATAGCCAGTATAATTCCCTGCTATTTGAATATTGTTGTTATT |
| | TTTAAATTTAAATGTAATACCAAAAGTAAAGTCAGAATTACTTTCATCCCTCATTTT |
| | TTGACATTTACTTAAGTTGTATCATCTTAACTTTACGTGATGGCATAAACCCTTCTG |
| | AGCTCTGAAATCTGTTCTTTGCACCTCCACTCTCGGGTGTGCTTTCTGTCTTGCTGG |
| | ATCTTGCCCAGCCCACTCTCTCCACCTCATTCTACACCGTCACACCCGCTTTTCCAA |
| | GTATGACCAATACACAGGAAGAGGATGGACATTGGCTAAGAGCTGAATTCTGATT |
| | CTGGCCTCGACTCTTTCAGTGTGGTGCTAGACAAGCTAGTTACAAAGCTAAGACAC |
| | ACTATCAGCTATACAACAGGAACAGCAATCGATAACCAGGAGTTGTTTCTAAACTT |
| | AATGGAGAAGGCAAGCAGGTGCCTAACGATGAGAATAAATGGCAAATTACTTAAC |
| | ATTATTCATGACTAGAACAATTAAAACAAGTTTATTTAAATATGTATTTATGCACAC |
| | ATAGACATACCTTTTATTATAAATATGCAATGTGCTAAAATTGTAATCATACACTA |
| | GTGTCTTAGCTTCTTATGTAAGATTTTTAAATTTATTATTGAATAATTTCCATTTTTC |
| | ATCATTAAAGATTAAGTTTGATTAATTAACTCTACATGACATGTTTTTAAATTTTAA |
| | TTGTCACCAAAGTAGTACTTTTTACATCTTTATTCAATATTTAACTTTACAAGTAAT |
| | TTTCAGTTTATTTTCTTAATTTCTATTCTAGATTCTGTTATCTAAATATACTTTATGC |
| | AAGCATAGTCAGCTATGACTAACTTAGGACCTCTTGCTATTCAATATTAGCACTAC |
| | ATATGCCTGTCTTCCAAAAAACAATCTATCCACCACAGTTTTCTTTTTTGGCAGACA |
| | TTTTTCCTCCATCTTTTGGACTGACTTACATCTGACCTTCCCACTTGGATTTTGCTCT |
| | GCTCTGTTTCTGTTTAACTGTGCCTTCAATTTTTCCAAGGTCACTTGAATTCTGAATC |
| | AACTTTCGAAGGAGTTGTCTATTTTCTTTTCATTTCACTTGCCCTCAACATACTTGGC |
| | T |
| Mouse CPS1 | TCCCTTTAGCAGGATATTTGATATAATAAATCTGACACCTTCTGGGCCCTCCTGTTT |
| RR40_v1 | CCAAGGGAATTTGATCTAGGAAGATGTGTTTCAGATTCTCGGAAACTGGAATAATG |
| SEQ ID | CTTTCACAAACTTTAAGTTTCATTATCCTAAAATGTTATCGCAGCTCGCCAGGAAAG |
| NO: 87 | GAACGCACAATTATTTCTCCCTCCAATGTCGAAGGTTGAGAATGGCATTATTATAA |
| | GGCATGAGTATTGTTGTTGTTGTTGCTGCTGCTGCTGCTGCTGCTGCTGCTGCTGTT |
| | CTTGTTCTTGTTGACGTGGTTATGGTAGTGAGATGGAGTTGGTGGTGGTGTTGTTTA |
| | TAAAGACTTTTTATTTTAGAAAATCAATGCTATGAAAGGAAAATGAGAATAAGCTA |
| | GAAAGGAAGGACAATAAAAGGAGAAAGAAAGAGAAGGCGGCAGACTGATCTCTG |
| | AACTCTACTAACTGCTAACAATTAGTCAAAGAACCTCCTTGGATATCAAATTTCTG |
| | ATATAGCTTTGAGTATTGGAGAATTGGTTGGTTTCTTTTTTCTGGTATTGTTCACTTG |
| | ATGTCATTCATCCTTAACCTGCTCTTGCCTTCACTAAGATCATTCACAGCTCAGAAG |
| | AATTAAATATATATTCCCATCAATTGATTCTGGGTTGGAAAATAAAAAGTGTTCTT |
| | AAAAACTTCAGGAAATTCAAAAGCAAAGCAAGGATCATTTTTTAAAAAGTTAAAG |
| | ACTGTGAAAAGATTTCAAAATGTTGTATTTAAAAGTTACCTGACTAACTGGGCATT |
| | TGGCGGGTGGGCCTGTAAAATCTCAACACTTGAGAGATGGGAAGGCCTGAAGTTC |
| | AAGGCCAGTCTTTAGCTGCTACATAGTAAGTTTAAGACTAAACTGTGCTATGTTAG |
| | ACCTTGTCTCAAAAATGAAAAGCAAATGTTATTGTGGTGAGAAGCTGAGGCTGAA |
| | GTAGTGTTCTAGGAGTCTGACAAAAAGAATGATTACACATGTTTAAAGAGCAGGG |
| | GAAATGACATAATCAAAGTACTGTGTTAGTATAAATCGTACATTAACGTGTTGCTT |
| | GGAAAAGGGCTTTTCAAATGTGAATAATAATTATGTTAACAATGGTAAATGCATTC |
| | TTTAAATTATTAAATATTCTGACTCAGTCACTTGCCCATGTCTGGTTTCTACAATTG |
| | TCTTCTCTCTCTCTCTCTCTCTCTCTCTCTCTCTCTCTCTCTCTCTCTCTCTCTCTCTCT |
| | CTCTCTCTCAGAGCTGAGGACTAAACCCAAGATCTTGCACTTACTAGGCAAGCGCT |
| | CTACCACTGAGCTAAATCCCCAACCCCTTCTACTGTTGTCTAATCAATTTATTTGTG |
| | TTTGATTGACTCAGCAACACTTATCTTATTACAATCCATCTATCTTTTAAATCAAAG |
| | ATAAATGAAGGGCTCTTAGGGAACAGGCTTATAACATAAAAGTGCAAAGGCCCCA |
| | GTATAAAATGTTACTACTCTCATAGGAACTGAGGACTCTCTAAGAAGTGGAACCAA |
| | CTCATCTCTAAATCTCAAACACATACAACTGAAGATCATATAGATAGATTGGTTCA |
| | CAGACAAACCTTTAAATAAAGGACAAAAATACTTTAAATAAAGACTTTTATTGCCT |
| | GTGGTTGAATTAGGGAAAGACTGGAAGAAGCTGAGGAGGAGGGCGACCCTGTAGG |
| | AGGACCAGCAGTCTCAATTAACCTGGTCCTCCGAGATCTCTCAAACACTGGACCAT |
| | CATGCACCAACTGATATGAGGCCCCCAACACATACACAGCAGAGGACTTCAGGAT |
| | TTGGTTTCAGCCAGAGAAGATGTACCTAACCCTCAAGAGACTGGAGGCCCCAAAG |
| | AGTTTAGAGTTCTGGTTGGGTGTGGGGTGGGTGATAGGGACATACTCGTGGAGACA |
| | GGGGGGCGGGGAGGAGGTATGGGATGTGGAAAATTAGGAGGGTGGACCAGGTAG |
| | GGAATAAATCTGCAGTGTAAATTAATTAATTAATTTAAAAAAACTTTTGTTGGCAC |
| | TTTATTTTTTTTTCTTTTCCTCTCCAGAGGCTGAGGCTGGACCACCTGACCATAACA |
| | AAAGCTCCATGCCTTCGTCCAGACACTTTTCAGGACGGTAATTTTTATTCACACGTG |
| | AATCAAAACACCTTGGTTGTTCTTTGAACACAGCACAAAAGATTTGTTGTCCATTA |
| | GGATGGCGAAAAAAGAAAAAACTTCCATTTTAATTTTCTTTTTGGTAGACAAGGTA |
| | AGAGAAATAAATGTACTTATTGTGAAGATATAGTCCAACTTGTCTTGATTATAGAA |
| | AATTAAAATGTTATAGTGACCTGAGGAGTAGGAATGCAACTATGGTAATAAGAAG |
| | TTTTAATATCTCAAAACTAAGTTCTGAGTTGTCTGCACATACAAACTGCCTTTGTAG |
| | CTACTTCACTTCACGTCGACATCAGGAGAAGTACCATGAAAGTCTATGTTTTAACG |
| | AGGAAGCACTTTCTTAAATAAGATTTACATTGAAGTAAATGCTTCTATAAGACAAA |
| | AGTAGAAAATATATTAATTTAGCATCATTGCTTCATTTACATGGTCCTTTTTCCGCC |
| | CTTAAAGTTTGAAAGTAGAATTTTGAGATCATATTCTAGGTGTGACAGGGACGGTG |
| | ATAGGCCAAAATGGCTTTTGAGCATTACTGTGAACAGAGATTTCTGGCAGAGACTA |
| | GGGAGAGGAAGATCCAGTTCTGTGAATTCTTCACTAGGACGGCTGGCTGGACTCCA |
| | GCCTGTTCTAGTACTCACAATCCCACATGATACTTTTCTCCTCTAGCAATACAAATT |
| | TCACCAGAAAAAATTACTAAGGACATGATAGAGTGGGGGGCAAAGAAGAAGAAA |
| | AAAAAAAATCCCGGGGGGGGAGGGGGACCACATGATTGTAAAATAATACAGCTAA |
| | AATACATAACTGATTCTTTACAGTGGCTTGACACTCCCTGTGTCAAAAACCATCCG |
| | CCTTGTGTTTACTAAAGTTTTCTCTGTACAAAATGAAGTTGACAGGGGAAAAATAA |
| | ATAGAGCAGACTGTGACTCACTCAGCCAACATAAATTAATGCAATGCTTTGCCCTG |
| | TGAGGAAATCTGCCGCTGAATAACACTGAACTTTCTCTGTTTGCCTGTATTCGATGA |
| | AATCTTTGAATTCCTACAGAATGCTGTTCTTTTTTTTTTTTTTTCTGACATGAGACAA |
| | ATTCTTTATAAACATCTTGTTTGGATCAGCCACCACATTGTAACATTGTTTCTTACA |
| | CTAAGGCTACTATTCTATAAAACTGTTACATATCTCCACTCTAACTACAGTGCAAA |
| | GCTAAAAAAAAATTAAAAAAAATAAAAAAATAAAAAAAAGCAACACCTTCAGTTT |
| | TCTAAGACTTTACTCTCATTGCTTCCACATTTTGGATGTATAATCCTAAATTAATCT |
| | CTTCCTTGCCTCTTTTTAGCATCAACAAATAGGATATTTCCTAATGTAGCAAGCATT |
| | AGTTCTATGAGCCATGAGGGGTAGTTTTTAACCCCTCCTTCATTCAATGTTAACTAA |
| | AAGTCTTTTACATGCCAAGAGGGCTACAGTCAAGCCACTGTAAAGAATCAGTTATG |
| | TATTTTAGCTGTATTATTTTATTTATTTTATTACTTTATTTATTATTTATTTTCAAAAG |
| | TTCTGAAAATAAAAATGAGCTCAGAAACTCTCTCTGCAAACTCACTCTCAAAGAGC |
| | CTTTACAGTTTATTCTTAGAAAGAGCCATGTCGTATCTAGTCTCAAGACTAAACAAT |
| | AAAGAAAGACAAAGGGAACTGTAAACCTACAAATCTCAAGACCCCAAGACCCAAG |
| | AACAACAGACCAAATCAATATTCCTCTCCAGCAGCCACACGACGATGAAGTGTGA |
| | GCATGTGTCTGTACACTTCACAAAATACAAAGCCCCCTGCATCAGCTTTCATATCCC |
| | AAAGGAGTTGACCCACACTTCTAGGAAGCCCTTCACATCCTACAACTTCTCAGAAA |
| | ACAAGAAGAATCAGATTTTTTTGAGCAGACAATTGTGGCTGGAAGTTGAGTTATTA |
| | TTACTGAGCAAATTGACTAAACTTTTAACATATGCAGGACTAAAAGATAAAGTATA |
| | AAATGAAAACTCATATATATGAGTAACTTTTTAAAACAAATGTTTTCTAAAAATAT |
| | ATGAAAGATAAGTCCAAGATCAAATGTGTTTCATGGACTTAGATTAATAGTCAATG |
| | GTAGAAACATGAGTCAAAACATTTTTTCATAAAATAACAAATCTTAAATTAAAACC |
| | CCAAATACTCATGAAACAACAAATCTTTAGCTCTTATCCCACCTAACAAATATACC |
| | ACAATATAATTACTACTTGACAGAGAGAAAAGTAAAGGTTCATACCAGATTATGTT |
| | AAAGTTATAGGGATTTGTAATCAATTTTCTCAAGATCTCCTCATCAGAATCCAATG |
| | AGTCACTCAGCTCACCACTGTTTCTTCTTTTCCAAATCTTGACTGTGAGCAGCTAAG |
| | AAAAGATGAGCCACATCAGAATCAAGGCAAGCAAAGTTTTGACTGTTCAAGCTTA |
| | GACTCAGGATGGCTGGAAACAAGACTTCCATGTCCAAGCCCAAGTCAGGACAGTC |
| | CTTTTTTAAAGCAGATTAATCTTTTTCTTCAGACTTGAAAACTCTGTGCTATGTCTA |
| | GGAAAGTAACTACTCCGCTACCTTGATACTGAACTCTGTGTTTCTCCCAGACCTAA |
| | GATAAGACACCTTTCTTTATCTCCTCTGGGGAAAGAAAATGGCTTCAAAGTCCAGT |
| | CCAGGAGACAAGCCATACGGCAATGCCAATAGTATCAGAGGCCTTGTTGGATCAC |
| | CTGACTTAGAATTACTATAGGAGGCAATGTAACAAACACTTCAATTTTTAAAAATC |
| | CAAAATTAGCATATTTCCTTAGTAAAAATGGTCACGAATGTCTTACAGAATGTCAC |
| | TATTAAGTGACAACTATTGGTTTGCACATTAACTAATGCTTCTGGATGATTTAACCA |
| | ACAGAAATGTGCTACAACTTACAACTTTAATCTC |
| Mouse CPS1 | TTGGTCATACTTGGAAAAGCGGGTGTGACGGTGTAGAATGAGGTGGAGAGAGTGG |
| RR41_v1 | GCTGGGCAAGATCCAGCAAGACAGAAAGCACACCCGAGAGTGGAGGTGCAAAGA |
| SEQ ID | ACAGATTTCAGAGCTCAGAAGGGTTTATGCCATCACGTAAAGTTAAGATGATACAA |
| NO: 88 | CTTAAGTAAATGTCAAAAAATGAGGGATGAAAGTAATTCTGACTTTACTTTTGGTA |
| | TTACATTTAAATTTAAAAATAACAACAATATTCAAATAGCAGGGAATTATACTGGC |
| | TATCTTGTTTTTGTAATTCAGATCAGGTAAATTGGATTGAGACTTGACCAGACTTAA |
| | CTACAATAGCAAATCATGTTCTCACTGTTTTCTATCTTATACCTTATTGTTTATAATT |
| | TTTCATTCCAAAGCCACAGGATCTGAGTACTTTAGTCTCACTGAATTCTTATATTTG |
| | TTTGGAAATAAGATGTAGAGTAACTAAAAGAATTCAGGAAATGCTTACTAAGAAA |
| | TGCTAGGATATCAATCAATATATATCAGGAAACAGTATTGGCTTTAACTAAAAATG |
| | ATTTCAAGAAAATTCACAACATCTAAGTGCCTTACCTACATTTTTAGAAGGCAAAT |
| | AGTATAAATTATATAGATAGTTTTAGATAGATTAAAAAATAAACCTTTAAAATAGT |
| | TTTCTGGTTCTTTTGTTTTGTATATATAAAAATCTGGCATCTGGATAAATATAGAGT |
| | CATCTCGTTATTTATGAGTTATTCAGTACTAAATAGCCTTTAGTAAAAGTTTTTAGT |
| | TTAGCTTTTCTTTAAAAAGGAAAAAAGGGTCAATCTAGACCTGAGTTCCACATAAT |
| | T |
| Human CPS1 | TTCTTATCAAATACCTTGTCATTGCAACAACTTGTAGTCCATAAAAAACATAAGGA |
| RR89_v1 | CTCTTTGATCTAACCCTCTACTAAGTCCTTCTACTAAAATGTATAGAATGGAATTTT |
| SEQ ID NO: | TTTTTAAATATCCAGTTTTTGCTTTAAAATGTTCAGTTTAATGGTTTAAATTTAAAA |
| 89 | CTTTAAAGATTTTTGAGAATTGTTCATGTGTGACAATCACATAATTTGTCAAGTAAA |
| | TCAGTCATCATTTGCACTATACAGATGACAAAACTTAGGTTCAGAGAATATGTGAC |
| | TTGTCCAAGAACAGGGCATTAGGAAATGCATAACAGGGGTTAGATTCCAGATCTTT |
| | TTGTGCCCAATCCAGAGTATATCCCACTATTACACTGTTACAGCCTGCTGAATGTAA |
| | TAATGAGTGCTGAACATTTTCAGTACTAATGGAAAAAGATTATGGTATCTAAAACT |
| | TTTTAGTATTTAGTATTTTATGGTTGTTGATCATTTATACCCTTGATATCAACTTTCT |
| | TTAAAATATAGAGCACCTTGAAATTCTTTCCTCTTTCCTGTAAGTAAAATACAACTG |
| | TGCCGATTGGCAAAGCACTCTATAAATATTAGCCGCTGACAAATACCCAGGGAAG |
| | GGCCACATAAAATTATAAGTGCGTCATTGCTGTCGTCAAACCCACCGTCTCTGTCA |
| | TCAAAGAGAGAGGAAAAGCAAACCTCTAAATATGTCGAAAAAGCAACCCAATTTA |
| | CAGGGCAATGATTATAGATGTGACTACAGGGTGTCTTTCCCAATCCATTTACCATCT |
| | ATAACTGGACATAATGCTACTAACCTTTAAAGTGACTGTTCTCAGTGCCCTTCACTG |
| | ACCATTATGTAACTACATCCTAATGATTTTAACTATCTTTAAAATATGATGTCCATC |
| | TAAAAATAGTAAATTTGGATATATTTTTCTAAGACAGTTGAGTTTTTAAAGTGAAG |
| | GCAGGAAGAAAAGTAGATAGAGGACTATTTCACAATAACTGAATAATAACACAAG |
| | TCCCCTTGGGCTGTTCTATGGAATTTTAATTCACTATTGTGAAGTTCAAGAACTTCT |
| | ATTTTTTGTGCTTGGCATTCCTCCAAAACTCTGGCCAGTAATGATTGAGTGTGAGTA |
| | ATGGCTTTTACACCATGAAATGATGGCTTAGTGCTCCTACAAATTTGTCATTCTTAG |
| | CTGTGCACTGTGACAGACCCTTGAAACAGATCTGTCACCATGAGCTAGAACTGCTT |
| | TCACAGTAATCCATCCACATAATTAGACCATTTTGTCGTGGTTGTGATTGTGGTGGT |
| | GGTTGTATGGAGCACTTATTGTGATCTTGGAGAATTGGATTCTAATTTCAATCATAT |
| | CTGTGCCATTTTAAGTAAGTACTCAATTTCGCGGTTTCTGGGGTCTGATGAAGATGC |
| | ACCTATCCTTACCTACATACCTACCTTCTCACAGAGTGAGAAGAAGATATTCCAAG |
| | TCACCCAACTTTGAAGTATTAGATGGCACATATAATCATAGGGGATAACTTTCATT |
| | ACTATTGATTTTCTCTGAACTGATACATGAGTACCTTGAGGTATTACTAACAATAAA |
| | TTCTCATAGTTTACATCTAGTTACAATGTCTTTGATGTTAGTTTAGAATATTCTCCTT |
| | TCCCATTCTTTTGCTATAATTAAGCTAAATTCAAGAAAATATATATAACTAGACATA |
| | ATGCCACTAACCTTTAGTGACTGCTCCCAGTGCCCTTTATGGCCATTATGTAACTAC |
| | ATCCTAATGATTTTAACTATCTTTAAAACATGATGTCCATTTA |
| Human CPS1 | TTATGGTATTGCTGTTGAATCATTTATCTACGTAAACTATTTGCACTTCAGAAAAAG |
| RR90_v1 | TAGAGGCCCCACATGTTTTTTTTCCATTTTCACCAAGGCCTAAGGCTATATAACATA |
| SEQ ID NO: | CGTGCTTTCTGGCTTACCTGATGTTTCTAAGCTATTAGTTCGCCTCAGTACTCATCCT |
| 90 | CTTAAAAAATAAATCTGAACTAAGCACTGAAAAGTAGAAAGCCTAAAGCCAGAAT |
| | TTATTTCAGGTAATTATTGTGTAGATAATATGGTAAAAAGTTAAAATCAAGTAAAT |
| | GAAATCTTATTCTTTTATTCTTGGAATTACACTATTAAGATATGGATTTTTGAAATT |
| | TAGAATTTCAGCACAATTTGTCTTTGAAATATTTCATGAGACTTCTCTGCAGTTACA |
| | AGGCCTAACACTTCAAAATTCTGAAGCACTGTAACAGCAGTAATCAATTAGGCCTT |
| | ACCACATTCTCAGTTAATAATCTTACAACATTCCAAGTTAATATTAAGTCATCGGTC |
| | TTTGGTTAAACATAAGCCAATGTTAAGGGGACAAAATCAAAAGGTTAATTTACTTA |
| | AGTACTTTTATGTTTGAACATTTATAAACAAAAGATTAAGATTTATGAATCATTTTT |
| | ATTCTAATAAGGTTTCTCATTGTAAATAAAATACAGCATTTATAAAAATTTCATTAC |
| | AAAGTATGTTCATAAAGTGTAGGTTTATTAATATGCCTTACTGAGCATCTGCTATGT |
| | GGCAAGTTGTTCAGAATCCTGGAGGAACCTACATATCATAAGCTAATTTTTACTGT |
| | ATGGTGGTGATAAATTAAAATGGTCTTATATTTCAGTCTGCATTATAGAAGAAACT |
| | TAGTAAAATTCTTTCATAATGTAGCAACCCTATTGATTCATTTATGAGAACTTCAAA |
| | TTAGTCAGTTACCTCCCTATGAAATTTAAAAGATCAATGTATAGCATTTAACAAGTT |
| | GAAAAAAATAAAAAGCAAACAGAGTAGCCTTGAAAATATTTTTATAAGTTCAACT |
| | CATTTAGATCACATGAAGAAAATCAAATGGAAGCTAAAGATCCCTAGGGCTAAGG |
| | AGCCTAACACAAACTTTGACCTTGGCCCGCGCAGCTTTTTTCGGGCCTCCGTCATTG |
| | CAAATGCCCCAGGAAATACAAATCCCATCAAGGGCAGTCAGCGTAGCATACCAAA |
| | CTCCTCCTGAAGGCTTGAAGTGAATTCTAGCATGCCCATTGCCTAAGAGATACTGA |
| | AAACTTTCTACAGCACCCCTCAATTTTTGGAACAGGTTGGAAATATGAAGAAAGTC |
| | TAATACAGATGTAAAGGCAGAGGAATAGTCTCCATTTCCATAGCAATAAGACCTAA |
| | TGTCACCTCTAGATCGTCAGAAAGTCTGGCTAGACATGAGCAAATTGGAATGAGGG |
| | AGAGGAAAGCACAGGTCAAGATTAGTACTAGTCCCTTGGGCAGTTCTACAGCCAG |
| | AATGCTTAGCTTCTGACCCAAAGCTGGGACTAGGGTACTGAGTGATGTGCTTAGGG |
| | CCTAACTCTCAAAGAGATACACCATGACACTAACAGTGACAGCCTCCTTAAATTTT |
| | GCATCTCAGGTACCTCACTCACTTAACCTAGTCTAGGACATTTAAACTATAGTTTTT |
| | TCAAGAGTTTTATGTCAAGAAGCTCATAATGTCAAGGCGTTGTTGGTGACTAATAA |
| | ATGAATCTATATTTTAGAAAATTGGGGATATAAAAAATGGAAAAAATTAAAATGTT |
| | CAAAAGAAAGGCCATCTCCCAATTAGAGAAGTATTGCATGCCAAAAGTATGTAAA |
| | GTTGTCCATCAGGCAAACTAATTCTGCAACAGCTCATTTGGGACTGAACACAATTA |
| | ATATTTCAAAAATGTAGTTTGCCATAGATGAAATCCGTCCTAAAATGTGTCTATAT |
| | GTAGTAAAAGTCCAATAAAATAGTTATTCTTAAAAGTGTGTGATAACCCCATCCCC |
| | ACTCTTACATCTAACGTGTACAGAGGCTCCAATATGATAATGAGTTATATTTCCTGT |
| | GACTATCTTTAAAGCTACTACAATGTAAGTAAATTATTCTAATTGTAGGAGTTTTTA |
| | TAGTTGCCAATTTCTAATCAAATGACAATAGCTCCTCAAGACGGAACTAAATAGAT |
| | ACTTCTATGTGAAAATCAGAATATAAAATCTTAAAAATGAATGCCTTTCAAAAGAA |
| | ATAATGACTACCAGTGCTCATTCGTAGCTGAAGTATCAGAGAGGACACTTGGATTG |
| | TTAGGTTCCTAGCTTAATTATAACACAGGTCACCATTGTTAAAAAATGAGTGGATT |
| | AATGTTAGAAGGAGTACTTATTGAACACTTTTCTATTCATAGGGAACTTAAGCTTTA |
| | CAATTTAATACTTTTGATTTCCAAATTCAT |

The present disclosure describes ASOs that increase the amount or stability of the target regRNA, thereby to increase expression of the target gene. This is different from the ASOs previously described that were designed to inhibit eRNAs (see, e.g., PCT Application Publication No. WO2013/177248 and PCT Application Publication No. WO2017/075406). Without wishing to be bound by theory, it is hypothesized that the ASOs' ability to upregulate regRNAs is attributable to the selection of a target sequence in the regRNA and/or the chemical modifications of the ASOs.

### Sequences of ASOs

As disclosed herein, ASOs that bind a sequence closer to the 5′ or 3′ end of the target CPS1 regRNA are more likely to upregulate the regRNA. Without wishing to be bound by theory, it is hypothesized that such ASO hybridizes to a terminal portion of the target CPS1 regRNA and prevents or slows 5′→3′ and/or 3′→5′ RNA degradation without blocking the functional region of the regRNA. In certain embodiments, the ASO disclosed herein is complementary to a sequence in the target regRNA that is no more than 300, 250, 200, 150, 100, 50, 40, 30, 20, or 10 nucleotides from the 5′ or 3′ end of the target regRNA. In certain embodiments, the ASO disclosed herein is complementary to a sequence in the target regRNA that is no more than 300, 250, 200, 150, 100, 50, 40, 30, 20, or 10 nucleotides from the 5′ end of the target regRNA (i.e., the 5′ most nucleotide of the regRNA sequence forming a duplex with the ASO is no more than 300, 250, 200, 150, 100, 50, 40, 30, 20, or 10 nucleotides from the 5′ end of the target regRNA). In certain embodiments, the ASO disclosed herein is complementary to a sequence in the target regRNA that is no more than 300, 250, 200, 150, 100, 50, 40, 30, 20, or 10 nucleotides from the 3′ end of the target regRNA (i.e., the 3′ most nucleotide of the regRNA sequence forming a duplex with the ASO is no more than 300, 250, 200, 150, 100, 50, 40, 30, 20, or 10 nucleotides from the 3′ end of the target regRNA).

In certain embodiments, the ASO is no more than 25, 30, 40, 50, 60, 70, 80, 90, or 100 nucleotides in length. In certain embodiments, the ASO is no more than 8, 10, 15, 20, 25, 30, 35, 40, 45, 50, 55, 60, 65, 70, 75, 80, 85, 90, 95, or 100 nucleotides in length. In certain embodiments, the ASO is at least 8, 10, 15, 20, 25, 30, 35, 40, 45, 50, 55, 60, 65, 70, 75, 80, 85, 90, 95, or 100 nucleotides in length. In certain embodiments, the ASO is at least 8, 9, 10, 11, 12, 13, 14, 15, 16, 17, 18, 19, 20, 21, 22, 23, 24, 25, 26, 27, 28, 29, or 30 nucleotides in length.

In certain embodiments, the ASO is designed to lack a stable secondary structure formed within itself or between other identical ASO molecules, thereby increasing the amount of the ASO in a single-stranded form available to hybridize with the target regRNA. Methods to predict secondary structures are known in the art (see, e.g., Seetin and Mathews, Methods Mol. Biol. (2012) 905:99-122; Zhao et al., PLoS Comput. Biol. (2021) 17(8):e1009291) and web-based programs (e.g., RNAfold) are available to public users.

For example, ASOs have been designed to target a human CPS1 eRNA. The nucleotide sequences of these ASOs are provided in Table 2 below.

TABLE 2
Exemplary ASO sequences targeting regRNAs

| | | SEQ |
|---|---|---|
| Name | Sequence | ID NO |
| hCPS1-ASO-1 | TGCAGGCACACACATCAGGC | 1 |
| hCPS1-ASO-2 | ATGCAGGCACACACATCAGGCT | 2 |
| hCPS1-ASO-3 | AATGCAGGCACACACATCAGGCTG | 3 |
| hCPS1-ASO-4 | GCACACACATCAGGCTGGGG | 4 |
| hCPS1-ASO-5 | AATGCAGGCACACACATCAG | 5 |
| hCPS1-ASO-6 | TGAATGCAGGCACACACATC | 6 |
| hCPS1-ASO-7 | GGGACCCTGTGTACTTGGAA | 7 |
| hCPS1-ASO-8 | GCAGAGTGACAGGCATGAAT | 8 |
| hCPS1-ASO-9 | TGCCATTGACTTGCATAATG | 9 |
| hCPS1-ASO-10 | GTCAATGCTCAGAACATAGT | 10 |
| hCPS1-ASO-11 | TAGATAATAACCCACCACAC | 11 |
| hCPS1-ASO-12 | GTCAAGTACTAGGTTGTATT | 12 |
| hCPS1-ASO-13 | TTAGCCTGTGAAATCCCTCA | 13 |
| hCPS1-ASO-14 | CTTATGAGTCACTCTCTGAT | 14 |
| hCPS1-ASO-15 | TCTTTGTGAGCCAGGTCATC | 15 |
| hCPS1-ASO-16 | CGCAGCTGAGCAGAGTGACA | 91 |
| hCPS1-ASO-17 | CAGTCCTCCTTCATATAAGC | 92 |
| hCPS1-ASO-18 | TGACCACATGTACACAGTCC | 93 |
| hCPS1-ASO-19 | GTTACACTTTTAGGACAGAT | 94 |
| hCPS1-ASO-20 | GGTAGGGAATGTTAAAGTCA | 95 |
| hCPS1-ASO-21 | TATAAGAGTTGGTTGGTAGG | 96 |
| hCPS1-ASO-22 | CAAAGGAGTTGAGGCTATAA | 97 |
| hCPS1-ASO-23 | GGGAGTTTACATTAGGATAG | 98 |
| hCPS1-ASO-24 | GGCTAGACTGAAATCTATAA | 99 |
| hCPS1-ASO-25 | GCTAGTTTCATACTTGAGTG | 100 |
| hCPS1-ASO-26 | CACAATGGAGGTTGGAACAG | 101 |
| hCPS1-ASO-27 | TCCATCAATTATAATTCCAC | 102 |
| hCPS1-ASO-28 | ATTAGAGACTTGGAAAATCC | 103 |
| hCPS1-ASO-29 | GGTCCATGTCTTTAGTCATC | 104 |
| hCPS1-ASO-30 | AGACTTGCAAACTACGGTGT | 105 |
| hCPS1-ASO-31 | TTTTATCAGTAAAGGGTCCC | 106 |
| hCPS1-ASO-32 | AGTCTAGAGAGAAATGTGGG | 107 |
| hCPS1-ASO-33 | GGATAGAATTCTCCTACTCT | 108 |
| hCPS1-ASO-34 | CAACAATCAACAATTCTAGG | 109 |
| hCPS1-ASO-35 | TTCCATAGAAATGTCAACAA | 110 |
| hCPS1-ASO-36 | GACTACAAAGCATGATGAAT | 111 |
| hCPS1-ASO-37 | CATTTATAGCAGTTGACTAC | 112 |
| hCPS1-ASO-38 | GCTTGACCAAGGAAAAGACT | 113 |
| hCPS1-ASO-39 | GTGGAAGTACTGAGGAGTCC | 114 |
| hCPS1-ASO-40 | TAATCAAATATGACAGTGGA | 115 |
| hCPS1-ASO-41 | CCTCCCTTCATCACTTATGT | 116 |
| hCPS1-ASO-42 | ACTATAATATATTTCCTCCC | 117 |
| hCPS1-ASO-43 | CCCTAAGGTACCCATGCAAT | 118 |
| hCPS1-ASO-44 | GTCATAGTCCCATTACCCTA | 119 |
| hCPS1-ASO-45 | GGAAACTTTTTAGGATGCAA | 120 |
| hCPS1-ASO-46 | CCTCATTATGGAAACTTTTC | 121 |
| hCPS1-ASO-47 | CGATATAAACTCAATCCTCA | 122 |
| hCPS1-ASO-48 | GTAGTGAGGGTTGTCTTTCC | 123 |
| hCPS1-ASO-49 | CTCTTGTCTTTTTATGTGTG | 124 |
| hCPS1-ASO-50 | CGCCTTGATCTCACCTCTTG | 125 |
| hCPS1-ASO-51 | GTCCAATACTAGTTTACGCC | 126 |
| hCPS1-ASO-52 | AGTACTTACTGGCTTAGTCC | 127 |
| hCPS1-ASO-53 | CTAGAGAACTGAGGACTGAG | 128 |
| hCPS1-ASO-54 | GGAAAACTCCCAACCTAGAG | 129 |
| hCPS1-ASO-55 | AAATGAATTGGAGGAGAATG | 130 |
| hCPS1-ASO-56 | GGCGAAATTTGTGTTGAAGA | 131 |
| hCPS1-ASO-57 | TTGAACAAGAAATAAAGGCG | 132 |
| hCPS1-ASO-58 | GTATTCATAATAGTCATGGA | 133 |
| hCPS1-ASO-59 | TCAGCCTTCATCCATAAATA | 134 |
| hCPS1-ASO-60 | TATGCCTTAAAATGTTCAGC | 135 |
| hCPS1-ASO-61 | CATGGGAGACTTAGTATGCC | 136 |
| hCPS1-ASO-62 | TGAAAAGGTACCATATCATG | 137 |
| hCPS1-ASO-63 | GTATAGAATGGGAAAATGAA | 138 |
| hCPS1-ASO-64 | AGCCTAGATGCAGGGTATAG | 139 |
| hCPS1-ASO-65 | ATGAAGTCATGTGGTATATT | 140 |
| hCPS1-ASO-66 | GCCGTGGCATGAGCAATAGC | 141 |
| hCPS1-ASO-67 | TGCAAATTCACCAAGTAGAT | 142 |
| hCPS1-ASO-68 | TGTGTTATACTGAAGGATGC | 143 |
| hCPS1-ASO-69 | GCATAGAATTATGTTGAGCT | 144 |
| hCPS1-ASO-70 | AGATCAAGTTCCTTGTGCAT | 145 |
| hCPS1-ASO-71 | CTGGTGCCATGAAAGTTGCC | 146 |
| hCPS1-ASO-72 | GGCTCATACTAGTTTATACA | 147 |
| hCPS1-ASO-73 | AAGCTAAACAAAATAGGCTC | 148 |
| hCPS1-ASO-74 | CATGGCTCCTGGTGATAGAA | 149 |
| hCPS1-ASO-75 | GATTTCAAAGTGCTTCATGG | 150 |
| hCPS1-ASO-76 | AGTCCTCACGCTTACTTAAT | 151 |
| hCPS1-ASO-77 | CAGGCACACACATCAGGCTG | 152 |
| hCPS1-ASO-78 | GCAGGCACACACATCAGGCT | 153 |
| hCPS1-ASO-79 | ATGCAGGCACACACATCAGG | 154 |
| hCPS1-ASO-80 | GAATGCAGGCACACACATCA | 155 |
| hCPS1-ASO-81 | TGCAGGCACACACATCAGGC | 156 |
| hCPS1-ASO-82 | TGCAGGCACACACATCAGGCTG | 157 |
| hCPS1-ASO-83 | CAGGCACACACATCAGGCTG | 158 |
| hCPS1-ASO-84 | GCAGGCACACACATCAGGCT | 159 |
| hCPS1-ASO-85 | ATGCAGGCACACACATCAGG | 160 |
| hCPS1-ASO-86 | TGCAGGCACACACATCAGGC | 161 |
| hCPS1-ASO-87 | TGCAGGCACACACATCAGG | 162 |
| hCPS1-ASO-88 | TGCAGGCACACACATCAG | 163 |
| hCPS1-ASO-89 | TGCAGGCACACACAT | 164 |
| hCPS1-ASO-91 | TGCAGGCACACACATCAGGC | 166 |
| hCPS1-ASO-92 | CAGGCATGAATGCAGGCACA | 167 |
| hCPS1-ASO-93 | ACAGGCATGAATGCAGGCAC | 168 |
| hCPS1-ASO-94 | GTGACAGGCATGAATGCAGG | 169 |
| hCPS1-ASO-95 | AGTGACAGGCATGAATGCAG | 170 |
| hCPS1-ASO-96 | GAGTGACAGGCATGAATGCA | 171 |
| hCPS1-ASO-97 | AATGCAGGCACACACA | 172 |
| hCPS1-ASO-98 | TGAATGCAGGCACACA | 173 |
| hCPS1-ASO-99 | CATGAATGCAGGCACA | 174 |
| hCPS1-ASO-100 | TGCAGGCACACACATC | 175 |
| hCPS1-ASO-101 | CAGGCACACACATCAG | 176 |
| hCPS1-ASO-102 | GGCACACACATCAGGC | 177 |
| hCPS1-ASO-103 | CACACACATCAGGCTG | 178 |
| hCPS1-ASO-104 | CACACATCAGGCTGGG | 179 |
| hCPS1-ASO-105 | TGCAGGCACACACATCAGGCTTTTTT | 180 |
| | TTTT | |
| hCPS1-ASO-106 | TTTTTTTTTTTGCAGGCACACACATC | 181 |
| | AGGC | |
| hCPS1-ASO-107 | ACATTACTTTAAATGATTAC | 182 |
| hCPS1-ASO-108 | GATGTTATTACAAAGTTCTT | 183 |
| hCPS1-ASO-109 | GGAGGCAATATTATGTTAAA | 184 |
| hCPS1-ASO-110 | GCATAATGCTTTTTGGAGGC | 185 |
| hCPS1-ASO-111 | CTTGCATAATGCTTTTTGGA | 186 |
| hCPS1-ASO-112 | ACTTGCATAATGCTTTTTGG | 187 |
| hCPS1-ASO-113 | GACTTGCATAATGCTTTTTG | 188 |
| hCPS1-ASO-114 | CCATTGACTTGCATAATGCT | 189 |
| hCPS1-ASO-115 | GCCATTGACTTGCATAATGC | 190 |
| hCPS1-ASO-116 | TCTAGCATTTATGTCAATGC | 191 |
| hCPS1-ASO-117 | CTCTGTTAACACTAAAGTAT | 192 |
| hCPS1-ASO-118 | TCTCTGTTAACACTAAAGTA | 193 |
| hCPS1-ASO-119 | ATCTCTGTTAACACTAAAGT | 194 |
| hCPS1-ASO-120 | GATAATAACCCACCACACAA | 195 |
| hCPS1-ASO-121 | GAAATGTGGAAATACAGAAT | 196 |
| hCPS1-ASO-122 | ATGTCAAGTACTAGGTTGTA | 197 |
| hCPS1-ASO-123 | CCTTATTGACATATCTGGTA | 198 |
| hCPS1-ASO-124 | TCAGGGCACTTTATTTTATG | 199 |
| hCPS1-ASO-125 | GAAATCCCTCAGGGCACTTT | 200 |
| hCPS1-ASO-126 | TGAAATCCCTCAGGGCACTT | 201 |
| hCPS1-ASO-127 | GTGAAATCCCTCAGGGCACT | 202 |
| hCPS1-ASO-128 | TGTGAAATCCCTCAGGGCAC | 203 |
| hCPS1-ASO-129 | CTGTGAAATCCCTCAGGGCA | 204 |
| hCPS1-ASO-130 | GCATTTAGCCTGTGAAATCC | 205 |
| hCPS1-ASO-131 | TAGAAGCATTTAGCCTGTGA | 206 |
| hCPS1-ASO-132 | GTCTTCTTTTCTATTAATAC | 207 |
| hCPS1-ASO-133 | AGTCACTCTCTGATAAAATG | 208 |
| hCPS1-ASO-134 | GTGAGCCAGGTCATCAAAGG | 209 |
| hCPS1-ASO-135 | TTTGTGAGCCAGGTCATCAA | 210 |
| hCPS1-ASO-136 | CATTCTTTGTGAGCCAGGTC | 211 |
| hCPS1-ASO-137 | GGAAGTACATTCTTTGTGAG | 212 |
| hCPS1-ASO-138 | ACTTGGAAGTACATTCTTTG | 213 |
| hCPS1-ASO-139 | GTGTACTTGGAAGTACATTC | 214 |
| hCPS1-ASO-140 | GACCCTGTGTACTTGGAAGT | 215 |
| hCPS1-ASO-141 | TGGGGACCCTGTGTACTTGG | 216 |
| hCPS1-ASO-142 | ATCAGGCTGGGGACCCTGTG | 217 |
| hCPS1-ASO-143 | ACACATCAGGCTGGGGACCC | 218 |
| hCPS1-ASO-144 | GGCACACACATCAGGCTGGG | 219 |
| hCPS1-ASO-145 | ATGAATGCAGGCACACACAT | 220 |
| hCPS1-ASO-146 | AGGCATGAATGCAGGCACAC | 221 |
| hCPS1-ASO-147 | TGACAGGCATGAATGCAGGC | 222 |
| hCPS1-ASO-148 | AGAGTGACAGGCATGAATGC | 223 |
| hCPS1-ASO-149 | GAGCAGAGTGACAGGCATGA | 224 |
| hCPS1-ASO-150 | CAGCTGAGCAGAGTGACAGG | 225 |
| hCPS1-ASO-151 | TGCACGCAGCTGAGCAGAGT | 226 |
| hCPS1-ASO-152 | TGTCTGCACGCAGCTGAGCA | 227 |
| hCPS1-ASO-153 | AAGCTGTCTGCACGCAGCTG | 228 |
| hCPS1-ASO-154 | ATATAAGCTGTCTGCACGCA | 229 |
| hCPS1-ASO-155 | CCTTCATATAAGCTGTCTGC | 230 |
| hCPS1-ASO-156 | GTCCTCCTTCATATAAGCTG | 231 |
| hCPS1-ASO-157 | TTCTTCTCTGACCACATGTA | 232 |
| hCPS1-ASO-158 | ACATTTTAAAAAATAAGCAT | 233 |
| hCPS1-ASO-159 | ACACTTTTAGGACAGATTAT | 234 |
| hCPS1-ASO-160 | TGTTACACTTTTAGGACAGA | 235 |
| hCPS1-ASO-161 | GGAAATGTTACACTTTTAGG | 236 |
| hCPS1-ASO-162 | GACATGAAAAAATTTAGTGG | 237 |
| hCPS1-ASO-163 | GGTTGGTAGGGAATGTTAAA | 238 |
| hCPS1-ASO-164 | TGGTTGGTAGGGAATGTTAA | 239 |
| hCPS1-ASO-165 | TTGGTTGGTAGGGAATGTTA | 240 |
| hCPS1-ASO-166 | GTTGGTTGGTAGGGAATGTT | 241 |
| hCPS1-ASO-167 | AGTTGGTTGGTAGGGAATGT | 242 |
| hCPS1-ASO-168 | GAGTTGGTTGGTAGGGAATG | 243 |
| hCPS1-ASO-169 | AGAGTTGGTTGGTAGGGAAT | 244 |
| hCPS1-ASO-170 | AAGAGTTGGTTGGTAGGGAA | 245 |
| hCPS1-ASO-171 | TAAGAGTTGGTTGGTAGGGA | 246 |
| hCPS1-ASO-172 | ATAAGAGTTGGTTGGTAGGG | 247 |
| hCPS1-ASO-173 | CTATAAGAGTTGGTTGGTAG | 248 |
| hCPS1-ASO-174 | GAGGGAGTTTACATTAGGAT | 249 |
| hCPS1-ASO-175 | TGGAAAAAAATATATTACAC | 250 |
| hCPS1-ASO-176 | GCTAGACTGAAATCTATAAC | 251 |
| hCPS1-ASO-177 | GTGTTCAGGCTAGACTGAAA | 252 |
| hCPS1-ASO-178 | AGTGTTCAGGCTAGACTGAA | 253 |
| hCPS1-ASO-179 | GAGTGTTCAGGCTAGACTGA | 254 |
| hCPS1-ASO-180 | TGAGTGTTCAGGCTAGACTG | 255 |
| hCPS1-ASO-181 | TTGAGTGTTCAGGCTAGACT | 256 |
| hCPS1-ASO-182 | CTTGAGTGTTCAGGCTAGAC | 257 |
| hCPS1-ASO-183 | ACTTGAGTGTTCAGGCTAGA | 258 |
| hCPS1-ASO-184 | TACTTGAGTGTTCAGGCTAG | 259 |
| hCPS1-ASO-185 | ATACTTGAGTGTTCAGGCTA | 260 |
| hCPS1-ASO-186 | CATACTTGAGTGTTCAGGCT | 261 |
| hCPS1-ASO-187 | TCATACTTGAGTGTTCAGGC | 262 |
| hCPS1-ASO-188 | TTCATACTTGAGTGTTCAGG | 263 |
| hCPS1-ASO-189 | GTTTCATACTTGAGTGTTCA | 264 |
| hCPS1-ASO-190 | GGCTAGTTTCATACTTGAGT | 265 |
| hCPS1-ASO-191 | GTGGCTAGTTTCATACTTGA | 266 |
| hCPS1-ASO-192 | AGTGGCTAGTTTCATACTTG | 267 |
| hCPS1-ASO-193 | GGAGGTTGGAACAGCCAATA | 268 |
| hCPS1-ASO-194 | CAATGGAGGTTGGAACAGCC | 269 |
| hCPS1-ASO-195 | ACAATGGAGGTTGGAACAGC | 270 |
| hCPS1-ASO-196 | CCACAATGGAGGTTGGAACA | 271 |
| hCPS1-ASO-197 | TCCACAATGGAGGTTGGAAC | 272 |
| hCPS1-ASO-198 | TTCCACAATGGAGGTTGGAA | 273 |
| hCPS1-ASO-199 | ATTCCACAATGGAGGTTGGA | 274 |
| hCPS1-ASO-200 | AATTCCACAATGGAGGTTGG | 275 |
| hCPS1-ASO-201 | TAATTCCACAATGGAGGTTG | 276 |
| hCPS1-ASO-202 | ATCCATCAATTATAATTCCA | 277 |
| hCPS1-ASO-203 | AATCCATCAATTATAATTCC | 278 |
| hCPS1-ASO-204 | AAATCCATCAATTATAATTC | 279 |
| hCPS1-ASO-205 | AAAATCCATCAATTATAATT | 280 |
| hCPS1-ASO-206 | GAAAATCCATCAATTATAAT | 281 |
| hCPS1-ASO-207 | GGAAAATCCATCAATTATAA | 282 |
| hCPS1-ASO-208 | TGGAAAATCCATCAATTATA | 283 |
| hCPS1-ASO-209 | TTGGAAAATCCATCAATTAT | 284 |
| hCPS1-ASO-210 | CTTGGAAAATCCATCAATTA | 285 |
| hCPS1-ASO-211 | ACTTGGAAAATCCATCAATT | 286 |
| hCPS1-ASO-212 | GACTTGGAAAATCCATCAAT | 287 |
| hCPS1-ASO-213 | AGACTTGGAAAATCCATCAA | 288 |
| hCPS1-ASO-214 | GAGACTTGGAAAATCCATCA | 289 |
| hCPS1-ASO-215 | AGAGACTTGGAAAATCCATC | 290 |
| hCPS1-ASO-216 | GAATTAGAGACTTGGAAAAT | 291 |
| hCPS1-ASO-217 | TGAATTAGAGACTTGGAAAA | 292 |
| hCPS1-ASO-218 | CTGAATTAGAGACTTGGAAA | 293 |
| hCPS1-ASO-219 | TCTGAATTAGAGACTTGGAA | 294 |
| hCPS1-ASO-220 | TTCTGAATTAGAGACTTGGA | 295 |
| hCPS1-ASO-221 | ATTCTGAATTAGAGACTTGG | 296 |
| hCPS1-ASO-222 | TATTCTGAATTAGAGACTTG | 297 |
| hCPS1-ASO-223 | GGAGAAGGCAGAAATTTTGG | 298 |
| hCPS1-ASO-224 | GTCCATGTCTTTAGTCATCA | 299 |
| hCPS1-ASO-225 | ACGGTGTGTGTGTGTATATA | 300 |
| hCPS1-ASO-226 | AACTACGGTGTGTGTGTGTA | 301 |
| hCPS1-ASO-227 | TGCAAACTACGGTGTGTGTG | 302 |
| hCPS1-ASO-228 | GACTTGCAAACTACGGTGTG | 303 |
| hCPS1-ASO-229 | ATGCAGACTTGCAAACTACG | 304 |
| hCPS1-ASO-230 | CCAGATGCAGACTTGCAAAC | 305 |
| hCPS1-ASO-231 | GGTCCCAGATGCAGACTTGC | 306 |
| hCPS1-ASO-232 | AAAGGGTCCCAGATGCAGAC | 307 |
| hCPS1-ASO-233 | CAGTAAAGGGTCCCAGATGC | 308 |
| hCPS1-ASO-234 | TATCAGTAAAGGGTCCCAGA | 309 |
| hCPS1-ASO-235 | GGCATGAGAGTTTTAATGGG | 310 |
| hCPS1-ASO-236 | GATAGAATTCTCCTACTCTA | 311 |
| hCPS1-ASO-237 | CCATAGAAATGTCAACAATC | 312 |
| hCPS1-ASO-238 | GCATGATGAATTAAACATGT | 313 |
| hCPS1-ASO-239 | CCATTTATAGCAGTTGACTA | 314 |
| hCPS1-ASO-240 | TTGACCAAGGAAAAGACTTC | 315 |
| hCPS1-ASO-241 | AGCTTGACCAAGGAAAAGAC | 316 |
| hCPS1-ASO-242 | AGTCCAGAGCTTGACCAAGG | 317 |
| hCPS1-ASO-243 | GAGGAGTCCAGAGCTTGACC | 318 |
| hCPS1-ASO-244 | AGTACTGAGGAGTCCAGAGC | 319 |
| hCPS1-ASO-245 | AGTGGAAGTACTGAGGAGTC | 320 |
| hCPS1-ASO-246 | TGACAGTGGAAGTACTGAGG | 321 |
| hCPS1-ASO-247 | TATGACAGTGGAAGTACTGA | 322 |
| hCPS1-ASO-248 | TCCTCCCTTCATCACTTATG | 323 |
| hCPS1-ASO-249 | ACCCATGCAATGAATACTAT | 324 |
| hCPS1-ASO-250 | TACCCATGCAATGAATACTA | 325 |
| hCPS1-ASO-251 | GTACCCATGCAATGAATACT | 326 |
| hCPS1-ASO-252 | GGTACCCATGCAATGAATAC | 327 |
| hCPS1-ASO-253 | AGGTACCCATGCAATGAATA | 328 |
| hCPS1-ASO-254 | AAGGTACCCATGCAATGAAT | 329 |
| hCPS1-ASO-255 | TAAGGTACCCATGCAATGAA | 330 |
| hCPS1-ASO-256 | CCTAAGGTACCCATGCAATG | 331 |
| hCPS1-ASO-257 | TTTAAATCAGTCATAGTCCC | 332 |
| hCPS1-ASO-258 | GCTGCACCCACTAATGGAAA | 333 |
| hCPS1-ASO-259 | GTGCTGCACCCACTAATGGA | 334 |
| hCPS1-ASO-260 | GGAAACTTTTCTTTTTAAGT | 335 |
| hCPS1-ASO-261 | ACGATATAAACTCAATCCTC | 336 |
| hCPS1-ASO-262 | TAGTGAGGGTTGTCTTTCCT | 337 |
| hCPS1-ASO-263 | TGTAGTGAGGGTTGTCTTTC | 338 |
| hCPS1-ASO-264 | GTGTAGTGAGGGTTGTCTTT | 339 |
| hCPS1-ASO-265 | GTGTGTAGTGAGGGTTGTCT | 340 |
| hCPS1-ASO-266 | TCTTGTCTTTTTATGTGTGT | 341 |
| hCPS1-ASO-267 | TACTAGTTTACGCCTTGATC | 342 |
| hCPS1-ASO-268 | GCAGTACTTACTGGCTTAGT | 343 |
| hCPS1-ASO-269 | GAAAACTCCCAACCTAGAGA | 344 |
| hCPS1-ASO-270 | TTGGAGGAGAATGAAAATAG | 345 |
| hCPS1-ASO-271 | CGAAATTTGTGTTGAAGATC | 346 |
| hCPS1-ASO-272 | CATAATAGTCATGGAAGTAT | 347 |
| hCPS1-ASO-273 | AATGTTCAGCCTTCATCCAT | 348 |
| hCPS1-ASO-274 | TCATGGGAGACTTAGTATGC | 349 |
| hCPS1-ASO-275 | CTAGATGCAGGGTATAGAAT | 350 |
| hCPS1-ASO-276 | CATGAAGTCATGTGGTATAT | 351 |
| hCPS1-ASO-277 | CCGTGGCATGAGCAATAGCC | 352 |
| hCPS1-ASO-278 | AAGTAGATAAAGAGGGGAAA | 353 |
| hCPS1-ASO-279 | GGCATGAATGCAGGCA | 354 |
| hCPS1-ASO-280 | CAGGCATGAATGCAGG | 355 |
| hCPS1-ASO-281 | GACAGGCATGAATGCA | 356 |
| hCPS1-ASO-282 | GTGACAGGCATGAATG | 357 |
| hCPS1-ASO-283 | CACATCAGGCTGGGGA | 358 |
| hCPS1-ASO-284 | CATCAGGCTGGGGACC | 359 |
| hCPS1-ASO-285 | TCAGGCTGGGGACCCT | 360 |
| hCPS1-ASO-286 | AGGCTGGGGACCCTGT | 361 |
| hCPS1-ASO-287 | CATGAATGCAGGCACA | 362 |
| hCPS1-ASO-288 | TGAATGCAGGCACACA | 363 |
| hCPS1-ASO-289 | AATGCAGGCACACACA | 364 |
| hCPS1-ASO-290 | TGCAGGCACACACATC | 365 |
| hCPS1-ASO-291 | CAGGCACACACATCAG | 366 |
| hCPS1-ASO-292 | GGCACACACATCAGGC | 367 |
| hCPS1-ASO-293 | CACACACATCAGGCTG | 368 |
| hCPS1-ASO-294 | CACACATCAGGCTGGG | 369 |
| hCPS1-ASO-295 | GGCATGAATGCAGGCA | 370 |
| hCPS1-ASO-296 | CAGGCATGAATGCAGG | 371 |
| hCPS1-ASO-297 | GACAGGCATGAATGCA | 372 |
| hCPS1-ASO-298 | GTGACAGGCATGAATG | 373 |
| hCPS1-ASO-299 | CACATCAGGCTGGGGA | 374 |
| hCPS1-ASO-300 | CATCAGGCTGGGGACC | 375 |
| hCPS1-ASO-301 | TCAGGCTGGGGACCCT | 376 |
| hCPS1-ASO-302 | AGGCTGGGGACCCTGT | 377 |
| hCPS1-ASO-303 | GCATGAATGCAGGCACAC | 378 |
| hCPS1-ASO-304 | GCACACACATCAGGCTGG | 379 |
| hCPS1-ASO-305 | ACACACATCAGGCTGGGG | 380 |
| hCPS1-ASO-306 | CACACACATCAGGCTGGG | 381 |
| hCPS1-ASO-307 | GGCATGAATGCAGGCACACA | 382 |
| hCPS1-ASO-308 | GGCATGAATGCAGGCACACA | 383 |
| hCPS1-ASO-309 | GGCACACACATCAGGCTGGG | 384 |
| hCPS1-ASO-310 | CACACACATCAGGCTGGGGA | 385 |
| hCPS1-ASO-311 | GCACACACATCAGGCTGGGG | 386 |
| hCPS1-ASO-312 | TGCAGGCACACACATCAGGC | 387 |
| hCPS1-ASO-313 | ATCAGGCTGGGGACCCTGTG | 388 |
| hCPS1-ASO-314 | GGCACACACATCAGGCTGGG | 389 |
| hCPS1-ASO-315 | ATGAATGCAGGCACACACAT | 390 |
| hCPS1-ASO-316 | TGCAGGCACACACATCAGGC | 391 |
| hCPS1-ASO-317 | GCAATGACAAGGTATTTGAT | 487 |
| hCPS1-ASO-318 | GGACTACAAGTTGTTGCAAT | 488 |
| hCPS1-ASO-319 | GTTAGATCAAAGAGTCCTTA | 489 |
| hCPS1-ASO-320 | TTAGTAGAGGGTTAGATCAA | 490 |
| hCPS1-ASO-321 | GTAGAAGGACTTAGTAGAGG | 491 |
| hCPS1-ASO-322 | GTCACACATGAACAATTCTC | 492 |
| hCPS1-ASO-323 | GATGACTGATTTACTTGACA | 493 |
| hCPS1-ASO-324 | TGTATAGTGCAAATGATGAC | 494 |
| hCPS1-ASO-325 | TTTTGTCATCTGTATAGTGC | 495 |
| hCPS1-ASO-326 | TCACATATTCTCTGAACCTA | 496 |
| hCPS1-ASO-327 | TTCTTGGACAAGTCACATAT | 497 |
| hCPS1-ASO-328 | TAATGCCCTGTTCTTGGACA | 498 |
| hCPS1-ASO-329 | CTGTTATGCATTTCCTAATG | 499 |
| hCPS1-ASO-330 | GAATCTAACCCCTGTTATGC | 500 |
| hCPS1-ASO-331 | GGCACAAAAAGATCTGGAAT | 501 |
| hCPS1-ASO-332 | GTAATAGTGGGATATACTCT | 502 |
| hCPS1-ASO-333 | AGGCTGTAACAGTGTAATAG | 503 |
| hCPS1-ASO-334 | TTATTACATTCAGCAGGCTG | 504 |
| hCPS1-ASO-335 | GAAAATGTTCAGCACTCATT | 505 |
| hCPS1-ASO-336 | GGTATAAATGATCAACAACC | 506 |
| hCPS1-ASO-337 | GAAAGTTGATATCAAGGGTA | 507 |
| hCPS1-ASO-338 | AGAATTTCAAGGTGCTCTAT | 508 |
| hCPS1-ASO-339 | TTTTACTTACAGGAAAGAGG | 509 |
| hCPS1-ASO-340 | CCAATCGGCACAGTTGTATT | 510 |
| hCPS1-ASO-341 | TAGAGTGCTTTGCCAATCGG | 511 |
| hCPS1-ASO-342 | GCGGCTAATATTTATAGAGT | 512 |
| hCPS1-ASO-343 | GGTATTTGTCAGCGGCTAAT | 513 |
| hCPS1-ASO-344 | TAATTTTATGTGGCCCTTCC | 514 |
| hCPS1-ASO-345 | GCAATGACGCACTTATAATT | 515 |
| hCPS1-ASO-346 | GTTTGACGACAGCAATGACG | 516 |
| hCPS1-ASO-347 | GAGACGGTGGGTTTGACGAC | 517 |
| hCPS1-ASO-348 | TTTGATGACAGAGACGGTGG | 518 |
| hCPS1-ASO-349 | TTCCTCTCTCTTTGATGACA | 519 |
| hCPS1-ASO-350 | AGGTTTGCTTTTCCTCTCTC | 520 |
| hCPS1-ASO-351 | CGACATATTTAGAGGTTTGC | 521 |
| hCPS1-ASO-352 | GGGTTGCTTTTTCGACATAT | 522 |
| hCPS1-ASO-353 | CCTGTAAATTGGGTTGCTTT | 523 |
| hCPS1-ASO-354 | CATCTATAATCATTGCCCTG | 524 |
| hCPS1-ASO-355 | AGACACCCTGTAGTCACATC | 525 |
| hCPS1-ASO-356 | TAGATGGTAAATGGATTGGG | 526 |
| hCPS1-ASO-357 | TATGTCCAGTTATAGATGGT | 527 |
| hCPS1-ASO-358 | AAGGTTAGTAGCATTATGTC | 528 |
| hCPS1-ASO-359 | CAGTCACTTTAAAGGTTAGT | 529 |
| hCPS1-ASO-360 | GTAGTTACATAATGGTCAGT | 530 |
| hCPS1-ASO-361 | ATCATTAGGATGTAGTTACA | 531 |
| hCPS1-ASO-362 | GCCTTCACTTTAAAAACTCA | 532 |
| hCPS1-ASO-363 | ACTTTTCTTCCTGCCTTCAC | 533 |
| hCPS1-ASO-364 | TAGTCCTCTATCTACTTTTC | 534 |
| hCPS1-ASO-365 | GTTATTGTGAAATAGTCCTC | 535 |
| hCPS1-ASO-366 | GGACTTGTGTTATTATTCAG | 536 |
| hCPS1-ASO-367 | ATTCCATAGAACAGCCCAAG | 537 |
| hCPS1-ASO-368 | GGAGGAATGCCAAGCACAAA | 538 |
| hCPS1-ASO-369 | CCAGAGTTTTGGAGGAATGC | 539 |
| hCPS1-ASO-370 | CAATCATTACTGGCCAGAGT | 540 |
| hCPS1-ASO-371 | GCCATTACTCACACTCAATC | 541 |
| hCPS1-ASO-372 | TCATTTCATGGTGTAAAAGC | 542 |
| hCPS1-ASO-373 | CACTAAGCCATCATTTCATG | 543 |
| hCPS1-ASO-374 | GACAAATTTGTAGGAGCACT | 544 |
| hCPS1-ASO-375 | ACAGTGCACAGCTAAGAATG | 545 |
| hCPS1-ASO-376 | AAGGGTCTGTCACAGTGCAC | 546 |
| hCPS1-ASO-377 | ACAGATCTGTTTCAAGGGTC | 547 |
| hCPS1-ASO-378 | CAGTTCTAGCTCATGGTGAC | 548 |
| hCPS1-ASO-379 | GGATGGATTACTGTGAAAGC | 549 |
| hCPS1-ASO-380 | TGGTCTAATTATGTGGATGG | 550 |
| hCPS1-ASO-381 | CCACGACAAAATGGTCTAAT | 551 |
| hCPS1-ASO-382 | TCCATACAACCACCACCACA | 552 |
| hCPS1-ASO-383 | CAATAAGTGCTCCATACAAC | 553 |
| hCPS1-ASO-384 | GAATCCAATTCTCCAAGATC | 554 |
| hCPS1-ASO-385 | GGCACAGATATGATTGAAAT | 555 |
| hCPS1-ASO-386 | GAGTACTTACTTAAAATGGC | 556 |
| hCPS1-ASO-387 | ACCGCGAAATTGAGTACTTA | 557 |
| hCPS1-ASO-388 | AGACCCCAGAAACCGCGAAA | 558 |
| hCPS1-ASO-389 | GGTGCATCTTCATCAGACCC | 559 |
| hCPS1-ASO-390 | GTAGGTAAGGATAGGTGCAT | 560 |
| hCPS1-ASO-391 | CACTCTGTGAGAAGGTAGGT | 561 |
| hCPS1-ASO-392 | GAATATCTTCTTCTCACTCT | 562 |
| hCPS1-ASO-393 | GGGTGACTTGGAATATCTTC | 563 |
| hCPS1-ASO-394 | TAATACTTCAAAGTTGGGTG | 564 |
| hCPS1-ASO-395 | TATGTGCCATCTAATACTTC | 565 |
| hCPS1-ASO-396 | CCCTATGATTATATGTGCCA | 566 |
| hCPS1-ASO-397 | GTAATGAAAGTTATCCCCTA | 567 |
| hCPS1-ASO-398 | GTACTCATGTATCAGTTCAG | 568 |
| hCPS1-ASO-399 | TTGTTAGTAATACCTCAAGG | 569 |
| hCPS1-ASO-400 | ACTAGATGTAAACTATGAGA | 570 |
| hCPS1-ASO-401 | AGACATTGTAACTAGATGTA | 571 |
| hCPS1-ASO-402 | TAGCAAAAGAATGGGAAAGG | 572 |
| hCPS1-ASO-403 | AAGGTTAGTGGCATTATGTC | 573 |
| hCPS1-ASO-404 | GGAGCAGTCACTAAAGGTTA | 574 |
| hCPS1-ASO-405 | GCACGTATGTTATATAGCCT | 575 |
| hCPS1-ASO-406 | GTAAGCCAGAAAGCACGTAT | 576 |
| hCPS1-ASO-407 | CTTAGAAACATCAGGTAAGC | 577 |
| hCPS1-ASO-408 | AGGCGAACTAATAGCTTAGA | 578 |
| hCPS1-ASO-409 | GGATGAGTACTGAGGCGAAC | 579 |
| hCPS1-ASO-410 | TTCAGTGCTTAGTTCAGATT | 580 |
| hCPS1-ASO-411 | GGCTTTCTACTTTTCAGTGC | 581 |
| hCPS1-ASO-412 | TCTGGCTTTAGGCTTTCTAC | 582 |
| hCPS1-ASO-413 | GACAAATTGTGCTGAAATTC | 583 |
| hCPS1-ASO-414 | CCTTGTAACTGCAGAGAAGT | 584 |
| hCPS1-ASO-415 | GAAGTGTTAGGCCTTGTAAC | 585 |
| hCPS1-ASO-416 | GCTGTTACAGTGCTTCAGAA | 586 |
| hCPS1-ASO-417 | ATTGATTACTGCTGTTACAG | 587 |
| hCPS1-ASO-418 | GTGGTAAGGCCTAATTGATT | 588 |
| hCPS1-ASO-419 | AACTGAGAATGTGGTAAGGC | 589 |
| hCPS1-ASO-420 | TAACCAAAGACCGATGACTT | 590 |
| hCPS1-ASO-421 | GGCTTATGTTTAACCAAAGA | 591 |
| hCPS1-ASO-422 | CCCCTTAACATTGGCTTATG | 592 |
| hCPS1-ASO-423 | TAACCTTTTGATTTTGTCCC | 593 |
| hCPS1-ASO-424 | CCTACACTTTATGAACATAC | 594 |
| hCPS1-ASO-425 | GCTCAGTAAGGCATATTAAT | 595 |
| hCPS1-ASO-426 | CTGAACAACTTGCCACATAG | 596 |
| hCPS1-ASO-427 | CTCCAGGATTCTGAACAACT | 597 |
| hCPS1-ASO-428 | TTATGATATGTAGGTTCCTC | 598 |
| hCPS1-ASO-429 | ATTTATCACCACCATACAGT | 599 |
| hCPS1-ASO-430 | GCAGACTGAAATATAAGACC | 600 |
| hCPS1-ASO-431 | CTTCTATAATGCAGACTGAA | 601 |
| hCPS1-ASO-432 | GGTTGCTACATTATGAAAGA | 602 |
| hCPS1-ASO-433 | TGAATCAATAGGGTTGCTAC | 603 |
| hCPS1-ASO-434 | GGTAACTGACTAATTTGAAG | 604 |
| hCPS1-ASO-435 | AAATTTCATAGGGAGGTAAC | 605 |
| hCPS1-ASO-436 | TTCAAGGCTACTCTGTTTGC | 606 |
| hCPS1-ASO-437 | GTGATCTAAATGAGTTGAAC | 607 |
| hCPS1-ASO-438 | GCTTCCATTTGATTTTCTTC | 608 |
| hCPS1-ASO-439 | CCCTAGGGATCTTTAGCTTC | 609 |
| hCPS1-ASO-440 | GGTCAAAGTTTGTGTTAGGC | 610 |
| hCPS1-ASO-441 | GAGGCCCGAAAAAAGCTGCG | 611 |
| hCPS1-ASO-442 | ATTTGCAATGACGGAGGCCC | 612 |
| hCPS1-ASO-443 | CTTGATGGGATTTGTATTTC | 613 |
| hCPS1-ASO-444 | TACGCTGACTGCCCTTGATG | 614 |
| hCPS1-ASO-445 | GAGGAGTTTGGTATGCTACG | 615 |
| hCPS1-ASO-446 | TCACTTCAAGCCTTCAGGAG | 616 |
| hCPS1-ASO-447 | GGCATGCTAGAATTCACTTC | 617 |
| hCPS1-ASO-448 | TCAGTATCTCTTAGGCAATG | 618 |
| hCPS1-ASO-449 | GGGTGCTGTAGAAAGTTTTC | 619 |
| hCPS1-ASO-450 | CATATTTCCAACCTGTTCCA | 620 |
| hCPS1-ASO-451 | GCCTTTACATCTGTATTAGA | 621 |
| hCPS1-ASO-452 | GACTATTCCTCTGCCTTTAC | 622 |
| hCPS1-ASO-453 | TGCTATGGAAATGGAGACTA | 623 |
| hCPS1-ASO-454 | TAGGTCTTATTGCTATGGAA | 624 |
| hCPS1-ASO-455 | CGATCTAGAGGTGACATTAG | 625 |
| hCPS1-ASO-456 | GACTTTCTGACGATCTAGAG | 626 |
| hCPS1-ASO-457 | ATTTGCTCATGTCTAGCCAG | 627 |
| hCPS1-ASO-458 | CCCTCATTCCAATTTGCTCA | 628 |
| hCPS1-ASO-459 | GTGCTTTCCTCTCCCTCATT | 629 |
| hCPS1-ASO-460 | TAATCTTGACCTGTGCTTTC | 630 |
| hCPS1-ASO-461 | GGACTAGTACTAATCTTGAC | 631 |
| hCPS1-ASO-462 | ACTGCCCAAGGGACTAGTAC | 632 |
| hCPS1-ASO-463 | AGCATTCTGGCTGTAGAACT | 633 |
| hCPS1-ASO-464 | TTGGGTCAGAAGCTAAGCAT | 634 |
| hCPS1-ASO-465 | CAGTACCCTAGTCCCAGCTT | 635 |
| hCPS1-ASO-466 | GCACATCACTCAGTACCCTA | 636 |
| hCPS1-ASO-467 | CTTTGAGAGTTAGGCCCTAA | 637 |
| hCPS1-ASO-468 | TAGTGTCATGGTGTATCTCT | 638 |
| hCPS1-ASO-469 | AGGAGGCTGTCACTGTTAGT | 639 |
| hCPS1-ASO-470 | GAGTGAGGTACCTGAGATGC | 640 |
| hCPS1-ASO-471 | CTAGGTTAAGTGAGTGAGGT | 641 |
| hCPS1-ASO-472 | GTTTAAATGTCCTAGACTAG | 642 |
| hCPS1-ASO-473 | GAGCTTCTTGACATAAAACT | 643 |
| hCPS1-ASO-474 | CAACGCCTTGACATTATGAG | 644 |
| hCPS1-ASO-475 | TAGTCACCAACAACGCCTTG | 645 |
| hCPS1-ASO-476 | AGATGGCCTTTCTTTTGAAC | 646 |
| hCPS1-ASO-477 | TCTAATTGGGAGATGGCCTT | 647 |
| hCPS1-ASO-478 | GCAATACTTCTCTAATTGGG | 648 |
| hCPS1-ASO-479 | TACTTTTGGCATGCAATACT | 649 |
| hCPS1-ASO-480 | GATGGACAACTTTACATACT | 650 |
| hCPS1-ASO-481 | AATTAGTTTGCCTGATGGAC | 651 |
| hCPS1-ASO-482 | GAGCTGTTGCAGAATTAGTT | 652 |
| hCPS1-ASO-483 | AGTCCCAAATGAGCTGTTGC | 653 |
| hCPS1-ASO-484 | TTAATTGTGTTCAGTCCCAA | 654 |
| hCPS1-ASO-485 | CATCTATGGCAAACTACATT | 655 |
| hCPS1-ASO-486 | GGACGGATTTCATCTATGGC | 656 |
| hCPS1-ASO-487 | TATAGACACATTTTAGGACG | 657 |
| hCPS1-ASO-488 | GGTTATCACACACTTTTAAG | 658 |
| hCPS1-ASO-489 | GTACACGTTAGATGTAAGAG | 659 |
| hCPS1-ASO-490 | TATTGGAGCCTCTGTACACG | 660 |
| hCPS1-ASO-491 | CTCATTATCATATTGGAGCC | 661 |
| hCPS1-ASO-492 | GTCACAGGAAATATAACTCA | 662 |

TABLE 3
Additional Chemical Modifications of selected hCPS1-ASOs
Key: MOE (M); DNA (d); LNA (L); PS (=); PO(-); 5-MethylCytosine (5C); GalNAc (ag); Teg-GalNAc
(TEG); cET (c); AlaGal-GalNAc (AlGal); cholesterol (CholTEG)

| SEQ | | |
|---|---|---|
| ID | | |
| NO | Name | Sequence |
| 16 | hCPS1- | MT=.MG=.M5C=.MA=.MG=.dG=.dC=.dA=.dC=.dA=.dC=.dA=.dC=.dA=.dT=.M5C=.MA=. |
| | ASO- | MG=.MG=.M5C |
| | 1a | |
| 17 | hCPS1- | MT=.MG=.M5C=.MA=.MG=.MG=.M5C=.MA=.M5C=.MA=.M5C=.MA=.M5C=.MA=.MT=. |
| | ASO- | M5C=.MA=.MG=.MG=.M5C |
| | 1b | |
| 18 | hCPS1- | MT=.MG=.M5C=.MA=.MG=.dG=.dC=.dA=.M5C=.dA=.dC=.MA=.dC=.dA=.dT=.M5C=.MA |
| | ASO- | =.MG=.MG=.M5C |
| | 1c | |
| 19 | ASO- | MT=.MG=.M5C=.MA=.MG=.LG=.dC=.dA=.L5C=.dA=.dC=.LA=.dC=.dA=.LT=.M5C=.MA= |
| | 1d | MG=.MG=.M5C |
| 20 | hCPS1- | MT=.MG=.M5C=.LA=.MG=.dG=.dC=.LA=.dC=.dA=.dC=.LA=.dC=.dA=.dT=.L5C=.MA=.M |
| | ASO- | G=.MG=.M5C |
| | 1e | |
| 21 | hCPS1- | MT=.MG=.M5C=.MA=.LG=.dG=.dC=.dA=.L5C=.dA=.dC=.dA=.L5C=.dA=.dT=.M5C=.LA=. |
| | ASO- | MG=.MG=.M5C |
| | 1f | |
| 22 | hCPS1- | MT=.MG=.M5C-.MA-.MG=.dG=.dC=.dA=.dC=.dA=.dC=.dA=.dC=.dA=.dT=.M5C-.MA- |
| | ASO- | MG=.MG=.M5C |
| | 1g | |
| 23 | hCPS1- | MA=.MT=.MG=.M5C=.MA=.MG=.dG=.dC=.dA=.dC=.dA=.dC=.dA=.dC=.dA=.dT=.M5C=. |
| | ASO-2 | MA=.MG=.MG=.M5C=.MT |
| 24 | hCPS1- | MA=.MA=.MT=.MG=.M5C=.MA=.MG=.dG=.dC=.dA=.dC=.dA=.dC=.dA=.dC=.dA=.dT=.M |
| | ASO-3 | 5C=.MA=.MG=.MG=.M5C=.MT=.MG |
| 392 | hCPS1- | MT=.MG=.M5C=.MA=.MG=.LG=.M5C=.MA=.L5C=.MA=.M5C=.LA=.M5C=.MA=.LT=.M |
| | ASO- | 5C=.MA=.MG=.MG=.M5C |
| | 81a | |
| 393 | hCPS1- | MT=.MG=.M5C=.LA=.MG=.LG=.M5C=.LA=.M5C=.LA=.M5C=.LA=.M5C=.LA=.MT=.L5C |
| | ASO- | =.MA=.LG=.MG=.M5C |
| | 81b | |
| 394 | hCPS1- | MT=.MG=.L5C=.MA=.LG=.MG=.L5C=.MA=.L5C=.MA=.L5C=.MA=.L5C=.MA=.LT=.M5C |
| | ASO- | =.LA=.MG=.MG=.M5C |
| | 81c | |
| 395 | hCPS1- | MT=.MG=.L5C=.MA=.MG=.LG=.M5C=.MA=.L5C=.MA=.M5C=.LA=.M5C=.MA=.LT=.M5 |
| | ASO- | C=.MA=.LG=.MG=.M5C |
| | 81d | |
| 396 | hCPS1- | MT=.MG=.M5C=.MA=.LG=.MG=.M5C=.MA=.L5C=.MA=.M5C=.MA=.L5C=.MA=.MT=.M |
| | ASO- | 5C=.LA=.MG=.MG=.M5C |
| | 81e | |
| 397 | hCPS1- | MT=.MG=.M5C=.LA=.MG=.MG=.M5C=.LA=.M5C=.MA=.M5C=.LA=.M5C=.MA=.MT=.L |
| | ASO- | 5C=.MA=.MG=.MG=.M5C |
| | 81f | |
| 398 | hCPS1- | MT=.MG=.M5C=.MA=.MG=.LG=.M5C=.MA=.M5C=.MA=.L5C=.MA=.M5C=.MA=.MT=.L |
| | ASO- | 5C=.MA=.MG=.MG=.M5C |
| | 81g | |
| 399 | hCPS1- | MT=.MG=.M5C=.MA=.LG=.MG=.M5C=.MA=.M5C=.LA=.M5C=.MA=.M5C=.MA=.LT=.M |
| | ASO- | 5C=.MA=.MG=.MG=.M5C |
| | 81h | |
| 400 | hCPS1- | LT=.MG=.L5C=.MA=.LG=.dG=.dC=.dA=.dC=.dA=.dC=.dA=.dC=.dA=.dT=.L5C=.MA=.LG |
| | ASO- | =.MG=.L5C |
| | 81i | |
| 401 | hCPS1- | LT=.MG=.L5C=.MA=.LG=.MG=.M5C=.MA=.M5C=.MA=.M5C=.MA=.M5C=.MA=.MT=.L |
| | ASO- | 5C=.MA=.LG=.MG=.L5C |
| | 81j | |
| 402 | hCPS1- | MT=.MG=.M5C=.MA=.MG=.MG=.dC=.dA=.M5C=.dA=.dC=.MA=.dC=.dA=.MT=.M5C=.M |
| | ASO- | A=.MG=.MG=.M5C |
| | 81k | |
| 403 | hCPS1- | MT=.MG=.M5C=.MA-.MG=.dG=.d5C=.dA=.d5C=.dA=.d5C=.dA=.d5C=.dA=.dT=.M5C- |
| | ASO- | MA=.MG=.MG=.M5C |
| | 81l | |
| 404 | hCPS1- | MT=.MG=.M5C-.MA-.MG=.dG=.d5C=.dA=.d5C=.dA=.d5C=.dA=.d5C=.dA=.dT=.M5C- |
| | ASO- | .MA-.MG=.MG=.M5C |
| | 81m | |
| 405 | hCPS1- | MT=.MG-.M5C-.MA-.MG=.dG=.d5C=.dA=.d5C=.dA=.d5C=.dA=.d5C=.dA=.dT=.M5C-.MA- |
| | ASO- | .MG-.MG=.M5C |
| | 81n | |
| 406 | hCPS1- | MT-.MG-.M5C-.MA-.MG=.dG=.d5C=.dA=.d5C=.dA=.d5C=.dA=.d5C=.dA=.dT=.M5C-.MA- |
| | ASO- | .MG-.MG-.M5C |
| | 81o | |
| 407 | hCPS1- | MT=.MG=.M5C=.MA=.MG=.MG=.dC=.dA=.dC=.dA=.dC=.dA=.dC=.dA=.dT=.dC=.MA=.M |
| | ASO- | G=.MG=.M5C=.MT=.MG |
| | 82a | |
| 408 | hCPS1- | MT=.MG=.L5C=.MA=.LG=.dG=.dC=.dA=.dC=.dA=.dC=.dA=.dC=.dA=.dT=.L5C=.MA=.LG |
| | ASO- | =.MG=.M5C |
| | 86a | |
| 409 | hCPS1- | MT=.MG=.M5C-.MA-.MG=.dG=.d5C=.dA=.d5C=.dA=.d5C=.dA=.d5C=.dA=.dT=.M5C- |
| | ASO- | .MA-.MG=.MG=.M5C-.[TEG] |
| | 86b | |
| 410 | hCPS1- | MT=.MG=.M5C-.MA-.MG=.dG=.dC=.dA=.dC=.dA=.dC=.dA=.dC=.dA=.dT=.M5C-.MA- |
| | ASO- | .MG=.MG |
| | 87a | |
| 411 | hCPS1- | MT=.MG=.M5C-.MA-.MG=.dG=.dC=.dA=.dC=.dA=.dC=.dA=.dC=.dA=.dT=.M5C-.MA-.MG |
| | ASO- | |
| | 88a | |
| 412 | hCPS1- | MT=.MG=.M5C-.MA-.MG=.dG=.dC=.dA=.dC=.dA=.dC=.dA=.dC=.dA=.dT |
| | ASO- | |
| | 89a | |
| 414 | hCPS1- | MT=.MG=.M5C-.MA-.MG=.dG=.d5C=.dA=.d5C=.dA=.d5C=.dA=.d5C=.dA=.dT=.M5C- |
| | ASO- | .MA-.MG=.MG=.M5C-.[AlGal] |
| | 91a | |
| 415 | hCPS1- | MT=.MG=.M5C-.MA-.MG-.dG-.d5C-.dA-.d5C-.dA-.d5C-.dA-.d5C-.dA-.dT-.M5C-.MA- |
| | ASO- | MG=.MG=.M5C-.[TEG] |
| | 91b | |
| 416 | hCPS1- | dT=.dG=.d5C=.dA=.dG=.dG=.d5C=.dA=.d5C=.dA=.d5C=.dA=.d5C=.dA=.dT=.d5C=.dA=.dG |
| | ASO- | =.dG=.d5C-.[TEG] |
| | 91c | |
| 417 | hCPS1- | CholTEG- |
| | ASO- | .MT=.MG=.M5C=.MA=.MG=.dG=.d5C=.dA=.d5C=.dA=.d5C=.dA=.d5C=.dA=.dT=.M5C=.M |
| | 91d | A=.MG=.MG=.M5C |
| 418 | hCPS1- | CholTEG-.MT=.MG=.M5C=.MA- |
| | ASO- | .MG=.dG=.d5C=.dA=.d5C=.dA=.d5C=.dA=.d5C=.dA=.dT=.M5C-.MA=.MG=.MG=.M5C |
| | 91e | |
| 419 | hCPS1- | CholTEG-.MT=.MG=.M5C-.MA- |
| | ASO- | .MG=.dG=.d5C=.dA=.d5C=.dA=.d5C=.dA=.d5C=.dA=.dT=.M5C-.MA-.MG=.MG=.M5C |
| | 91f | |
| 420 | hCPS1- | CholTEG-.MT-.MG-.M5C-.MA- |
| | ASO- | .MG=.dG=.d5C=.dA=.d5C=.dA=.d5C=.dA=.d5C=.dA=.dT=.M5C-.MA-.MG-.MG-.M5C |
| | 91g | |
| 421 | hCPS1- | CholTEG=.MT=.MG=.M5C=.MA=.MG=.dG=.d5C=.dA=.d5C=.dA=.d5C=.dA=.d5C=.dA=.dT |
| | ASO- | =.M5C=.MA=.MG=.MG=.M5C |
| | 91h | |
| 422 | hCPS1- | CholTEG=.MT=.MG=.M5C=.MA- |
| | ASO- | .MG=.dG=.d5C=.dA=.d5C=.dA=.d5C=.dA=.d5C=.dA=.dT=.M5C-.MA=.MG=.MG=.M5C |
| | 91i | |
| 423 | hCPS1- | CholTEG=.MT=.MG=.M5C-.MA- |
| | ASO- | .MG=.dG=.d5C=.dA=.d5C=.dA=.d5C=.dA=.d5C=.dA=.dT=.M5C-.MA-.MG=.MG=.M5C |
| | 91j | |
| 424 | hCPS1- | CholTEG=.MT-.MG-.M5C-.MA- |
| | ASO- | MG=.dG=.d5C=.dA=.d5C=.dA=.d5C=.dA=.d5C=.dA=.dT=.M5C-.MA-.MG-.MG-.M5C |
| | 91k | |
| 425 | hCPS1- | cA=.cA=.cT=.dG=.d5C=.dA=.dG=.dG=.d5C=.dA=.d5C=.dA=.d5C=.cA=.c5C=.cA |
| | ASO- | |
| | 97a | |
| 426 | hCPS1- | cT=.cG=.cA=.dA=.dT=.dG=.d5C=.dA=.dG=.dG=.d5C=.dA=.d5C=.cA=.c5C=.cA |
| | ASO- | |
| | 98a | |
| 427 | hCPS1- | c5C=.cA=.cT=.dG=.dA=.dA=.dT=.dG=.d5C=.dA=.dG=.dG=.d5C=.cA=.c5C=.cA |
| | ASO- | |
| | 99a | |
| 428 | hCPS1- | cT=.cG=.c5C=.dA=.dG=.dG=.d5C=.dA=.d5C=.dA=.d5C=.dA=.d5C=.cA=.cT=.c5C |
| | ASO- | |
| | 100a | |
| 429 | hCPS1- | c5C=.cA=.cG=.dG=.d5C=.dA=.d5C=.dA=.d5C=.dA=.d5C=.dA=.dT=.c5C=.cA=.cG |
| | ASO- | |
| | 101a | |
| 430 | hCPS1- | cG=.cG=.c5C=.dA=.d5C=.dA=.d5C=.dA=.d5C=.dA=.dT=.d5C=.dA=.cG=.cG=.c5C |
| | ASO- | |
| | 102a | |
| 431 | hCPS1- | c5C=.cA=.c5C=.dA=.d5C=.dA=.d5C=.dA=.dT=.d5C=.dA=.dG=.dG=.c5C=.cT=.cG |
| | ASO- | |
| | 103a | |
| 432 | hCPS1- | c5C=.cA=.c5C=.dA=.d5C=.dA=.dT=.d5C=.dA=.dG=.dG=.d5C=.dT=.cG=.cG=.cG |
| | ASO- | |
| | 104a | |
| 433 | hCPS1- | MT-.MG-.M5C-.MA-.MG-.dG-.d5C-.dA-.d5C-.dA-.d5C-.dA-.d5C-.dA-.dT-.M5C-.MA-.MG- |
| | ASO- | MG-.M5C-.dT-.dT-.dT-.dT-.dT-dT-dT-dT-dT-dT-[BioTEG] |
| | 105a | |
| 434 | hCPS1- | [BioTEG]P.dT-.dT-.dT-.dT-.dT-.dT-.dT-dT-dT-dT-MT-MG-.M5C-.MA-MG-.dG-.d5C- |
| | ASO- | .dA-.d5C-.dA-.d5C-.dA-.d5C-.dA-.dT-.M5C-.MA-.MG-.MG-.M5C |
| | 106a | |
| 435 | hCPS1- | cG=.cG=.c5C=.dA=.dT=.dG=.dA=.dA=.dT=.dG=.d5C=.dA=.dG=.cG=.c5C=.cA |
| | ASO- | |
| | 279a | |
| 436 | hCPS1- | c5C=.cA=.cG=.dG=.d5C=.dA=.dT=.dG=.dA=.dA=.dT=.dG=.d5C=.cA=.cG=.cG |
| | ASO- | |
| | 280a | |
| 437 | hCPS1- | cG=.cA=.c5C=.dA=.dG=.dG=.d5C=.dA=.dT=.dG=.dA=.dA=.dT=.cG=.c5C=.cA |
| | ASO- | |
| | 281a | |
| 438 | hCPS1- | cG=.cT=.cG=.dA=.d5C=.dA=.dG=.dG=.d5C=.dA=.dT=.dG=.dA=.cA=.cT=.cG |
| | ASO- | |
| | 282a | |
| 439 | hCPS1- | c5C=.cA=.c5C=.dA=.dT=.d5C=.dA=.dG=.dG=.d5C=.dT=.dG=.dG=.cG=.cG=.cA |
| | ASO- | |
| | 283a | |
| 440 | hCPS1- | c5C=.cA=.cT=.d5C=.dA=.dG=.dG=.d5C=.dT=.dG=.dG=.dG=.dG=.cA=.c5C=.c5C |
| | ASO- | |
| | 284a | |
| 441 | hCPS1- | cT=.c5C=.cA=.dG=.dG=.d5C=.dT=.dG=.dG=.dG=.dG=.dA=.d5C=.c5C=.c5C=.cT |
| | ASO- | |
| | 285a | |
| 442 | hCPS1- | cA=.cG=.cG=.d5C=.dT=.dG=.dG=.dG=.dG=.dA=.d5C=.d5C=.d5C=.cT=.cG=.cT |
| | ASO- | |
| | 286a | |
| 443 | hCPS1- | L5C=.LA=.LT=.dG=.dA=.dA=.dT=.dG=.d5C=.dA=.dG=.dG=.d5C=.LA=.L5C=.LA |
| | ASO- | |
| | 287a | |
| 444 | hCPS1- | LT=.LG=.LA=.dA=.dT=.dG=.d5C=.dA=.dG=.dG=.d5C=.dA=.d5C=.LA=.L5C=.LA |
| | ASO- | |
| | 288a | |
| 445 | hCPS1- | LA=.LA=.LT=.dG=.d5C=.dA=.dG=.dG=.d5C=.dA=.d5C=.dA=.d5C=.LA=.L5C=.LA |
| | ASO- | |
| | 289a | |
| 446 | hCPS1- | LT=.LG=.L5C=.dA=.dG=.dG=.d5C=.dA=.d5C=.dA=.d5C=.dA=.d5C=.LA=.LT=.L5C |
| | ASO- | |
| | 290a | |
| 447 | hCPS1- | L5C=.LA=.LG=.dG=.d5C=.dA=.d5C=.dA=.d5C=.dA=.d5C=.dA=.dT=.L5C=.LA=.LG |
| | ASO- | |
| | 291a | |
| 448 | hCPS1- | LG=.LG=.L5C=.dA=.d5C=.dA=.d5C=.dA=.d5C=.dA=.dT=.d5C=.dA=.LG=.LG=.L5C |
| | ASO- | |
| | 292a | |
| 449 | hCPS1- | L5C=.LA=.L5C=.dA=.d5C=.dA=.d5C=.dA=.dT=.d5C=.dA=.dG=.dG=.L5C=.LT=.LG |
| | ASO- | |
| | 293a | |
| 450 | hCPS1- | L5C=.LA=.L5C=.dA=.d5C=.dA=.dT=.d5C=.dA=.dG=.dG=.d5C=.dT=.LG=.LG=.LG |
| | ASO- | |
| | 294a | |
| 451 | hCPS1- | LG=.LG=.L5C=.dA=.dT=.dG=.dA=.dA=.dT=.dG=.d5C=.dA=.dG=.LG=.L5C=.LA |
| | ASO- | |
| | 295a | |
| 452 | hCPS1- | L5C=.LA=.LG=.dG=.d5C=.dA=.dT=.dG=.dA=.dA=.dT=.dG=.d5C=.LA=.LG=.LG |
| | ASO- | |
| | 296a | |
| 453 | hCPS1- | LG=.LA=.L5C=.dA=.dG=.dG=.d5C=.dA=.dT=.dG=.dA=.dA=.dT=.LG=.L5C=.LA |
| | ASO- | |
| | 297a | |
| 454 | hCPS1- | LG=.LT=.LG=.dA=.d5C=.dA=.dG=.dG=.d5C=.dA=.dT=.dG=.dA=.LA=.LT=.LG |
| | ASO- | |
| | 298a | |
| 455 | hCPS1- | L5C=.LA=.L5C=.dA=.dT=.d5C=.dA=.dG=.dG=.d5C=.dT=.dG=.dG=.LG=.LG=.LA |
| | ASO- | |
| | 299a | |
| 456 | hCPS1- | L5C=.LA=.LT=.d5C=.dA=.dG=.dG=.d5C=.dT=.dG=.dG=.dG=.dG=.LA=.L5C=.L5C |
| | ASO- | |
| | 300a | |
| 457 | hCPS1- | LT=.L5C=.LA=.dG=.dG=.d5C=.dT=.dG=.dG=.dG=.dG=.dA=.d5C=.L5C=.L5C=.LT |
| | ASO- | |
| | 301a | |
| 458 | hCPS1- | LA=.LG=.LG=.d5C=.dT=.dG=.dG=.dG=.dG=.dA=.d5C=.d5C=.d5C=.LT=.LG=.LT |
| | ASO- | |
| | 302a | |
| 459 | hCPS1- | MG=.M5C=.MA=.MT=.dG=.dA=.dA=.dT=.dG=.d5C=.dA=.dG=.dG=.d5C=.MA=.M5C=.MA |
| | ASO- | =.M5C |
| | 303a | |
| 460 | hCPS1- | LG=.M5C=.LA=.MT=.dG=.dA=.dA=.dT=.dG=.d5C=.dA=.dG=.dG=.d5C=.MA=.L5C=.MA=. |
| | ASO- | L5C |
| | 303b | |
| 461 | hCPS1- | MG=.M5C=.MA=.M5C=.dA=.d5C=.dA=.d5C=.dA=.dT=.d5C=.dA=.dG=.dG=.M5C=.MT=.M |
| | ASO- | G=.MG |
| | 304a | |
| 462 | hCPS1- | LG=.M5C=.LA=.M5C=.dA=.d5C=.dA=.d5C=.dA=.dT=.d5C=.dA=.dG=.dG=.M5C=.LT=.MG |
| | ASO- | =.LG |
| | 304b | |
| 463 | hCPS1- | MA=.M5C=.MA=.M5C=.dA=.d5C=.dA=.dT=.d5C=.dA=.dG=.dG=.d5C=.dT=.MG=.MG=.MG |
| | ASO- | =.MG |
| | 305a | |
| 464 | hCPS1- | LA=.M5C=.LA=.M5C=.dA=.d5C=.dA=.dT=.d5C=.dA=.dG=.dG=.d5C=.dT=.MG=.LG=.MG=. |
| | ASO- | LG |
| | 305b | |
| 465 | hCPS1- | L5C=.MA=.M5C=.MA=.d5C=.dA=.d5C=.dA=.dT=.d5C=.dA=.dG=.dG=.d5C=.MT=.MG=.M |
| | ASO- | G=.LG |
| | 306a | |
| 466 | hCPS1- | L5C=.MA=.L5C=.MA=.d5C=.dA=.d5C=.dA=.dT=.d5C=.dA=.dG=.dG=.d5C=.MT=.LG=.MG |
| | ASO- | =.LG |
| | 306b | |
| 467 | hCPS1- | MG=.MG=.M5C=.MA=.MT=.dG=.dA=.dA=.dT=.dG=.d5C=.dA=.dG=.dG=.d5C=.MA=.M5C |
| | ASO- | =.MA=.M5C=.MA |
| | 307a | |
| 468 | hCPS1- | LG=.MG=.L5C=.MA=.MT=.dG=.dA=.dA=.dT=.dG=.d5C=.dA=.dG=.dG=.d5C=.MA=.M5C=. |
| | ASO- | LA=.M5C=.LA |
| | 307b | |
| 469 | hCPS1- | LG=.MG=.L5C=.MA=.MT=.dG=.dA=.dA=.LT=.dG=.d5C=.LA=.dG=.dG=.d5C=.MA=.M5C= |
| | ASO- | .LA=.M5C=.LA |
| | 308a | |
| 470 | hCPS1- | MG=.MG=.M5C=.MA-.MT-.dG=.dA=.dA=.dT=.dG=.d5C=.dA=.dG=.dG=.d5C=.MA-.M5C- |
| | ASO- | .MA=.M5C=.MA |
| | 308b | |
| 471 | hCPS1- | LG=.MG=.L5C=.MA=.M5C=.dA=.d5C=.dA=.d5C=.dA=.dT=.d5C=.dA=.dG=.dG=.M5C=.MT |
| | ASO- | =.LG=.MG=.LG |
| | 309a | |
| 472 | hCPS1- | LG=.MG=.L5C=.MA=.M5C=.dA=.d5C=.dA=.L5C=.dA=.dT=.L5C=.dA=.dG=.dG=.M5C=.M |
| | ASO- | T=.LG=.MG=.LG |
| | 309b | |
| 473 | hCPS1- | MG=.MG=.M5C=.MA-.M5C-.dA=.d5C=.dA=.d5C=.dA=.dT=.d5C=.dA=.dG=.dG=.M5C- |
| | ASO- | .MT-.MG=.MG=.MG |
| | 309c | |
| 474 | hCPS1- | M5C=.MA=.M5C=.MA=.M5C=.dA=.d5C=.dA=.dT=.d5C=.dA=.dG=.dG=.d5C=.dT=.MG=.M |
| | ASO- | G=.MG=.MG=.MA |
| | 310a | |
| 475 | hCPS1- | L5C=.MA=.L5C=.MA=.M5C=.dA=.d5C=.dA=.dT=.d5C=.dA=.dG=.dG=.d5C=.dT=.MG=.MG |
| | ASO- | =.LG=.MG=.LA |
| | 310b | |
| 476 | hCPS1- | L5C=.MA=.L5C=.MA=.M5C=.dA=.d5C=.dA=.LT=.d5C=.dA=.LG=.dG=.d5C=.dT=.MG=.M |
| | ASO- | G=.LG=.MG=.LA |
| | 310c | |
| 477 | hCPS1- | M5C=.MA=.M5C=.MA-.M5C-.dA=.d5C=.dA=.dT=.d5C=.dA=.dG=.dG=.d5C=.dT=.MG- |
| | ASO- | MG-.MG=.MG=.MA |
| | 310d | |
| 478 | hCPS1- | MG=.M5C=.MA=.M5C=.MA=.d5C=.dA=.d5C=.dA=.dT=.d5C=.dA=.dG=.dG=.d5C=.MT=.M |
| | ASO- | G=.MG=.MG=.MG |
| | 311a | |
| 479 | hCPS1- | LG=.M5C=.LA=.M5C=.MA=.d5C=.dA=.d5C=.dA=.dT=.d5C=.dA=.dG=.dG=.d5C=.MT=.MG |
| | ASO- | =.LG=.MG=.LG |
| | 311b | |
| 480 | hCPS1- | MG=.M5C=.MA=.M5C-.MA-.d5C=.dA=.d5C=.dA=.dT=.d5C=.dA=.dG=.dG=.d5C=.MT- |
| | ASO- | MG-.MG=.MG=.MG |
| | 311c | |
| 481 | hCPS1- | dT=.dG=.d5C=.dA=.dG=.dG=.d5C=.dA=.d5C=.dA=.d5C=.dA=.d5C=.dA=.dT=.d5C=.dA=.dG |
| | ASO- | =.dG=.d5C |
| | 312a | |
| 482 | hCPS1- | MA=.MT=.M5C=.MA=.MG=.dG=.d5C=.dT=.dG=.dG=.dG=.dG=.dA=.d5C=.d5C=.M5C=.MT |
| | ASO- | =.MG=.MT=.MG-.[TEG] |
| | 313a | |
| 483 | hCPS1- | MG=.MG=.M5C=.MA=.M5C=.dA=.d5C=.dA=.d5C=.dA=.dT=.d5C=.dA=.dG=.dG=.M5C=.M |
| | ASO- | T=.MG=.MG=.MG-.[TEG] |
| | 314a | |
| 484 | hCPS1- | MA=.MT=.MG=.MA=.MA=.dT=.dG=.d5C=.dA=.dG=.dG=.d5C=.dA=.d5C=.dA=.M5C=.MA |
| | ASO- | =.M5C=.MA=.MT-.[TEG] |
| | 315a | |
| 485 | hCPS1- | MT=.MG=.M5C=.MA=.MG=.dG=.d5C=.dA=.d5C=.dA=.d5C=.dA=.d5C=.dA=.dT=.M5C=.M |
| | ASO- | A=.MG=.MG=.M5C-.[TEG] |
| | 316a | |
| 486 | hCPS1- | MT=.MG=.M5C-.MA-.MG=.dG=.mC=.dA=.d5C=.dA=.d5C=.dA=.d5C=.dA=.dT=.M5C-.MA- |
| | ASO- | MG=.MG=.M5C-.[TEG] |
| | 316b | |

In some embodiments, hCPS1-ASO-317-492 (SEQ ID NOs: 487-662) comprise pS bonds, 5-MethylCytosines and fully MOE nucleotides.

### Hybridization and ΔG

The term “hybridizing” or “hybridizes” as used herein is to be understood as two nucleic acid strands (e.g., an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the free energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem, Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present disclosure hybridize to a target nucleic acid with estimated ΔG° values below −10 kcal/mol for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal/mol, such as below −15 kcal/mol, such as below −20 kcal/mol and such as below −25 kcal/mol for oligonucleotides that are 8-30 nucleotides in length. In some embodiments the oligonucleotides hybridize to a target nucleic acid with an estimated ΔG° value of −10 to −60 kcal/mol, such as −12 to −40 kcal/mol, −15 to −30 kcal/mol, −16 to −27 kcal/mol, or −18 to −25 kcal/mol.

### Duplex Region

The phrase “duplex region” refers to the region in two complementary or substantially complementary polynucleotides that form base pairs with one another, either by Watson-Crick base pairing or any other manner that allows for a stabilized duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” has 19 base pairs. The remaining bases may, for example, exist as 5′ and 3′ overhangs. Further, within the duplex region, 100% complementarity is not required; substantial complementarity is allowable within a duplex region. Substantial complementarity refers to 70% or greater complementarity. For example, a mismatch in a duplex region consisting of 19 base pairs results in 94.7% complementarity, rendering the duplex region substantially complementary. Duplex regions can be formed by two separate oligonucleotide strands, as well as by single oligonucleotide strands that can form hairpin structures comprising a duplex region.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of a CPS1 regRNA, such as an eRNA or paRNA. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides. Generally, the duplex structure is between 15 and 50 base pairs in length, e.g., between, 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-30, 18-30, 18-29, 18-28, 18-27, 18-26, 18-25, 18-24, 18-23, 18-22, 18-21, 18-20, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-30, 19-30, 19-29, 19-28, 19-27, 19-26, 19-25, 19-24, 19-23, 19-22, 19-21, 19-20, 20-50, 20-49, 20-48, 20-47, 20-46, 20-45, 20-44, 20-43, 20-42, 20-41, 20-40, 20-39, 20-38, 20-37, 20-36, 20-35, 20-34, 20-33, 20-32, 20-31, 20-30, 20-30, 20-29, 20-28, 20-27, 20-26, 20-25, 20-24, 20-23, 20-22, 20-21, 21-50, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-30, 21-29, 21-28, 21-27, 21-26, 21-25, 21-24, 21-23, 21-22, 22-50, 22-49, 22-48, 22-47, 22-46, 22-45, 22-44, 22-43, 22-42, 22-41, 22-40, 22-39, 22-38, 22-37, 22-36, 22-35, 22-34, 22-33, 22-32, 22-31, 22-30, 22-29, 22-28, 22-27, 22-26, 22-25, 22-24, 22-23, 23-50, 23-49, 23-48, 23-47, 23-46, 23-45, 23-44, 23-43, 23-42, 23-41, 23-40, 23-39, 23-38, 23-37, 23-36, 23-35, 23-34, 23-33, 23-32, 23-31, 23-30, 23-29, 23-28, 23-27, 23-26, 23-25, or 23-24 base pairs in length. Ranges and lengths intermediate to the above recited ranges and lengths are also contemplated to be part of the disclosure.

Similarly, the region of complementarity to the target sequence can be between 15 and 50 nucleotides in length, e.g., between 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-30, 18-30, 18-29, 18-28, 18-27, 18-26, 18-25, 18-24, 18-23, 18-22, 18-21, 18-20, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-30, 19-30, 19-29, 19-28, 19-27, 19-26, 19-25, 19-24, 19-23, 19-22, 19-21, 19-20, 20-50, 20-49, 20-48, 20-47, 20-46, 20-45, 20-44, 20-43, 20-42, 20-41, 20-40, 20-39, 20-38, 20-37, 20-36, 20-35, 20-34, 20-33, 20-32, 20-31, 20-30, 20-30, 20-29, 20-28, 20-27, 20-26, 20-25, 20-24, 20-23, 20-22, 20-21, 21-50, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-30, 21-29, 21-28, 21-27, 21-26, 21-25, 21-24, 21-23, 21-22, 22-50, 22-49, 22-48, 22-47, 22-46, 22-45, 22-44, 22-43, 22-42, 22-41, 22-40, 22-39, 22-38, 22-37, 22-36, 22-35, 22-34, 22-33, 22-32, 22-31, 22-30, 22-29, 22-28, 22-27, 22-26, 22-25, 22-24, 22-23, 23-50, 23-49, 23-48, 23-47, 23-46, 23-45, 23-44, 23-43, 23-42, 23-41, 23-40, 23-39, 23-38, 23-37, 23-36, 23-35, 23-34, 23-33, 23-32, 23-31, 23-30, 23-29, 23-28, 23-27, 23-26, 23-25, or 23-24 nucleotides in length. Ranges and lengths intermediate to the above recited ranges and lengths are also contemplated to be part of the disclosure.

### Chemical Modifications of ASOs

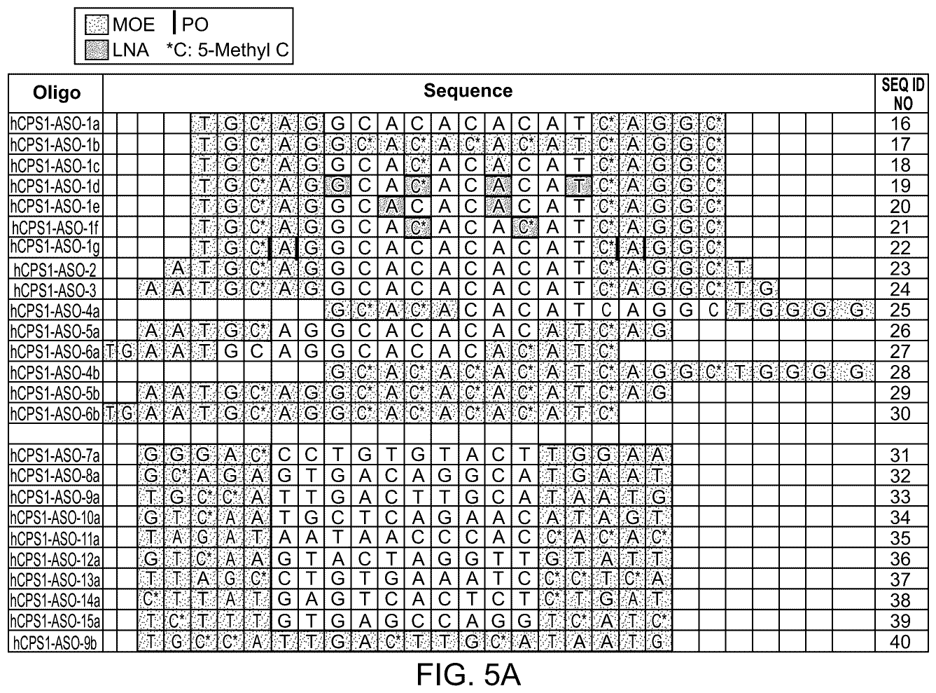

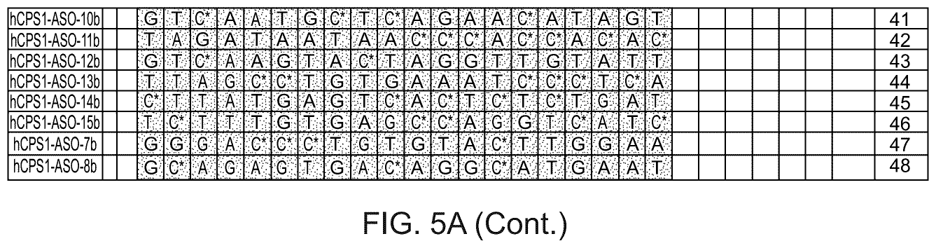

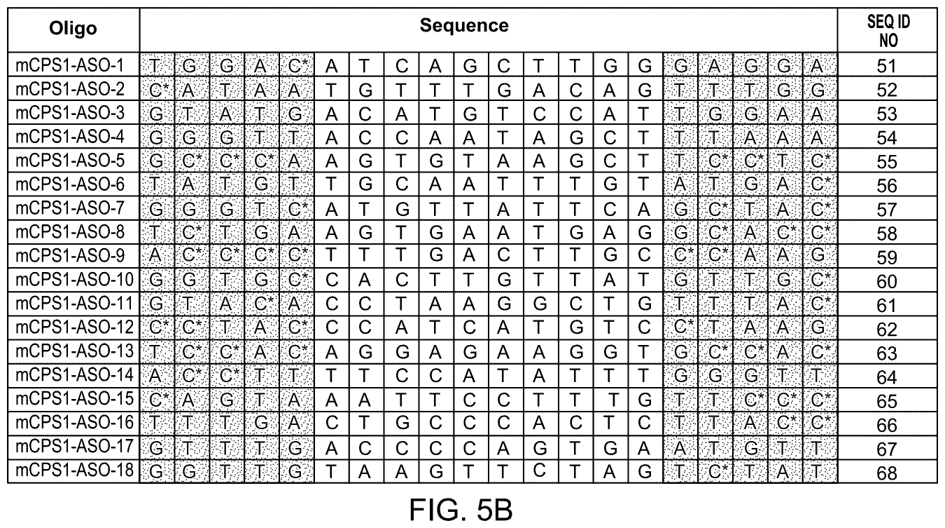

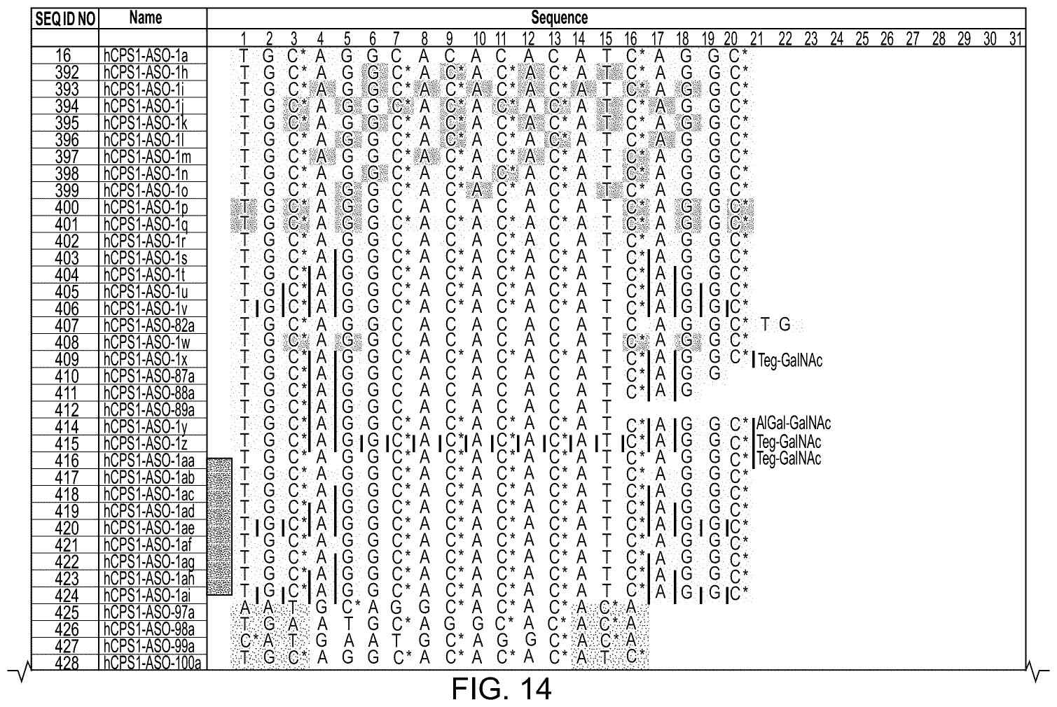

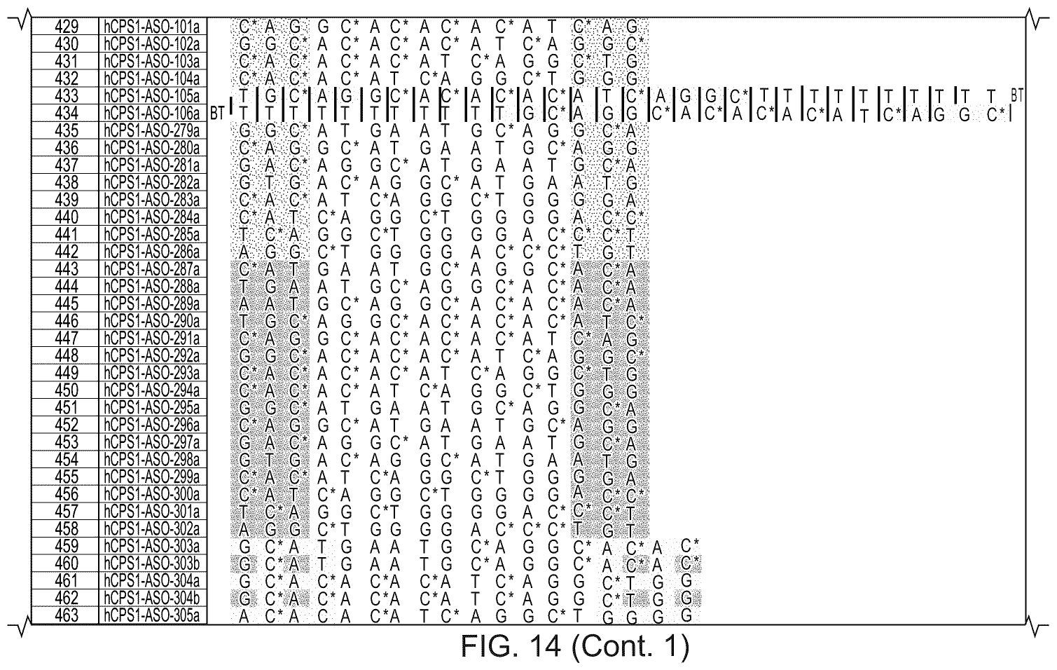

In certain embodiments, the ASO does not consist of only DNA. In certain embodiments, the ASO comprises at least one chemical modification relative to a natural nucleotide (e.g., ribonucleotide). Various chemical modifications can be included in the ASOs of the present disclosure. The modifications can include one or more modifications in a ribose group, one or more modifications in a phosphate group, one or more modifications in a nucleobase, one or more terminal modifications, or a combination thereof. In some embodiments, an exemplary ASO sequence targeting a regRNA as shown in Table 2 is chemically modified. For example, hCPS1-ASO-1 may be chemically modified to comprise the modifications of any one of hCPS1-ASO1-1a to hCPS1-ASO1-1g as shown in FIG. 5A. Such modifications can be, but are not limited to, 2′-O-(2-methoxyethyl) (2′-MOE, MOE), locked nucleic acid (LNA), 5-methyl on the cytidine, cET, phosphorothioate (PS) linkage, and/or a phosphodiester (PO) linkage, or any combination thereof. Chemical modifications of RNA are known in the art and described in, for example, PCT Application Publication No. WO2013/177248. In certain embodiments, each cytidine in the ASO is modified by 5-methyl. Exemplary ASOs comprising chemical modifications are shown in FIGS. 5A and 14.

Various chemical modifications for use with ASOs of the present disclosure include, but are not limited to: 3′-terminal deoxy-thymine (dT) nucleotides, 2′-O-methyl modified nucleotides, 2′-fluoro modified nucleotides, 2′-deoxy-modified nucleotides, locked nucleotides, unlocked nucleotides, conformationally restricted nucleotides, constrained ethyl nucleotides, abasic nucleotides, 2′-amino-modified nucleotides, 2′-O-allyl-modified nucleotides, 2′-C-alkyl-modified nucleotides, 2′-hydroxyl-modified nucleotides, 2′-methoxyethyl modified nucleotides, 2′-O-alkyl-modified nucleotides, morpholino nucleotides, phosphoramidates, non-natural base comprising nucleotides, tetrahydropyran modified nucleotides, 1,5-anhydrohexitol modified nucleotides, cyclohexenyl modified nucleotides, nucleotides comprising a phosphorothioate group, nucleotides comprising a methylphosphonate group, nucleotides comprising a 5′-phosphate, and nucleotides comprising a 5′-phosphate mimic.

In certain embodiments, the ASO comprises an RNA polynucleotide chemically modified to be resistant to one or more nuclear RNases (e.g., the exosome complex). In some embodiments, all nucleotide bases are modified in the ASO. In certain embodiments, the chemical modifications comprises β-D-ribonucleosides, 2′-modified nucleosides (e.g., 2′-O-(2-methoxyethyl) (2′-MOE), 2′-O—CH₃, or 2′-fluoro-arabino (FANA)), bicyclic sugar modified nucleosides (e.g., having a constrained ethyl or locked nucleic acid (LNA)), and/or one or more modified internucleotide bonds (e.g., phosphorothioate internucleotide linkage). In certain embodiments, the chemical modification comprises 2′-MOE and a phosphorothioate internucleotide bond. In certain embodiments, at least 8, 9, 10, 11, 12, 13, 14, 15, 16, 17, 18, 19, 20, or more consecutive nucleotides of the ASO are modified by 2′-MOE. In certain embodiments, each nucleotide of the ASO is modified by 2′-MOE. In certain embodiments, at least 7, 8, 9, 10, 11, 12, 13, 14, 15, 16, 17, 18, 19, 20, or more consecutive internucleotide bonds of the ASO are phosphorothioate internucleotide bonds. In certain embodiments, each internucleotide bond of the ASO is a phosphorothioate internucleotide bond.

Internucleotide linkage modifications that can be used with the ASOs of the present disclosure include, but are not limited to, phosphorothioate “PS” (P(S)), phosphoramidate (P(NR1R2) such as dimethylaminophosphoramidate(P(N(CH3)2)), phosphonocarboxylate (P(CH2)nCOOR) such as phosphonoacetate “PACE” (P(CH2COO—)), thiophosphonocarboxylate ((S)P(CH2)nCOOR) such as thiophosphonoacetate “thioPACE” ((S)P(CH2COO—)), alkylphosphonate (P(C1-3alkyl) such as methylphosphonate —P(CH3), boranophosphonate (P(BH3)), and phosphorodithioate (P(S)2).

The chemical structures can also be described in writing. In such cases, ‘M’ indicates MOE; ‘d’ indicates DNA, ‘L’ indicates LNA, ‘=’ indicates a phosphorothioate (PS) linkage, ‘—’ indicates a phosphodiester (PO) linkage; ‘5C’ indicates 5-MethylCytosine, ‘ag’ indicates GalNAc, ‘tg’ or “TEG” indicates Teg-GalNAc, ‘ag’ or ‘AlGal’ indicates GalNAc, ‘BioTEG’ indicated biotin, ‘CholTEG’ indicates cholesterol, ‘c’ indicates cET, and ‘\hat{ }’ indicates FANA.

To avoid ambiguity, this LNA has the formula:

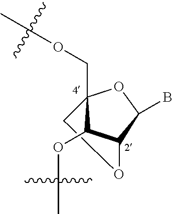

wherein B is the particular designated base.

Exemplary written descriptions of selected ASOs are provided in Table 3, with corresponding FIGS. 5A and 14 providing visual representation of the modifications.

In some embodiments, the ASO comprises a sequence selected from the group consisting of SEQ ID NOs: 1-15, 91-391, or 487-662. In some embodiments, the ASO comprises a sequence and chemical modification selected from the group consisting of SEQ ID NOs: 16-48, 392-480, or 482-486. In some embodiments, the ASO comprises a sequence and/or chemical modification selected from the group consisting of SEQ ID NOs: 1-48 or 91-662.

In some embodiments, the regulatory RNA comprises a sequence selected from the group consisting of SEQ ID NOs: 80-88. In some embodiments, the ASO comprises a sequence selected from the group consisting of SEQ ID NOs: 51-68.

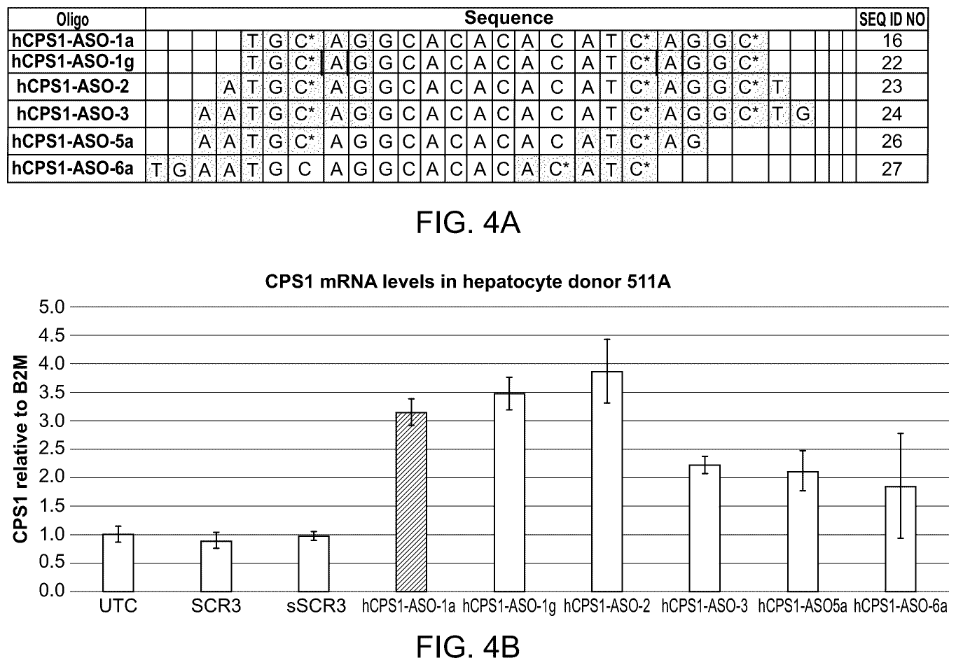

In some embodiments, the ASO comprises a sequence set forth in Table 2. In some embodiments, the ASO comprises a sequence and/or the chemical modification set forth in Table 3, and FIGS. 4A, 5A, 5B, and 14.

In certain embodiments, the ASO comprises one or more chemical modifications at the 5′ end, the 3′ end, or both. Without wishing to be bound by theory, chemical modifications at one or both termini of a polynucleotide (e.g., polyribonucleotide) may stabilize the polynucleotide. In certain embodiments, the ASO comprises one or more chemical modifications in at least 1, 2, 3, 4, or 5 nucleotides at the 5′ end of the ASO. In certain embodiments, the ASO comprises one or more chemical modifications in at least 1, 2, 3, 4, or 5 nucleotides at the 3′ end of the ASO. In certain embodiments, the ASO comprises one or more chemical modifications in at least 1, 2, 3, 4, or 5 nucleotides at the 5′ end of the ASO and one or more chemical modifications in at least 1, 2, 3, 4, or 5 nucleotides at the 3′ end of the ASO.

### High Affinity Modified Nucleosides

A high affinity modified nucleoside is a modified nucleoside which, when incorporated into the oligonucleotide enhances the affinity of the oligonucleotide for its complementary target, for example as measured by the melting temperature (Tm). A high affinity modified nucleoside of the present disclosure preferably result in an increase in melting temperature between +0.5 to +12° C., such as between +1.5 to +10° C. or +3 to +8° C. per modified nucleoside. Numerous high affinity modified nucleosides are known in the art and include for example, many 2′ substituted nucleosides as well as locked nucleic acids (LNA) (see e.g. Freier & Altmann, Nucl. Acid Res., 1997, 25, 4429-4443 and Uhlmann, Curr. Opinion in Drug Development, 2000, 3(2), 293-213), each of which is hereby incorporated by reference.

### Sugar Modifications

The ASOs described herein may comprise one or more nucleosides which have a modified sugar moiety, i.e. a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance. Such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (see e.g., PCT Application Publication No. WO2011/017521) or tricyclic nucleic acids (see e.g., PCT Application Publication No. WO2013/154798), both of which are hereby incorporated by reference. Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

In some embodiments, oligonucleotides comprise modified sugar moieties, such as any one of a 2′-O-methyl (2′OMe) moiety, a 2′-O-methoxyethyl moiety, a bicyclic sugar moiety, PNA (e.g., an oligonucleotide comprising one or more N-(2-aminoethyl)-glycine units linked by amide bonds or carbonyl methylene linkage as repeating units in place of a sugar-phosphate backbone), locked nucleoside (LNA) (e.g., an oligonucleotide comprising one or more locked ribose, and can be a mixture of 2′-deoxy nucleotides or 2′OMe nucleotides), c-ET (e.g., an oligonucleotide comprising one or more cET sugar), cMOE (e.g., an oligonucleotide comprising one or more cMOE sugar), morpholino oligomer (e.g., an oligonucleotide comprising a backbone comprising one or more phosphorodiamidate morpholiono oligomers), 2′-deoxy-2′-fluoro nucleoside (e.g., an oligonucleotide comprising one or more 2′-fluoro-β-D-arabinonucleoside), tcDNA (e.g., an oligonucleotide comprising one or more tcDNA modified sugar), constrained ethyl 2′-4′-bridged nucleic acid (cEt), S-cEt, ethylene bridged nucleic acid (ENA) (e.g., an oligonucleotide comprising one or more ENA modified sugar), hexitol nucleic acids (HNA) (e.g., an oligonucleotide comprising one or more HNA modified sugar), or tricyclic analog (tcDNA) (e.g., an oligonucleotide comprising one or more tcDNA modified sugar).

In some embodiments, oligonucleotides comprise nucleobase modifications selected from the group consisting of 2-thiouracil (“2-thioU”), 2-thiocytosine (“2-thioC”). 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”) 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allyl U”), 5-allylcytosine (“5-allylC”), 5-aminoallyluracil (“5-aminoallylU”), 5-aminoallyl-cytosine (“5-aminoallylC”), an abasic nucleotide, Z base, P base, Unstructured Nucleic Acid (“UNA”), isoguanine (“isoG”), and isocytosine (“isoC”), glycerol nucleic acid (GNA), thiomorpholino (C4H9NS) or thiophosphoramidate morpholinos (TMOs). Synthesis of glycerol nucleic acid (GNA) (also known as glycol nucleic acids) is described in Zhang et al, Current Protocols in Nucleic Acid Chemistry 4.40.1-4.40.18, September 2010, hereby incorporated by reference. Synthesis of thiophosphoramidate morpholino oligonucleotides is described in Langer et al, J. Am. Chem. Soc. 2020, 142, 38, 16240-16253

### 2′ Sugar Modified Nucleosides

A 2′ sugar modified nucleoside is a nucleoside which has a substituent other than H or —OH at the 2′ position (2′ substituted nucleoside) or comprises a 2′ linked biradical capable of forming a bridge between the 2′ carbon and a second carbon in the ribose ring, such as LNA (2′-4′ biradical bridged) nucleosides.

Without wishing to be bound by theory, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937, each of which are hereby incorporated by reference.

### Locked Nucleic Acid Nucleosides (LNA Nucleoside)

A “LNA nucleoside” is a 2′-sugar modified nucleoside which comprises a biradical linking the C2′ and C4′ of the ribose sugar ring of said nucleoside (also referred to as a “2′-4′ bridge”), which restricts or locks the conformation of the ribose ring. In other words, a locked nucleoside is a nucleoside comprising a bicyclic sugar moiety comprising a 4′-CH₂—O-2′ bridge. This structure effectively “locks” the ribose in the 3′-endo structural conformation. The addition of locked nucleosides to oligonucleotides has been shown to increase oligonucleotide stability in serum, and to reduce off-target effects (Grunweller, A. et al., (2003) Nucleic Acids Research 31(12):3185-3193). These nucleosides are also sometimes termed bridged nucleic acid or bicyclic nucleic acid (BNA). The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex. Exemplary LNA nucleosides include beta-D-oxy-LNA, 6′-methyl-beta-D-oxy LNA such as (S)-6′-methyl-beta-D-oxy-LNA (ScET) and ENA.

Examples of bicyclic nucleosides for use in the polynucleotides of the invention include without limitation nucleosides comprising a bridge between the 4′ and the 2′ ribosyl ring atoms. In certain embodiments, the polynucleotide agents of the invention include one or more bicyclic nucleosides comprising a 4′ to 2′ bridge. Examples of such 4′ to 2′ bridged bicyclic nucleosides, include but are not limited to 4′-(CH₂)—O-2′ (LNA); 4′-(CH₂)₂—S-2′; 4′-(CH₂)2-O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)₂-2′ (and analogs thereof; see e.g., U.S. Pat. No. 8,278,425); 4′-CH₂—O—N(CH₃)₂-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. patents and US patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see PCT Publication No. WO 99/14226, contents of which are incorporated by reference herein).

An oligonucleotide of the invention can also be modified to include one or more constrained ethyl nucleosides. As used herein, a “constrained ethyl nucleoside” or “cEt” is a locked nucleoside comprising a bicyclic sugar moiety comprising a 4′-CH(CH₃)—O-2′ bridge. In some embodiments, a constrained ethyl nucleoside is in the S conformation referred to herein as “S-cEt.”

An oligonucleotide of the invention may also include one or more “conformationally restricted nucleosides” (“CRN”). CRN are nucleoside analogs with a linker connecting the C2′ and C4′ carbons of ribose or the -C3 and -C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT Publication No. WO 2013/036868, the entire contents of each of which are hereby incorporated herein by reference.

In some embodiments, an oligonucleotide of the invention comprises one or more monomers that are UNA (unlocked nucleoside) nucleosides. UNA is unlocked acyclic nucleoside, wherein any of the bonds of the sugar has been removed, forming an unlocked “sugar” residue. In one example, UNA also encompasses monomer with bonds between C1′-C4′ have been removed (i.e., the covalent carbon-oxygen-carbon bond between the C1′ and C4′ carbons). In another example, the C2′-C3′ bond (i.e., the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

The ribose molecule may also be modified with a cyclopropane ring to produce a tricyclodeoxynucleic acid (tricyclo DNA). The ribose moiety may be substituted for another sugar such as 1,5,-anhydrohexitol, threose to produce a threose nucleoside (TNA), or arabinose to produce an arabino nucleoside. The ribose molecule can also be replaced with non-sugars such as cyclohexene to produce cyclohexene nucleoside or glycol to produce glycol nucleosides.

Potentially stabilizing modifications to the ends of nucleoside molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other alternatives chemistries of an oligonucleotide of the invention include a 5′ phosphate or 5′ phosphate mimic, e.g., a 5′-terminal phosphate or phosphate mimic of an oligonucleotide. Suitable phosphate mimics are disclosed in, for example US Patent Publication No. 2012/0157511, the entire contents of which are incorporated herein by reference.

Additional non-limiting, exemplary LNA nucleosides are disclosed in PCT Publication Nos. WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, Mitsuoka et al., Nucleic Acids Research 2009, 37(4), 1225-1238, and Wan and Seth, J. Medical Chemistry 2016, 59, 9645-9667, each of which are hereby incorporated by reference.

In some embodiments, the length of the ASO is 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl.

In some embodiments, the nucleotide sugar modification is 2′-O—C1-4alkyl such as 2′-O-methyl (2′-OMe), 2′-deoxy (2′-H), 2-O—C1-3alkyl-O—C1-3alkyl such as 2′-methoxyethyl (“2′-MOE”), 2′-fluoro (“2′-F”), 2′-amino (“2-NH2”), 2′-arabinosyl (“2′-arabino”) nucleotide, 2′-F-arabinosyl (“2′-F-arabino”) nucleotide, 2′-locked nucleic acid (“LNA”) nucleotide, 2′-amido bridge nucleic acid (AmNA), 2′-unlocked nucleic acid (“ULNA”) nucleotide, a sugar in L form (“L-sugar”), or 4′-thioribosyl nucleotide.

### Mixmers and Gapmers

The ASO can have a mixmer and/or gapmer structure, for example, in a pattern disclosed by the ASOs in FIG. 5A, FIG. 5B, or FIG. 14.

In certain embodiments, the ASO is a mixmer. As used herein, the term “mixmer” refers to an oligonucleotide comprising an alternating composition of DNA monomers and nucleoside analogue monomers across at least a portion of the oligonucleotide sequence. In certain embodiments, the ASO is a mixmer based on the gapmer structure, comprising a mixture of DNA nucleotides and 2′-MOE nucleotides in the gap, flanked by RNA sequences in the wings. Mixmers may be designed to comprise a mixture of affinity enhancing nucleotide analogues, such as in non-limiting example 2′-O-alkyl-RNA monomers, 2′-amino-DNA monomers, 2′-fluoro-DNA monomers, LNA monomers, arabino nucleic acid (ANA) monomers, 2′-fluoro-ANA monomers, HNA monomers, INA monomers, 2′-MOE-RNA (2′-O-methoxyethyl-RNA), 2′Fluoro-DNA, and LNA. In some embodiments, the mixmer is incapable of recruiting RNase H. In some embodiments, the mixmer comprises one type of affinity enhancing nucleotide analogue together with DNA and/or RNA.

Multiple different modifications can be interspaced in a mixmer. For example, the ASO can comprise LNA modification in a plurality of nucleotides and a different modification in some or all of the rest of the nucleotides. In some embodiments, any two adjacent LNA-modified nucleotides are separated by at least 1, 2, 3, 4, or 5 nucleotides. Throughout the ASO, the distance between adjacent LNA-modified nucleotides can either be constant (e.g., any two adjacent LNA-modified nucleotides are separated by 1, 2, 3, 4, or 5 nucleotides) or variable. In some embodiments, the length of the ASO is 3×n, 3×n−1, or 3×n−2 nucleotides (n is an integer of 6 or greater), wherein (a) (i) the nucleotides at positions 3×m−2 (m is an integer from 1 to n) are ribonucleotides comprising a first modification (e.g., LNA), (ii) the nucleotides at positions 3×m−1 (m is an integer from 1 to n) are ribonucleotides comprising a first modification (e.g., LNA), or (iii) the nucleotides at positions 3×m (m is an integer from 1 to n) are ribonucleotides comprising a first modification (e.g., LNA); and (b) the nucleotides at the remaining positions comprise a second, different modification (e.g., 2′-O-methoxyethyl). The ASO called hCPS1-ASO-1d herein has such a structure. In some embodiments, the length of the ASO is 2×n or 2×n−1 nucleotides (n is an integer of 9 or greater), wherein (a) (i) the nucleotides at positions 2×m−1 (m is an integer from 1 to n) are ribonucleotides comprising a first modification (e.g., LNA), or (ii) the nucleotides at positions 2×m (m is an integer from 1 to n) are ribonucleotides comprising a first modification (e.g., LNA); and (b) the nucleotides at the remaining positions comprise a second, different modification (e.g., 2′-O-methoxyethyl). The ASO called hCPS1-ASO-1e herein has such a structure. Similar modification patterns, for example, where the first modification is repeated every 4, 5, or more nucleotides, are also contemplated herein.

In some embodiments, the ASO further comprises a GalNAc or Teg-GalNAc moiety at the 5′ or 3′ end of the ASO. In some embodiments, the ASO further comprises a cholesterol or biotin moiety at the 5′ or 3′ end of the ASO.

In certain embodiments, the ASO comprises a DNA sequence (e.g., having at least 5, 6, 7, 8, 9, 10, 11, 12, 13, 14, or 15 contiguous nucleotides of unmodified DNA) flanked by RNA sequences. Such structure is known as “gapmer,” in which the internal DNA region is referred to as the “gap” and the external RNA regions is referred to as the “wings” (see, e.g., PCT Application Publication No. WO2013/177248). Gapmers were known to facilitate degradation of the target RNA by recruiting nuclear RNAses (e.g., RNase H). Surprisingly, in the present disclosure, it has been discovered that a gapmer binding a regRNA (e.g., hCPS1-ASO-1a), like regRNAs having the same sequence but having different chemical modifications (e.g., hCPS1-ASO-1g), can also increase target gene expression.

In certain embodiments, the gapmer is about 15, 16, 17, 18, 19, 20, 21, 22, 23, 24, 25, 26, 27, 28, 29, 30, or more nucleotides in length. In certain embodiments, the gap is about 5, 6, 7, 8, 9, 10, 11, 12, 13, 14, 15, or more nucleotides in length. In certain embodiments, one or both wings are about 2, 3, 4, 5, 6, 7, 8, 9, 10, or more nucleotides in length. In certain embodiments, one or both wings comprises RNA modifications, for example, β-D-ribonucleosides, 2′-modified nucleosides (e.g., 2′-O-(2-methoxyethyl) (2′-MOE), 2′-O—CH₃, or 2′-fluoro-arabino (FANA)), and bicyclic sugar modified nucleosides (e.g., having a constrained ethyl or locked nucleic acid (LNA)). In certain embodiments, each ribonucleotide in the gapmer is modified by 2′-MOE. In certain embodiments, the gapmer comprises one or more modified internucleotide bonds, e.g., phosphorothioate (PS) internucleotide linkage. In certain embodiments, each two adjacent nucleotides in the gapmer are linked by a phosphorothioate internucleotide bond.

In certain embodiments, the ASO does not comprise 7 or more, 8 or more, 9 or more, 10 or more, 10 or more, 11 or more, 12 or more, 13 or more, 14 or more, or 15 or more contiguous nucleotides of unmodified DNA. In some embodiments, such a DNA sequence is disrupted by modified (e.g., 2′-MOE modified) ribonucleotides every 2, 3, 4, 5, or more nucleotides. In some embodiments, the ASO comprises only ribonucleotides and no deoxyribonucleotides.

The structural features of mixmer and gapmer can be combined. In certain embodiments, the ASO has a structure similar to that of a mixmer disclosed herein (e.g., one having interspaced modifications), except that the second modification in the gap is changed to a third modification (e.g., deoxyribonucleotide). In certain embodiments, the ASO has a structure similar to that of a gapmer disclosed herein, except that in the gap the nucleotides are modified in a mixmer pattern.

In certain embodiments, the ASO further comprises a ligand moiety, e.g., a ligand moiety that specifically targets a tissue or organ in a subject. For example, N-acetylgalactosamine (GalNAc) specifically targets liver. In certain embodiments, the ligand moiety comprises GalNAc. In certain embodiments, the ligand moiety comprises a three-cluster GalNAc moiety, commonly denoted GAlNAc3. Other types of GalNAc moieties are one-cluster, two cluster or four cluster GAlNAc, denoted as GAlNAc1, GAlNAc2, or GAlNAc4. In certain embodiments, the ligand moiety comprises GalNAc1, GALNAc2, GAlNAc3, or GalNAc4.

### III. Pharmaceutical Compositions

In certain embodiments, the ASOs disclosed herein can be present in pharmaceutical compositions. The pharmaceutical composition can be formulated for use in a variety of drug delivery systems. One or more pharmaceutically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer, Science 249:1527-1533, 1990, hereby incorporated by reference in its entirety.

Exemplary carriers and pharmaceutical formulations suitable for delivering nucleic acids are described in Durymanov and Reineke (2018) Front. Pharmacol. 9:971; Barba et al. (2019) Pharmaceutics 11(8): 360; Ni et al. (2019) Life (Basel) 9(3): 59. It is understood that the presence of a ligand moiety conjugated to the ASO may circumvent the need for a carrier for delivery to a tissue or organ targeted by the ligand moiety.

The delivery of an oligonucleotide of the disclosure to a cell e.g., a cell within a subject, such as a human subject e.g., a subject in need thereof, such as a subject having a CPS1 related disorder or other urea cycle disorders can be achieved in a number of different ways. For example, delivery may be performed by contacting a cell with an oligonucleotide of the disclosure either in vitro or in vivo. In vivo delivery may also be performed directly by administering a composition comprising an oligonucleotide to a subject. These alternatives are discussed further below.

In general, any method of delivering a nucleic acid molecule (in vitro or in vivo) can be adapted for use with an oligonucleotide of the disclosure (see e.g., Akhtar S. and Julian R L., (1992) Trends Cell. Biol. 2(5):139-144 and PCT Publication No. WO 94/02595, which are incorporated herein by reference in their entireties). For in vivo delivery, factors to consider in order to deliver an oligonucleotide molecule include, for example, biological stability of the delivered molecule, prevention of non-specific effects, and accumulation of the delivered molecule in the target tissue. The non-specific effects of an oligonucleotide can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the oligonucleotide molecule to be administered.

For administering an oligonucleotide systemically for the treatment of a disease, the oligonucleotide can include alternative nucleobases, alternative sugar moieties, and/or alternative internucleoside linkages, or alternatively delivered using a drug delivery system; both methods act to prevent the rapid degradation of the oligonucleotide by endo- and exo-nucleases in vivo. Modification of the oligonucleotide or the pharmaceutical carrier can also permit targeting of the oligonucleotide composition to the target tissue and avoid undesirable off-target effects. Oligonucleotide molecules can be modified by chemical conjugation to lipophilic groups such as cholesterol to enhance cellular uptake and prevent degradation. In an alternative embodiment, the oligonucleotide can be delivered using drug delivery systems such as a nanoparticle, a lipid nanoparticle, a polyplex nanoparticle, a lipoplex nanoparticle, a dendrimer, a polymer, liposomes, or a cationic delivery system. Positively charged cationic delivery systems facilitate binding of an oligonucleotide molecule (negatively charged) and also enhance interactions at the negatively charged cell membrane to permit efficient uptake of an oligonucleotide by the cell. Cationic lipids, dendrimers, or polymers can either be bound to an oligonucleotide, or induced to form a vesicle or micelle that encases an oligonucleotide. The formation of vesicles or micelles further prevents degradation of the oligonucleotide when administered systemically. In general, any methods of delivery of nucleic acids known in the art may be adaptable to the delivery of the oligonucleotides of the disclosure. Methods for making and administering cationic oligonucleotide complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al., (2007) J. Hypertens. 25:197-205, which are incorporated herein by reference in their entirety). Some non-limiting examples of drug delivery systems useful for systemic delivery of oligonucleotides include DOTAP (Sorensen, D R., et al (2003), supra; Verma, U N. et al., (2003), supra), Oligofectamine™, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, an oligonucleotide forms a complex with cyclodextrin for systemic administration. Methods for administration and pharmaceutical compositions of oligonucleotides and cyclodextrins can be found in U.S. Pat. No. 7,427,605, which is herein incorporated by reference in its entirety. In some embodiments the oligonucleotides of the disclosure are delivered by polyplex or lipoplex nanoparticles. Methods for administration and pharmaceutical compositions of oligonucleotides and polyplex nanoparticles and lipoplex nanoparticles can be found in U.S. Patent Publication Nos. 2017/0121454; 2016/0369269; 2016/0279256; 2016/0251478; 2016/0230189; 2015/0335764; 2015/0307554; 2015/0174549; 2014/0342003; 2014/0135376; and 2013/0317086, which are herein incorporated by reference in their entirety.

In some embodiments, the compounds described herein may be administered in combination with additional therapeutics. Examples of additional therapeutics include standard of care urea cycle disorder treatments such as low protein diet or nitrogen scavengers including, but not limited to, glycerol phenylbutyrate, sodium benzoate, phenylbutyrate, or phenylacetate.

### Membranous Molecular Assembly Delivery Methods

Oligonucleotides of the disclosure can also be delivered using a variety of membranous molecular assembly delivery methods including liquid nanoparticles (LNPs), polymeric, biodegradable microparticles, or microcapsule delivery devices known in the art. For example, a colloidal dispersion system may be used for targeted delivery of an oligonucleotide agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUVs), which range in diameter from 0.2-4.0 μm can encapsulate a substantial percentage of an aqueous buffer containing large macromolecules. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide are delivered into the cell where the oligonucleotide can specifically bind to a target RNA. In some cases, the liposomes are also specifically targeted, e.g., to direct the oligonucleotide to particular cell types. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

A liposome containing an oligonucleotide can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The oligonucleotide preparation is then added to the micelles that include the lipid component. The cationic groups on the lipid interact with the oligonucleotide and condense around the oligonucleotide to form a liposome. After condensation, the detergent is removed, e.g., by dialysis, to yield a liposomal preparation of oligonucleotide.

If necessary, a carrier compound that assists in condensation can be added during the condensation reaction, e.g., by controlled addition. For example, the carrier compound can be a polymer other than a nucleic acid (e.g., spermine or spermidine). The pH can also be adjusted to favor condensation.

Methods for producing stable polynucleotide delivery vehicles, which incorporate a polynucleotide/cationic lipid complex as a structural component of the delivery vehicle, are further described in, e.g., WO 96/37194, the entire contents of which are incorporated herein by reference. Liposome formation can also include one or more aspects of exemplary methods described in Feigner, P. L. et al., (1987) Proc. Natl. Acad. Sci. USA 8:7413-7417; U.S. Pat. Nos. 4,897,355; 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75: 4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169). These methods are readily adapted to packaging oligonucleotide preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems comprising non-ionic surfactant and cholesterol. Non-ionic liposomal formulations comprising NOVASOME™ I (glyceryl dilaurate/cholesterol/polyoxyethylene-10-stearyl ether) and NOVASOME™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P.Pharma. Sci., 4(6):466).

Liposomes may also be sterically stabilized liposomes, comprising one or more specialized lipids that result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes comprising one or more glycolipids are known in the art. Papahadjopoulos et al. (Ann. N.Y. Acad. Sci., (1987), 507:64) reported the ability of monosialoganglio side G^{M1}, galactocerebroside sulfate, and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver oligonucleotides to macrophages.

Further advantages of liposomes include: liposomes obtained from natural phospholipids are biocompatible and biodegradable; liposomes can incorporate a wide range of water and lipid soluble drugs; liposomes can protect encapsulated oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of oligonucleotide (see, e.g., Feigner, P. L. et al., (1987) Proc. Natl. Acad. Sci. USA 8:7413-7417, and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. LIPOFECTIN™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (TRANSFECTAM™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer oligonucleotide into the skin. In some implementations, liposomes are used for delivering oligonucleotide to epidermal cells and also to enhance the penetration of oligonucleotide into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2, 405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems comprising non-ionic surfactant and cholesterol. Non-ionic liposomal formulations comprising NOVASOME I (glyceryl dilaurate/cholesterol/polyoxyethylene-10-stearyl ether) and NOVASOME II (glyceryl distearate/cholesterol/polyoxyethylene-10-stearyl ether) were used to deliver a drug into the dermis of mouse skin. Such formulations with oligonucleotides are useful for treating a dermatological disorder.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include oligonucleotides can be made highly deformable. Such deformability can enable the liposomes to penetrate through pore that are smaller than the average radius of the liposome. For example, transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes can be made by adding surface edge activators, usually surfactants, to a standard liposomal composition. Transfersomes that include oligonucleotides can be delivered, for example, subcutaneously by infection in order to deliver oligonucleotides to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading. Transfersomes have been used to deliver serum albumin to the skin. The transfersome-mediated delivery of serum albumin has been shown to be as effective as subcutaneous injection of a solution containing serum albumin.

Other formulations amenable to the present disclosure are described in PCT Publication Nos. WO 2009/088891, WO 2009/132131, and WO 2008/042973, which are hereby incorporated by reference in their entirety.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The oligonucleotides for use in the methods of the disclosure can also be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

### Lipid Nanoparticle-Based Delivery Methods

Oligonucleotides of in the disclosure may be fully encapsulated in a lipid formulation, e.g., a lipid nanoparticle (LNP), or other nucleic acid-lipid particle. LNPs are useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present disclosure typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present disclosure are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

Non-limiting examples of cationic lipids include N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyetetrahydro-3aH-cyclopenta[d][1,3]dioxol-5-amine (ALN100), (6Z,9Z,28Z,31Z)-heptatriaconta-6,9,28,31-tetraen-19-yl4-(dimethylamino)bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can comprise, for example, from about 20 mol % to about 50 mol % or about 40 mol % of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 60 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C₁₂), a PEG-dimyristyloxypropyl (C₁₄), a PEG-dipalmityloxypropyl (C₁₆), or a PEG-distearyloxypropyl (C₁₈). The conjugated lipid that prevents aggregation of particles can be, for example, from 0 mol % to about 20 mol % or about 2 mol % of the total lipid present in the particle.

In some embodiments, the nucleic acid-lipid particle further includes cholesterol at, e.g., about 10 mol % to about 60 mol % or about 50 mol % of the total lipid present in the particle.

The ASO may also be delivered in a lipidoid. The synthesis of lipidoids has been extensively described and formulations containing these compounds are particularly suited for delivery of modified nucleic acid molecules or ASOs (see Mahon et al, Bioconjug Chem. 2010 21: 1448-1454; Schroeder et al, J Intern Med. 2010 267:9-21; Akinc et al, Nat Biotechnol. 2008 26:561-569; Love et al, Proc Natl Acad Sci USA. 2010 107: 1864-1869; Siegwart et al, Proc Natl Acad Sci USA. 2011 108: 12996-3001; all of which are incorporated herein in their entireties).

Lipid compositions for RNA delivery are disclosed in PCT Publication Nos. WO2012/170930A1, WO2013/149141A1, and WO2014/152211A1, each of which is hereby incorporated by reference.

### IV. Therapeutic Applications

The present disclosure provides methods for treating diseases and disorders associated with decreased gene expression (e.g., decreased CPS1 gene expression). The method employs an ASO that hybridizes with a regulatory RNA transcribed from a regulatory element of the target gene (e.g., CPS1) or a pharmaceutical composition comprising the ASO. The oligonucleotide compositions described herein are useful in the methods of the disclosure and, while not bound by theory, are believed to exert their desirable effects through their ability to modulate the level, status, and/or activity of CPS1, e.g., by increasing the level of the CPS1 protein in a cell in a subject (e.g., a mammal, a primate, or a human).

Also provided herein are methods of treating or preventing hyperammonemia in a subject, including administering an ASO described herein to the subject. The disclosure further provides methods of treating or preventing hyperammonemia in a subject that has or is at risk of developing hyperammonemia, including administering an ASO described herein to the subject. In some embodiments, the subject having or at risk of developing hyperammonemia has one or more of the following: a urea cycle disorder, hepatic encephalopathy, acute liver failure, liver cirrhosis, non-alcoholic fatty liver disease, renal dysfunction and/or failure, propionic acidemia, methylmalonic acidemia, isovaleric acidemia, a urinary tract infection, intestinal bacterial overgrowth, a fatty acid oxidation disorder, and a systemic infection. In some embodiments, the subject has or is at risk of developing hyperammonemia associated with the intake of a drug (e.g., valproic acid, carbamazepine, fluorouracil, sulfadiazine, ribavirin, salicylates, and glycine). In some embodiments, the methods include administering an ASO described herein to the subject. In certain embodiments, treatment with an ASO described herein results in decreased ammonia levels (e.g., blood ammonia levels, plasma ammonia levels, systemic ammonia levels, or cerebrospinal fluid ammonia levels) in a subject as compared to a subject (e.g., the same subject prior to the treatment) that has not been treated with the ASO. In some embodiments, the reduction in ammonia levels (e.g., blood ammonia levels, systemic ammonia levels, or cerebrospinal fluid ammonia levels) is a reduction of at least about 5%, 10%, 15%, 20%, 25%, 30%, 40%, 50%, 60%, 70%, 80%, 90%, or 100%, relative to the ammonia levels (e.g., blood ammonia levels, systemic ammonia levels, or cerebrospinal fluid ammonia levels) in the subject (e.g., as compared to the same subject prior to administration of the ASO).

Methods of measuring ammonia levels (e.g., in a plasma sample) are known in the art and include enzymatic kinetic assays in which ammonia reacts with α-ketoglutarate and reduced nicotinamide adenine dinucleotide phosphate (NADPH) in the presence of L-glutamate dehydrogenase resulting in glutamate and NADP⁺. The amount of oxidized NADPH is then measured (e.g., photometrically) and is equivalent to the amount of ammonia.

An aspect of the present disclosure relates to methods of treating disorders (e.g., a urea cycle disorder) in a subject (e.g., a human subject) in need thereof. In some embodiments, provided herein are methods of treating a urea cycle disorder in a subject (e.g., a human subject) in need thereof, including administering an ASO provided herein to the subject. In some embodiments, the urea cycle disorder is carbamoyl-phosphate synthetase 1 (CPS1) deficiency, ornithine transcarbamylase (OTC) deficiency, citrullinemia type I (argininosuccinate synthetase (ASS) deficiency), argininosuccinate lyase (ASL) deficiency, N-acetyl glutamate synthetase (NAGS) deficiency, arginase deficiency (hyperargininemia, ARG1 deficiency), ornithine translocase deficiency (ORNT1 deficiency, hyperornithinemia-hyperammonemia-homocitrullinuria syndrome (HHH)), or citrin deficiency. In some embodiments, the urea cycle disorder is not N-acetyl glutamate synthetase (NAGS) deficiency. In some embodiments, administration of an ASO provided herein to a subject having a urea cycle disorder reduces the risk, frequency and/or severity of a hyperammonemic crisis in the subject (e.g., as compared to a subject that is not administered the ASO (e.g., the same subject prior to administration of the ASO)).

In some embodiments, the urea cycle disorder is Carbamoylphosphate synthetase I deficiency (CPS1 deficiency). In some embodiments, the urea cycle disorder is hyperammonemia. In some embodiments, the urea cycle disorder is Ornithine transcarbamylase deficiency (OTC deficiency). In some embodiments, the urea cycle disorder is Citrullinemia type I (ASS1 deficiency). In some embodiments, the urea cycle disorder is Argininosuccinic aciduria (ASL deficiencydeficiency). In some embodiments, the urea cycle disorder is Arginase deficiency (hyperargininemia, ARG1 deficiency). In some embodiments, the urea cycle disorder is Ornithine translocase deficiency (ORNT1 deficiency, hyperornithinemia-hyperammonemia-homocitrullinuria syndrome). In some embodiments, the urea cycle disorder is Citrin deficiency.

Another aspect of the disclosure includes methods of increasing the level of CPS1 in a cell of a subject identified as having a CPS1 related disorder (e.g., CPS1 deficiency) or other urea cycle disorder. Still another aspect includes a method of increasing expression of CPS1 in a cell in a subject. The methods may include contacting a cell with an ASO described herein, in an amount effective to increase expression of CPS1 in the cell, thereby increasing expression of CPS1 in the cell. In certain embodiments, the methods include contacting a cell with an ASO described herein, in an amount effective to increase expression of CPS1 in the cell, thereby increasing expression of OTC in the cell. In certain embodiments, the methods include contacting a cell with an ASO described herein, in an amount effective to increase expression of CPS1 in the cell, thereby increasing expression of other urea cycle genes in the cell, such as OTC, ASS1, ASL, ARG1, or ORNT1.

Based on the above methods, further aspects of the present disclosure include an ASO or oligonucleotide of the disclosure, or a composition comprising such an ASO or oligonucleotide, for use in therapy, or for use as a medicament, or for use in treating a CPS1 related disorder (e.g., CPS1 deficiency) or other urea cycle disorder in a subject in need thereof, or for use in increasing the level of CPS1 in a cell of a subject identified as having a CPS1 related disorder or other urea cycle disorder, or for use in increasing expression of CPS1 in a cell in a subject. The uses include the contacting of a cell with the ASO or oligonucleotide, in an amount effective to increase expression of CPS1 in the cell, thereby increasing expression of CPS1 in the cell. Embodiments described below in relation to the methods of the present disclosure are also applicable to these further aspects.

Contacting of a cell with an ASO or oligonucleotide may be done in vitro, ex vivo, or in vivo. Contacting a cell in vivo with the oligonucleotide includes contacting a cell or group of cells within a subject, e.g., a human subject, with the ASO or oligonucleotide. Combinations of in vitro and in vivo methods of contacting a cell are also possible. Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc3 ligand, or any other ligand that directs the oligonucleotide to a site of interest. The cell can be a liver cell (e.g., a hepatocyte).

Administration of the ASO or oligonucleotide or pharmaceutical composition disclosed herein could be intravenous, intraarterial, intraperitoneal, intramuscular, subcutaneous, intrapleural, intrathecal, intracavitary, by perfusion through a catheter or by direct intralesional injection. In certain embodiments, the ASO or oligonucleotide or pharmaceutical composition is administered systemically. In certain embodiments, the ASO or oligonucleotide or pharmaceutical composition is administered by a parenteral route. For example, in certain embodiments, the ASO or oligonucleotide or pharmaceutical composition is administered by intravenously (e.g., by intravenous infusion), for example, with a prefilled bag, a prefilled pen, or a prefilled syringe. In other embodiments, the ASO or oligonucleotide or pharmaceutical composition is administered locally to an organ or tissue in which an increase in the target gene expression is desirable (e.g., liver).

In some embodiments, the ASO or oligonucleotide is administered to a subject such that the ASO or oligonucleotide is delivered to a specific site within the subject. Such targeted delivery can be achieved by either systemic administration or local administration. The increase of expression of CPS1 may be assessed using measurements of the level or change in the level of CPS1 mRNA or CPS1 protein in a sample derived from a specific site within the subject. In some embodiments, the methods include measuring the levels of ammonia in a sample (e.g., blood, urine, plasma or cerebrospinal fluid) derived from the subject. Methods of measuring ammonia levels are well known in the art and described above. In certain embodiments, the methods include a clinically relevant increase of expression of CPS1, e.g., as demonstrated by a clinically relevant outcome after treatment of a subject with an agent to increase the expression of CPS1.

In other embodiments, the oligonucleotide is administered in an amount and for a time effective to result in reduction (e.g., by 10%, 20%, 30%, 40%, 50%, 60%, 70%, 80%, 90%, or 100%) of one or more symptoms of a CPS1 related disorder or a urea cycle disorder, such as high ammonia level in the blood.

### Increase of CPS1 Expression Level

The therapeutic methods disclosed herein, using an ASO that targets CPS1, are designed to increase CPS1 expression level in a subject as compared to an untreated subject (e.g., the same subject prior to administration of the ASO or the pharmaceutical composition). Increasing expression of a CPS1 gene includes any level of increasing of a CPS1 gene, e.g., at least partial increase of the expression of a CPS1 gene. Increase may be assessed by an increase in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control). In certain embodiments, the method causes a clinically relevant increase of expression of CPS1, e.g. as demonstrated by a clinically relevant outcome after treatment of a subject with an agent to increase the expression of CPS1.

In certain embodiments, the methods disclosed herein increases CPS1 gene expression by at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, relative to the pre-dose baseline level. In certain embodiments, the methods disclosed herein increases CPS1 gene expression by at least 2 fold, at least 3 fold, at least 4 fold, at least 5 fold, at least 6 fold, at least 7 fold, at least 8 fold, at least 9 fold, or at least 10 fold relative to the pre-dose baseline level. In certain embodiments, the subject has a deficiency in CPS1 expression, and the method disclosed herein restores the CPS1 expression level or activity to at least 20%, at least 30%, at least 40%, at least 50%, at least 60%, at least 70%, at least 80%, at least 90%, or at least 100% of the average CPS1 expression level or activity in subjects of the species of like age and gender.

The expression of a CPS1 gene may be assessed based on the level of any variable associated with CPS1 gene expression, e.g., CPS1 mRNA level or CPS1 protein level. It is understood that CPS1 is a chromosome-2 gene in certain mammals (e.g., human and mouse). In certain embodiments, the expression level or activity of CPS1 herein refers to the average expression level or activity in the liver.

In certain embodiments, surrogate markers can be used to detect an increase of CPS1 expression level. For example, effective treatment of a CPS1 related disorder, as demonstrated by acceptable diagnostic and monitoring criteria with an agent to increase CPS1 expression can be understood to demonstrate a clinically relevant increase in CPS1.

Increase of the expression of a CPS1 gene may be manifested by an increase of the amount of mRNA expressed by a first cell or group of cells (such cells may be present, for example, in a sample derived from a subject) in which a CPS1 gene is transcribed and which has or have been treated (e.g., by contacting the cell or cells with an oligonucleotide of the disclosure, or by administering an oligonucleotide of the disclosure to a subject in which the cells are or were present) such that the expression of a CPS1 gene is increased, as compared to a second cell or group of cells substantially identical to the first cell or group of cells but which has not or have not been so treated (control cell(s) not treated with an oligonucleotide or not treated with an oligonucleotide targeted to the gene of interest).

In other embodiments, increase of the expression of a CPS1 gene may be assessed in terms of an increase of a parameter that is functionally linked to CPS1 gene expression, e.g., CPS1 protein expression or CPS1 activity. CPS1 increase may be determined in any cell expressing CPS1, either endogenous or heterologous from an expression construct, and by any assay known in the art.

An increase of CPS1 expression may be manifested by an increase in the level of the CPS1 protein that is expressed by a cell or group of cells (e.g., the level of protein expressed in a sample derived from a subject), relative to a control cell or a control group of cells. An increase of CPS1 expression may also be manifested by an increase in the level of the CPS1 mRNA level in a treated cell or group of cells, relative to a control cell or a control group of cells.

A control cell or group of cells that may be used to assess the increase of the expression of a CPS1 gene includes a cell or group of cells that has not yet been contacted with an oligonucleotide of the disclosure. For example, the control cell or group of cells may be derived from an individual subject (e.g., a human or animal subject) prior to treatment of the subject with an oligonucleotide.

The level of CPS1 mRNA that is expressed by a cell or group of cells may be determined using any method known in the art for assessing mRNA expression. In one embodiment, the level of expression of CPS1 in a sample is determined by detecting a transcribed polynucleotide, or portion thereof, e.g., mRNA of the CPS1 gene. RNA may be extracted from cells using RNA extraction techniques including, for example, using acid phenol/guanidine isothiocyanate extraction (RNAzol B; Biogenesis), RNEASY™ RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating CPS1 mRNA may be detected using methods described in PCT Publication No. WO 2012/177906, the entire contents of which are hereby incorporated herein by reference. In some embodiments, the level of expression of CPS1 is determined using a nucleic acid probe. The term “probe,” as used herein, refers to any molecule that is capable of selectively binding to a specific CPS1 sequence, e.g. to an mRNA or polypeptide. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses, and probe arrays. One method for the determination of mRNA levels involves contacting the isolated mRNA with a nucleic acid molecule (probe) that can hybridize to CPS1 mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an AFFYMETRIX gene chip array. A skilled artisan can readily adapt known mRNA detection methods for use in determining the level of CPS1 mRNA.

An alternative method for determining the level of expression of CPS1 in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression of CPS1 is determined by quantitative fluorogenic RT-PCR (i.e., the TAQMAN™ System) or the DUAL-GLO® Luciferase assay.

The expression levels of CPS1 mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722; 5,874,219; 5,744,305; 5,677,195; and 5,445,934, which are incorporated herein by reference. The determination of CPS1 expression level may also comprise using nucleic acid probes in solution.

In some embodiments, the level of mRNA expression is assessed using branched DNA (bDNA) assays or real time RT PCR or qPCR. Such methods can also be used for the detection of CPS1 nucleic acids.

The level of CPS1 protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of CPS1 proteins.

### V. Additional Embodiments

Embodiment 1 An ASO complementary to at least 8 contiguous nucleotides of a regulatory RNA (regRNA) of Carbamoyl-Phosphate Synthetase 1 (CPS1), wherein the ASO is complementary to a sequence in the regRNA that is no more than 200 nucleotides from a terminus of the regRNA.

Embodiment 2 The ASO of embodiment 1, wherein the ASO is complementary to a sequence in the regRNA that is no more than 200 nucleotides from the 3′ end of the regRNA.

Embodiment 3 The ASO of embodiment 1, wherein the ASO is complementary to a sequence in the regRNA that is no more than 200 nucleotides from the 5′ end of the regRNA.

Embodiment 4 The ASO of embodiments 1 or 2, wherein the ASO comprises a RNA polynucleotide comprising one or more chemical modifications.

Embodiment 5 The ASO of embodiment 1, wherein at least 3, 4, or 5 nucleotides at the 5′ end and at least 3, 4, or 5 nucleotides at the 3′ end of the ASO comprise ribonucleotides with one or more chemical modifications.

Embodiment 6 The ASO of embodiments 1 or 2, wherein the one or more chemical modifications comprise 2′-O-methoxyethyl, 5-methyl cytidine, locked nucleic acid (LNA), and phosphorothioate internucleotide bond.

Embodiment 7 The ASO of embodiments 1-3, wherein the ASO does not comprise 8 or more contiguous nucleotides of unmodified DNA.

Embodiment 8 An ASO complementary to at least 8 contiguous nucleotides of a regulatory RNA of a CPS1 gene, wherein the ASO does not comprise 8 or more contiguous nucleotides of unmodified DNA.

Embodiment 9 The ASO of embodiments 1-8, wherein the ASO does not comprise a deoxyribonucleotide.

Embodiment 10 The ASO of embodiments 1-9, wherein the ASO does not comprise an unmodified ribonucleotide.

Embodiment 11 The ASO of embodiments 1-10, wherein the length of the ASO is 3×n+2 nucleotides (n is an integer of 6 or greater), wherein the nucleotides at positions 3×m are ribonucleotides modified by LNA (m is an integer from 1 to n) and the nucleotides at the remaining positions are ribonucleotides modified by 2′-O-methoxyethyl.

Embodiment 12 The ASO of embodiments 1-11, wherein each nucleotide of the ASO is a ribonucleotide modified by 2′-O-methoxyethyl.

Embodiment 13 The ASO of embodiments 1-12, wherein each cytidine in the ASO is modified by 5-methyl.

Embodiment 14 The ASO of embodiments 1-13, wherein the ASO is no more than 50, 40, 30, or 25 nucleotides in length.

Embodiment 15 The ASO of embodiments 1-14, wherein the regRNA is an eRNA.

Embodiment 16 A pharmaceutical composition comprising the ASO of any one of embodiments 1-15 and a pharmaceutically acceptable carrier or excipient carrier.

Embodiment 17 A method of increasing the amount and/or stability of a regulatory RNA in a cell, the method comprising contacting the cell with the ASO of any one of embodiments 1-15 that hybridizes with the regulatory RNA.

Embodiment 18 A method of increasing transcription of a target gene in a cell, the method comprising contacting the cell with the ASO of any one of embodiments 1-15 that hybridizes with a regulatory RNA of the target gene.

Embodiment 19 The method of embodiments 17 or 18, wherein the cell is a mammalian cell.

Embodiment 20 The method of embodiment 19, wherein the cell is a human cell.

Embodiment 21 The method of embodiments 17-20, wherein the target gene is CPS1.

### EXAMPLES

### Example 1: Modulation of CPS1 Expression Using regRNA-Targeting ASOs

This example was designed to assess modulation of CPS1 expression in human hepatocytes using ASOs targeting eRNAs transcribed from an enhancer of human CPS1.

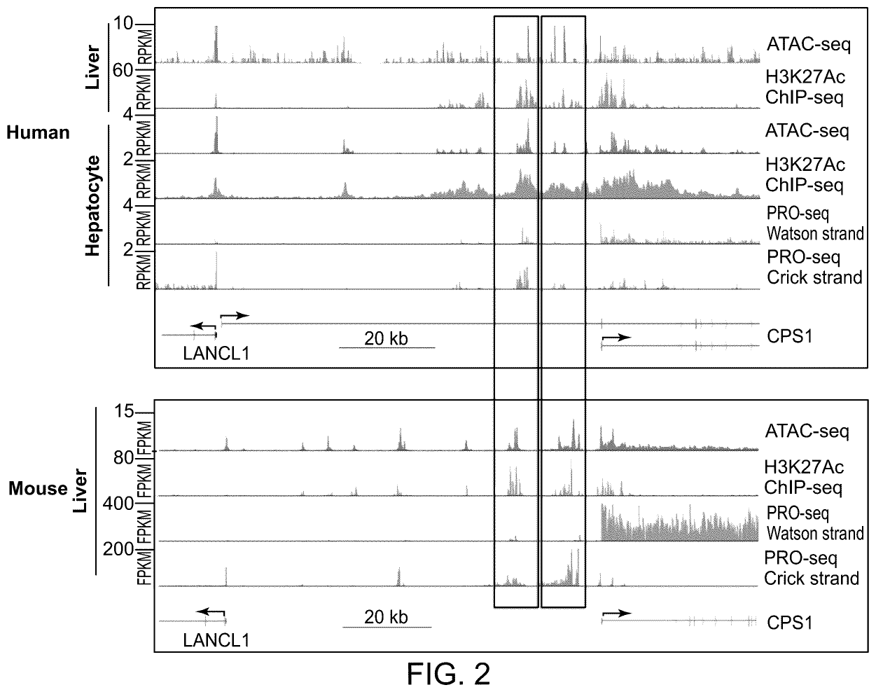

Regulatory RNAs (regRNAS) within enhancers of CPS1 were identified bioinformatically using ATAC-seq and H3K27Ac ChIP-SEQ data from human and mouse data. Two regRNAs were identified (RR44_v1 and RR44_v2) as conserved between human and mouse, as shown in FIG. 2.

330 unique antisense oligonucleotides (ASOs) targeting CPS1 were designed, 94 of which target the CPS1 regRNA described above (RR44).

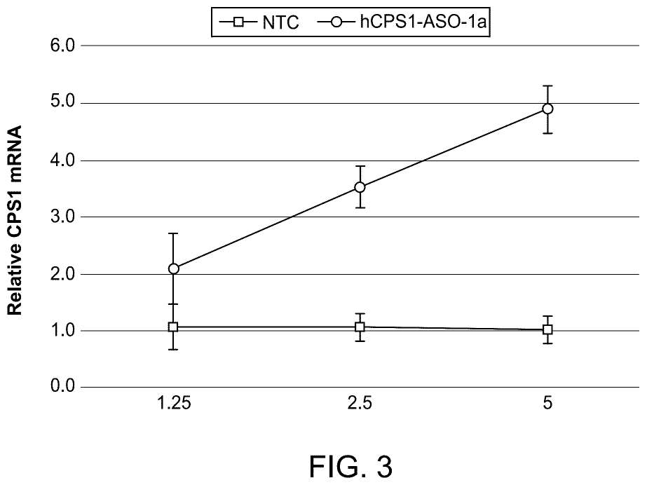

Donor-derived hepatocytes were cultured in vitro. Cells were plated in growth media and treated 4-6 hours after plating with final concentrations of 1.25 μM, 2.5 μM, or 5 μM, or hCPS1-ASO-1a or control (see FIG. 5A for human CPS1 sequences and chemical modifications of selected ASOs). Cells were collected 48 hr post treatment and processed for RNA isolation, cDNA synthesis and QPCR analysis. A Tagman probe at 60× was used for CPS1 expression. CPS1 levels were normalized to B2M expression. FIG. 3 shows CPS1 mRNA after treatment with hCPS1-ASO-1a, demonstrating the ASO targeting regRNA RR44 increased human CPS1 mRNA in a dose-dependent matter.

Using hCPS1-ASO-1a as a starting point, additional ASOs were designed by base-walking. These ASOs are shown in FIG. 4A. Using the methods described above, these additional ASOs were assessed for effect on CPS1 mRNA, and results are shown in FIG. 4B. Additional ASOs are shown in FIG. 5A.

The majority of regRNAs do not have large sequence areas that are conserved between human and mouse genomes. For in vivo proof of concept, regRNAs around the mouse CPS1 region were identified and ASOs targeting those mouse regRNA (promoter and enhancer) were designed and screened in both wildtype (B6EiC3SnF1/J, [WT]) primary mouse hepatocytes and CPS1 deficient donor (B6EiC3Sn alA-CPS1^{spf-ash}/J, [CPS1D]) primary mouse hepatocytes.

Primary hepatocytes were isolated from mice of mouse strains B6EiC3SnF1/J (control WT) and OTC deficient donor (B6EiC3Sn alA-OTC1^{spf-ash}/J, catalog: 001811) from JAX lab. The spf^{ash} mouse has a variant c.386G>A, p.Arg129His in the OTC gene that impacts splicing, resulting in decreased OTC mRNA levels (5˜12% of wt control) in spf/ash livers. Thus, male spf^{ash} mice have a mild biochemical phenotype with low OTC activity (5%-10% of wild-type).

Primary hepatocytes were seeded at 20,000 cells per well on day 0. Cells were treated with ASO on day 2 at concentrations of 1.25 μM, 2.5 μM, and 5 μM. Cells were incubated for 2 days and lysate was collected on Day 2 post treatment for mRNA analysis. A Taqman probe was used for determining mouse CPS1 and OTC expression. Ppia and Hprt were used as housekeeper genes for gene expression normalization. Statistics were performed using one-way ANOVA in Prism (GraphPad)

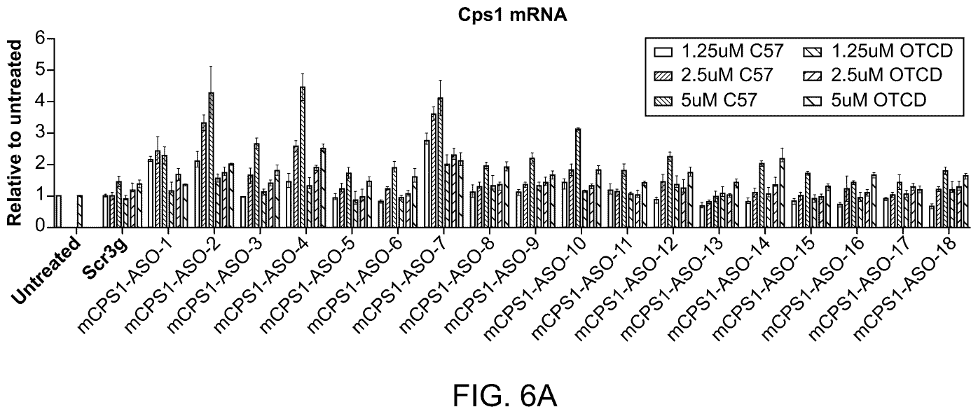

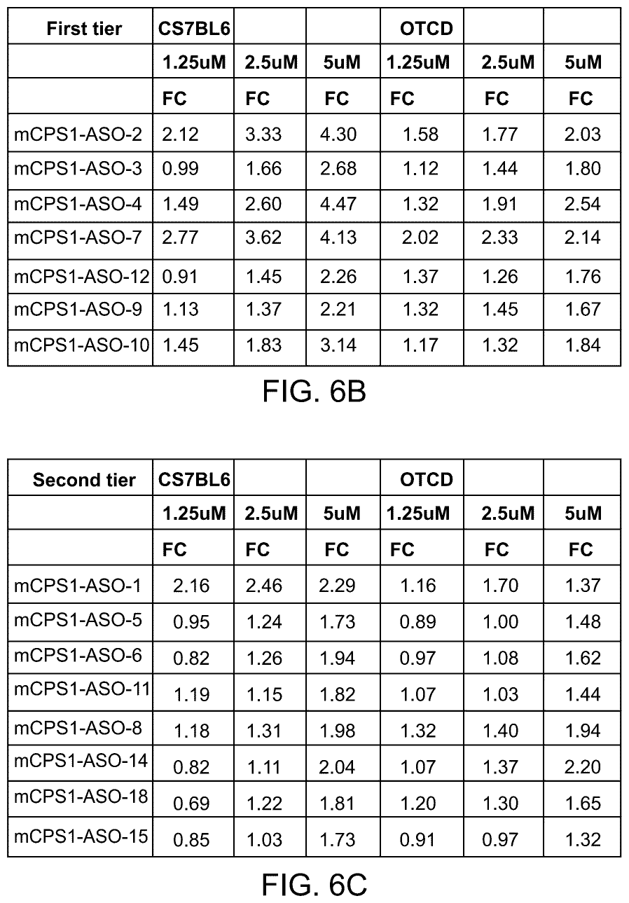

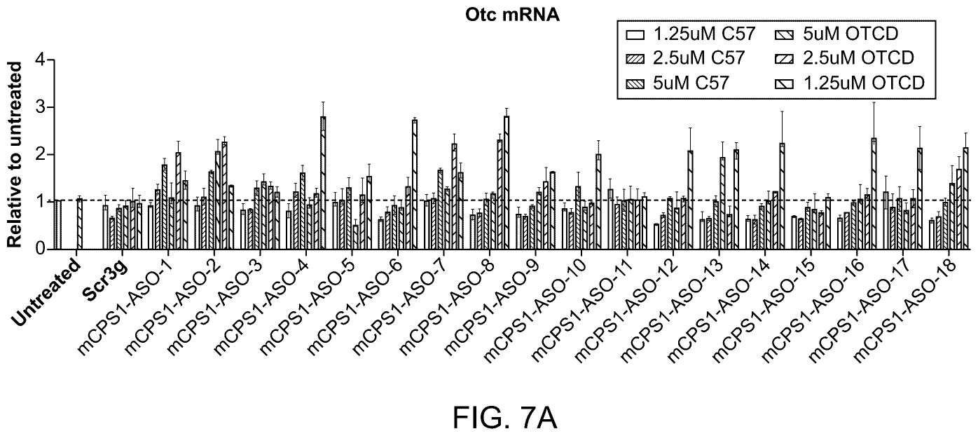

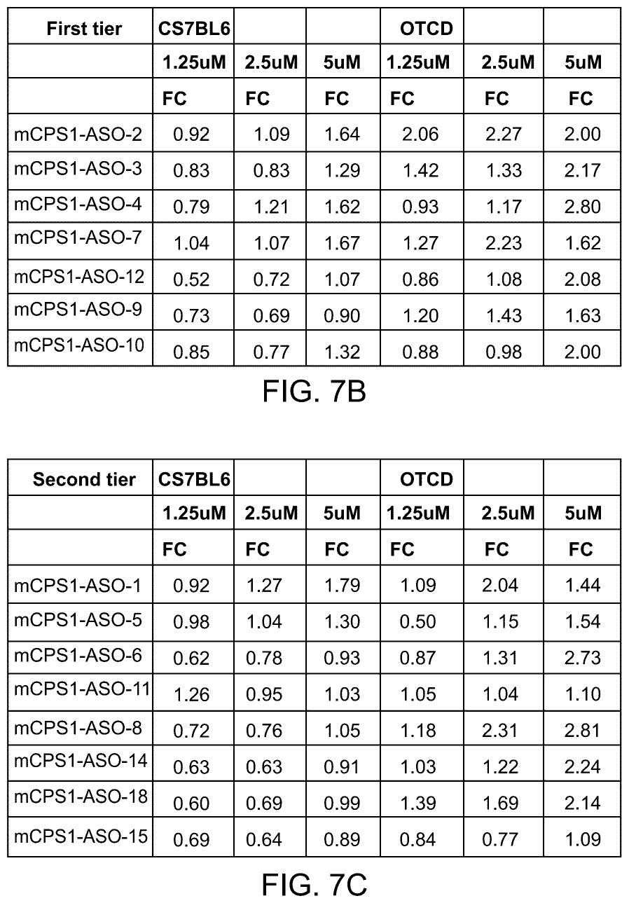

CPS1 mRNA levels in treated wildtype and OTC-deficient hepatocytes in vitro, are shown in FIG. 6A, heatmaps are shown in FIG. 6B and FIG. 6C. As shown, a subset of the identified ASOs upregulate CPS1 mRNA by 2-fold in treated hepatocytes. Additionally, it was observed that ASOs that upregulate CPS1 may also increases expression of OTC (OTC mRNA levels shown in FIG. 7A and heatmaps are shown in FIG. 7B and FIG. 7C). Thus, ASO targeting regRNA can be used to increase CPS1 expression in diseased mice liver cells. ASO mediated CPS1 upregulation in OTC deficient mouse cells allow these to be tested in a disease model, and have an in vivo phenotypic readout.

### In Vivo Modulation of CPS1

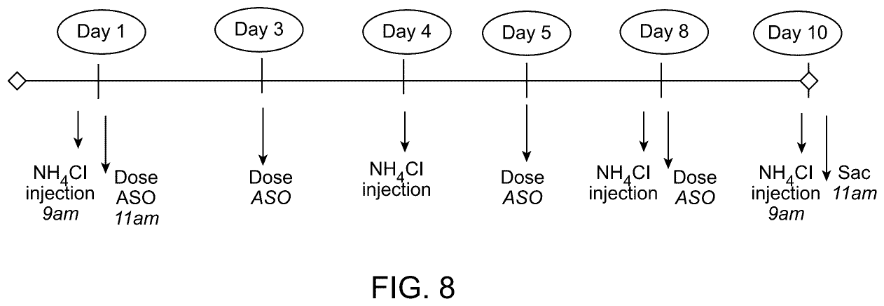

Male B6EiC3Sn a/A-Otc^{spf-ash}/J Mice (homozygous), 4-5 mice/group, were fasted, then challenged with 0.75M NH₄Cl injected intraperitoneally. Two hours later, a dose of 200 mg/kg/wk subcutaneous injection of GalNAc-conjugated ASO was administered. Mice were again dosed with GalNAc-conjugated ASO about 48 hours later. About 24 hours later, mice were challenged with 0.75M NH₄Cl injected intraperitoneally, and about 24 hours subsequently were again dosed with GalNAc-conjugated ASO. After 72 hours, mice were injected with 0.75M NH₄Cl intraperitoneally and dosed with GalNAc-conjugated ASO. 48 hours later, mice were injected with 0.75M NH₄Cl intraperitoneally, and 2 hours later, were sacrificed. The timeline is depicted in FIG. 8. Serum was collected 30-60 minutes post-NH₄Cl injection, and terminal plasma samples were analyzed by IDEXX for ammonia levels. Liver, serum, and urine were collected following sacrifice.

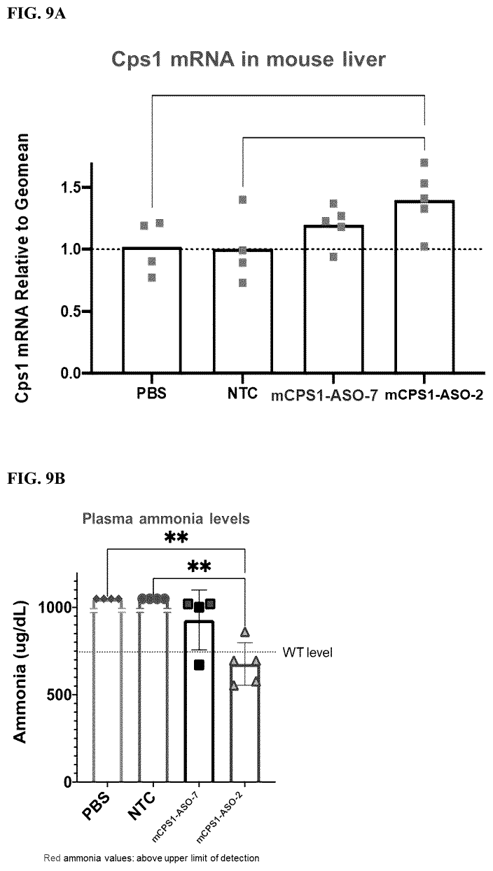

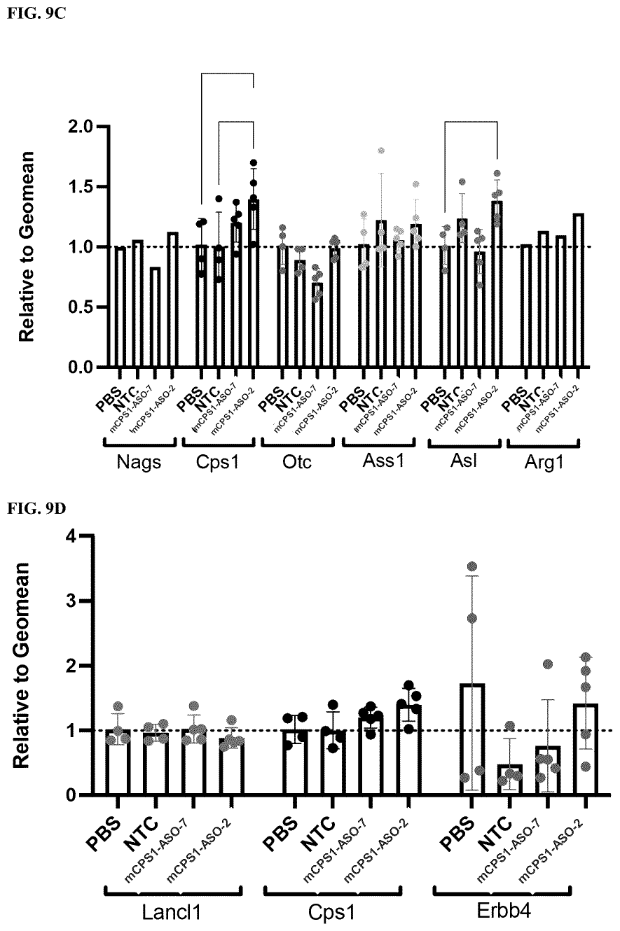

As shown in FIG. 9A, ASO treatment increased CPS1 mRNA in the mouse liver by about 1.4-fold. Additionally, plasma ammonia levels were decreased following treatment, indicated increased ureagenesis (FIG. 9B). ASOs targeting CPS1 regRNA additionally increased a number of other urea cycle genes, including ASS1, ASL, and ARG1 in OTC-deficient mice (FIG. 9C). Importantly, ASOs targeting CPS1 regRNA did not influence expression levels neighboring genes (FIG. 9D).

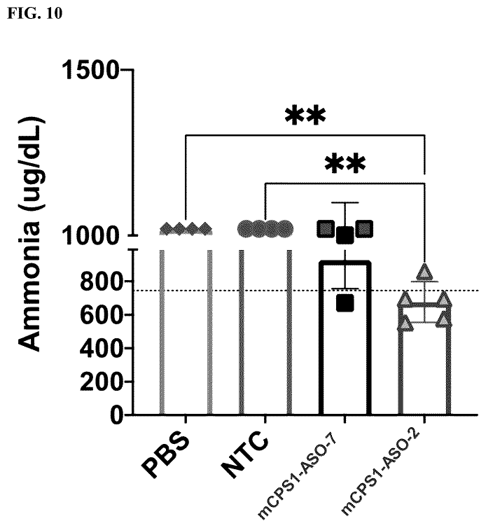

In an additional experiment, Male B6EiC3Sn a/A-Otc^{spf-ash/J} Mice (homozygous), 4-5 mice/group, were fasted, then challenged with 0.75M NH₄Cl injected intraperitoneally and subcutaneously injected with GalNAc-conjugated ASO. Terminal plasma samples collected 60 min after NH₄Cl injection was analyzed by IDEXX for ammonia levels, with an upper detection limit of 1020 μg/dL. As shown in FIG. 10, CPS1 regRNA targeting mCPS1-ASO-2 decreased ammonia back to wildtype levels in the OTC deficient mice.

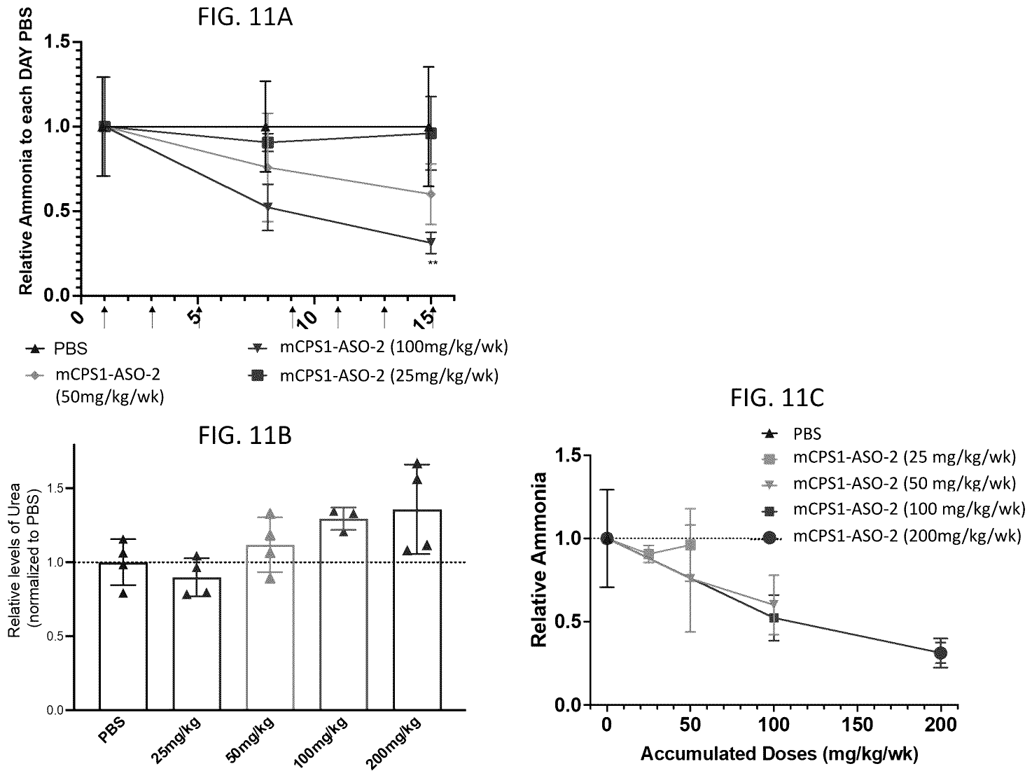

As shown in FIGS. 11A and 11B, there was a dose dependent impact on plasma ammonia (FIG. 11A) and urea (FIG. 11B) in vivo in male OTCD mice (Otc^{spf/ash}) after the ASO treatment. The ammonia challenge was given after overnight fasting of the mice Briefly, mice received a 0.75 M solution of ammonium chloride via IP injection. Interim plasma was collected at 30 minutes post each ammonium challenge (±5%). Ammonia levels were measured in fresh plasma samples by Beckman Coulter AU® chemistry analyzers. In addition, the reduction in ammonia correlated with the total ASO administered (FIG. 11C). Without wishing to be bound by theory this data suggests dose accumulation of the ASO and thus supports a long duration of action of the ASO. In addition, the data suggests that ASO efficacy can be achieved by splitting the dose over time, allowing for a greater safety margin during dosing. For example, at least a 1 month dosing interval may be used.

A second in vivo experiment was performed to assess the ASO duration of action. Male B6EiC3Sn a/A-Otc^{spf-ash/J} Mice (homozygous) or wild type (C57BL/6) mice, 5 mice/group, were fasted, then challenged with 0.75M NH₄Cl injected intraperitoneally. Two hours later, a dose of 100 mg/kg/wk subcutaneous injection of GalNAc-conjugated ASO was administered (day 1). WT and one group of OTC-deficient mice were administered PBS as control groups. Mice were again dosed with GalNAc-conjugated ASO on days 3 and 5. Ammonia was administered intraperitoneally to mice on days 8, 11, 15, 19, 22, and 26. Circulating ammonia was measured post-IP ammonia challenge. Mice were sacrificed on day 26 and livers were collected and processed for mRNA and protein expression. mRNA geomean was quantified by Gusb, Gapdh, Actb, Ppia, and Hprt genes. Statistics were done by two way ANOVA. Two way ANOVA, *: P<0.05, **: P<0.01, ***: P<0.001, ****: P<0.0001

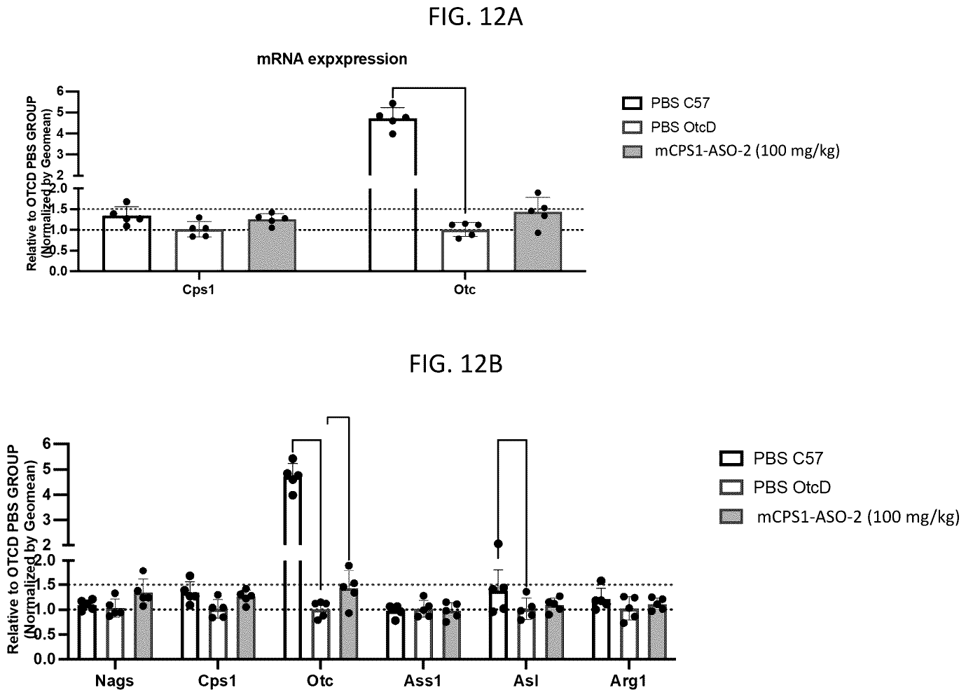

As shown in FIG. 12A, Cps1 ASO treatment increased mouse Otc and Cps1 mRNA expression in the OTC deficient mice (1.26 FC of Cps1 and 1.43 FC of Otc). WT C57 mice mRNA is shown on the left bar, Otc^{spf-ash/J} treated with PBS is shown in middle bar, and Otc^{spf-ash/J} treated with ASO is shown on the right. In addition, ASO treatment increased additional urea cycle genes, such as Nags, Ass1, Asl, and Arg1 (FIG. 12B). WT C57 mice mRNA is shown on the left bar, Otc^{spf-ash/J} treated with PBS is shown in middle bar, and Otc^{spf-ash/J} treated with ASO is shown on the right.

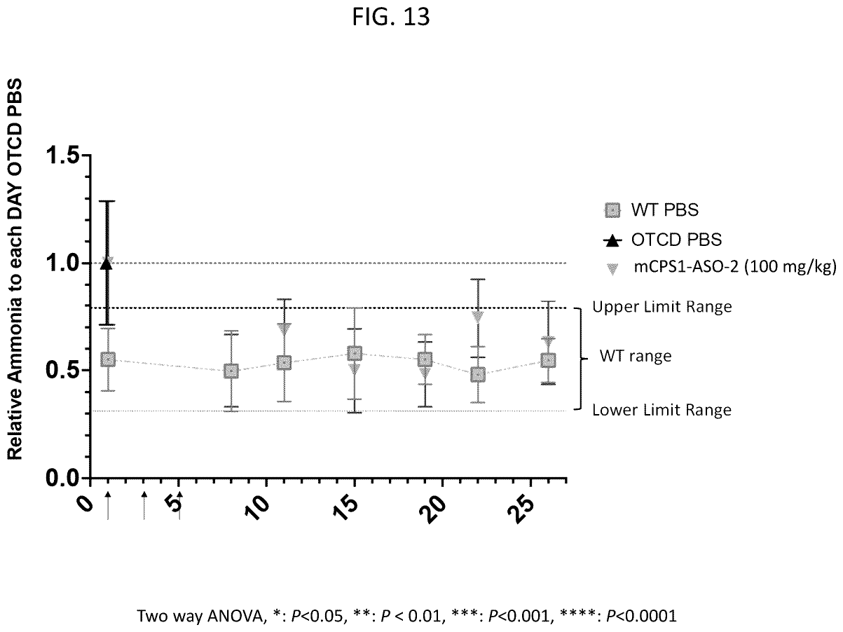

In the PBS control group, mouse Otc deficient livers showed hyperammonia 30 min after ammonia treatment (FIG. 13, see black triangle at 1.0). Ammonia levels were consistent through the study. Treatment with mouse Cps1 ASO (upside down triangles) decreased ammonia to WT levels (squares) and maintained the wild type (WT) levels throughout the study (FIG. 13). ASO was administered on days 1, 3, and 5 but the duration of action was approximately 4 weeks.

The in vivo assay was repeated with hCPS1-ASO-1x in Yecuris mice (Fah^{-/-};Rag2^{-/-};Il2rg^{-/-} (FRG) mice with OTC deficient humanized liver). Male humanized Yecuris mice were repopulated with human ornithine transcarbamylase deficient hepatocytes (OTCD) or with healthy wild-type hepatocytes (WT), 5 mice/group. After fasting overnight, 0.75M ¹⁵NH₄Cl was injected intraperitoneally. Two hours later, multiple doses of GalNAc-conjugated hCPS1-ASO-1x were subcutaneously injected on days 1, 8, and 15. In some groups, a single injection of 5, 20, and 50 mg/kg/week doses were performed. WT and one group of OTCD Yecuris FRG mice were administered with PBS as control groups. Ammonia challenge and ureagenesis assay in humanized Yecuris mice were performed in a fasted state, i.e. food withdrawal overnight, prior to the ammonia challenge. After fasting overnight on days 1, 8, 15, and 22 (terminal harvest) the animals were challenged with ¹⁵NH₄Cl by intraperitoneal injection. After 30 minutes, urines and plasmas were collected.

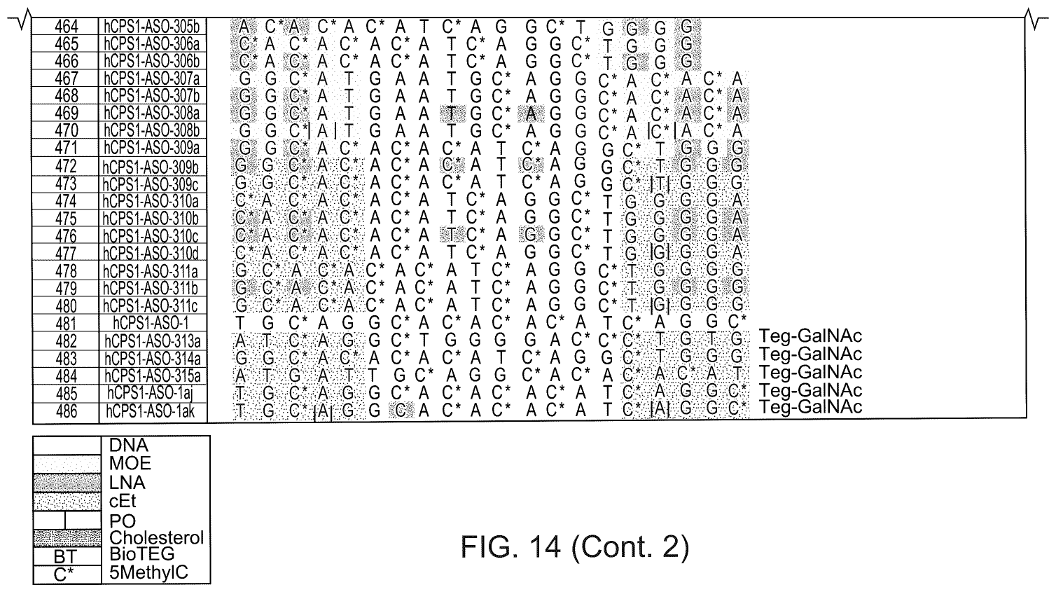

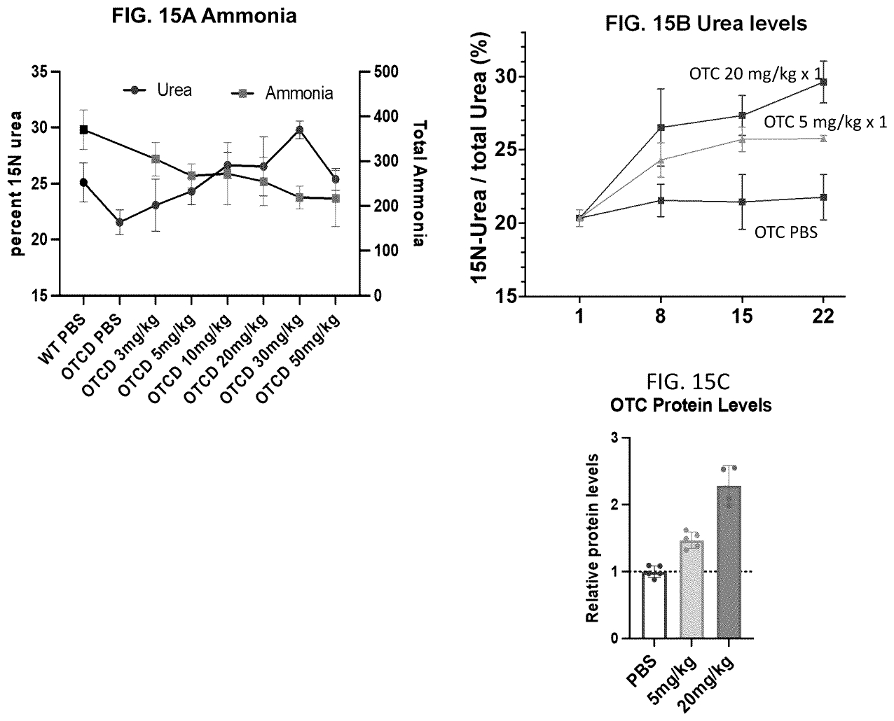

As shown in FIGS. 15A and 15B, hCPS1-ASO-1× decreased in vivo plasma ammonia and increased in vivo urea in the OTC^{def} mice with humanized liver. hCPS1-ASO-1g also increased OTC protein levels after treatment at 5 mg/kg and 20 mg/kg (FIG. 15C).

### In Vitro Modulation of Ureagenesis in Primary Human Hepatocytes

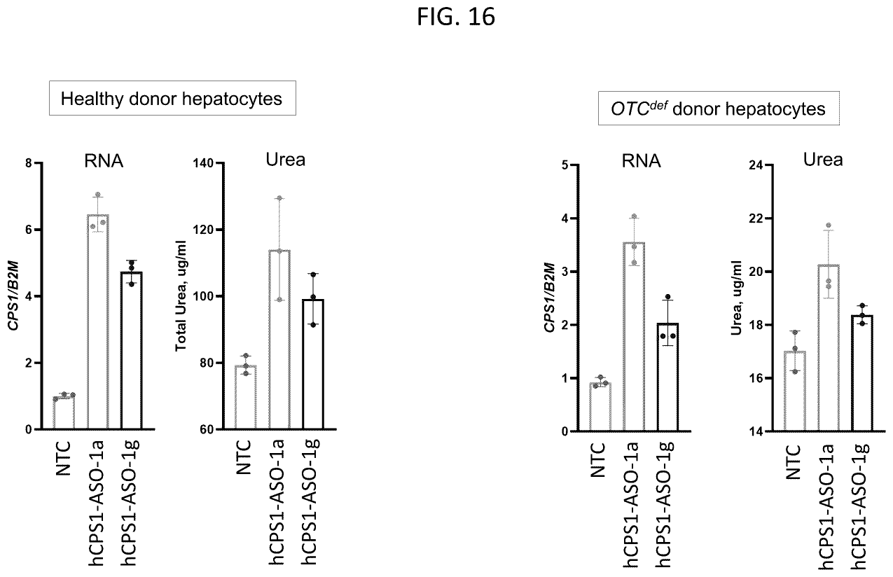

ASOs were also tested for their ability to agonize ureagenesis in human cells in vitro. Healthy donor primary human hepatocytes and OTC^{def} primary human hepatocytes were plated and 1.25 uM of hCPS1-ASO-1a or hCPS1-ASO-1g were incubated with the cells for two days. On the second day 2 M NH₄C1 was added to the cell mixture. Cells and cell media were collected on Day 3 for mRNA and urea quantification. As shown in FIG. 16, both ASOs increased CPS1 mRNA and ureagenesis in both healthy and OTC^{def} primary human hepatocytes.

### Synthesis and Characterization of Additional ASOs

Additional ASOs were synthesized and characterized. Hepatocytes were treated with 5 uM ASO as described above and CPS1 mRNA fold change was determined.

Table 4 proves the mRNA FC and standard deviation in CPS1 mRNA.

TABLE 4

| Name | FC | SD | Name | FC | SD | Name | FC | SD |
|---|---|---|---|---|---|---|---|---|
| hCPS1- | 5.5803 | 0.9290 | hCPS1- | 2.4815 | 0.2779 | hCPS1- | 1.0451 | 0.2891 |
| ASO-1a | | | ASO-85b | | | ASO-204 | | |
| hCPS1- | 1.6607 | 0.2690 | hCPS1- | 2.5546 | 0.1486 | hCPS1- | 1.0432 | 0.1968 |
| ASO-16 | | | ASO-86 | | | ASO-205 | | |
| hCPS1- | 1.1878 | 0.2382 | hCPS1- | 1.2337 | 0.1193 | hCPS1- | 0.9000 | 0.1529 |
| ASO-17 | | | ASO-1ab | | | ASO-206 | | |
| hCPS1- | 1.3738 | 0.1466 | hCPS1- | 1.3452 | 0.4155 | hCPS1- | 1.0064 | 0.2810 |
| ASO-18 | | | ASO-1ac | | | ASO-207 | | |
| hCPS1- | 1.3322 | 0.2929 | hCPS1- | 1.7071 | 0.3347 | hCPS1- | 0.8499 | 0.1017 |
| ASO-19 | | | ASO-1ad | | | ASO-208 | | |
| hCPS1- | 1.4049 | 0.2328 | hCPS1- | 1.4598 | 0.2254 | hCPS1- | 0.8872 | 0.2187 |
| ASO-20 | | | ASO-1ae | | | ASO-209 | | |
| hCPS1- | 3.6049 | 0.0613 | hCPS1- | 1.1069 | 0.2738 | hCPS1- | 1.0426 | 0.2646 |
| ASO-21 | | | ASO-1af | | | ASO-210 | | |
| hCPS1- | 1.2367 | 0.1154 | hCPS1- | 1.2439 | 0.3771 | hCPS1- | 0.8667 | 0.1797 |
| ASO-22 | | | ASO-1ag | | | ASO-211 | | |
| hCPS1- | 1.0846 | 0.1572 | hCPS1- | 1.9014 | 0.3037 | hCPS1- | 1.1604 | 0.2568 |
| ASO-23 | | | ASO-1ah | | | ASO-212 | | |
| hCPS1- | 1.0683 | 0.1120 | hCPS1- | 3.0003 | 0.6407 | hCPS1- | 1.0240 | 0.2239 |
| ASO-24 | | | ASO-1ai | | | ASO-213 | | |
| hCPS1- | 0.9967 | 0.1307 | hCPS1- | 1.4093 | 0.0630 | hCPS1- | 0.9408 | 0.2219 |
| ASO-25 | | | ASO-92 | | | ASO-214 | | |
| hCPS1- | 1.1141 | 0.2129 | hCPS1- | 1.3278 | 0.1805 | hCPS1- | 1.2719 | 0.3355 |
| ASO-26 | | | ASO-93 | | | ASO-215 | | |
| hCPS1- | 1.2348 | 0.2667 | hCPS1- | 1.1871 | 0.0806 | hCPS1- | 0.9536 | 0.2038 |
| ASO-27 | | | ASO-94 | | | ASO-216 | | |
| hCPS1- | 3.7994 | 0.7319 | hCPS1- | 1.0937 | 0.1027 | hCPS1- | 0.9589 | 0.2111 |
| ASO-28 | | | ASO-95 | | | ASO-217 | | |
| hCPS1- | 1.2569 | 0.2946 | hCPS1- | 1.0708 | 0.1544 | hCPS1- | 0.9624 | 0.1812 |
| ASO-29 | | | ASO-96 | | | ASO-218 | | |
| hCPS1- | 0.9996 | 0.1342 | hCPS1- | 1.8697 | 0.2390 | hCPS1- | 1.0837 | 0.3110 |
| ASO-30 | | | ASO-97 | | | ASO-219 | | |
| hCPS1- | 1.0879 | 0.2887 | hCPS1- | 2.2671 | 0.0950 | hCPS1- | 1.0236 | 0.1605 |
| ASO-31 | | | ASO-98 | | | ASO-220 | | |
| hCPS1- | 1.0803 | 0.0754 | hCPS1- | 3.2286 | 0.3834 | hCPS1- | 1.0461 | 0.2903 |
| ASO-32 | | | ASO-99 | | | ASO-221 | | |
| hCPS1- | 1.0982 | 0.2301 | hCPS1- | 1.6813 | 0.1274 | hCPS1- | 1.0435 | 0.2238 |
| ASO-33 | | | ASO-100 | | | ASO-222 | | |
| hCPS1- | 0.9376 | 0.0691 | hCPS1- | 2.8205 | 0.0827 | hCPS1- | 1.1306 | 0.4103 |
| ASO-34 | | | ASO-101 | | | ASO-223 | | |
| hCPS1- | 4.5563 | 0.0988 | hCPS1- | 3.1424 | 0.1637 | hCPS1- | 1.0954 | 0.1792 |
| ASO-35 | | | ASO-102 | | | ASO-224 | | |
| hCPS1- | 1.1589 | 0.1449 | hCPS1- | 3.7109 | 0.4564 | hCPS1- | 0.8416 | 0.2138 |
| ASO-36 | | | ASO-103 | | | ASO-225 | | |
| hCPS1- | 1.0707 | 0.1475 | hCPS1- | 5.0641 | 0.2305 | hCPS1- | 1.0220 | 0.2833 |
| ASO-37 | | | ASO-104 | | | ASO-226 | | |
| hCPS1- | 1.0941 | 0.1276 | hCPS1- | 1.4662 | 0.1834 | hCPS1- | 0.8141 | 0.1635 |
| ASO-38 | | | ASO-107 | | | ASO-227 | | |
| hCPS1- | 1.1150 | 0.1947 | hCPS1- | 1.3163 | 0.1848 | hCPS1- | 0.7884 | 0.1280 |
| ASO-39 | | | ASO-108 | | | ASO-228 | | |
| hCPS1- | 1.1812 | 0.0713 | hCPS1- | 1.1404 | 0.1269 | hCPS1- | 0.8821 | 0.1602 |
| ASO-40 | | | ASO-109 | | | ASO-229 | | |
| hCPS1- | 1.1376 | 0.1669 | hCPS1- | 1.4157 | 0.2399 | hCPS1- | 1.0366 | 0.2360 |
| ASO-41 | | | ASO-110 | | | ASO-230 | | |
| hCPS1- | 3.9849 | 0.9906 | hCPS1- | 1.3974 | 0.0526 | hCPS1- | 1.0731 | 0.3336 |
| ASO-42 | | | ASO-111 | | | ASO-231 | | |
| hCPS1- | 1.1340 | 0.1748 | hCPS1- | 1.3309 | 0.2948 | hCPS1- | 1.0336 | 0.2169 |
| ASO-43 | | | ASO-112 | | | ASO-232 | | |
| hCPS1- | 0.9795 | 0.1452 | hCPS1- | 1.1250 | 0.0133 | hCPS1- | 1.0184 | 0.2427 |
| ASO-44 | | | ASO-113 | | | ASO-233 | | |
| hCPS1- | 1.0825 | 0.3286 | hCPS1- | 1.4471 | 0.3902 | hCPS1- | 1.0379 | 0.2667 |
| ASO-45 | | | ASO-114 | | | ASO-234 | | |
| hCPS1- | 1.3008 | 0.2339 | hCPS1- | 1.3250 | 0.2205 | hCPS1- | 0.7109 | 0.1642 |
| ASO-46 | | | ASO-115 | | | ASO-235 | | |
| hCPS1- | 1.2834 | 0.2346 | hCPS1- | 1.4592 | 0.2191 | hCPS1- | 0.9437 | 0.2016 |
| ASO-47 | | | ASO-116 | | | ASO-236 | | |
| hCPS1- | 1.0953 | 0.2896 | hCPS1- | 1.5146 | 0.1541 | hCPS1- | 1.1237 | 0.2948 |
| ASO-48 | | | ASO-117 | | | ASO-237 | | |
| hCPS1- | 1.4947 | 0.3320 | hCPS1- | 1.4798 | 0.3053 | hCPS1- | 0.8325 | 0.1677 |
| ASO-49 | | | ASO-118 | | | ASO-238 | | |
| hCPS1- | 1.2992 | 0.2852 | hCPS1- | 1.4687 | 0.3357 | hCPS1- | 1.2847 | 0.3268 |
| ASO-50 | | | ASO-119 | | | ASO-239 | | |
| hCPS1- | 1.0101 | 0.2333 | hCPS1- | 1.4309 | 0.2206 | hCPS1- | 1.0368 | 0.1643 |
| ASO-51 | | | ASO-120 | | | ASO-240 | | |
| hCPS1- | 1.2026 | 0.2065 | hCPS1- | 1.5330 | 0.4945 | hCPS1- | 1.0348 | 0.2319 |
| ASO-52 | | | ASO-121 | | | ASO-241 | | |
| hCPS1- | 1.3176 | 0.2462 | hCPS1- | 2.1728 | 1.0205 | hCPS1- | 1.2009 | 0.3035 |
| ASO-53 | | | ASO-122 | | | ASO-242 | | |
| hCPS1- | 1.2888 | 0.1512 | hCPS1- | 1.3792 | 0.2117 | hCPS1- | 0.8364 | 0.1646 |
| ASO-54 | | | ASO-123 | | | ASO-243 | | |
| hCPS1- | 0.9070 | 0.0979 | hCPS1- | 1.6351 | 0.2666 | hCPS1- | 0.9177 | 0.1890 |
| ASO-55 | | | ASO-124 | | | ASO-244 | | |
| hCPS1- | 2.2957 | 0.4780 | hCPS1- | 1.9894 | 0.2327 | hCPS1- | 0.9563 | 0.2149 |
| ASO-56 | | | ASO-125 | | | ASO-245 | | |
| hCPS1- | 1.3780 | 0.1587 | hCPS1- | 1.8741 | 0.1928 | hCPS1- | 1.0945 | 0.1746 |
| ASO-57 | | | ASO-126 | | | ASO-246 | | |
| hCPS1- | 1.1027 | 0.1230 | hCPS1- | 1.9805 | 0.5252 | hCPS1- | 1.1518 | 0.1876 |
| ASO-58 | | | ASO-127 | | | ASO-247 | | |
| hCPS1- | 1.2449 | 0.2215 | hCPS1- | 1.7071 | 0.2968 | hCPS1- | 1.1365 | 0.2040 |
| ASO-59 | | | ASO-128 | | | ASO-248 | | |
| hCPS1- | 1.4067 | 0.1655 | hCPS1- | 1.8255 | 0.4447 | hCPS1- | 0.9208 | 0.2208 |
| ASO-60 | | | ASO-129 | | | ASO-249 | | |
| hCPS1- | 1.2124 | 0.0876 | hCPS1- | 1.9578 | 0.6166 | hCPS1- | 1.1458 | 0.2996 |
| ASO-61 | | | ASO-130 | | | ASO-250 | | |
| hCPS1- | 0.9895 | 0.1303 | hCPS1- | 1.3484 | 0.2757 | hCPS1- | 0.8471 | 0.1852 |
| ASO-62 | | | ASO-131 | | | ASO-251 | | |
| hCPS1- | 2.3654 | 0.4539 | hCPS1- | 1.4400 | 0.3570 | hCPS1- | 0.7147 | 0.0925 |
| ASO-63 | | | ASO-132 | | | ASO-252 | | |
| hCPS1- | 1.1365 | 0.1718 | hCPS1- | 1.4004 | 0.1358 | hCPS1- | 0.6961 | 0.1808 |
| ASO-64 | | | ASO-133 | | | ASO-253 | | |
| hCPS1- | 0.8489 | 0.1813 | hCPS1- | 1.5631 | 0.1290 | hCPS1- | 0.8060 | 0.1913 |
| ASO-65 | | | ASO-134 | | | ASO-254 | | |
| hCPS1- | 1.2409 | 0.1946 | hCPS1- | 1.6723 | 0.4246 | hCPS1- | 0.8039 | 0.1878 |
| ASO-66 | | | ASO-135 | | | ASO-255 | | |
| hCPS1- | 0.9222 | 0.1245 | hCPS1- | 1.6023 | 0.3159 | hCPS1- | 0.7368 | 0.0548 |
| ASO-67 | | | ASO-136 | | | ASO-256 | | |
| hCPS1- | 1.3275 | 0.1510 | hCPS1- | 1.5022 | 0.2462 | hCPS1- | 0.9517 | 0.2562 |
| ASO-68 | | | ASO-137 | | | ASO-257 | | |
| hCPS1- | 1.0944 | 0.2374 | hCPS1- | 1.9038 | 0.5748 | hCPS1- | 1.0658 | 0.3429 |
| ASO-69 | | | ASO-138 | | | ASO-258 | | |
| hCPS1- | 1.5761 | 0.1388 | hCPS1- | 1.6131 | 0.1660 | hCPS1- | 0.8157 | 0.2459 |
| ASO-70 | | | ASO-139 | | | ASO-259 | | |
| hCPS1- | 1.3363 | 0.1613 | hCPS1- | 1.6504 | 0.2242 | hCPS1- | 1.1626 | 0.2553 |
| ASO-71 | | | ASO-140 | | | ASO-260 | | |
| hCPS1- | 1.0412 | 0.1404 | hCPS1- | 1.8803 | 0.2397 | hCPS1- | 0.9754 | 0.1961 |
| ASO-72 | | | ASO-141 | | | ASO-261 | | |
| hCPS1- | 1.3314 | 0.2056 | hCPS1- | 3.0769 | 0.3542 | hCPS1- | 1.3364 | 0.3546 |
| ASO-73 | | | ASO-142 | | | ASO-262 | | |
| hCPS1- | 1.2915 | 0.1170 | hCPS1- | 1.9543 | 0.5131 | hCPS1- | 1.0346 | 0.1123 |
| ASO-74 | | | ASO-143 | | | ASO-263 | | |
| hCPS1- | 1.2395 | 0.0952 | hCPS1- | 3.5634 | 0.5168 | hCPS1- | 0.6237 | 0.0401 |
| ASO-75 | | | ASO-144 | | | ASO-264 | | |
| hCPS1- | 1.0942 | 0.2650 | hCPS1- | 2.8153 | 0.5824 | hCPS1- | 0.7730 | 0.1091 |
| ASO-76 | | | ASO-145 | | | ASO-265 | | |
| hCPS1- | 1.1870 | 0.1452 | hCPS1- | 1.5756 | 0.0004 | hCPS1- | 0.8882 | 0.1967 |
| ASO-1b | | | ASO-146 | | | ASO-266 | | |
| hCPS1- | 1.4590 | 0.2142 | hCPS1- | 1.8075 | 0.1998 | hCPS1- | 0.7796 | 0.2146 |
| ASO-1c | | | ASO-147 | | | ASO-267 | | |
| hCPS1- | 1.0277 | 0.1069 | hCPS1- | 1.6902 | 0.0950 | hCPS1- | 0.9467 | 0.1832 |
| ASO-1d | | | ASO-148 | | | ASO-268 | | |
| hCPS1- | 1.0992 | 0.1400 | hCPS1- | 1.5828 | 0.0817 | hCPS1- | 0.9570 | 0.1712 |
| ASO-le | | | ASO-149 | | | ASO-269 | | |
| hCPS1- | 1.6978 | 0.4026 | hCPS1- | 1.6598 | 0.1305 | hCPS1- | 0.8511 | 0.1498 |
| ASO-1f | | | ASO-150 | | | ASO-270 | | |
| hCPS1- | 4.4990 | 0.6087 | hCPS1- | 1.7341 | 0.2274 | hCPS1- | 0.9859 | 0.1688 |
| ASO-1g | | | ASO-151 | | | ASO-271 | | |
| hCPS1- | 4.3014 | 0.9287 | hCPS1- | 1.7582 | 0.2844 | hCPS1- | 0.7428 | 0.0842 |
| ASO-2 | | | ASO-152 | | | ASO-272 | | |
| hCPS1- | 2.2218 | 0.1531 | hCPS1- | 1.8459 | 0.3518 | hCPS1- | 0.8121 | 0.1444 |
| ASO-3 | | | ASO-153 | | | ASO-273 | | |
| hCPS1- | 1.0275 | 0.1642 | hCPS1- | 1.5900 | 0.4826 | hCPS1- | 0.7497 | 0.1493 |
| ASO-9a | | | ASO-154 | | | ASO-274 | | |
| hCPS1- | 1.1384 | 0.2750 | hCPS1- | 1.3183 | 0.0726 | hCPS1- | 0.7799 | 0.1717 |
| ASO-10a | | | ASO-155 | | | ASO-275 | | |
| hCPS1- | 1.1334 | 0.1617 | hCPS1- | 0.9952 | 0.1123 | hCPS1- | 0.8826 | 0.1542 |
| ASO-11a | | | ASO-156 | | | ASO-276 | | |
| hCPS1- | 0.8162 | 0.0866 | hCPS1- | 1.2972 | 0.1294 | hCPS1- | 0.8896 | 0.2650 |
| ASO-12a | | | ASO-157 | | | ASO-277 | | |
| hCPS1- | 1.5297 | 0.1882 | hCPS1- | 1.7248 | 0.2074 | hCPS1- | 0.4696 | 0.0748 |
| ASO-13a | | | ASO-158 | | | ASO-278 | | |
| hCPS1- | 0.9744 | 0.0844 | hCPS1- | 1.7892 | 0.3807 | hCPS1- | 1.3629 | 0.1492 |
| ASO-14a | | | ASO-159 | | | ASO-279 | | |
| hCPS1- | 0.9225 | 0.1756 | hCPS1- | 1.6078 | 0.2263 | hCPS1- | 1.9970 | 0.4340 |
| ASO-15a | | | ASO-160 | | | ASO-280 | | |
| hCPS1- | 0.6872 | 0.0821 | hCPS1- | 1.0960 | 0.6012 | hCPS1- | 1.9249 | 0.2595 |
| ASO-7a | | | ASO-161 | | | ASO-281 | | |
| hCPS1- | 1.5925 | 0.1816 | hCPS1- | 1.5522 | 0.6413 | hCPS1- | 1.4252 | 0.4261 |
| ASO-4a | | | ASO-162 | | | ASO-282 | | |
| hCPS1- | 2.1196 | 0.3491 | hCPS1- | 1.1633 | 0.0408 | hCPS1- | 4.4672 | 1.1138 |
| ASO-5a | | | ASO-163 | | | ASO-283 | | |
| hCPS1- | 1.8529 | 0.9210 | hCPS1- | 1.3349 | 0.1053 | hCPS1- | 5.0837 | 1.5527 |
| ASO-6a | | | ASO-164 | | | ASO-284 | | |
| hCPS1- | 1.5270 | 0.8361 | hCPS1- | 1.6669 | 0.2143 | hCPS1- | 4.7339 | 0.3981 |
| ASO-8a | | | ASO-165 | | | ASO-285 | | |
| hCPS1- | 0.8992 | 0.0590 | hCPS1- | 1.3915 | 0.2569 | hCPS1- | 1.5091 | 0.3739 |
| ASO-9b | | | ASO-166 | | | ASO-286 | | |
| hCPS1- | 0.9770 | 0.0428 | hCPS1- | 1.7175 | 0.2493 | hCPS1- | 1.3469 | 0.3479 |
| ASO-10b | | | ASO-167 | | | ASO-287 | | |
| hCPS1- | 1.6000 | 0.2631 | hCPS1- | 1.1979 | 0.1337 | hCPS1- | 1.6093 | 0.2169 |
| ASO-11b | | | ASO-168 | | | ASO-288 | | |
| hCPS1- | 0.9917 | 0.1514 | hCPS1- | 1.3335 | 0.2080 | hCPS1- | 0.9802 | 0.3187 |
| ASO-12b | | | ASO-169 | | | ASO-289 | | |
| hCPS1- | 1.1117 | 0.1612 | hCPS1- | 1.0882 | 0.3444 | hCPS1- | 1.6650 | 0.1183 |
| ASO-13b | | | ASO-170 | | | ASO-290 | | |
| hCPS1- | 0.8017 | 0.0536 | hCPS1- | 1.2852 | 0.1472 | hCPS1- | 1.7705 | 0.4831 |
| ASO-14b | | | ASO-171 | | | ASO-291 | | |
| hCPS1- | 0.8779 | 0.0632 | hCPS1- | 1.3309 | 0.1877 | hCPS1- | 1.8768 | 0.3481 |
| ASO-15b | | | ASO-172 | | | ASO-292 | | |
| hCPS1- | 0.9488 | 0.0891 | hCPS1- | 1.3430 | 0.2467 | hCPS1- | 1.8295 | 0.3331 |
| ASO-7b | | | ASO-173 | | | ASO-293 | | |
| hCPS1- | 0.9687 | 0.1444 | hCPS1- | 1.2633 | 0.2020 | hCPS1- | 4.2131 | 0.7748 |
| ASO-4b | | | ASO-174 | | | ASO-294 | | |
| hCPS1- | 0.9448 | 0.1081 | hCPS1- | 1.6397 | 0.3383 | hCPS1- | 0.8242 | 0.1736 |
| ASO-5b | | | ASO-175 | | | ASO-295 | | |
| hCPS1- | 0.8277 | 0.0428 | hCPS1- | 1.3926 | 0.5390 | hCPS1- | 1.7239 | 0.4255 |
| ASO-6b | | | ASO-176 | | | ASO-296 | | |
| hCPS1- | 0.9161 | 0.1199 | hCPS1- | 1.4242 | 0.2749 | hCPS1- | 2.0747 | 0.4485 |
| ASO-8b | | | ASO-177 | | | ASO-297 | | |
| hCPS1- | 4.1549 | 0.9198 | hCPS1- | 1.4453 | 0.2574 | hCPS1- | 1.1081 | 0.1802 |
| ASO-77 | | | ASO-178 | | | ASO-298 | | |
| hCPS1- | 2.2972 | 0.1418 | hCPS1- | 1.2086 | 0.0863 | hCPS1- | 5.1515 | 0.9679 |
| ASO-78 | | | ASO-179 | | | ASO-299 | | |
| hCPS1- | 2.4319 | 0.2248 | hCPS1- | 1.2183 | 0.1814 | hCPS1- | 4.8213 | 0.9887 |
| ASO-79 | | | ASO-180 | | | ASO-300 | | |
| hCPS1- | 2.7917 | 0.6061 | hCPS1- | 1.3878 | 0.1419 | hCPS1- | 3.3943 | 0.6421 |
| ASO-80 | | | ASO-181 | | | ASO-301 | | |
| hCPS1- | 1.1727 | 0.1382 | hCPS1- | 1.3813 | 0.1038 | hCPS1- | 1.8365 | 0.4610 |
| ASO-1h | | | ASO-182 | | | ASO-302 | | |
| hCPS1- | 1.4355 | 0.1558 | hCPS1- | 1.2326 | 0.1733 | hCPS1- | 1.3137 | 0.2222 |
| ASO-1i | | | ASO-183 | | | ASO-303a | | |
| hCPS1- | 1.2027 | 0.0988 | hCPS1- | 1.3169 | 0.3607 | hCPS1- | 1.7427 | 0.3256 |
| ASO-1j | | | ASO-184 | | | ASO-303b | | |
| hCPS1- | 1.1188 | 0.0499 | hCPS1- | 1.4697 | 0.2622 | hCPS1- | 2.2107 | 0.3595 |
| ASO-1k | | | ASO-185 | | | ASO-304a | | |
| hCPS1- | 1.3171 | 0.1662 | hCPS1- | 1.2855 | 0.3984 | hCPS1- | 2.0165 | 0.7982 |
| ASO-1l | | | ASO-186 | | | ASO-304b | | |
| hCPS1- | 1.1873 | 0.1099 | hCPS1- | 1.3655 | 0.2214 | hCPS1- | 1.8751 | 0.3138 |
| ASO-1m | | | ASO-187 | | | ASO-305a | | |
| hCPS1- | 1.1683 | 0.1096 | hCPS1- | 1.3777 | 0.1068 | hCPS1- | 2.9614 | 0.4636 |
| ASO-1n | | | ASO-188 | | | ASO-305b | | |
| hCPS1- | 1.3191 | 0.1220 | hCPS1- | 1.2786 | 0.1922 | hCPS1- | 2.9192 | 0.8811 |
| ASO-1o | | | ASO-189 | | | ASO-306a | | |
| hCPS1- | 1.9562 | 0.0841 | hCPS1- | 1.0252 | 0.0880 | hCPS1- | 3.4468 | 1.3494 |
| ASO-1p | | | ASO-190 | | | ASO-306b | | |
| hCPS1- | 1.2673 | 0.1820 | hCPS1- | 1.2291 | 0.1588 | hCPS1- | 1.3314 | 0.3165 |
| ASO-1q | | | ASO-191 | | | ASO-307a | | |
| hCPS1- | 1.5857 | 0.1339 | hCPS1- | 1.4614 | 0.2198 | hCPS1- | 1.2205 | 0.1267 |
| ASO-1r | | | ASO-192 | | | ASO-307 | | |
| hCPS1- | 2.5762 | 0.3147 | hCPS1- | 1.4984 | 0.4280 | hCPS1- | 1.2918 | 0.2042 |
| ASO-1s | | | ASO-193 | | | ASO-308a | | |
| hCPS1- | 2.4333 | 0.2508 | hCPS1- | 0.9625 | 0.3146 | hCPS1- | 1.1019 | 0.1488 |
| ASO-1t | | | ASO-194 | | | ASO-308 | | |
| hCPS1- | 2.9405 | 0.2842 | hCPS1- | 1.4616 | 0.2171 | hCPS1- | 1.9776 | 0.5046 |
| ASO-1u | | | ASO-195 | | | ASO-309a | | |
| hCPS1- | 1.9457 | 0.2076 | hCPS1- | 1.3635 | 0.0990 | hCPS1- | 1.4482 | 0.2178 |
| ASO-1v | | | ASO-196 | | | ASO-309b | | |
| hCPS1- | 2.5019 | 0.3142 | hCPS1- | 1.5088 | 0.2155 | hCPS1- | 1.8643 | 0.3592 |
| ASO-82a | | | ASO-197 | | | ASO-309c | | |
| hCPS1- | 1.6054 | 0.1333 | hCPS1- | 1.5691 | 0.1943 | hCPS1- | 2.1756 | 0.8122 |
| ASO-83a | | | ASO-198 | | | ASO-310a | | |
| hCPS1- | 1.3049 | 0.1062 | hCPS1- | 0.8877 | 0.2414 | hCPS1- | 2.1692 | 0.3493 |
| ASO-83b | | | ASO-199 | | | ASO-310b | | |
| hCPS1- | 1.6594 | 0.1400 | hCPS1- | 0.9393 | 0.1766 | hCPS1- | 1.5170 | 0.2149 |
| ASO-84a | | | ASO-200 | | | ASO-310c | | |
| hCPS1- | 1.6155 | 0.2525 | hCPS1- | 0.8805 | 0.1631 | hCPS1- | 1.7083 | 0.2543 |
| ASO-84b | | | ASO-201 | | | ASO-310d | | |
| hCPS1- | 3.1551 | 0.2265 | hCPS1- | 0.9079 | 0.1671 | hCPS1- | 3.7975 | 0.7950 |
| ASO-85a | | | ASO-202 | | | ASO-311a | | |
| hCPS1- | 1.2847 | 0.4866 | hCPS1- | 0.8525 | 0.1637 | hCPS1- | 2.2173 | 0.2349 |
| ASO-1 | | | ASO-203 | | | ASO-311b | | |
| | | | | | | hCPS1- | 3.8806 | 0.8311 |
| | | | | | | ASO-311c | | |

hCPS1-ASO-1a and hCPS1-ASO1g were tested in hepatocytes at increasing concentrations. 0.156 to 5 uM ASO was incubated with hepatocytes as described above. As shown in Table 5, both hCPS1-ASO-1a and hCPS1-ASO1g induced a dose dependent increase in CPS1 mRNA.

TABLE 5

| Name | FC | SD | Dose (uM) |
|---|---|---|---|
| hCPS1-ASO-1a | 1.6220 | 0.0932 | 0.156 |
| hCPS1-ASO-1a | 1.9973 | 0.2021 | 0.313 |
| hCPS1-ASO-1a | 2.4574 | 0.1968 | 0.625 |
| hCPS1-ASO-1a | 3.7565 | 0.4598 | 1.25 |
| hCPS1-ASO-1a | 4.9565 | 0.7826 | 2.5 |
| hCPS1-ASO-1a | 4.9868 | 0.4258 | 5 |
| hCPS1-ASO-1g | 1.5646 | 0.0907 | 0.156 |
| hCPS1-ASO-1g | 1.7147 | 0.1318 | 0.313 |
| hCPS1-ASO-1g | 1.9748 | 0.3676 | 0.625 |
| hCPS1-ASO-1g | 3.3634 | 0.7101 | 2.5 |
| hCPS1-ASO-1g | 3.7516 | 0.7574 | 5 |

Additional chemical modifications were made to selected ASOs. The chemical modifications are provided in FIG. 14. Hepatocytes were incubated with 1.25 to 5 uM of the selected ASO as described above. The highest CSP1 mRNA fold change (FC) and standard deviation (SD) is provided in Table 6.

TABLE 6

| Name | FC | DS | Dose (uM) |
|---|---|---|---|
| hCPS1-ASO-1a | 5.5803 | 0.9290 | 5 uM |
| hCPS1-ASO-1b | 1.1870 | 0.1452 | 5 uM |
| hCPS1-ASO-1c | 1.4590 | 0.2142 | 5 uM |
| hCPS1-ASO-1d | 1.0277 | 0.1069 | 5 uM |
| hCPS1-ASO-1e | 1.0992 | 0.1400 | 5 uM |
| hCPS1-ASO-1f | 1.6978 | 0.4026 | 5 uM |
| hCPS1-ASO-1g | 4.4990 | 0.6087 | 5 uM |
| hCPS1-ASO-2 | 4.3014 | 0.9287 | 5 uM |
| hCPS1-ASO-3 | 2.2218 | 0.1531 | 5 uM |
| hCPS1-ASO-1h | 1.1727 | 0.1382 | 1 |
| hCPS1-ASO-1i | 1.4355 | 0.1558 | 5 uM |
| hCPS1-ASO-1j | 1.2027 | 0.0988 | 5 uM |
| hCPS1-ASO-1k | 1.1188 | 0.0499 | 0.2 |
| hCPS1-ASO-1l | 1.3171 | 0.1662 | 5 uM |
| hCPS1-ASO-1m | 1.1873 | 0.1099 | 5 uM |
| hCPS1-ASO-1n | 1.1683 | 0.1096 | 1 |
| hCPS1-ASO-1o | 1.3191 | 0.1220 | 5 uM |
| hCPS1-ASO-1p | 1.9562 | 0.0841 | 5 uM |
| hCPS1-ASO-1q | 1.2673 | 0.1820 | 0.2 |
| hCPS1-ASO-1r | 1.5857 | 0.1339 | 5 |
| hCPS1-ASO-1s | 2.5762 | 0.3147 | 5 |
| hCPS1-ASO-1t | 2.4333 | 0.2508 | 5 |
| hCPS1-ASO-1u | 2.9405 | 0.2842 | 5 |
| hCPS1-ASO-1v | 1.9457 | 0.2076 | 5 |
| hCPS1-ASO-82a | 2.5019 | 0.3142 | 5 |
| hCPS1-ASO-82a | 2.5546 | 0.1486 | 5 |
| hCPS1-ASO-86a | 1.2337 | 0.1193 | 1.25 |
| hCPS1-ASO-86b | 1.3452 | 0.4155 | 2.5 |
| hCPS1-ASO-87a | 1.7071 | 0.3347 | 5 |
| hCPS1-ASO-88a | 1.4598 | 0.2254 | 5 |
| hCPS1-ASO-89a | 1.1069 | 0.2738 | 5 |
| hCPS1-ASO-91a | 1.9014 | 0.3037 | 5 |
| hCPS1-ASO-91b | 3.0003 | 0.6407 | 2.5 |
| hCPS1-ASO-91c | 1.8697 | 0.2390 | 2.5 |
| hCPS1-ASO-91d | 2.2671 | 0.0950 | 2.5 |
| hCPS1-ASO-91e | 3.2286 | 0.3834 | 2.5 |
| hCPS1-ASO-91f | 1.6813 | 0.1274 | 1.25 |
| hCPS1-ASO-91g | 2.8205 | 0.0827 | 2.5 |
| hCPS1-ASO-91h | 3.1424 | 0.1637 | 2.5 |
| hCPS1-ASO-91i | 3.7109 | 0.4564 | 1.25 |
| hCPS1-ASO-91j | 5.0641 | 0.2305 | 5 uM |
| hCPS1-ASO-279a | 1.3629 | 0.1492 | 5 uM |
| hCPS1-ASO-280a | 1.9970 | 0.4340 | 5 uM |
| hCPS1-ASO-281a | 1.9249 | 0.2595 | 5 uM |
| hCPS1-ASO-282a | 1.4252 | 0.4261 | 5 uM |
| hCPS1-ASO-283a | 4.4672 | 1.1138 | 5 uM |
| hCPS1-ASO-284a | 5.0837 | 1.5527 | 5 uM |
| hCPS1-ASO-285a | 4.7339 | 0.3981 | 5 uM |
| hCPS1-ASO-286a | 1.5091 | 0.3739 | 5 uM |
| hCPS1-ASO-287a | 1.3469 | 0.3479 | 5 uM |
| hCPS1-ASO-288a | 1.6093 | 0.2169 | 5 uM |
| hCPS1-ASO-289a | 0.9802 | 0.3187 | 5 uM |
| hCPS1-ASO-290a | 1.6650 | 0.1183 | 5 uM |
| hCPS1-ASO-291a | 1.7705 | 0.4831 | 5 uM |
| hCPS1-ASO-292a | 1.8768 | 0.3481 | 5 uM |
| hCPS1-ASO-293a | 1.8295 | 0.3331 | 5 uM |
| hCPS1-ASO-294a | 4.2131 | 0.7748 | 5 uM |
| hCPS1-ASO-295a | 0.8242 | 0.1736 | 5 uM |
| hCPS1-ASO-296a | 1.7239 | 0.4255 | 5 uM |
| hCPS1-ASO-297a | 2.0747 | 0.4485 | 5 uM |
| hCPS1-ASO-298a | 1.1081 | 0.1802 | 5 uM |
| hCPS1-ASO-299a | 5.1515 | 0.9679 | 5 uM |
| hCPS1-ASO-300a | 4.8213 | 0.9887 | 5 uM |
| hCPS1-ASO-301a | 3.3943 | 0.6421 | 5 uM |
| hCPS1-ASO-302a | 1.8365 | 0.4610 | 5 uM |
| hCPS1-ASO-303a | 1.3137 | 0.2222 | 5 uM |
| hCPS1-ASO-303b | 1.7427 | 0.3256 | 5 uM |
| hCPS1-ASO-304a | 2.2107 | 0.3595 | 5 uM |
| hCPS1-ASO-304b | 2.0165 | 0.7982 | 5 uM |
| hCPS1-ASO-305a | 1.8751 | 0.3138 | 5 uM |
| hCPS1-ASO-305b | 2.9614 | 0.4636 | 5 uM |
| hCPS1-ASO-306a | 2.9192 | 0.8811 | 5 uM |
| hCPS1-ASO-306b | 3.4468 | 1.3494 | 5 uM |
| hCPS1-ASO-307a | 1.3314 | 0.3165 | 5 uM |
| hCPS1-ASO-307b | 1.2205 | 0.1267 | 5 uM |
| hCPS1-ASO-308a | 1.2918 | 0.2042 | 5 uM |
| hCPS1-ASO-308b | 1.1019 | 0.1488 | 5 uM |
| hCPS1-ASO-309a | 1.9776 | 0.5046 | 5 uM |
| hCPS1-ASO-309b | 1.4482 | 0.2178 | 5 uM |
| hCPS1-ASO-309c | 1.8643 | 0.3592 | 5 uM |
| hCPS1-ASO-310a | 2.1756 | 0.8122 | 5 uM |
| hCPS1-ASO-310b | 2.1692 | 0.3493 | 5 uM |
| hCPS1-ASO-310c | 1.5170 | 0.2149 | 5 uM |
| hCPS1-ASO-310d | 1.7083 | 0.2543 | 5 uM |
| hCPS1-ASO-311a | 3.7975 | 0.7950 | 5 uM |
| hCPS1-ASO-311b | 2.2173 | 0.2349 | 5 uM |
| hCPS1-ASO-311c | 3.880599933 | 0.8311 | 5 uM |
| hCPS1-ASO-312a | 1.284688861 | 0.4866 | 5 uM |

In sum, 109 initial ASOs targeting CPS1 RR44 were made, of which 76 ASOs comprising 69 gapmers and 7 steric ASOs passed the first pass screen. These 76 ASOs were selected for tiling, and 9 ASOs, comprising 4 gapmers and 5 sterics were selected from this screen. A further 24 ASOs comprising 12 gapmers and 12 mixmers with additional chemistries were made via fine tuning. hCPS1-ASO-1g (SEQ ID NO: 22) was selected for further characterization. PO bonds, a GalNAc moiety and additional 5meC moieties were added for in vivo studies, resulting in hCPS1-ASO-1x (SEQ ID NO: 409).

### Example 2: Synthesis and Characterization of Additional CPS1 regRNA-Targeting ASOs

Two additional regRNAs, RR89 and RR90, were identified. ASOs targeting the new regRNA were synthesized and tested at 5 uM in hepatocytes as described above. The CPS1 mRNA fold increase after treatment with these ASOs is provided in Table 7 below.

TABLE 7

| RR | Name | FC | SD | RR | Name | FC | SD |
|---|---|---|---|---|---|---|---|
| RR89 | hCPS1-ASO-317 | 1.5775 | 0.8135 | RR90 | hCPS1-ASO-405 | 1.2012 | 0.2487 |
| RR89 | hCPS1-ASO-318 | 1.5826 | 0.7218 | RR90 | hCPS1-ASO-406 | 1.2865 | 0.2028 |
| RR89 | hCPS1-ASO-319 | 1.5962 | 0.4788 | RR90 | hCPS1-ASO-407 | 1.1295 | 0.2439 |
| RR89 | hCPS1-ASO-320 | 1.4931 | 0.7690 | RR90 | hCPS1-ASO-408 | 1.0533 | 0.2725 |
| RR89 | hCPS1-ASO-321 | 1.6746 | 0.6312 | RR90 | hCPS1-ASO-409 | 0.8087 | 0.1096 |
| RR89 | hCPS1-ASO-322 | 2.0095 | 0.7188 | RR90 | hCPS1-ASO-410 | 1.3942 | 0.5000 |
| RR89 | hCPS1-ASO-323 | 2.0440 | 1.0473 | RR90 | hCPS1-ASO-411 | 1.3583 | 0.3396 |
| RR89 | hCPS1-ASO-324 | 2.3892 | 0.8168 | RR90 | hCPS1-ASO-412 | 1.6266 | 0.7518 |
| RR89 | hCPS1-ASO-325 | 1.5915 | 0.7186 | RR90 | hCPS1-ASO-413 | 1.2538 | 0.4311 |
| RR89 | hCPS1-ASO-326 | 2.1136 | 1.1464 | RR90 | hCPS1-ASO-414 | 1.1461 | 0.3094 |
| RR89 | hCPS1-ASO-327 | 1.9483 | 0.9495 | RR90 | hCPS1-ASO-415 | 0.9143 | 0.0766 |
| RR89 | hCPS1-ASO-328 | 1.6812 | 0.5726 | RR90 | hCPS1-ASO-416 | 1.1560 | 0.1866 |
| RR89 | hCPS1-ASO-329 | 1.8316 | 0.9072 | RR90 | hCPS1-ASO-417 | 1.0591 | 0.2657 |
| RR89 | hCPS1-ASO-330 | 2.0059 | 0.8976 | RR90 | hCPS1-ASO-418 | 1.3887 | 0.3178 |
| RR89 | hCPS1-ASO-331 | 1.8741 | 0.7026 | RR90 | hCPS1-ASO-419 | 1.3402 | 0.4910 |
| RR89 | hCPS1-ASO-332 | 2.1500 | 0.7416 | RR90 | hCPS1-ASO-420 | 1.5140 | 0.3829 |
| RR89 | hCPS1-ASO-333 | 1.5499 | 0.5157 | RR90 | hCPS1-ASO-421 | 1.0943 | 0.2253 |
| RR89 | hCPS1-ASO-334 | 1.5552 | 0.4804 | RR90 | hCPS1-ASO-422 | 1.1711 | 0.1122 |
| RR89 | hCPS1-ASO-335 | 1.9576 | 0.5176 | RR90 | hCPS1-ASO-423 | 0.9694 | 0.1022 |
| RR89 | hCPS1-ASO-336 | 1.8716 | 0.6998 | RR90 | hCPS1-ASO-424 | 1.2236 | 0.1582 |
| RR89 | hCPS1-ASO-337 | 1.8125 | 0.8488 | RR90 | hCPS1-ASO-425 | 1.2610 | 0.1906 |
| RR89 | hCPS1-ASO-338 | 2.0401 | 0.7645 | RR90 | hCPS1-ASO-426 | 1.3614 | 0.2645 |
| RR89 | hCPS1-ASO-339 | 2.2799 | 1.3896 | RR90 | hCPS1-ASO-427 | 1.0732 | 0.1397 |
| RR89 | hCPS1-ASO-340 | 2.4647 | 1.1151 | RR90 | hCPS1-ASO-428 | 1.0589 | 0.2379 |
| RR89 | hCPS1-ASO-341 | 1.6513 | 0.6047 | RR90 | hCPS1-ASO-429 | 0.8780 | 0.1622 |
| RR89 | hCPS1-ASO-342 | 3.0261 | 1.3769 | RR90 | hCPS1-ASO-430 | 1.1569 | 0.1560 |
| RR89 | hCPS1-ASO-343 | 1.6381 | 0.7347 | RR90 | hCPS1-ASO-431 | 1.1585 | 0.1097 |
| RR89 | hCPS1-ASO-344 | 1.5409 | 0.4495 | RR90 | hCPS1-ASO-432 | 1.2950 | 0.2285 |
| RR89 | hCPS1-ASO-345 | 2.0952 | 1.0805 | RR90 | hCPS1-ASO-433 | 1.0298 | 0.2219 |
| RR89 | hCPS1-ASO-346 | 2.3178 | 1.3366 | RR90 | hCPS1-ASO-434 | 1.3012 | 0.0903 |
| RR89 | hCPS1-ASO-347 | 1.4951 | 0.6377 | RR90 | hCPS1-ASO-435 | 1.0841 | 0.1667 |
| RR89 | hCPS1-ASO-348 | 1.6709 | 0.5697 | RR90 | hCPS1-ASO-436 | 1.1873 | 0.2434 |
| RR89 | hCPS1-ASO-349 | 1.1956 | 0.5132 | RR90 | hCPS1-ASO-437 | 0.8902 | 0.4503 |
| RR89 | hCPS1-ASO-350 | 1.3203 | 0.7134 | RR90 | hCPS1-ASO-438 | 1.1951 | 0.1350 |
| RR89 | hCPS1-ASO-351 | 1.7942 | 1.1274 | RR90 | hCPS1-ASO-439 | 0.8887 | 0.0839 |
| RR89 | hCPS1-ASO-352 | 1.5520 | 0.9718 | RR90 | hCPS1-ASO-440 | 1.0615 | 0.1012 |
| RR89 | hCPS1-ASO-353 | 1.7568 | 1.2231 | RR90 | hCPS1-ASO-441 | 0.8649 | 0.0311 |
| RR89 | hCPS1-ASO-354 | 1.8507 | 1.1719 | RR90 | hCPS1-ASO-442 | 0.9772 | 0.1905 |
| RR89 | hCPS1-ASO-355 | 1.5305 | 0.8114 | RR90 | hCPS1-ASO-443 | 0.8815 | 0.2444 |
| RR89 | hCPS1-ASO-356 | 1.7731 | 0.9631 | RR90 | hCPS1-ASO-444 | 1.3442 | 0.1924 |
| RR89 | hCPS1-ASO-357 | 1.2436 | 0.7147 | RR90 | hCPS1-ASO-445 | 0.8889 | 0.0616 |
| RR89 | hCPS1-ASO-358 | 1.6246 | 1.3366 | RR90 | hCPS1-ASO-446 | 1.3171 | 0.2598 |
| RR89 | hCPS1-ASO-359 | 1.5335 | 1.0744 | RR90 | hCPS1-ASO-447 | 1.0407 | 0.0687 |
| RR89 | hCPS1-ASO-360 | 1.9214 | 1.5074 | RR90 | hCPS1-ASO-448 | 1.1217 | 0.1056 |
| RR89 | hCPS1-ASO-361 | 1.4754 | 0.9292 | RR90 | hCPS1-ASO-449 | 1.1537 | 0.3244 |
| RR89 | hCPS1-ASO-362 | 1.6906 | 1.0116 | RR90 | hCPS1-ASO-450 | 1.3109 | 0.2590 |
| RR89 | hCPS1-ASO-363 | 1.3134 | 0.7014 | RR90 | hCPS1-ASO-451 | 1.5129 | 0.6513 |
| RR89 | hCPS1-ASO-364 | 1.7029 | 0.7542 | RR90 | hCPS1-ASO-452 | 1.2111 | 0.1128 |
| RR89 | hCPS1-ASO-365 | 1.6591 | 0.7996 | RR90 | hCPS1-ASO-453 | 1.0163 | 0.1088 |
| RR89 | hCPS1-ASO-366 | 1.6470 | 1.0509 | RR90 | hCPS1-ASO-454 | 1.3094 | 0.3247 |
| RR89 | hCPS1-ASO-367 | 1.8307 | 1.2695 | RR90 | hCPS1-ASO-455 | 1.1397 | 0.2210 |
| RR89 | hCPS1-ASO-368 | 1.6084 | 0.9115 | RR90 | hCPS1-ASO-456 | 1.0946 | 0.1733 |
| RR89 | hCPS1-ASO-369 | 1.3892 | 0.8035 | RR90 | hCPS1-ASO-457 | 1.0506 | 0.1601 |
| RR89 | hCPS1-ASO-370 | 1.5032 | 0.7262 | RR90 | hCPS1-ASO-458 | 1.2491 | 0.2422 |
| RR89 | hCPS1-ASO-371 | 1.4850 | 0.5957 | RR90 | hCPS1-ASO-459 | 1.3670 | 0.2012 |
| RR89 | hCPS1-ASO-372 | 1.5812 | 0.5242 | RR90 | hCPS1-ASO-460 | 1.3623 | 0.1823 |
| RR89 | hCPS1-ASO-373 | 1.6841 | 0.7860 | RR90 | hCPS1-ASO-461 | 1.0625 | 0.2351 |
| RR89 | hCPS1-ASO-374 | 1.4621 | 0.9788 | RR90 | hCPS1-ASO-462 | 1.2013 | 0.4743 |
| RR89 | hCPS1-ASO-375 | 1.6176 | 1.1216 | RR90 | hCPS1-ASO-463 | 1.0886 | 0.1494 |
| RR89 | hCPS1-ASO-376 | 1.3686 | 0.7403 | RR90 | hCPS1-ASO-464 | 1.1047 | 0.2209 |
| RR89 | hCPS1-ASO-377 | 1.2268 | 0.8146 | RR90 | hCPS1-ASO-465 | 1.1645 | 0.1735 |
| RR89 | hCPS1-ASO-378 | 1.6610 | 1.1474 | RR90 | hCPS1-ASO-466 | 1.4413 | 0.3431 |
| RR89 | hCPS1-ASO-379 | 1.4860 | 0.8718 | RR90 | hCPS1-ASO-467 | 1.2275 | 0.0838 |
| RR89 | hCPS1-ASO-380 | 1.5339 | 0.5719 | RR90 | hCPS1-ASO-468 | 1.3235 | 0.1612 |
| RR89 | hCPS1-ASO-381 | 1.3295 | 0.5757 | RR90 | hCPS1-ASO-469 | 0.8282 | 0.2019 |
| RR89 | hCPS1-ASO-382 | 1.9644 | 0.7430 | RR90 | hCPS1-ASO-470 | 0.9232 | 0.2007 |
| RR89 | hCPS1-ASO-383 | 1.8440 | 1.1617 | RR90 | hCPS1-ASO-471 | 1.3966 | 0.5929 |
| RR89 | hCPS1-ASO-384 | 1.5065 | 0.9131 | RR90 | hCPS1-ASO-472 | 1.0118 | 0.3935 |
| RR89 | hCPS1-ASO-385 | 1.5340 | 0.7326 | RR90 | hCPS1-ASO-473 | 0.9936 | 0.0465 |
| RR89 | hCPS1-ASO-386 | 1.6840 | 0.7312 | RR90 | hCPS1-ASO-474 | 1.3558 | 0.1254 |
| RR89 | hCPS1-ASO-387 | 1.3766 | 0.5754 | RR90 | hCPS1-ASO-475 | 1.2423 | 0.2156 |
| RR89 | hCPS1-ASO-388 | 1.4547 | 0.5771 | RR90 | hCPS1-ASO-476 | 1.9336 | 1.7330 |
| RR89 | hCPS1-ASO-389 | 1.3246 | 0.8197 | RR90 | hCPS1-ASO-477 | 0.9444 | 0.2228 |
| RR89 | hCPS1-ASO-390 | 2.5864 | 1.2608 | RR90 | hCPS1-ASO-478 | 1.3043 | 0.4730 |
| RR89 | hCPS1-ASO-391 | 1.5723 | 0.9236 | RR90 | hCPS1-ASO-479 | 1.0763 | 0.2153 |
| RR89 | hCPS1-ASO-392 | 1.4165 | 1.0256 | RR90 | hCPS1-ASO-480 | 1.0685 | 0.0883 |
| RR89 | hCPS1-ASO-393 | 1.6100 | 0.9882 | RR90 | hCPS1-ASO-481 | 1.0366 | 0.2728 |
| RR89 | hCPS1-ASO-394 | 1.2672 | 0.8009 | RR90 | hCPS1-ASO-482 | 1.3154 | 0.2555 |
| RR89 | hCPS1-ASO-395 | 1.6338 | 0.5694 | RR90 | hCPS1-ASO-483 | 1.2040 | 0.0888 |
| RR89 | hCPS1-ASO-396 | 1.5087 | 0.7520 | RR90 | hCPS1-ASO-484 | 1.4609 | 0.3332 |
| RR89 | hCPS1-ASO-397 | 1.7948 | 0.8470 | RR90 | hCPS1-ASO-485 | 1.4426 | 0.3264 |
| RR89 | hCPS1-ASO-398 | 1.6142 | 0.8862 | RR90 | hCPS1-ASO-486 | 1.1654 | 0.3570 |
| RR89 | hCPS1-ASO-399 | 1.6498 | 0.8071 | RR90 | hCPS1-ASO-487 | 1.2836 | 0.1139 |
| RR89 | hCPS1-ASO-400 | 1.3005 | 0.7143 | RR90 | hCPS1-ASO-488 | 1.1598 | 0.5098 |
| RR89 | hCPS1-ASO-401 | 1.2663 | 0.6460 | RR90 | hCPS1-ASO-489 | 1.1434 | 0.1147 |
| RR89 | hCPS1-ASO-402 | 1.3756 | 0.5260 | RR90 | hCPS1-ASO-490 | 1.1133 | 0.1801 |
| RR89 | hCPS1-ASO-403 | 0.9677 | 0.3162 | RR90 | hCPS1-ASO-491 | 1.1545 | 0.0996 |
| RR89 | hCPS1-ASO-404 | 1.3644 | 0.4530 | RR90 | hCPS1-ASO-492 | 1.8287 | 0.8381 |

### Example 3: In Vivo Characterization of Selected ASOs in Non-Human Primates

Male cynomolgus monkeys (NHP), 2-4 years old, were subcutaneously injected with either 20 mg/kg or 50 mg/kg of hCPS1-ASO-1x on Day 0, and repeated doses were given to some groups on Day 21. Three NHPs were randomly assigned as one group. PBS was administered to one group as a negative control. Ammonia challenge and ureagenesis assay in NHPs were performed in an overnight fasted state prior to the ammonia challenge. 200 mg/kg of ¹⁵NH₄Cl solution was subcutaneously injected into NHPs and multiple blood draws were performed over 0-120 min and immediately plasmas were obtained by centrifugation.

hCPS1-ASO-1x decreased ammonia production in NHPs by a statistically significant amount as compared to PBS only treatment by Day 27. FIG. 17 provides the ammonia AUC both over time and per dose.

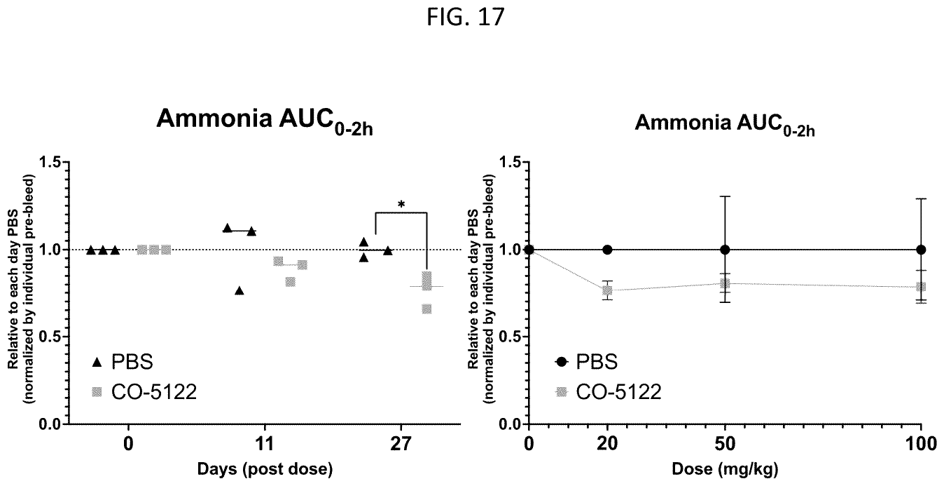

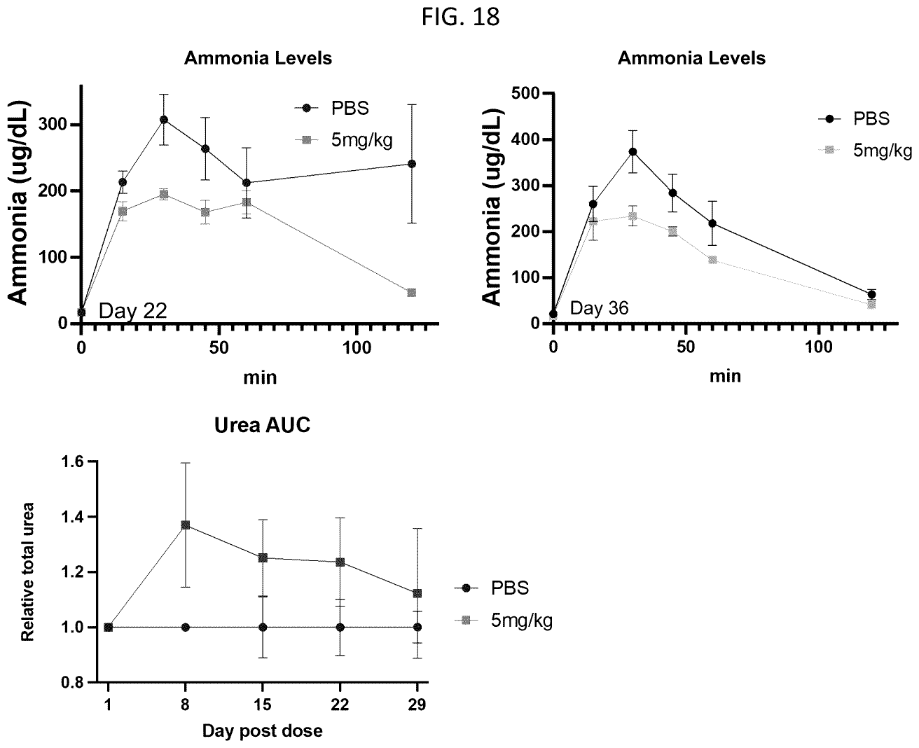

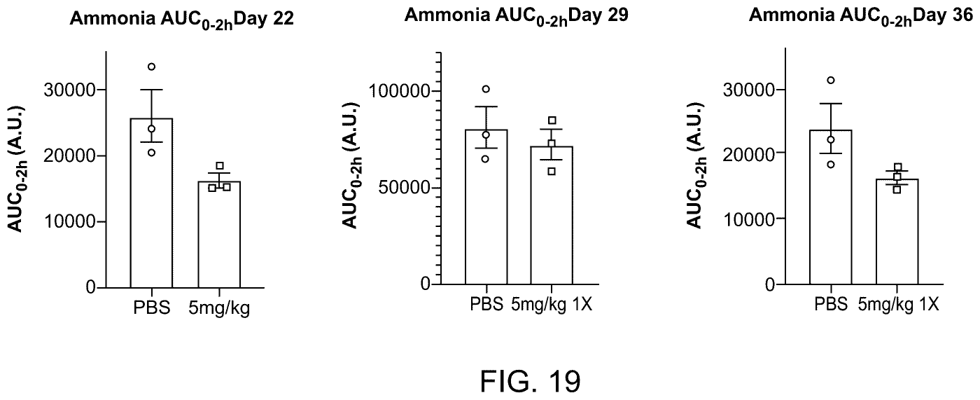

A second in vivo NHP study was performed to assess the efficacy with a lower dose of hCPS1-ASO-1x. Female cynomolgus monkeys (NHP), 2-4 years old, were subcutaneously injected with either 5 mg/kg or 15 mg/kg of hCPS1-ASO-1x on Day 1, and repeated doses were given on Day 36. Each group was consisted with three NHPs. One group was administered with PBS as a negative control. Ammonia challenge and ureagenesis assay in NHPs were performed in an overnight fasted state prior to the ammonia challenge. 200 mg/kg of ¹⁵NH₄Cl solution was subcutaneously injected into NHPs and several blood draws were performed over 0-120 min and immediately plasmas were obtained by centrifugation. ¹³C-ureagenesis assay was performed in an overnight fated state prior to sodium acetate delivery. 55 mg/kg of ¹³C-sodium acetate was provided by oral gavage and multiple blood draws were performed over 0-240 min and plasmas were immediately obtained by centrifugation. FIG. 18 shows that hCPS1-ASO-1x decreased ammonia in NHPs after a single dose of 5 mg/kg for up to 5 weeks (shown are ammonia levels on day 22 and Day 36). In addition, urea levels increased in the ASOs treated NHPs up to 29 days post dose. FIG. 19 provides the ammonia AUC quantification on Days 22, 29 and 36.

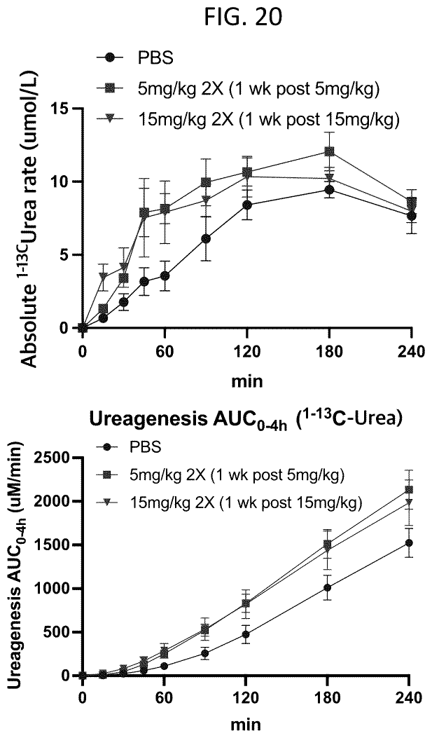

Combination of hCPS1-ASO-1x with ¹³C-sodium acetate treatment enhanced ureagenesis in NHPs at day 43 as compared to hCPS1-ASO-1x alone (FIG. 20).

In sum, upregulation of CPS1 increased the output of the urea cycle in mouse, NHP, and human hepatocytes, both in vitro and in vivo. Increasing CPS1 mRNA in the mouse OTC^{def} model via ASO treatment also increased the metabolism of ammonia to urea and the NHP data and humanized mouse liver data support this finding.

### INCORPORATION BY REFERENCE

Unless stated to the contrary, the entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. An antisense oligonucleotide (ASO) targeting a regulatory RNA (regRNA) of carbamoyl phosphate synthase 1 (CPS1) comprising a nucleotide sequence complementary to at least 8 contiguous nucleotides of any one of SEQ ID NOs: 49, 50, 69-90.

2. The ASO of claim 1, wherein the ASO comprises a nucleotide sequence and optionally a chemical modification of any one of SEQ ID NOs: 1-48, 51-68, or 91-662.

3. An antisense oligonucleotide (ASO) comprising a nucleotide sequence of TGCAGGCACACACATCAGGC (SEQ ID NO: 1).

4. The ASO of claim 3, wherein the 3′ end of the ASO is conjugated to a ligand moiety.

5. The ASO of claim 4, wherein the ligand moiety comprises N-acetylgalactosamine (GalNAc).

6. The ASO of claim 5, wherein the ligand moiety comprises a three-cluster GalNAc moiety (GalNAc3).

7. The ASO of claim 3, wherein one or more of the nucleotides of the ASO are chemically modified.

8. The ASO of claim 3, wherein the ASO comprises MT=MG=M5C-MA-MG=dG=d5C=dA=d5C=dA=d5C=dA=d5C=dA=dT=M5C-MA-MG=MG=M5C (SEQ ID NO: 404), wherein MT, MG, MA, and M5C are 2′-O-methoxyethyl thymidine, 2′-O-methoxyethyl guanosine, 2′-O-methoxyethyl adenosine, and 2′-O-methoxyethyl 5-methyl cytidine ribonucleosides, respectively; dA, dG, dT, and d5C are 2′-deoxy adenosine, 2′-deoxy guanosine, 2′-deoxy thymidine and 2′-deoxy 5-methyl cytidine ribonucleosides, respectively, “=” is a phosphorothioate linkage, and “-” is a phosphodiester linkage.

9. The ASO of claim 8, wherein the 3′ end of the ASO is conjugated to a ligand moiety.

10. The ASO of claim 9, wherein the ligand moiety comprises N-acetylgalactosamine (GalNAc).

11. The ASO of claim 10, wherein the ligand moiety comprises a three-cluster GalNAc moiety (GalNAc3).

12. The ASO of claim 3, wherein the ASO comprises MT=MG=M5C-MA-MG=dG=d5C=dA=d5C=dA=d5C=dA=d5C=dA=dT=M5C-MA-MG=MG=M5C-[TEG] (SEQ ID NO: 409), wherein MT, MG, MA, and M5C are 2′-O-methoxyethyl thymidine, 2′-O-methoxyethyl guanosine, 2′-O-methoxyethyl adenosine, and 2′-O-methoxyethyl 5-methyl cytidine ribonucleosides; dA, dG, dT, and d5C are 2′-deoxy adenosine, 2′-deoxy guanosine, 2′-deoxy thymidine and 2′-deoxy 5-methyl cytidine ribonucleosides, “=” is a phosphorothioate linkage, “-” is a phosphodiester linkage, and [TEG] is a ligand moiety comprising a triethyleneglycol linked to N-acetylgalactosamine (GalNAc).

13. The ASO of claim 12, wherein the ligand moiety comprises a three-cluster GalNAc (GalNAc3).

14. An antisense oligonucleotide (ASO) comprising MT=MG=M5C-MA-MG=dG=d5C=dA=d5C=dA=d5C=dA=d5C=dA=dT=M5C-MA-MG=MG=M5C-[TEG] (SEQ ID NO: 409), wherein MT, MG, MA, and M5C are 2′-O-methoxyethyl thymidine, 2′-O-methoxyethyl guanosine, 2′-O-methoxyethyl adenosine, and 2′-O-methoxyethyl 5-methyl cytidine ribonucleosides; dA, dG, dT, and d5C are 2′-deoxy adenosine, 2′-deoxy guanosine, 2′-deoxy thymidine and 2′-deoxy 5-methyl cytidine ribonucleosides, “=” is a phosphorothioate linkage, “-” is a phosphodiester linkage, and [TEG] is a ligand moiety comprising a triethyleneglycol linked to N-acetylgalactosamine (GalNAc).

15. The ASO of claim 14, wherein the ligand moiety comprises a three-cluster GalNAc moiety (GalNAc3).

16. An antisense oligonucleotide (ASO) comprising MT=MG=M5C-MA-MG=dG=d5C=dA=d5C=dA=d5C=dA=d5C=dA=dT=M5C-MA-MG=MG=M5C (SEQ ID NO: 404), wherein MT, MG, MA, and M5C are 2′-O-methoxyethyl thymidine, 2′-O-methoxyethyl guanosine, 2′-O-methoxyethyl adenosine, and 2′-O-methoxyethyl 5-methyl cytidine ribonucleosides; dA, dG, dT, and d5C are 2′-deoxy adenosine, 2′-deoxy guanosine, 2′-deoxy thymidine and 2′-deoxy 5-methyl cytidine ribonucleosides, “=” is a phosphorothioate linkage, “-” is a phosphodiester linkage.

17. A pharmaceutical composition comprising the ASO of claim 1.

18. A pharmaceutical composition comprising the ASO of claim 3.

19. A pharmaceutical composition comprising the ASO of claim 14.

20. A pharmaceutical composition comprising the ASO of claim 16.

21. A method of modulating expression of CPS1 in a cell, comprising contacting the cell with the ASO of claim 1.

22. A method of increasing expression of CPS1 in a human cell, comprising contacting the cell with the ASO of claim 3.

23. A method of treating a urea cycle disorder comprising administering to a subject in need thereof an effective amount of the ASO of claim 1.

24. The method of claim 23, wherein the urea cycle disorder comprises carbamoylphosphate synthetase 1 (CPS1) deficiency, ornithine transcarbamylase deficiency (OTC) deficiency, argininosuccinate synthetase 1 (ASS1) deficiency, argininosuccinate lyase (ASL) deficiency, arginase 1 deficiency, ornithine translocase (ORNT1) deficiency, hyperornithinemia-hyperammonemia-homocitrullinuria (HHH) syndrome, or citrin deficiency.

25. A method of treating a urea cycle disorder comprising administering to a subject in need thereof an effective amount of the ASO of claim 3.

26. A method of treating or preventing hyperammonemia in a subject comprising administering to a subject in need thereof an effective amount of the ASO of claim 1.

27. The method of claim 26, wherein the subject comprises one or more of: a urea cycle disorder, hepatic encephalopathy, acute liver failure, liver cirrhosis, non-alcoholic fatty liver disease, renal dysfunction or failure, propionic acidemia, methylmalonic acidemia, isovaleric acidemia, a urinary tract infection, intestinal bacterial overgrowth, a fatty acid oxidation disorder, and a systemic infection, or wherein the subject has or is at risk of developing hyperammonemia associated with the intake of a drug.

28. The method of claim 27, wherein the drug is valproic acid, carbamazepine, fluorouracil, sulfadiazine, ribavirin, salicylates, or glycine.

29. A method of treating or preventing hyperammonemia in a subject comprising administering to a subject in need thereof an effective amount of the ASO of claim 3.

30. A method of increasing the level of CPS1 in a cell comprising contacting the cell with an ASO of claim 1.


SEQUENCE LISTING (662 sequences)

SEQ ID NO: 1 - RNA, 20 nt, synthetic construct
  source 1..20: mol_type = other RNA
tgcaggcacacacatcaggc

SEQ ID NO: 2 - RNA, 22 nt, synthetic construct
  source 1..22: mol_type = other RNA
atgcaggcacacacatcaggct

SEQ ID NO: 3 - RNA, 24 nt, synthetic construct
  source 1..24: mol_type = other RNA
aatgcaggcacacacatcaggctg

SEQ ID NO: 4 - RNA, 20 nt, synthetic construct
  source 1..20: mol_type = other RNA
gcacacacatcaggctgggg

SEQ ID NO: 5 - RNA, 20 nt, synthetic construct
  source 1..20: mol_type = other RNA
aatgcaggcacacacatcag

SEQ ID NO: 6 - RNA, 20 nt, synthetic construct
  source 1..20: mol_type = other RNA
tgaatgcaggcacacacatc

SEQ ID NO: 7 - RNA, 20 nt, synthetic construct
  source 1..20: mol_type = other RNA
gggaccctgtgtacttggaa

SEQ ID NO: 8 - RNA, 20 nt, synthetic construct
  source 1..20: mol_type = other RNA
gcagagtgacaggcatgaat

SEQ ID NO: 9 - RNA, 20 nt, synthetic construct
  source 1..20: mol_type = other RNA
tgccattgacttgcataatg

SEQ ID NO: 10 - RNA, 20 nt, synthetic construct
  source 1..20: mol_type = other RNA
gtcaatgctcagaacatagt

SEQ ID NO: 11 - RNA, 20 nt, synthetic construct
  source 1..20: mol_type = other RNA
tagataataacccaccacac

SEQ ID NO: 12 - RNA, 20 nt, synthetic construct
  source 1..20: mol_type = other RNA
gtcaagtactaggttgtatt

SEQ ID NO: 13 - RNA, 20 nt, synthetic construct
  source 1..20: mol_type = other RNA
ttagcctgtgaaatccctca

SEQ ID NO: 14 - RNA, 20 nt, synthetic construct
  source 1..20: mol_type = other RNA
cttatgagtcactctctgat

SEQ ID NO: 15 - RNA, 20 nt, synthetic construct
  source 1..20: mol_type = other RNA
tctttgtgagccaggtcatc

SEQ ID NO: 16 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,16,20): mod_base = OTHER; note = 5-methyl on the cytidine
  misc_feature 6..15: note = DNA
  misc_feature order(1..5,16..20): note = RNA
tgcaggcacacacatcaggc

SEQ ID NO: 17 - DNA, 20 bp, synthetic construct
  source 1..20: mol_type = other DNA
  modified_base 1..20: mod_base = OTHER; note = 2-prime-O-(2-Methoxyethyl) nucleotide
  modified_base order(3,7,9,11,13,16,20): mod_base = OTHER; note = 5-methyl on the cytidine
tgcaggcacacacatcaggc

SEQ ID NO: 18 - DNA, 20 bp, synthetic construct
  source 1..20: mol_type = other DNA
  modified_base order(1..5,9,12,16..20): mod_base = OTHER; note = 2-prime-O-(2-Methoxyethyl) nucleotide
  modified_base order(3,9,16,20): mod_base = OTHER; note = 5-methyl on the cytidine
  misc_feature order(6..8,10,11,13..15): note = DNA
  misc_feature order(1..5,9,12,16..20): note = RNA
tgcaggcacacacatcaggc

SEQ ID NO: 19 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,9,16,20): mod_base = OTHER; note = 5-methyl on the cytidine
  modified_base order(6,9,12,15): mod_base = OTHER; note = Locked nucleic acid
  misc_feature order(1..5,9,12,1..20): note = RNA
  misc_feature order(7,8,10,11,13,14): note = DNA
tgcaggcacacacatcaggc

SEQ ID NO: 20 - DNA, 20 bp, synthetic construct
  source 1..20: mol_type = other DNA
  modified_base order(1..5,17..20): mod_base = OTHER; note = 2-prime-O-(2-Methoxyethyl) nucleotide
  modified_base order(3,16,20): mod_base = OTHER; note = 5-methyl on the cytidine
  modified_base order(8,12,16): mod_base = OTHER; note = Locked nucleic acid
  misc_feature order(6,7,9..11,13..15): note = DNA
  misc_feature order(1..5,8,12,16..20): note = RNA
tgcaggcacacacatcaggc

SEQ ID NO: 21 - DNA, 20 bp, synthetic construct
  source 1..20: mol_type = other DNA
  modified_base order(1..5,16,18..20): mod_base = OTHER; note = 2-prime-O-(2-Methoxyethyl) nucleotide
  modified_base order(3,9,13,16,20): mod_base = OTHER; note = 5-methyl on the cytidine
  modified_base order(9,13,17): mod_base = OTHER; note = Locked nucleic acid
  misc_feature order(1..5,16,18..20): note = RNA
  misc_feature order(6..8,10..12,14,15): note = DNA
tgcaggcacacacatcaggc

SEQ ID NO: 22 - DNA, 20 bp, synthetic construct
  source 1..20: mol_type = other DNA
  modified_base order(1..5,16,18..20): mod_base = OTHER; note = 2-prime-O-(2-Methoxyethyl) nucleotide
  modified_base order(3,16,20): mod_base = OTHER; note = 5-methyl on the cytidine
  modified_base order(3^4,4^5): mod_base = OTHER; note = Phosphodiester linkage
  modified_base order(16^17,17^18): mod_base = OTHER; note = Phosphodiester linkage
  misc_feature order(1..5,16..20): note = RNA
  misc_feature 6..15: note = DNA
tgcaggcacacacatcaggc

SEQ ID NO: 23 - DNA, 22 bp, synthetic construct
  source 1..22: mol_type = other DNA
  modified_base order(1..6,17..22): mod_base = OTHER; note = 2-prime-O-(2-Methoxyethyl) nucleotide
  modified_base order(4,17,21): mod_base = OTHER; note = 5-methyl on the cytidine
  misc_feature order(1..6,17..22): note = RNA
  misc_feature 7..16: note = DNA
atgcaggcacacacatcaggct

SEQ ID NO: 24 - DNA, 24 bp, synthetic construct
  source 1..24: mol_type = other DNA
  modified_base order(1..7,18..24): mod_base = OTHER; note = 2-prime-O-(2-Methoxyethyl) nucleotide
  modified_base order(5,18,22): mod_base = OTHER; note = 5-methyl on the cytidine
  misc_feature order(1..7,18..24): note = RNA
  misc_feature 8..17: note = DNA
aatgcaggcacacacatcaggctg

SEQ ID NO: 25 - DNA, 20 bp, synthetic construct
  source 1..20: mol_type = other DNA
  modified_base order(1..5,16,20): mod_base = OTHER; note = 2-prime-O-(2-Methoxyethyl) nucleotide
  modified_base order(2,4): mod_base = OTHER; note = 5-methyl on the cytidine
  misc_feature order(1..5,16..20): note = RNA
  misc_feature 6..15: note = DNA
gcacacacatcaggctgggg

SEQ ID NO: 26 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(5,18): mod_base = OTHER; note = 5-methyl on the cytidine
  misc_feature order(1..5,16..20): note = RNA
  misc_feature 6..15: note = DNA
aatgcaggcacacacatcag

SEQ ID NO: 27 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(17,20): mod_base = OTHER; note = 5-methyl on the cytidine
  misc_feature order(1..5,16..20): note = RNA
  misc_feature 6..15: note = DNA
tgaatgcaggcacacacatc

SEQ ID NO: 28 - DNA, 20 bp, synthetic construct
  source 1..20: mol_type = other DNA
  modified_base 1..20: mod_base = OTHER; note = 2-prime-O-(2-Methoxyethyl) nucleotide
  modified_base order(2,4,6,8,11,15): mod_base = OTHER; note = 5-methyl on the cytidine
gcacacacatcaggctgggg

SEQ ID NO: 29 - DNA, 20 bp, synthetic construct
  source 1..20: mol_type = other DNA
  modified_base 1..20: mod_base = OTHER; note = 2-prime-O-(2-Methoxyethyl) nucleotide
  modified_base order(5,9,11,13,15,18): mod_base = OTHER; note = 5-methyl on the cytidine
aatgcaggcacacacatcag

SEQ ID NO: 30 - DNA, 20 bp, synthetic construct
  source 1..20: mol_type = other DNA
  modified_base 1..20: mod_base = OTHER; note = 2-prime-O-(2-Methoxyethyl) nucleotide
  modified_base order(7,11,13,15,17,20): mod_base = OTHER; note = 5-methyl on the cytidine
tgaatgcaggcacacacatc

SEQ ID NO: 31 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base 5: mod_base = OTHER; note = 5-methyl on the cytidine
  misc_feature order(1..5,16..20): note = RNA
  misc_feature 6..15: note = DNA
gggaccctgtgtacttggaa

SEQ ID NO: 32 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base 2: mod_base = OTHER; note = 5-methyl on the cytidine
  misc_feature order(1..5,16..20): note = RNA
  misc_feature 6..15: note = DNA
gcagagtgacaggcatgaat

SEQ ID NO: 33 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base 3..4: mod_base = OTHER; note = 5-methyl on the cytidine
  misc_feature order(1..5,16..20): note = RNA
  misc_feature order(6,15): note = DNA
tgccattgacttgcataatg

SEQ ID NO: 34 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base 3: mod_base = OTHER; note = 5-methyl on the cytidine
  misc_feature order(1..5,16..20): note = RNA
  misc_feature 6..15: note = DNA
gtcaatgctcagaacatagt

SEQ ID NO: 35 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(16,18,20): mod_base = OTHER; note = 5-methyl on the cytidine
  misc_feature order(1..5,16..20): note = RNA
  misc_feature 6..15: note = DNA
tagataataacccaccacac

SEQ ID NO: 36 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base 3: mod_base = OTHER; note = 5-methyl on the cytidine
  misc_feature order(1..5,16..20): note = RNA
  misc_feature 6..15: note = DNA
gtcaagtactaggttgtatt

SEQ ID NO: 37 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(5,16,17,19): mod_base = OTHER; note = 5-methyl on the cytidine
  misc_feature order(1..5,16..20): note = RNA
  misc_feature 6..15: note = DNA
ttagcctgtgaaatccctca

SEQ ID NO: 38 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(1,16): mod_base = OTHER; note = 5-methyl on the cytidine
  misc_feature order(1..5,16..20): note = RNA
  misc_feature 6..15: note = DNA
cttatgagtcactctctgat

SEQ ID NO: 39 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(2,17,20): mod_base = OTHER; note = 5-methyl on the cytidine
  misc_feature order(1..5,16..20): note = RNA
  misc_feature 6..15: note = DNA
tctttgtgagccaggtcatc

SEQ ID NO: 40 - DNA, 20 bp, synthetic construct
  source 1..20: mol_type = other DNA
  modified_base 1..20: mod_base = OTHER; note = 2-prime-O-(2-Methoxyethyl) nucleotide
  modified_base order(3,4,14): mod_base = OTHER; note = 5-methyl on the cytidine
tgccattgacttgcataatg

SEQ ID NO: 41 - DNA, 20 bp, synthetic construct
  source 1..20: mol_type = other DNA
  modified_base 1..20: mod_base = OTHER; note = 2-prime-O-(2-Methoxyethyl) nucleotide
  modified_base order(3,8,10,15): mod_base = OTHER; note = 5-methyl on the cytidine
gtcaatgctcagaacatagt

SEQ ID NO: 42 - DNA, 20 bp, synthetic construct
  source 1..20: mol_type = other DNA
  modified_base 1..20: mod_base = OTHER; note = 2-prime-O-(2-Methoxyethyl) nucleotide
  modified_base order(11..13,15,16,18,20): mod_base = OTHER; note = 5-methyl on the cytidine
tagataataacccaccacac

SEQ ID NO: 43 - DNA, 20 bp, synthetic construct
  source 1..20: mol_type = other DNA
  modified_base 1..20: mod_base = OTHER; note = 2-prime-O-(2-Methoxyethyl) nucleotide
  modified_base order(3,9): mod_base = OTHER; note = 5-methyl on the cytidine
gtcaagtactaggttgtatt

SEQ ID NO: 44 - DNA, 20 bp, synthetic construct
  source 1..20: mol_type = other DNA
  modified_base order(5,6,15..17,19): mod_base = OTHER; note = 5-methyl on the cytidine
  modified_base 1..20: mod_base = OTHER; note = 2-prime-O-(2-Methoxyethyl) nucleotide
ttagcctgtgaaatccctca

SEQ ID NO: 45 - DNA, 20 bp, synthetic construct
  source 1..20: mol_type = other DNA
  modified_base 1..20: mod_base = OTHER; note = 2-prime-O-(2-Methoxyethyl) nucleotide
  modified_base order(1,10,12,14,16): mod_base = OTHER; note = 5-methyl on the cytidine
cttatgagtcactctctgat

SEQ ID NO: 46 - DNA, 20 bp, synthetic construct
  source 1..20: mol_type = other DNA
  modified_base 1..20: mod_base = OTHER; note = 2-prime-O-(2-Methoxyethyl) nucleotide
  modified_base order(2,11,12,17,20): mod_base = OTHER; note = 2-prime-O-(2-Methoxyethyl) nucleotide
tctttgtgagccaggtcatc

SEQ ID NO: 47 - DNA, 20 bp, synthetic construct
  source 1..20: mol_type = other DNA
  modified_base 1..20: mod_base = OTHER; note = 2-prime-O-(2-Methoxyethyl) nucleotide
  modified_base order(5..7,14): mod_base = OTHER; note = 5-methyl on the cytidine
gggaccctgtgtacttggaa

SEQ ID NO: 48 - DNA, 20 bp, synthetic construct
  source 1..20: mol_type = other DNA
  modified_base 1..20: mod_base = OTHER; note = 2-prime-O-(2-Methoxyethyl) nucleotide
  modified_base order(2,10,14): mod_base = OTHER; note = 5-methyl on the cytidine
gcagagtgacaggcatgaat

SEQ ID NO: 49 - RNA, 2000 nt, synthetic construct
  source 1..2000: mol_type = other RNA
gcctgatgtgtgtgcctgcattcatgcctgtcactctgctcagctgcgtgcagacagcttatatgaaggaggactgtgtacatgtggtcagagaagaaaaaaatgcttattttttaaaatgttttgaaaaataatctgtcctaaaagtgtaacatttccactaaattttttcatgtctgactttaacattccctaccaaccaactcttatagcctcaactcctttgatctatcctaatgtaaactccctcactaatgtgtaatatatttttttccagtttggttatagatttcagtctagcctgaacactcaagtatgaaactagccactattttatttattggctgttccaacctccattgtggaattataattgatggattttccaagtctctaattcagaatatttaatcaaattttccaaaatttctgccttctcctccttcattctgatgactaaagacatggaccataaaatatatatacatatatacatacatatgtatatgtatgtatatacacatatatatatgtatgtatatatacatacatatatatatgtatatatacacacacatatacatatgtatgtatatatacacacacacatatatatatgtatgtatatatatacacacacatatatatatgtatgtatatatatacacacacatatatatatgtatgtatatatatacacacacacaccgtagtttgcaagtctgcatctgggaccctttactgataaaaatgaaagcccattaaaactctcatgcccacatttctctctagactttagagtaggagaattctatcctagaattgttgattgttgacatttctatggaaaaacatgtttaattcatcatgctttgtagtcaactgctataaatggtgatgaagtcttttccttggtcaagctctggactcctcagtacttccactgtcatatttgattaaaacataagtgatgaagggaggaaatatattatagtattcattgcatgggtaccttagggtaatgggactatgactgatttaaatttattttgcatcctaaaaagtttccattagtgggtgcagcacaccagcatggcacatgtatacatatgtaactaacctgcacaatgtgcacatgtaccctaaaacttaaagtataataataaaagaaaaaaaaacttaaaaagaaaagtttccataatgaggattgagtttatatcgttttcttaattaaggaaagacaaccctcactacacacacacacacacacacacacacacacataaaaagacaagaggtgagatcaaggcgtaaactagtattggactaagccagtaagtactgcttctcagtcctcagttctctaggttgggagttttccttttgctattttcattctcctccaattcatttttcaatgatcttcaacacaaatttcgcctttatttcttgttcaaaatacttccatgactattatgaatactatttatggatgaaggctgaacattttaaggcatactaagtctcccatgatatggtaccttttcattttcccattctataccctgcatctaggcttttaaaaaatataccacatgacttcatgcctctaggctattgctcatgccacggcatcccttctgggttttttcccctctttatctacttggtgaatttgcatccttcagtataacacagctcaacataattctatgcttaaactttactgaaaatgcacaaggaacttgatctttctttcttattttccccattttatttattttcccattgcatttgggcaactttcatggcaccagttattttattaattatttgtataaactagtatgagcctattttgtttagctttctatcaccaggagccatgaagcactttgaaatcaaatactgtttgtacttgaatatattaagtaagcgtgaggacttaaattaaactttgttcctctttaaaaaaatccttaccccaactctagcactga

SEQ ID NO: 50 - RNA, 561 nt, synthetic construct
  source 1..561: mol_type = other RNA
atgaaaatggaatattttgtaatcatttaaagtaatgttttaaaagaactttgtaataacatcaaaatacatttaacataatattgcctccaaaaagcattatgcaagtcaatggcaattttcaaaactatgttctgagcattgacataaatgctagaagataatactttagtgttaacagagattatgtttgtgtggtgggttattatctattattctgtatttccacatttctttaaatatcaaatgaaagtgaatacaacctagtacttgacattttaacattttcaataccagatatgtcaataaggaattcataaaataaagtgccctgagggatttcacaggctaaatgcttctattttttaaaatgtattaatagaaaagaagactaaatctaaaaaaaaaaattcattttatcagagagtgactcataagtttagaaaacctttgatgacctggctcacaaagaatgtacttccaagtacacagggtccccagcctgatgtgtgtgcctgcattcatgcctgtcactctgctcagctgcgtgcagacagctta

SEQ ID NO: 51 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base 5: mod_base = OTHER; note = 5-methyl on the cytidine
  misc_feature order(1..5,16..20): note = RNA
  misc_feature 6..15: note = DNA
tggacatcagcttgggagga

SEQ ID NO: 52 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base 1: mod_base = OTHER; note = 5-methyl on the cytidine
  misc_feature order(1..5,16..20): note = RNA
  misc_feature 6..15: note = DNA
cataatgtttgacagtttgg

SEQ ID NO: 53 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  misc_feature order(1..5,16..20): note = RNA
  misc_feature 6..15: note = DNA
gtatgacatgtccattggaa

SEQ ID NO: 54 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  misc_feature order(1..5,16..20): note = RNA
  misc_feature 6..15: note = DNA
gggttaccaatagctttaaa

SEQ ID NO: 55 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(2..4,17,18,20): mod_base = OTHER; note = 5-methyl on the cytidine
  misc_feature order(1..5,16..20): note = RNA
  misc_feature 6..15: note = DNA
gcccaagtgtaagcttcctc

SEQ ID NO: 56 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base 20: mod_base = OTHER; note = 5-methyl on the cytidine
  misc_feature order(1..5,16..20): note = RNA
  misc_feature 6..15: note = DNA
tatgttgcaatttgtatgac

SEQ ID NO: 57 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(5,7,20): mod_base = OTHER; note = 5-methyl on the cytidine
  misc_feature 6..15: note = DNA
  misc_feature order(1..5,16..20): note = RNA
gggtcatgttattcagctac

SEQ ID NO: 58 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(2,17,19,20): mod_base = OTHER; note = 5-methyl on the cytidine
  misc_feature order(1..5,16..20): note = RNA
  misc_feature 6..15: note = DNA
tctgaagtgaatgaggcacc

SEQ ID NO: 59 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  misc_feature 6..15: note = DNA
  misc_feature order(1..5,16..20): note = RNA
  modified_base order(2..5,16,17,20): mod_base = OTHER; note = 5-methyl on the cytidine
acccctttgacttgcccaag

SEQ ID NO: 60 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  misc_feature order(1..5,16..20): note = RNA
  modified_base order(5,20): mod_base = OTHER; note = 5-methyl on the cytidine
  misc_feature 6..15: note = DNA
ggtgccacttgttatgttgc

SEQ ID NO: 61 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(4,12): mod_base = OTHER; note = 5-methyl on the cytidine
  misc_feature 6..15: note = RNA
  misc_feature order(1..5,16..20): note = DNA
gtacacctaaggctgtttac

SEQ ID NO: 62 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  misc_feature order(1..5,16..20): note = RNA
  misc_feature 6..15: note = DNA
  modified_base order(1,2,5,16): mod_base = OTHER; note = 5-methyl on the cytidine
cctacccatcatgtcctaag

SEQ ID NO: 63 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(2,3,5,17,18,20): mod_base = OTHER; note = 5-methyl on the cytidine
  misc_feature order(1..5,16..20): note = RNA
  misc_feature 6..15: note = DNA
tccacaggagaaggtgccac

SEQ ID NO: 64 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  misc_feature 6..15: note = DNA
  misc_feature order(1..5,16..20): note = RNA
  modified_base 2..3: mod_base = OTHER; note = 5-methyl on the cytidine
accttttccatatttgggtt

SEQ ID NO: 65 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(1,18..20): mod_base = OTHER; note = 5-methyl on the cytidine
  misc_feature order(1..5,16..20): note = RNA
  misc_feature 6..15: note = DNA
cagtaaattcctttgttccc

SEQ ID NO: 66 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  misc_feature order(1..5,16..20): note = RNA
  modified_base 19..20: mod_base = OTHER; note = 5-methyl on the cytidine
  misc_feature 6..15: note = DNA
tttgactgcccactcttacc

SEQ ID NO: 67 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  misc_feature order(1..5,16..20): note = RNA
  misc_feature 6..15: note = DNA
gtttgaccccagtgaatgtt

SEQ ID NO: 68 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base 17: mod_base = OTHER; note = 5-methyl on the cytidine
  misc_feature order(1..5,16..20): note = order(1..5,16..20)
  misc_feature 6..15: note = DNA
ggttgtaagttctagtctat

SEQ ID NO: 69 - RNA, 2001 nt, synthetic construct
  source 1..2001: mol_type = other RNA
tagatagaaaggagcagccagagatgtgcctcaggctgaagccagtgctgcaaatgggacagcgctctcaagctacattatatgcatttcattatccaacaatttgttctgggaggacagtttgattggccaggcaagaaattcaaaatgagagacttttaaatatcacaactcagctaaaagagctcttctgttttctgacctgagaaattcacttctatcagtctcaataaagatgcttgtaatattaggtgatgcagactctgaacatttgtccctctggactgagtgcactcccgatgatctgagtcttggggctatgcctcaggcttcacaattcctagtgtgaaagctacttgcatatccacccctccatccagctgggttttgttctgcttttcttgtgtaactaatgactgtgagtcaaaaagatgggaggtcaactacttgcctttgaccattaaataggtgtttaaactaagctaactaaggaataacattgtttttccatttcgtgatatcgttttccattttatcctagattcttaactaggatgtgaagaaaataatagtagtcaatattataaagtttaaaataatacaaagtagtcgatattataaacacagacatatttctagaatggaacaataagatagtaggcttctatgttttattttaaaaggttccgaaattacccacttaccatttgtgtgaccttgaacaagttgctttaactccgaacctcagtttcttcatatacaaaatgggaatacacctactatggaggtttgctgatagaatttaaacagaataagttacgcaaaatatctggtgttagtaaagaattctctcagtgactcatagcaagagtataaattggtctagtcactttgtagcatgatttgtcattatacagtgaaatgtaaaatgtgcataccttacagcaattacattttttcaggatgaaactcagaaaaatactaaaacatgtgcacaaaatgatagatacatacatagatatatagatattcattgcagcataaatgaaccagtgggaataatatttaaatgcatgaatgttcttttcccttttgaaattattttactgaccttctccacaaactcacctatttcttttgggtggtttggaattattacaacaggaaggcaaagagaaagaaaaagttaatatttatcttctggcaaggcaacgggatttgcagtaagaacacagtgctaacaagtagatttaatcagggagaggggagttcgttaaattcttaaactgctttaacttatcataactcaaactagaggattcagattatatgaatttttcacatctggtgaagtggtaggaagaagaatggaaacacagacgtcacatcagcgtcctttatttccacttccactttcaacccctgatccaggaccactctttaaggcagtaaaggagcaatgtagtagaaatgacagcagcaggccagcagatacctattcctgagacagagcccatggcattcagggcctggaaactggggacccaaaataacccaagtctggttgtgacaacacagaggttctgagagtacaggaagaattaagaagcctggggacaaggtagaaaaccactgcaggtagatcataacagacaagggacaaatgaccagaccccctaacgccccaaagagcagatgtcaacagcacagagagcagaccagctcagctgctctgcagatatcaaggaggactcagaggatacatcaatctgattaccctttccttctgcaccactaaatcatgtaaactaaacaaccagatgccctcttgaagaggaatagggtaagcaggtcaaacttggaaaagtggaacaaattgaccaagcaaggacagggttacctaaatggagtattggactgcattactggactcaagacaaaacttaccagattagactcgtagatcagatcagttatttaaaaaataaataaaataaggta

SEQ ID NO: 70 - RNA, 1001 nt, synthetic construct
  source 1..1001: mol_type = other RNA
gtccatcctgactcctggcagggaccagggccatccagttatactacggtgttctttcaccacctcagtgatggcagctggctggagtccaaagcgtttcagtactcccaagctctgactacttttttctcttcttttcttcatcttgccctacacatttatccaaagaaaatatttaggatgtgaaagaaggggtgtagataagaaatgaatcaaaggagaaatgtgtgattataaaacaataaaatacgtaactgaaccttactgtggcttaacacacagccaatagtgccaaagatcatataacttctgtctgctgaagttttctctttattaaatggcattgacagagaaaaactataaagaataattaattgaaatagactgcaaatcacttagccaacataaaatgatgcaatattttatctgatgaagaaacctaaagttaaatattggttagatcaattttatcaatttgtctatatcctatcaatccttcaaagccatatgaaatcatactcttttaatgaactttatgtatgtatatcttgtttgtatcatacaattaatatactgtccacgtactcttatttcacacattaatatcttcctggttgaaatcattaaatagctccacactgcttaactacagtgcgaagttaaaaatataatatattcatttttctaaagctttaggcttttgtttgcttcattcacgttgtgtgatctaaattagtatcttcactgtctctgtttaatatcgacaaattagaaagagtaacaaccatgacctctcatagtccataagtaataattaattcttcaattccttctataaatacttattaaagcccttttacatgccaagcatggtgctagatccgagaataaaaataagtctggaacacagacttgtccctcaaaagctccctcttgagaagcctttcagttttattcttagaagtggacaaaaatgtgtattttctactctcaagattgaa

SEQ ID NO: 71 - RNA, 1001 nt, synthetic construct
  source 1..1001: mol_type = other RNA
aaattaaacatatttgaggatcacagagaagcatgtggtgcaacatgtcttgaagttaccgacttcgtgaagtgtaaggggcagtagtttacactggaatgacgtagagaacaggtcacctacgcacattcctactccacccccttttgtagaaaatttgttatactgaacctgatttcttccatggcctcctattttcagcggaatgtgattcaggaagatacattccaccctcctggaaatcggaaataggacccgtgctttcacaaacattaagttccatcttccctaaaactttattgcaattccccaggaatggatcacagagctatttcttcctccaggtttgtagatttgtaagttgagtcatagggcaggagtacctttttttattttatataggctaaaaatttgttggagaagattagtgatctggagaggggaaaaaaaagaagaacaagaaggagatgaagaaggggaggaagaagagaaagaaagacaaaaggaagccaaccctaatccttgaattctacaaactactaacaattagtcaaaagtgcctacttggatttcaatacctgacatgcctttgagtgttgaagaattaaatgttttattctggtattgttttttgatgtcatttatttttaacctatacttgcccctatcaagatcattctcagttgatagtaagaaaaatatgtaatgttgttgctaatttatcttgcattaggtaattgattatttcaaaagagcattcttgaacatcttgtaaactaaaaaagcaaagaactcggtatgaggaagttaaaaactctgtcaagatttcaaaaatttgtgtataaaaattaattgccatgttagaagtcgaggtcaagcagtaatgatagaaattttgtgaaatgatgattagacataaaatgtaagacaaatgagagaaacaaaggtacacaatagaaaaagtattattagtactatattagtgactctgaatggcataa

SEQ ID NO: 72 - RNA, 3744 nt, synthetic construct
  source 1..3744: mol_type = other RNA
gtgcagaaggaaagggtaatcagattgatgtatcctctgagtcctccttgatatctgcagagcagctgagctggtctgctctctgtgctgttgacatctgctctttggggcgttagggggtctggtcatttgtcccttgtctgttatgatctacctgcagtggttttctaccttgtccccaggcttcttaattcttcctgtactctcagaacctctgtgttgtcacaaccagacttgggttattttgggtccccagtttccaggccctgaatgccatgggctctgtctcaggaataggtatctgctggcctgctgctgtcatttctactacattgctcctttactgccttaaagagtggtcctggatcaggggttgaaagtggaagtggaaataaaggacgctgatgtgacgtctgtgtttccattcttcttcctaccacttcaccagatgtgaaaaattcatataatctgaatcctctagtttgagttatgataagttaaagcagtttaagaatttaacgaactcccctctccctgattaaatctacttgttagcactgtgttcttactgcaaatcccgttgccttgccagaagataaatattaactttttctttctctttgccttcctgttgtaataattccaaaccacccaaaagaaataggtgagtttgtggagaaggtcagtaaaataatttcaaaagggaaaagaacattcatgcatttaaatattattcccactggttcatttatgctgcaatgaatatctatatatctatgtatgtatctatcattttgtgcacatgttttagtatttttctgagtttcatcctgaaaaaatgtaattgctgtaaggtatgcacattttacatttcactgtataatgacaaatcatgctacaaagtgactagaccaatttatactcttgctatgagtcactgagagaattctttactaacaccagatattttgcgtaacttattctgtttaaattctatcagcaaacctccatagtaggtgtattcccattttgtatatgaagaaactgaggttcggagttaaagcaacttgttcaaggtcacacaaatggtaagtgggtaatttcggaaccttttaaaataaaacatagaagcctactatcttattgttccattctagaaatatgtctgtgtttataatatcgactactttgtattattttaaactttataatattgactactattattttcttcacatcctagttaagaatctaggataaaatggaaaacgatatcacgaaatggaaaaacaatgttattccttagttagcttagtttaaacacctatttaatggtcaaaggcaagtagttgacctcccatctttttgactcacagtcattagttacacaagaaaagcagaacaaaacccagctggatggaggggtggatatgcaagtagctttcacactaggaattgtgaagcctgaggcatagccccaagactcagatcatcgggagtgcactcagtccagagggacaaatgttcagagtctgcatcacctaatattacaagcatctttattgagactgatagaagtgaatttctcaggtcagaaaacagaagagctcttttagctgagttgtgatatttaaaagtctctcattttgaatttcttgcctggccaatcaaactgtcctcccagaacaaattgttggataatgaaatgcatataatgtagcttgagagcgctgtcccatttgcagcactggcttcagcctgaggcacatctctggctgctcctttctatctataagttaaagcaatggcctcagcaagcagtacactctgtacagcagctgcaactcgttatcggctttgtccagaataacaaacagaacaaacacattttgcaagggcttttacccctcttatgtagagaaggaggaattaagcaatcagtgaaatgctgattgatcagttgtgctgactaagagaagtccaaggccacattagtactattaagccctaaaaaacaaagcaaaaagaaaatgttcttagaactttatttaaaccagtatttctgtgatgtgaagtacatttcccccaaagggaaaaatgggaaaatatttgcatttcacttagtacactttactcatccagactgctttgtaatgtaatgatggttaagtatttttacaaagataattcatgttttttggcagattttggttgatcatggagtatcagggttgtgtgaaccaaaggtgtgatgcagagcagctttagtaaaccatcccattcctatgccacccgtttgatttcagtaccaactgctgtggccacatctagaccactctttaccagaaactgtgccaccttcaaaattccagcatccttttcccctcctcccttattttttgatctcatatactcaagtattcttagtgtaacaattctttgagcattctttcttatctagcttaaaatctagggtctaccattataatcattcctttcaagataaccttaacttccacaccccaatttctaactggcctggcaaaactcagacctttggtgcaaaatcctccatctctatgcctgagcccttgtgtttgagaatggctgaagaaaaccatgtgcatggacattctgatgtcactataaattcatggtcacggatctcaaatgggagcccaagactactggctttcctgctgtggttgtctaatgacagcatgatcttactcttcacagatattagtttaaactctccaaccttcaattgaaaagtctacatcttactttattagaaagtagaaacaatcaagggttatgctcccattttcacactaccaagtaaacaaaccttcagtaagagaagatatatttcctatatctcctcctaccctcctgttaaaagggtgcaaacgtcccccctcctaacaaaaaccaatccttcttcatgctctttagatgccgtgttgtctctctttctcaaagtcttcattttttggttatactctttctctcatgcaaatttttctctctttttcaagtagagcaggctcagcagcctacaaacgtgtcttgggcacctttcatcaaaaaataaaaaacataaaaacaacaaacgaaacccttcttggctccacattctcatccagtttctattctttcacttagctgcttttcttctcatgttttgtctattttttaacatcctagtcactctctgatctacttcagtttgacttctacctccatcacttgagtaaattcaatacatggtttcaagtatattctagccctagcctgatccacagggaaggttctggaccacaagcaacatggcagagttgttcactcttgaggtaaaggagtttggcacctcatgcccctataatggattaaagaaacactccttaatcggggttagtccctgggaaaggttttaggtgtgagcaattagcaaccatcacccacagtggctgggagaggggggcactagcccaataaagaggatctaagtagtgcacccaagcaggtctaccacagttagcttcctatgtgggctcacgttgcatgcagtgcatctctatacagcaattaatctatttcataatattaataaaataatttcactttccatgttaaaatccttcaatgacttcctattgcactataaatggcaaatcaatatgttaacatgccctgccaacacctacagggtctggggtctgattgcctttcagatctctc

SEQ ID NO: 73 - RNA, 1948 nt, synthetic construct
  source 1..1948: mol_type = other RNA
gaaagaacaccgtagtataactggatggccctggtccctgccaggagtcaggatggactttcagtgttgaagagccatcttggcctgttactagcgctgccccacaccattgtgtttgcctaagagctgagccaaagaatttacatatgtatgagccaaggtctgaaaatagaagttaaaaaaatggtgctaaattgacatatttgctattttatttttttggtcagttaatatgataaacttttatttaaatgtaaacattattttaaaaagcactttgttttaaataactgattttcattgaaattaattctggcctctaagcagagttagctacaaagacaatttgtaaaaatgcagaaaattcagagtttaacttggagttgtttaaagtttattttcatatttgagtctgatatttcagttcactgtaatatttcaatttatttaaaatattatcaagacaaatattgggttaccttcacaatatttttttcatttttcttaccccatatattaaaaatgaacaaaaatgaaagaattatatagtctagtggatggcaattctcttggtacagtactcacaggatgtcagaagtgctttgatttgcatgcaaatagaaactgcattcctaaaagacccttaattgaagatattaataattttcattaggttttaagtcttttaaccttatcatatggatagaaaagtaaaagatgtgtttaataaaatgtcttttacttatttttatttgtcatttatttaaaggtttattagtgaatctgtttacctggaaaatgtgcaattctatttgcaaatttggagcactacatatatttaagagaatccttagtttctgttgggagtggcaatattttataactgggccttcatattcctatgttacagccctggtccctgaaagtcctacagctatctttgtttcaagtgtgtatgtgttataataagataagtgttaattagttaatcaagtactaattaacctttttaatttatagaaaaacagacatttatggacaagtaaatttttggggtcaaagtctttaataatttgaagaatgcagttatttttagcacaaatattctaatgtgaaaagaatttctttttctgagttatatatttttatgccattcagagtcactaatatagtactaataatactttttctattgtgtacctttgtttctctcatttgtcttacattttatgtctaatcatcatttcacaaaatttctatcattactgcttgacctcgacttctaacatggcaattaatttttatacacaaatttttgaaatcttgacagagtttttaacttcctcataccgagttctttgcttttttagtttacaagatgttcaagaatgctcttttgaaataatcaattacctaatgcaagataaattagcaacaacattacatatttttcttactatcaactgagaatgatcttgataggggcaagtataggttaaaaataaatgacatcaaaaaacaataccagaataaaacatttaattcttcaacactcaaaggcatgtcaggtattgaaatccaagtaggcacttttgactaattgttagtagtttgtagaattcaaggattagggttggcttccttttgtctttctttctcttcttcctccccttcttcatctccttcttgttcttcttttttttcccctctccagatcactaatcttctccaacaaatttttagcctatataaaataaaaaaaggtactcctgccctatgactcaacttacaaatctacaaacctggaggaagaaatagctctgtgatccattcctggggaattgcaataaagttttagggaagatggaacttaatgtttgtgaaagcacgggtcctatttccgatttccaggagggtggaatgtatcttcctgaatcacattccgctg

SEQ ID NO: 74 - RNA, 1858 nt, synthetic construct
  source 1..1858: mol_type = other RNA
cattccgctgaaaataggaggccatggaagaaatcaggttcagtataacaaattttctacaaaagggggtggagtaggaatgtgcgtaggtgacctgttctctacgtcattccagtgtaaactactgccccttacacttcacgaagtcggtaacttcaagacatgttgcaccacatgcttctctgtgatcctcaaatatgtttaatttaaagagggtccagtagtgtcctggcacatgatctggattgccatagataaccatctacctcacagctagggttgctctttagaatcttgcaaaatcatttgtttactcttgacaaaagttaagaaaacaagcccatcagagttgtttgttctgtcagcatgttagaagatggttttgttgcaatgataatcgttgtgcaaagaagactgatgatgatttttttttacattttcttaacagtatttgctatttagaatgaatgttgtctaattatttagccattttattttgtaaaatttatgttgtaggcatatttagaccaagttataagaaaatgcttcagccaaaattaagtgttgagtttgatttgtgtaattgttagtttctttactagttgttccatcatttacacaattatttctatttgaaatgcagtaattgttcagaacttatatttctatactgatgtctactaacagctttagatcaaatattaaataactcaagaataatgagatgatcttggcttacttagatatttggtttttatatctatagaacaaaggaattagaaaataattttgaagatttcatccagctatgtaaaactatctagggaatacatttactaggttttcaattttctacaaaacatctttcagcagaaagcaatcctgtttcctgatatacaatgtctgatacatagaaactactcagtacataattcctgaattgattattcttttggaaatcctagatttgatttctgaacaatcataaacatttaatggcatgaaattacccagattccatggttctggaatacataacttcaagcaataagatgcaagatagaaacatataagacattctttgctattttaggtaagtccagctgaatcagttaatcagctagaaacgtggctcacagatgaattagttttattattaggtggattactgaaaaattaatagctttatttccgtattaccttatcatttatttaatataaaacataataaaccagagaagttgtggttactttcttctgtctcattgttcagtttttactgaagttatgttttactgattttcacgaaatgcaaatatttctgagcatcagaaatccagcttttgctacctcccactcatgtattcttcccatcaaattgaatcttaaccaggtcactctttcctgttcattccatgctgttgcccctgttttctccagatataattgacatgctccaagaggatgaacattgcctaagacttgatttctggtcttggctttgcaacttactagctgtgtgaccttgaacaagctacttaattctctgagactcatcattattgtttataaaatagggatagcattagcttatttcaagaattgtttttaaaattagcaaggaaggcataccaatgcctagctcaggggatcaacaagtggcaagtacctcaactttcaggcaagatcatacttatcatcagtggagccaatagaacagatttatttaaataccctattttcacttactggcataacagcaagtggtgtacatatttatatatctctctatatagatgcagataatatactaatatattgtggttatatcttccaatgttttagtttctattgaggg

SEQ ID NO: 75 - RNA, 2000 nt, synthetic construct
  source 1..2000: mol_type = other RNA
tcatataagctgtctgcacgcagctgagcagagtgacaggcatgaatgcaggcacacacatcaggctggggaccctgtgtacttggaagtacattctttgtgagccaggtcatcaaaggttttctaaacttatgagtcactctctgataaaatgaatttttttttttagatttagtcttcttttctattaatacattttaaaaaatagaagcatttagcctgtgaaatccctcagggcactttattttatgaattccttattgacatatctggtattgaaaatgttaaaatgtcaagtactaggttgtattcactttcatttgatatttaaagaaatgtggaaatacagaataatagataataacccaccacacaaacataatctctgttaacactaaagtattatcttctagcatttatgtcaatgctcagaacatagttttgaaaattgccattgacttgcataatgctttttggaggcaatattatgttaaatgtattttgatgttattacaaagttcttttaaaacattactttaaatgattacaaaatattccattttcattgctgctacaatgcatgtcaatattgccatatttgattactatttacaaatttctctccaaagagagtgtactcacctacataccagtgcaatatatgggtgtccctttaaaaatgtcctcaccaacattatgtattatctttcattttatttgctccgctgtagaaaatattattttgctttcatttctattttaaatcactggtgaagttgaatacttcacagtatgtttttaattgatactattcttttatcaactactgtttacatttttgtttccattaactcatttatattaatcattacgaaaatgtctttaaaggaaacataaagaaacactgacagcctgtgtctatactgatttaaacagtccacttctcaaacagtccactcttcatttaaacagtccactgacataagcaagggaagacctgctacagattgaaaaaaaaaatgagaacaggatgtcttagagaataaaggtcttaggttttggaatgaagagaaaataagggtgttagtaattcctttgaaggtacagagacttccaactgccctcttgaaatgttcctaagtaatgaatcagaaacaactctaagaagttggtgcatctatcaaaaagtctctaacatagcaagcatgcaagagatgcttctggaaaaatgactgaatgaatggattgctgtgtgcttaattcctgcctgatcagtgatgattatttaagcaggaacaaataaaattacatcagttaagcatgagaggaaataaggatgcttaggagtcatgaaaaaaaaaacctggcaatcaataattctataaagtctatttttgaagagatacattaaagcaaggagtaaggtatgactggagagcaaacaaattcagagtaaaaggaggtgcaataactccctgaagaaagttctactgttttgtgtttgaccagaaatgtgtgaaaatagaatataaaaggtagaagtaattgggttcagttctatgatttttattagccttagtatcataacaaggaacacggtcacaattaaagatatggatgaagaaggcatctctgagggaaagtggtgtggattgtacccatgtaaaaggcacacattttctcctatcacagaaactaggacaagctggtaggaggattcctcaaagtttggccaggaagaagacttaaagttgtttgaaaaccttcccaatatgtcaaggattgaaaatgacagacagatatcatgttgttagatgatgagggttcaaactaaccagatatccttggaaatccaatcaagtaaaacctattcattaagatccaattggcattttgaggaatagaaagtgaggatcaaattatgatgaatatgcttatcaatctaacatttgataacataaagtttggagtgatacaaaagaaggtgaaaaatgaagctg

SEQ ID NO: 76 - RNA, 2000 nt, synthetic construct; identical to SEQ ID NO: 49
  source 1..2000: mol_type = other RNA

SEQ ID NO: 77 - RNA, 3864 nt, synthetic construct
  source 1..3864: mol_type = other RNA
tcttcctatgggaaaaaataatttcctctatcagaggaaaaatttattactattatgataataccccttcagacctttttctagtcacttacaggtaaaaattgtagtactggttggtacatagtgtattttattaatgttttactgcgtatattgttgtataaattgctttccgcactcattggcatgccttagaattctttttatttaggcttcatgactaatttgtagtggaacctcaagaagatcacttatgtgcttcactttccacatatgtaaaatgggaataacaacagaatctgcctcatagtactgctgaagggtgacatggggaacacctgtgaggcattcagaacagagcctgtcatacaataaatgccatgtcagtgtttgctcttattctcagtatacagagatctgctttgatcttttcatcttctgcaaaatattccacaaattggcatattgtgttttgtaataatctctattgattattttcaagcaagatttggaattatccgcagtagtttcaaatgtacaaaaaagtaccttattttatttattttttaaataactgatctgatctacgagtctaatctggtaagttttgtcttgagtccagtaatgcagtccaatactccatttaggtaaccctgtccttgcttggtcaatttgttccacttttccaagtttgacctgcttaccctattcctcttcaagagggcatctggttgtttagtttacatgatttagtggtgcagaaggaaagggtaatcagattgatgtatcctctgagtcctccttgatatctgcagagcagctgagctggtctgctctctgtgctgttgacatctgctctttggggcgttagggggtctggtcatttgtcccttgtctgttatgatctacctgcagtggttttctaccttgtccccaggcttcttaattcttcctgtactctcagaacctctgtgttgtcacaaccagacttgggttattttgggtccccagtttccaggccctgaatgccatgggctctgtctcaggaataggtatctgctggcctgctgctgtcatttctactacattgctcctttactgccttaaagagtggtcctggatcaggggttgaaagtggaagtggaaataaaggacgctgatgtgacgtctgtgtttccattcttcttcctaccacttcaccagatgtgaaaaattcatataatctgaatcctctagtttgagttatgataagttaaagcagtttaagaatttaacgaactcccctctccctgattaaatctacttgttagcactgtgttcttactgcaaatcccgttgccttgccagaagataaatattaactttttctttctctttgccttcctgttgtaataattccaaaccacccaaaagaaataggtgagtttgtggagaaggtcagtaaaataatttcaaaagggaaaagaacattcatgcatttaaatattattcccactggttcatttatgctgcaatgaatatctatatatctatgtatgtatctatcattttgtgcacatgttttagtatttttctgagtttcatcctgaaaaaatgtaattgctgtaaggtatgcacattttacatttcactgtataatgacaaatcatgctacaaagtgactagaccaatttatactcttgctatgagtcactgagagaattctttactaacaccagatattttgcgtaacttattctgtttaaattctatcagcaaacctccatagtaggtgtattcccattttgtatatgaagaaactgaggttcggagttaaagcaacttgttcaaggtcacacaaatggtaagtgggtaatttcggaaccttttaaaataaaacatagaagcctactatcttattgttccattctagaaatatgtctgtgtttataatatcgactactttgtattattttaaactttataatattgactactattattttcttcacatcctagttaagaatctaggataaaatggaaaacgatatcacgaaatggaaaaacaatgttattccttagttagcttagtttaaacacctatttaatggtcaaaggcaagtagttgacctcccatctttttgactcacagtcattagttacacaagaaaagcagaacaaaacccagctggatggaggggtggatatgcaagtagctttcacactaggaattgtgaagcctgaggcatagccccaagactcagatcatcgggagtgcactcagtccagagggacaaatgttcagagtctgcatcacctaatattacaagcatctttattgagactgatagaagtgaatttctcaggtcagaaaacagaagagctcttttagctgagttgtgatatttaaaagtctctcattttgaatttcttgcctggccaatcaaactgtcctcccagaacaaattgttggataatgaaatgcatataatgtagcttgagagcgctgtcccatttgcagcactggcttcagcctgaggcacatctctggctgctcctttctatctataagttaaagcaatggcctcagcaagcagtacactctgtacagcagctgcaactcgttatcggctttgtccagaataacaaacagaacaaacacattttgcaagggcttttacccctcttatgtagagaaggaggaattaagcaatcagtgaaatgctgattgatcagttgtgctgactaagagaagtccaaggccacattagtactattaagccctaaaaaacaaagcaaaaagaaaatgttcttagaactttatttaaaccagtatttctgtgatgtgaagtacatttcccccaaagggaaaaatgggaaaatatttgcatttcacttagtacactttactcatccagactgctttgtaatgtaatgatggttaagtatttttacaaagataattcatgttttttggcagattttggttgatcatggagtatcagggttgtgtgaaccaaaggtgtgatgcagagcagctttagtaaaccatcccattcctatgccacccgtttgatttcagtaccaactgctgtggccacatctagaccactctttaccagaaactgtgccaccttcaaaattccagcatccttttcccctcctcccttattttttgatctcatatactcaagtattcttagtgtaacaattctttgagcattctttcttatctagcttaaaatctagggtctaccattataatcattcctttcaagataaccttaacttccacaccccaatttctaactggcctggcaaaactcagacctttggtgcaaaatcctccatctctatgcctgagcccttgtgtttgagaatggctgaagaaaaccatgtgcatggacattctgatgtcactataaattcatggtcacggatctcaaatgggagcccaagactactggctttcctgctgtggttgtctaatgacagcatgatcttactcttcacagatattagtttaaactctccaaccttcaattgaaaagtctacatcttactttattagaaagtagaaacaatcaagggttatgctcccattttcacactaccaagtaaacaaaccttcagtaagagaagatatatttcctatatctcctcctaccctcctgttaaaagggtgcaaacgtcccccctcctaacaaaaaccaatccttcttcatgctctttagatgccgtgttgtctctctttctcaaagtcttcattttttggttatactctttctctcatgcaaatttttctctctttttcaagtagagcaggctcagcagcctacaaacgtgtcttgggcacctttcatcaaaaaataaaaaacataaaaa

SEQ ID NO: 78 - RNA, 2963 nt, synthetic construct
  source 1..2963: mol_type = other RNA
tgtttgttctgtttgttattctggacaaagccgataacgagttgcagctgctgtacagagtgtactgcttgctgaggccattgctttaacttatagatagaaaggagcagccagagatgtgcctcaggctgaagccagtgctgcaaatgggacagcgctctcaagctacattatatgcatttcattatccaacaatttgttctgggaggacagtttgattggccaggcaagaaattcaaaatgagagacttttaaatatcacaactcagctaaaagagctcttctgttttctgacctgagaaattcacttctatcagtctcaataaagatgcttgtaatattaggtgatgcagactctgaacatttgtccctctggactgagtgcactcccgatgatctgagtcttggggctatgcctcaggcttcacaattcctagtgtgaaagctacttgcatatccacccctccatccagctgggttttgttctgcttttcttgtgtaactaatgactgtgagtcaaaaagatgggaggtcaactacttgcctttgaccattaaataggtgtttaaactaagctaactaaggaataacattgtttttccatttcgtgatatcgttttccattttatcctagattcttaactaggatgtgaagaaaataatagtagtcaatattataaagtttaaaataatacaaagtagtcgatattataaacacagacatatttctagaatggaacaataagatagtaggcttctatgttttattttaaaaggttccgaaattacccacttaccatttgtgtgaccttgaacaagttgctttaactccgaacctcagtttcttcatatacaaaatgggaatacacctactatggaggtttgctgatagaatttaaacagaataagttacgcaaaatatctggtgttagtaaagaattctctcagtgactcatagcaagagtataaattggtctagtcactttgtagcatgatttgtcattatacagtgaaatgtaaaatgtgcataccttacagcaattacattttttcaggatgaaactcagaaaaatactaaaacatgtgcacaaaatgatagatacatacatagatatatagatattcattgcagcataaatgaaccagtgggaataatatttaaatgcatgaatgttcttttcccttttgaaattattttactgaccttctccacaaactcacctatttcttttgggtggtttggaattattacaacaggaaggcaaagagaaagaaaaagttaatatttatcttctggcaaggcaacgggatttgcagtaagaacacagtgctaacaagtagatttaatcagggagaggggagttcgttaaattcttaaactgctttaacttatcataactcaaactagaggattcagattatatgaatttttcacatctggtgaagtggtaggaagaagaatggaaacacagacgtcacatcagcgtcctttatttccacttccactttcaacccctgatccaggaccactctttaaggcagtaaaggagcaatgtagtagaaatgacagcagcaggccagcagatacctattcctgagacagagcccatggcattcagggcctggaaactggggacccaaaataacccaagtctggttgtgacaacacagaggttctgagagtacaggaagaattaagaagcctggggacaaggtagaaaaccactgcaggtagatcataacagacaagggacaaatgaccagaccccctaacgccccaaagagcagatgtcaacagcacagagagcagaccagctcagctgctctgcagatatcaaggaggactcagaggatacatcaatctgattaccctttccttctgcaccactaaatcatgtaaactaaacaaccagatgccctcttgaagaggaatagggtaagcaggtcaaacttggaaaagtggaacaaattgaccaagcaaggacagggttacctaaatggagtattggactgcattactggactcaagacaaaacttaccagattagactcgtagatcagatcagttatttaaaaaataaataaaataaggtacttttttgtacatttgaaactactgcggataattccaaatcttgcttgaaaataatcaatagagattattacaaaacacaatatgccaatttgtggaatattttgcagaagatgaaaagatcaaagcagatctctgtatactgagaataagagcaaacactgacatggcatttattgtatgacaggctctgttctgaatgcctcacaggtgttccccatgtcacccttcagcagtactatgaggcagattctgttgttattcccattttacatatgtggaaagtgaagcacataagtgatcttcttgaggttccactacaaattagtcatgaagcctaaataaaaagaattctaaggcatgccaatgagtgcggaaagcaatttatacaacaatatacgcagtaaaacattaataaaatacactatgtaccaaccagtactacaatttttacctgtaagtgactagaaaaaggtctgaaggggtattatcataatagtaataaatttttcctctgatagaggaaattattttttcccataggaagatcttaaacttatttgtaatgtttcaattttttcacatgatgaacaaagcaatacactgctcatgttactgaaactcgataaaatatatgaagctaaaattggtcatctaaaagtatgataatatataatatttattttctactttatctccaatatgcttatcatacagaaattataacaaaatgcaagcaaatgttttggataatttgaaagttaataagttgtacactgtagccctaccatccccactgatgtcaaagggctgatttttaattatgcaatagtaattaaatagggatcaatgtcattacctgagaaaacac

SEQ ID NO: 79 - RNA, 5263 nt, synthetic construct
  source 1..5263: mol_type = other RNA
aattctaagtcaggtggtccaacaaggcctctcatcctattggtattacagtatgacctgtctcctgaactttgaagccatttctttccttcagaggagataagaaaatgtgtcttatcttaagtctggaagaaacacggagcccagtatcaagggagtggagtagttcttttcccagacgtagcacagggtttttcaagcctgaagaaaaagattaatctccttaaaacaaggctttgttgacttgggtctggtcatggcaatcctgtttccagccatcctgtgtataagctctaacggtcagaagtttgtttgctttgattttgatgtggctcatcttttcttagctgctcatattcaagatttggaaaagaaggaacggtggtgagctgagcaattaattggattctgataaggagttctggagaaattttattattcctgagttttaaacccgtgtaactttaatataactttggtatgagcctttacattttttctctgtgtttttgtaattatattgtggcattcttattgggtgtggtagtagcaataaattataaatgtatgaatttatacattatggatttataatttcataagtttttgagattttaagacttactattttttacacgatgccttatgtatttatagcatttaatattaatctaaattcattttgagatatattttatcatgcttttatcttttatatgttattagaaagcacttggtattttaaaaatatatattaatgtgagatttcacaatttataccttgtctttgtagtcacatgcgtagaggatatttacaattttactcaagttaatttgctcagtgttgatcatcagaccttttagccagtattggctgggaaaaaaagtgtaattctttttgttctcttgagaattttttggatctaaagtgattcctagggtgtgggtcaacttctatgatatgaaagatgacatggaggttttttattttgtgaagtgcattaataggtgatcacatttcatcctgggagagctgctggagaggaatattgatttagcctgttatggttgagtctttggggtcttgagcttcttagatttttagttctttttgttattctttattgttcaatcttgagagtagaaaatacacatttttgtccacttctaagaataaaactgaaaggcttctcaagagggagcttttgagggacaagtctgtgttccagacttatttttattctcggatctagcaccatgcttggcatgtaaaagggctttaataagtatttatagaaggaattgaagaattaattattacttatggactatgagaggtcatggttgttactctttctaatttgtcgatattaaacagagacagtgaagatactaatttagatcacacaacgtgaatgaagcaaacaaaagcctaaagctttagaaaaatgaatatattatatttttaacttcgcactgtagttaagcagtgtggagctatttaatgatttcaaccaggaagatattaatgtgtgaaataagagtacgtggacagtatattaattgtatgatacaaacaagatatacatacataaagttcattaaaagagtatgatttcatatggctttgaaggattgataggatatagacaaattgataaaattgatctaaccaatatttaactttaggtttcttcatcagataaaatattgcatcattttatgttggctaagtgatttgcagtctatttcaattaattattctttatagtttttctctgtcaatgccatttaataaagagaaaacttcagcagacagaagttatatgatctttggcactattggctgtgtgttaagccacagtaaggttcagttacgtattttattgttttataatcacacatttctcctttgattcatttcttatctacaccccttctttcacatcctaaatattttctttggataaatgtgtagggcaagatgaagaaaagaagagaaaaaagtagtcagagcttgggagtactgaaacgctttggactccagccagctgccatcactgaggtggtgaaagaacaccgtagtataactggatggccctggtccctgccaggagtcaggatggactttcagtgttgaagagccatcttggcctgttactagcgctgccccacaccattgtgtttgcctaagagctgagccaaagaatttacatatgtatgagccaaggtctgaaaatagaagttaaaaaaatggtgctaaattgacatatttgctattttatttttttggtcagttaatatgataaacttttatttaaatgtaaacattattttaaaaagcactttgttttaaataactgattttcattgaaattaattctggcctctaagcagagttagctacaaagacaatttgtaaaaatgcagaaaattcagagtttaacttggagttgtttaaagtttattttcatatttgagtctgatatttcagttcactgtaatatttcaatttatttaaaatattatcaagacaaatattgggttaccttcacaatatttttttcatttttcttaccccatatattaaaaatgaacaaaaatgaaagaattatatagtctagtggatggcaattctcttggtacagtactcacaggatgtcagaagtgctttgatttgcatgcaaatagaaactgcattcctaaaagacccttaattgaagatattaataattttcattaggttttaagtcttttaaccttatcatatggatagaaaagtaaaagatgtgtttaataaaatgtcttttacttatttttatttgtcatttatttaaaggtttattagtgaatctgtttacctggaaaatgtgcaattctatttgcaaatttggagcactacatatatttaagagaatccttagtttctgttgggagtggcaatattttataactgggccttcatattcctatgttacagccctggtccctgaaagtcctacagctatctttgtttcaagtgtgtatgtgttataataagataagtgttaattagttaatcaagtactaattaacctttttaatttatagaaaaacagacatttatggacaagtaaatttttggggtcaaagtctttaataatttgaagaatgcagttatttttagcacaaatattctaatgtgaaaagaatttctttttctgagttatatatttttatgccattcagagtcactaatatagtactaataatactttttctattgtgtacctttgtttctctcatttgtcttacattttatgtctaatcatcatttcacaaaatttctatcattactgcttgacctcgacttctaacatggcaattaatttttatacacaaatttttgaaatcttgacagagtttttaacttcctcataccgagttctttgcttttttagtttacaagatgttcaagaatgctcttttgaaataatcaattacctaatgcaagataaattagcaacaacattacatatttttcttactatcaactgagaatgatcttgataggggcaagtataggttaaaaataaatgacatcaaaaaacaataccagaataaaacatttaattcttcaacactcaaaggcatgtcaggtattgaaatccaagtaggcacttttgactaattgttagtagtttgtagaattcaaggattagggttggcttccttttgtctttctttctcttcttcctccccttcttcatctccttcttgttcttcttttttttcccctctccagatcactaatcttctccaacaaatttttagcctatataaaataaaaaaaggtactcctgccctatgactcaacttacaaatctacaaacctggaggaagaaatagctctgtgatccattcctggggaattgcaataaagttttagggaagatggaacttaatgtttgtgaaagcacgggtcctatttccgatttccaggagggtggaatgtatcttcctgaatcacattccgctgaaaataggaggccatggaagaaatcaggttcagtataacaaattttctacaaaagggggtggagtaggaatgtgcgtaggtgacctgttctctacgtcattccagtgtaaactactgccccttacacttcacgaagtcggtaacttcaagacatgttgcaccacatgcttctctgtgatcctcaaatatgtttaatttaaagagggtccagtagtgtcctggcacatgatctggattgccatagataaccatctacctcacagctagggttgctctttagaatcttgcaaaatcatttgtttactcttgacaaaagttaagaaaacaagcccatcagagttgtttgttctgtcagcatgttagaagatggttttgttgcaatgataatcgttgtgcaaagaagactgatgatgatttttttttacattttcttaacagtatttgctatttagaatgaatgttgtctaattatttagccattttattttgtaaaatttatgttgtaggcatatttagaccaagttataagaaaatgcttcagccaaaattaagtgttgagtttgatttgtgtaattgttagtttctttactagttgttccatcatttacacaattatttctatttgaaatgcagtaattgttcagaacttatatttctatactgatgtctactaacagctttagatcaaatattaaataactcaagaataatgagatgatcttggcttacttagatatttggtttttatatctatagaacaaaggaattagaaaataattttgaagatttcatccagctatgtaaaactatctagggaatacatttactaggttttcaattttctacaaaacatctttcagcagaaagcaatcctgtttcctgatatacaatgtctgatacatagaaactactcagtacataattcctgaattgattattcttttggaaatcctagatttgatttctgaacaatcataaacatttaatggcatgaaattacccagattccatggttctggaatacataacttcaagcaataagatgcaagatagaaacatataagacattctttgctattttaggtaagtccagctgaatcagttaatcagctagaaacgtggctcacagatgaattagttttattattaggtggattactgaaaaattaatagctttatttccgtattaccttatcatttatttaatataaaacataataaaccagagaagttgtggttactttc

SEQ ID NO: 80 - RNA, 1413 nt, synthetic construct
  source 1..1413: mol_type = other RNA
cactgctggttccacctccttctctcaccatggggagaaaaaggtgacctcttctggcattgcaagcacaacctgactcacagggcaatgcttttgtatctgagtgtctttcacaatccatttactatctgtaactagctgttctgttactaacctttaaaaactgctcccatgcccttcaatgacaattatgtaaatatactctaatgacttttaactatctttaaaatactgtgcccacatcaaaaagaattaatttggatgtgtatttcttagaaaattgagttttaaaagagaaggcaaaaaaaaaatcatacataaaggactatttcaaaataagtgactaagaacacaggtcccccttggactctgtgggactttaattcactaccatgaagcttaagaacttccatttttcatgcctggcattcctccaaagttctggccagtaatgactgtatgtgagtaatggcttttgcaccttgaaatgatggcttattcctcctgaaaatttgtcattcttagctatgaactatggaagacccttgaaacagatctaccaccatgagctagaactgctttcccaggggtcggtccacataattagctcatgttgttgtgtggtaattgctgtgtagagtgcttgctgagtctagggatctgatttataattattgtggcctccactattcattgctctgtttttggtatttaacagagacctgctcatccttagctacccacttctgtgagaataaagtggtccaggtgacacagttcagaaatatgacaaagcacttaaactgtagatggtactgttgtccttacagtttcttctcttcaagatggcatgtgatgtcttcaaggcattgaaacacttaactaaagctaaaccaccattctctgtatatcaaggtactatgtccttattgatgctttagaatatccattctcctttctggcttccatcttattatcaaaatgaatccaacatggctttcttaggctttgtaataaattctcagggtagcaggctcaagttggtatcatgtcattgatgtaagcataagtgtctctctccctttccctcccactcctcctccccttccccatcctcctcttcctctccctaccctccatgccaccatcctttccttccaccctctttttctctcccaccagtcttcttatgtctctctcctgtccctggaatctataagaacctatagaaattgcctgttcccacacatgccagaatccaaagcccctgaaatcctgaccaaggcattataagcaaaagaatctcttttggtatagcattggaaatgtaaatgagctaaatacctaataaaaaatggaaaaaaaaaaagagtcaaatgtcaaaaaaaaaaaaaaaggaatctaaggagcacc

SEQ ID NO: 81 - RNA, 1777 nt, synthetic construct
  source 1..1777: mol_type = other RNA
acttcttttgcttcataatcatgttacaagaggcccttgacctgaggtaccctccactaaatacacatttgatttgcacagtggggtcattggctctggcaaacagcagtgtcaacattgctaatctgaaacaacaatctctcatgcccctctctgccccaccttcaaacacacccacagcaagttctgacaaacactttatatgtgccgaactgagcattctttatcacttgagtagacatgacaaccagctaaccttgcaggaacgtccctctctcctggcttctttctagtgagaatggaatccagctcaccttatcctaaaatacttggtgtagaacatgaaattgttttggagttttcttcatatatatttagatctagatagagcccatgcttatcttaagttaacgaaagagaaattcaaattaaaacaaaaaaaaaaacaaacaaacaaacaattgctcaatccctcacacttgttagctctgggcctggtctttaaccccggttttccatttctactttagttcttttctcagtcagcagggtttgctaagattcatgcttgctttacattttctacaaaagcccagagactgaaaccatgtgttttaagtggttttaactcaaaatgttcttttttgcttgttgagaactcataacccaacttgatagaatgttctttctaattataaagtattgtaatgaggtccctgtatccaagaagcaccatttaatgtccgctctgatctctgagggttttatttttacatcaatccagttctgaccacccagacatgaaagtgccttggtcactgagccagcagcaccctccagttggcaaaagcaaggtcatgagaaatggtgacaaatttcagagcttcttactcctcatttggattcaaagtccctaaaatggaaatataattcttttaccacttcttgaactactctatcaggcagggttcaaaaaataaaatccactttgagagtttctttaaatagtaacacccttaataatatcaaaacatgatttgcataattgcactctattaactaatatgagtgtatttcttgtgaatagttatgttgaaaagacgttcttggaagcatcaaaaatattaactaaaatatttaaagaatgatttaagctccatcaaaattttaaaatgtttactcaaaagatactgttaagaaagtgaaaaataaggcatagacttaaaagcaaatattcacaaatgatattattggtaaaaaaaaaaaaaattgtatccaggccagatcaagagcttttagaagtagatagcaagatgaatgagccaacttaaaataagcagaataggagctggaaaatgctcagtgggaaaatgcttgctatgcaaacatgaggatcagatctcagatacccaacacccctatagctcacagccaacaattggactgagctccaggggacctggatggaggagatggagaagggattgaaggagctaggggtttgcagccccatggagggagcaagaagtgtcaacaggccagataccctagagcttccagggactggatcaaccaccaaagaatacacatggagcagcccatggcgctggccacatatgtggcagaagatggcctggttgaacatcagtgagaggagaggcccttgggcctgaaggtgttcaatgtcccagtgtaggagaatgccagggagggagaatgggagtgagggagtcggggagcaccctcatagaggcatgggagggggaatgggataggg

SEQ ID NO: 82 - RNA, 2348 nt, synthetic construct
  source 1..2348: mol_type = other RNA
agttttggcacaccattttaatgagacagcagatgcaaaagtgcttacaacagctgccctaagtagacattttatcatcctccctcacagcagccattctgtgccttcttctgcatggctatttagcaattgtaagtctctagaaccattcaatggtattctgtacagttaatgtttttaattatattctgcttcagactttgattcttttgtaagctttttctttacataaaatttaccaactgcttcttttccttcttggacttacacatgtctatcataaccacactgactttatttggagacaaagtaataaggtaactaaactttacttactcattatagtgttaaggaatcaacctcatgtctcacacagactaggtaactgttctgtcactgagctgattcctagcctttctaatgttctatgagtgattattataatctacgaaagactaagggcacagtaaggaacaacacaaaattttctgcctccactgaacttgtattgtaaatgttataatgaaatgtagggaccatgaatgaataatggatatggtatctccaaatcttatgcatcgtcattttctaattcctgtatcctgatattttgtgggaaacttgctaccctgtttgactgatgtcttggtgggacagtctttgatcttactccaatctcgttctctttgccaacttgtagacacctgatcatgcttgtccaatcagactctcaggaaaactgaatataattggactatgttcaactcaaagatggctctttgacaaaggccattgactcctgccacctagtcctcagggactcatttgtccttatttgttccaaaggcctctttccttggctactccttgtatgcaaaatcttcactgtttcttgcaagttattttaagtgtaattttgtgttggctggaatccagttattctaatacattaaatttggcaaaggaaagagcatagcatttctttatttctccaagtcaactgtcttatccttttatccactgggcttgtaagaaattaaattgaaactttagtggggattcccctctatgattccgtggctgagaaccagtagccagtttctgaggtgtccactgtggccgcctttgttctatgccagggataagaagatcgttagagagactggttatcagggagctaaggttcgcttctacaggtgcaacgtgcaaagcctactgtatgtaagtctgtattatgcctgggagactttgtaccatcgggctgattactctgatgcaaagcctccctcagacagttaacttatcttaaagcctctgttatcgttcatgaacagagggcaaactcatgtccattctgtcttgttctcccacctgtctcttaagttgctctggattgctacattaaactctgaggaacaatgaacccattcttccatttagtatgaatttagtaagtgtctgatccattccaaattctctatgagactctgaaaagacaaagggaaactccatttagttattgttctacaggagtacattttgggttggagaaagggaagattaagtaagctagtggctaagcaacagggcagtgatgaactcgacaagaaccatgacaacataacaatagaaaagatctgagtctatttggagaatgtcatgtcagacatggactacaaaaggagagaacggctgggttggacctttgggaggatgtgggaatttgcttactatacaggtgaccagtaatggcacataccaaatcatagagggaggaggaaagaaaggaaggaaaagagcaggttataaagaaggaggaagaaacaacatgggaaaaaaacagggtcagagaagaggcaagaataagaacaagagagaaggaaggagggagaggaaaaggaaaaggaagaaagaaaagaaaaaaaaaacagaagaaggctggggtggggggggttatgttgtcctgtaacaagtagcacactatagctgacaggattagggtgaagtccactaagaggcttgggtgagacagacataggaaacaggtagacagaaaaacccagaaggaacattgtcatcttgctctcctgtgtgaaaacggaagctttgcctctgtgaatccaagaaagagacgtacctctgttcttatctacacaaaagagaaggtggcagaaacctcatcttactgtcactgagaggactaaatggacttttgaatcaaaagcacttagcacagtacctaccacacagcagaaatcaagggcttttacctgtcctccttcttccaaacctcacaccctgaaaatccatcacaaaccctgtggaaccataaaaagatatccacccct

SEQ ID NO: 83 - RNA, 5638 nt, synthetic construct
  source 1..5638: mol_type = other RNA
cctctatctccactctcattaatatgggtgtcctattgctcttattttcttattaaaagaaaagggggacacagacacacacataaatatatacaaacacatgcacacacacatacatatatacacacatgtacatacactccaagatgttttgatgaaaaaggcagttataaattagaatctggatgagaatcaagccaggcaggttttatttcttgctatctgttggtttggttttacttgggtttggagactagggctgtgttttgagatgggagacaatgaacaaatatctggatatttacctggaaagtggagttgtaaatttttatgatagaaagggtataaccaaggagtaaagactcaaagagtggccaaaggggggatatgaagaagatggaaagtaagtcatttctgatctgccagacatacttccactagtataactggaaggcagaaggactagacaacctttccttcctgcaagttgataaattcagtagtgtaaagagtagagcacagccatctcatgacttccatttttcagtgatgtggggtgcagtttttcactgaaggttgcaaggatgcaaaccctttaaaggatttgacctgtgataggctgcccatgacccagtagatgccctgtacctatgctcatactggcagcactaagtaaactcagtgagtttaaaaaacaaaacgaaatataaagctttgggagggaggggatggtggtgggcaggaaggaggagttggagaggggagaatggaaggagactagatcaaaacatgttatgtgcctgaatgaattctcaaacagtaacaaaagaaaactccttaggagtaaactagatcaaaacatgttatgtgcattcatgaattcccaaacagtaacaaaagaaaagtctcaggaaactagggccatgttctccttacagtaagatcctcagtagtatcagattttcacccgcgatagcaatggtgaattcatatgacatcagaatgtacatgaatatggctttctccagctattttcaaaaacaagtgtgcagcctagcaggtggcatattactcaccgagagttcaagcattacctagcaatataggaagaaatggggtgtgaaatatgagactctagaaagaaataactacaatagtagaccctaaattctaagacaaatgaaaagattgctcaaaagtgaggagatgtctcagtcagtggtggcacatacctttaatcccagcaccctagaggcaggggaatctctgagttcgaggacagacaagtaagtctccagagcaagttctgggacagccaaggctacacagagaatccctgacacatgcatgtgcacatatgtacctacgtgtacacaccacacacacacacacacacacacacacacacacacaaaaatcacagctctaaaaactactgaggcagattctgaaagatccatacagtaaatatatgaattcaaaaaataggaaaaaagatgagaaaactaagacatttggattttaaaggtggctctgtttctgataaagatcatctgggagttggggggggcataattaagctgttacgctgacaatcttttttcatttcacacaattctacctctctgtggtcaaccaacacagtccaaatactatgaatcctgtttataaggcataaccaccatcaatatgagaaaatcataataaacaaagcaaatttctttcctttctttaggcaaaattgaacatcacagaaatattagtataaataaagttctaagagtaatctttgttgagattttcttttttcattttacagggttaatagcactcatgtggccttgaacctctctatgccagtacaattgatcaatcagcatttcaccgattgcttcactcctccttctccccataagaggagagaaacccttgcaaaacgtgtttgttctgtttgttattctggataaagccgataacgagttgcagctgctgaacagggtgtactgcttgccgaggccacggctgtaacttatagatagaacagagcagccaaagatgtgcctcgggctggagccagctctgcaaataggccacactctgggctatgatacatgcatttcattagctgacaatttactcctggagaagcttgattggcgacagactcaaaaagagagaatttgaaatatcactcctcagctcaaagtgtttttctctgtttctgacctgaaggattggcttatgtcagtctcaatcaagatgcctttagtagtacttgaggctgacttggaacaatatgggtccctctggacagagtgtacacctgatgattcgtgtcttggggcaatgcctgaggccgcacagtccctagtgctgaaactgagctcacgcccaactcaactgagttttgtgcctcttccctgtgtacttaagggctgtgggccaaaaagacatgcagccaacaatctgccctgaaccattaaacagctctctaaaccgagctgatggggaacagagtatcattgtccattatttataatgtgatcttctacttttatcctcaattattaagacacaaggaaaacaagaacagtcaacattataaggattgggtcaaaatgaatacatgctttgagcataaaccagaatcaagataactcttgtgcttgcttgaaaaaagttttcaagccttcatcctttagcaagttgctctgactcctcagacctctgtttgttcacaggtagaatgggaatgatcctcacagctgtggagggttatccagagacttctgataaagtaagttatatgaaatgtctaccattagcaaggaatactctaaagttgtctagcagaaatgacgagcaggttcagccagtcaaggttcctgtcaccaagcctgaagactaacttcatccccgggatccacataatggaaggagagtgagaaccccacagttgtcctctacatacacacacgcacatacaccacaaaataagatgtaataaaatatgttctaatgcaatttttcattatatagtggaatatcaattgtgtatatcctatagtaattgtacttttcaatatgtaacttaaagagatacaaaaatgtgtgcaaaaattatatatgtttttacaataagaatatatatatatacacactgtgccattaatgaatcaagccaaataacattttaatgtattagtgttcttttctgttttgaaagtcatcttactggccttgcccgcttatatgtttgaactccttttggattattgtaactggaaaacaatgtgaggtctgggatccagataaaaagactccctgaggcagattactccacacacctaagtatcacatggctgaaccagtatccagtaccacactcagtgaacagactgagcagcaaacaaggcgctgagggccaatacagggcccccaacaattcatccctctgcagaaatcatggatggtccagagcacaccactggtccctaccacctttcccctctctaccacccaatcaatcacatgagctaaacaactgtcttgaattggaatggcgagccagttccctaaatggagggcttggaccgcagagctgcacctaagaagagaatttagctcgttaaatgaaagatgtgtttccagtcaatgaaatagatcaggttaattatttaaaaatctccagcttcattgcacatgtgagtcgactgcagggaattcgaaaccttgctgcaaagtaatcaatagagatatttctaggcaaaaaaaaaaaaagtatgttggtgagcatggtggcacacgcctttaatcccagcagtcaggaggcaagtggagccatgagtgcgaggccagcctgatctacagagtgagttctgggacagccaggactacacagagacaccatctcaaaaaataataataataacaacaataataataaaagtatttggaccacaggctgcacgtctctctcacaagatcagagtgacctggcacagtgtcactgcataacagctctggtctgagggtttcaaagacattaactggtatcatgcataatgcaagatagatgctgttgttctttccattacacatataagggaactgaagcagagactcccttaagttgtcttctcaaggtttccctatttatttatgatggggcttaaattgaaaaagtcctaaggcacacagatgtgtcttttttttaagcaattatacagagcaatgcatctcttcaaacgttagtaatgcagacacatgccggcatttcatagtttcattagcaaatgactaggacaaaatctaaagaaatattgtaaagttcatcatgatgcttccctctgcacttaaaaatattatcacttcatagggaggtttgtttttaagtttatctctgataatttaattatctcactttggaaatatggcaatgtactgctcatttgactaaaattggataaaataaatgaagctaagaaggatggtatggtaatattagcaatagtttgtactttatttgaaatacgtttgtattatagaaataaaagtaaacatggggaagtatgttagatgctgttgaaaaacagtgccacattgtttccctaacattcccagtgattgtcaaagagctgctttctaattatagaatagtatttaaatagggaccaatgacattctaaagaacactaatagaaagtagttattattctcctgtatttctttaataataatagctactttctattagtgttctttagaatagaaccttggttgagtggttactctggtcagtctacccttgattttctgtcttggatgagtttgcgttgtgtgtctacaaaaaaatctcacaaacagcagcatatgaggaatcacattaaaacttctttgaaaaaagaaagtatttattataagcaacataatttccattgaaaagtaaaagaatggaaagtcaatttctaaaattaaatacataaggataactcacttcaataaattgagtaagttttgcaattatagaattatattttcctaaattctcatgaagaagtaaacatctgtaattccagaacttgggaagctaaaccaagaaaatcatgagtttggagctaacctgaacttcatagtcaccctgactcaaaacaaaagttctgattcctgataaagagcaatttgatactcattctatatgggttctattatgcaatctttaatatacaaaacagaatactttaaaatgacattattattgtgaattgatggaaaaaatagacaaagctttattataaaattaaatataagtttgtatgaaattcataatgtcctttaaaatgtgaatgacatattagaaaaaaagtcaccttatcaactgtgaatctaattttatatatagttacattatctttataactgttttaagtcctgtcagaaaaaaatgtataattaatgaaagatgatgactgacagcaccatctctcagatcaggaaaaccagctcagatgaaatgagaaaagaatacaatgttcactaccaaaaaatctatatcaggtagtttatttatttagatttttaacattgttcctggtctatatatcaaggtgatgcagatagatagatagatagatagata

SEQ ID NO: 84 - RNA, 2042 nt, synthetic construct
  source 1..2042: mol_type = other RNA
tttaaataattaacctgatctatttcattgactggaaacacatctttcatttaacgagctaaattctcttcttaggtgcagctctgcggtccaagccctccatttagggaactggctcgccattccaattcaagacagttgtttagctcatgtgattgattgggtggtagagaggggaaaggtggtagggaccagtggtgtgctctggaccatccatgatttctgcagagggatgaattgttgggggccctgtattggccctcagcgccttgtttgctgctcagtctgttcactgagtgtggtactggatactggttcagccatgtgatacttaggtgtgtggagtaatctgcctcagggagtctttttatctggatcccagacctcacattgttttccagttacaataatccaaaaggagttcaaacatataagcgggcaaggccagtaagatgactttcaaaacagaaaagaacactaatacattaaaatgttatttggcttgattcattaatggcacagtgtgtatatatatatattcttattgtaaaaacatatataatttttgcacacatttttgtatctctttaagttacatattgaaaagtacaattactataggatatacacaattgatattccactatataatgaaaaattgcattagaacatattttattacatcttattttgtggtgtatgtgcgtgtgtgtatgtagaggacaactgtggggttctcactctccttccattatgtggatcccggggatgaagttagtcttcaggcttggtgacaggaaccttgactggctgaacctgctcgtcatttctgctagacaactttagagtattccttgctaatggtagacatttcatataacttactttatcagaagtctctggataaccctccacagctgtgaggatcattcccattctacctgtgaacaaacagaggtctgaggagtcagagcaacttgctaaaggatgaaggcttgaaaacttttttcaagcaagcacaagagttatcttgattctggtttatgctcaaagcatgtattcattttgacccaatccttataatgttgactgttcttgttttccttgtgtcttaataattgaggataaaagtagaagatcacattataaataatggacaatgatactctgttccccatcagctcggtttagagagctgtttaatggttcagggcagattgttggctgcatgtctttttggcccacagcccttaagtacacagggaagaggcacaaaactcagttgagttgggcgtgagctcagtttcagcactagggactgtgcggcctcaggcattgccccaagacacgaatcatcaggtgtacactctgtccagagggacccatattgttccaagtcagcctcaagtactactaaaggcatcttgattgagactgacataagccaatccttcaggtcagaaacagagaaaaacactttgagctgaggagtgatatttcaaattctctctttttgagtctgtcgccaatcaagcttctccaggagtaaattgtcagctaatgaaatgcatgtatcatagcccagagtgtggcctatttgcagagctggctccagcccgaggcacatctttggctgctctgttctatctataagttacagccgtggcctcggcaagcagtacaccctgttcagcagctgcaactcgttatcggctttatccagaataacaaacagaacaaacacgttttgcaagggtttctctcctcttatggggagaaggaggagtgaagcaatcggtgaaatgctgattgatcaattgtactggcatagagaggttcaaggccacatgagtgctattaaccctgtaaaatgaaaaaagaaaatctcaacaaagattactcttagaactttatttatactaatatttctgtgatgttcaattttgcctaaagaaaggaaagaaatttgctttgtttattatgattttctcatattgatggtggttatgccttataaacaggattcatagtatttggactgt

SEQ ID NO: 85 - RNA, 983 nt, synthetic construct
  source 1..983: mol_type = other RNA
agaaagttcagtgttattcagcggcagatttcctcacagggcaaagcattgcattaatttatgttggctgagtgagtcacagtctgctctatttatttttcccctgtcaacttcattttgtacagagaaaactttagtaaacacaaggcggatggtttttgacacagggagtgtcaagccactgtaaagaatcagttatgtattttagctgtattattttacaatcatgtggtccccctccccccccgggatttttttttttcttcttctttgccccccactctatcatgtccttagtaattttttctggtgaaatttgtattgctagaggagaaaagtatcatgtgggattgtgagtactagaacaggctggagtccagccagccgtcctagtgaagaattcacagaactggatcttcctctccctagtctctgccagaaatctctgttcacagtaatgctcaaaagccattttggcctatcaccgtccctgtcacacctagaatatgatctcaaaattctactttcaaactttaagggcggaaaaaggaccatgtaaatgaagcaatgatgctaaattaatatattttctacttttgtcttatagaagcatttacttcaatgtaaatcttatttaagaaagtgcttcctcgttaaaacatagactttcatggtacttctcctgatgtcgacgtgaagtgaagtagctacaaaggcagtttgtatgtgcagacaactcagaacttagttttgagatattaaaacttcttattaccatagttgcattcctactcctcaggtcactataacattttaattttctataatcaagacaagttggactatatcttcacaataagtacatttatttctcttaccttgtctaccaaaaagaaaattaaaatggaagttttttcttttttcgccatcctaatggacaacaaatcttttgtgctgtgttcaaagaacaaccaaggtgtttt

SEQ ID NO: 86 - RNA, 1986 nt, synthetic construct
  source 1..1986: mol_type = other RNA
gagcagtttgttctgttcagcacattataagagggggagggttttggcaataataatcactgtgcaaagaagggctgctgatgaatttgctgttttcaaaaaacattttcgtagaaataatttactagttagaatgagcacatatctaatgatttagtcatcacactctatgaaatttacactgtaagcatgttttggtcaagttttaagaaacttctttgaccaggattagctaaactgacacagttagcatctttctaattgttttaatattcatcaagttatttttttaaaggtatcaattatgtggaactcaggtctagattgacccttttttcctttttaaagaaaagctaaactaaaaacttttactaaaggctatttagtactgaataactcataaataacgagatgactctatatttatccagatgccagatttttatatatacaaaacaaaagaaccagaaaactattttaaaggtttattttttaatctatctaaaactatctatataatttatactatttgccttctaaaaatgtaggtaaggcacttagatgttgtgaattttcttgaaatcatttttagttaaagccaatactgtttcctgatatatattgattgatatcctagcatttcttagtaagcatttcctgaattcttttagttactctacatcttatttccaaacaaatataagaattcagtgagactaaagtactcagatcctgtggctttggaatgaaaaattataaacaataaggtataagatagaaaacagtgagaacatgatttgctattgtagttaagtctggtcaagtctcaatccaatttacctgatctgaattacaaaaacaagatagccagtataattccctgctatttgaatattgttgttatttttaaatttaaatgtaataccaaaagtaaagtcagaattactttcatccctcattttttgacatttacttaagttgtatcatcttaactttacgtgatggcataaacccttctgagctctgaaatctgttctttgcacctccactctcgggtgtgctttctgtcttgctggatcttgcccagcccactctctccacctcattctacaccgtcacacccgcttttccaagtatgaccaatacacaggaagaggatggacattggctaagagctgaattctgattctggcctcgactctttcagtgtggtgctagacaagctagttacaaagctaagacacactatcagctatacaacaggaacagcaatcgataaccaggagttgtttctaaacttaatggagaaggcaagcaggtgcctaacgatgagaataaatggcaaattacttaacattattcatgactagaacaattaaaacaagtttatttaaatatgtatttatgcacacatagacataccttttattataaatatgcaatgtgctaaaattgtaatcatacactagtgtcttagcttcttatgtaagatttttaaatttattattgaataatttccatttttcatcattaaagattaagtttgattaattaactctacatgacatgtttttaaattttaattgtcaccaaagtagtactttttacatctttattcaatatttaactttacaagtaattttcagtttattttcttaatttctattctagattctgttatctaaatatactttatgcaagcatagtcagctatgactaacttaggacctcttgctattcaatattagcactacatatgcctgtcttccaaaaaacaatctatccaccacagttttcttttttggcagacatttttcctccatcttttggactgacttacatctgaccttcccacttggattttgctctgctctgtttctgtttaactgtgccttcaatttttccaaggtcacttgaattctgaatcaactttcgaaggagttgtctattttcttttcatttcacttgccctcaacatacttggct

SEQ ID NO: 87 - RNA, 5030 nt, synthetic construct
  source 1..5030: mol_type = other RNA
tccctttagcaggatatttgatataataaatctgacaccttctgggccctcctgtttccaagggaatttgatctaggaagatgtgtttcagattctcggaaactggaataatgctttcacaaactttaagtttcattatcctaaaatgttatcgcagctcgccaggaaaggaacgcacaattatttctccctccaatgtcgaaggttgagaatggcattattataaggcatgagtattgttgttgttgttgctgctgctgctgctgctgctgctgctgctgttcttgttcttgttgacgtggttatggtagtgagatggagttggtggtggtgttgtttataaagactttttattttagaaaatcaatgctatgaaaggaaaatgagaataagctagaaaggaaggacaataaaaggagaaagaaagagaaggcggcagactgatctctgaactctactaactgctaacaattagtcaaagaacctccttggatatcaaatttctgatatagctttgagtattggagaattggttggtttcttttttctggtattgttcacttgatgtcattcatccttaacctgctcttgccttcactaagatcattcacagctcagaagaattaaatatatattcccatcaattgattctgggttggaaaataaaaagtgttcttaaaaacttcaggaaattcaaaagcaaagcaaggatcattttttaaaaagttaaagactgtgaaaagatttcaaaatgttgtatttaaaagttacctgactaactgggcatttggcgggtgggcctgtaaaatctcaacacttgagagatgggaaggcctgaagttcaaggccagtctttagctgctacatagtaagtttaagactaaactgtgctatgttagaccttgtctcaaaaatgaaaagcaaatgttattgtggtgagaagctgaggctgaagtagtgttctaggagtctgacaaaaagaatgattacacatgtttaaagagcaggggaaatgacataatcaaagtactgtgttagtataaatcgtacattaacgtgttgcttggaaaagggcttttcaaatgtgaataataattatgttaacaatggtaaatgcattctttaaattattaaatattctgactcagtcacttgcccatgtctggtttctacaattgtcttctctctctctctctctctctctctctctctctctctctctctctctctctctctctctctctctcagagctgaggactaaacccaagatcttgcacttactaggcaagcgctctaccactgagctaaatccccaaccccttctactgttgtctaatcaatttatttgtgtttgattgactcagcaacacttatcttattacaatccatctatcttttaaatcaaagataaatgaagggctcttagggaacaggcttataacataaaagtgcaaaggccccagtataaaatgttactactctcataggaactgaggactctctaagaagtggaaccaactcatctctaaatctcaaacacatacaactgaagatcatatagatagattggttcacagacaaacctttaaataaaggacaaaaatactttaaataaagacttttattgcctgtggttgaattagggaaagactggaagaagctgaggaggagggcgaccctgtaggaggaccagcagtctcaattaacctggtcctccgagatctctcaaacactggaccatcatgcaccaactgatatgaggcccccaacacatacacagcagaggacttcaggatttggtttcagccagagaagatgtacctaaccctcaagagactggaggccccaaagagtttagagttctggttgggtgtggggtgggtgatagggacatactcgtggagacaggggggcggggaggaggtatgggatgtggaaaattaggagggtggaccaggtagggaataaatctgcagtgtaaattaattaattaatttaaaaaaacttttgttggcactttatttttttttcttttcctctccagaggctgaggctggaccacctgaccataacaaaagctccatgccttcgtccagacacttttcaggacggtaatttttattcacacgtgaatcaaaacaccttggttgttctttgaacacagcacaaaagatttgttgtccattaggatggcgaaaaaagaaaaaacttccattttaattttctttttggtagacaaggtaagagaaataaatgtacttattgtgaagatatagtccaacttgtcttgattatagaaaattaaaatgttatagtgacctgaggagtaggaatgcaactatggtaataagaagttttaatatctcaaaactaagttctgagttgtctgcacatacaaactgcctttgtagctacttcacttcacgtcgacatcaggagaagtaccatgaaagtctatgttttaacgaggaagcactttcttaaataagatttacattgaagtaaatgcttctataagacaaaagtagaaaatatattaatttagcatcattgcttcatttacatggtcctttttccgcccttaaagtttgaaagtagaattttgagatcatattctaggtgtgacagggacggtgataggccaaaatggcttttgagcattactgtgaacagagatttctggcagagactagggagaggaagatccagttctgtgaattcttcactaggacggctggctggactccagcctgttctagtactcacaatcccacatgatacttttctcctctagcaatacaaatttcaccagaaaaaattactaaggacatgatagagtggggggcaaagaagaagaaaaaaaaaaatcccggggggggagggggaccacatgattgtaaaataatacagctaaaatacataactgattctttacagtggcttgacactccctgtgtcaaaaaccatccgccttgtgtttactaaagttttctctgtacaaaatgaagttgacaggggaaaaataaatagagcagactgtgactcactcagccaacataaattaatgcaatgctttgccctgtgaggaaatctgccgctgaataacactgaactttctctgtttgcctgtattcgatgaaatctttgaattcctacagaatgctgttctttttttttttttttctgacatgagacaaattctttataaacatcttgtttggatcagccaccacattgtaacattgtttcttacactaaggctactattctataaaactgttacatatctccactctaactacagtgcaaagctaaaaaaaaattaaaaaaaataaaaaaataaaaaaaagcaacaccttcagttttctaagactttactctcattgcttccacattttggatgtataatcctaaattaatctcttccttgcctctttttagcatcaacaaataggatatttcctaatgtagcaagcattagttctatgagccatgaggggtagtttttaacccctccttcattcaatgttaactaaaagtcttttacatgccaagagggctacagtcaagccactgtaaagaatcagttatgtattttagctgtattattttatttattttattactttatttattatttattttcaaaagttctgaaaataaaaatgagctcagaaactctctctgcaaactcactctcaaagagcctttacagtttattcttagaaagagccatgtcgtatctagtctcaagactaaacaataaagaaagacaaagggaactgtaaacctacaaatctcaagaccccaagacccaagaacaacagaccaaatcaatattcctctccagcagccacacgacgatgaagtgtgagcatgtgtctgtacacttcacaaaatacaaagccccctgcatcagctttcatatcccaaaggagttgacccacacttctaggaagcccttcacatcctacaacttctcagaaaacaagaagaatcagatttttttgagcagacaattgtggctggaagttgagttattattactgagcaaattgactaaacttttaacatatgcaggactaaaagataaagtataaaatgaaaactcatatatatgagtaactttttaaaacaaatgttttctaaaaatatatgaaagataagtccaagatcaaatgtgtttcatggacttagattaatagtcaatggtagaaacatgagtcaaaacattttttcataaaataacaaatcttaaattaaaaccccaaatactcatgaaacaacaaatctttagctcttatcccacctaacaaatataccacaatataattactacttgacagagagaaaagtaaaggttcataccagattatgttaaagttatagggatttgtaatcaattttctcaagatctcctcatcagaatccaatgagtcactcagctcaccactgtttcttcttttccaaatcttgactgtgagcagctaagaaaagatgagccacatcagaatcaaggcaagcaaagttttgactgttcaagcttagactcaggatggctggaaacaagacttccatgtccaagcccaagtcaggacagtccttttttaaagcagattaatctttttcttcagacttgaaaactctgtgctatgtctaggaaagtaactactccgctaccttgatactgaactctgtgtttctcccagacctaagataagacacctttctttatctcctctggggaaagaaaatggcttcaaagtccagtccaggagacaagccatacggcaatgccaatagtatcagaggccttgttggatcacctgacttagaattactataggaggcaatgtaacaaacacttcaatttttaaaaatccaaaattagcatatttccttagtaaaaatggtcacgaatgtcttacagaatgtcactattaagtgacaactattggtttgcacattaactaatgcttctggatgatttaaccaacagaaatgtgctacaacttacaactttaatctc

SEQ ID NO: 88 - RNA, 843 nt, synthetic construct
  source 1..843: mol_type = other RNA
ttggtcatacttggaaaagcgggtgtgacggtgtagaatgaggtggagagagtgggctgggcaagatccagcaagacagaaagcacacccgagagtggaggtgcaaagaacagatttcagagctcagaagggtttatgccatcacgtaaagttaagatgatacaacttaagtaaatgtcaaaaaatgagggatgaaagtaattctgactttacttttggtattacatttaaatttaaaaataacaacaatattcaaatagcagggaattatactggctatcttgtttttgtaattcagatcaggtaaattggattgagacttgaccagacttaactacaatagcaaatcatgttctcactgttttctatcttataccttattgtttataatttttcattccaaagccacaggatctgagtactttagtctcactgaattcttatatttgtttggaaataagatgtagagtaactaaaagaattcaggaaatgcttactaagaaatgctaggatatcaatcaatatatatcaggaaacagtattggctttaactaaaaatgatttcaagaaaattcacaacatctaagtgccttacctacatttttagaaggcaaatagtataaattatatagatagttttagatagattaaaaaataaacctttaaaatagttttctggttcttttgttttgtatatataaaaatctggcatctggataaatatagagtcatctcgttatttatgagttattcagtactaaatagcctttagtaaaagtttttagtttagcttttctttaaaaaggaaaaaagggtcaatctagacctgagttccacataatt

SEQ ID NO: 89 - RNA, 1797 nt, synthetic construct
  source 1..1797: mol_type = other RNA
ttcttatcaaataccttgtcattgcaacaacttgtagtccataaaaaacataaggactctttgatctaaccctctactaagtccttctactaaaatgtatagaatggaatttttttttaaatatccagtttttgctttaaaatgttcagtttaatggtttaaatttaaaactttaaagatttttgagaattgttcatgtgtgacaatcacataatttgtcaagtaaatcagtcatcatttgcactatacagatgacaaaacttaggttcagagaatatgtgacttgtccaagaacagggcattaggaaatgcataacaggggttagattccagatctttttgtgcccaatccagagtatatcccactattacactgttacagcctgctgaatgtaataatgagtgctgaacattttcagtactaatggaaaaagattatggtatctaaaactttttagtatttagtattttatggttgttgatcatttatacccttgatatcaactttctttaaaatatagagcaccttgaaattctttcctctttcctgtaagtaaaatacaactgtgccgattggcaaagcactctataaatattagccgctgacaaatacccagggaagggccacataaaattataagtgcgtcattgctgtcgtcaaacccaccgtctctgtcatcaaagagagaggaaaagcaaacctctaaatatgtcgaaaaagcaacccaatttacagggcaatgattatagatgtgactacagggtgtctttcccaatccatttaccatctataactggacataatgctactaacctttaaagtgactgttctcagtgcccttcactgaccattatgtaactacatcctaatgattttaactatctttaaaatatgatgtccatctaaaaatagtaaatttggatatatttttctaagacagttgagtttttaaagtgaaggcaggaagaaaagtagatagaggactatttcacaataactgaataataacacaagtccccttgggctgttctatggaattttaattcactattgtgaagttcaagaacttctattttttgtgcttggcattcctccaaaactctggccagtaatgattgagtgtgagtaatggcttttacaccatgaaatgatggcttagtgctcctacaaatttgtcattcttagctgtgcactgtgacagacccttgaaacagatctgtcaccatgagctagaactgctttcacagtaatccatccacataattagaccattttgtcgtggttgtgattgtggtggtggttgtatggagcacttattgtgatcttggagaattggattctaatttcaatcatatctgtgccattttaagtaagtactcaatttcgcggtttctggggtctgatgaagatgcacctatccttacctacatacctaccttctcacagagtgagaagaagatattccaagtcacccaactttgaagtattagatggcacatataatcataggggataactttcattactattgattttctctgaactgatacatgagtaccttgaggtattactaacaataaattctcatagtttacatctagttacaatgtctttgatgttagtttagaatattctcctttcccattcttttgctataattaagctaaattcaagaaaatatatataactagacataatgccactaacctttagtgactgctcccagtgccctttatggccattatgtaactacatcctaatgattttaactatctttaaaacatgatgtccattta

SEQ ID NO: 90 - RNA, 2393 nt, synthetic construct
  source 1..2393: mol_type = other RNA
ttatggtattgctgttgaatcatttatctacgtaaactatttgcacttcagaaaaagtagaggccccacatgttttttttccattttcaccaaggcctaaggctatataacatacgtgctttctggcttacctgatgtttctaagctattagttcgcctcagtactcatcctcttaaaaaataaatctgaactaagcactgaaaagtagaaagcctaaagccagaatttatttcaggtaattattgtgtagataatatggtaaaaagttaaaatcaagtaaatgaaatcttattcttttattcttggaattacactattaagatatggatttttgaaatttagaatttcagcacaatttgtctttgaaatatttcatgagacttctctgcagttacaaggcctaacacttcaaaattctgaagcactgtaacagcagtaatcaattaggccttaccacattctcagttaataatcttacaacattccaagttaatattaagtcatcggtctttggttaaacataagccaatgttaaggggacaaaatcaaaaggttaatttacttaagtacttttatgtttgaacatttataaacaaaagattaagatttatgaatcatttttattctaataaggtttctcattgtaaataaaatacagcatttataaaaatttcattacaaagtatgttcataaagtgtaggtttattaatatgccttactgagcatctgctatgtggcaagttgttcagaatcctggaggaacctacatatcataagctaatttttactgtatggtggtgataaattaaaatggtcttatatttcagtctgcattatagaagaaacttagtaaaattctttcataatgtagcaaccctattgattcatttatgagaacttcaaattagtcagttacctccctatgaaatttaaaagatcaatgtatagcatttaacaagttgaaaaaaataaaaagcaaacagagtagccttgaaaatatttttataagttcaactcatttagatcacatgaagaaaatcaaatggaagctaaagatccctagggctaaggagcctaacacaaactttgaccttggcccgcgcagcttttttcgggcctccgtcattgcaaatgccccaggaaatacaaatcccatcaagggcagtcagcgtagcataccaaactcctcctgaaggcttgaagtgaattctagcatgcccattgcctaagagatactgaaaactttctacagcacccctcaatttttggaacaggttggaaatatgaagaaagtctaatacagatgtaaaggcagaggaatagtctccatttccatagcaataagacctaatgtcacctctagatcgtcagaaagtctggctagacatgagcaaattggaatgagggagaggaaagcacaggtcaagattagtactagtcccttgggcagttctacagccagaatgcttagcttctgacccaaagctgggactagggtactgagtgatgtgcttagggcctaactctcaaagagatacaccatgacactaacagtgacagcctccttaaattttgcatctcaggtacctcactcacttaacctagtctaggacatttaaactatagttttttcaagagttttatgtcaagaagctcataatgtcaaggcgttgttggtgactaataaatgaatctatattttagaaaattggggatataaaaaatggaaaaaattaaaatgttcaaaagaaaggccatctcccaattagagaagtattgcatgccaaaagtatgtaaagttgtccatcaggcaaactaattctgcaacagctcatttgggactgaacacaattaatatttcaaaaatgtagtttgccatagatgaaatccgtcctaaaatgtgtctatatgtagtaaaagtccaataaaatagttattcttaaaagtgtgtgataaccccatccccactcttacatctaacgtgtacagaggctccaatatgataatgagttatatttcctgtgactatctttaaagctactacaatgtaagtaaattattctaattgtaggagtttttatagttgccaatttctaatcaaatgacaatagctcctcaagacggaactaaatagatacttctatgtgaaaatcagaatataaaatcttaaaaatgaatgcctttcaaaagaaataatgactaccagtgctcattcgtagctgaagtatcagagaggacacttggattgttaggttcctagcttaattataacacaggtcaccattgttaaaaaatgagtggattaatgttagaaggagtacttattgaacacttttctattcatagggaacttaagctttacaatttaatacttttgatttccaaattcat

SEQ ID NO: 91 - RNA, 20 nt, synthetic construct
  source 1..20: mol_type = other RNA
cgcagctgagcagagtgaca

SEQ ID NO: 92 - RNA, 20 nt, synthetic construct
  source 1..20: mol_type = other RNA
cagtcctccttcatataagc

SEQ ID NO: 93 - RNA, 20 nt, synthetic construct
  source 1..20: mol_type = other RNA
tgaccacatgtacacagtcc

SEQ ID NO: 94 - RNA, 20 nt, synthetic construct
  source 1..20: mol_type = other RNA
gttacacttttaggacagat

SEQ ID NO: 95 - RNA, 20 nt, synthetic construct
  source 1..20: mol_type = other RNA
ggtagggaatgttaaagtca

SEQ ID NO: 96 - RNA, 20 nt, synthetic construct
  source 1..20: mol_type = other RNA
tataagagttggttggtagg

SEQ ID NO: 97 - RNA, 20 nt, synthetic construct
  source 1..20: mol_type = other RNA
caaaggagttgaggctataa

SEQ ID NO: 98 - RNA, 20 nt, synthetic construct
  source 1..20: mol_type = other RNA
gggagtttacattaggatag

SEQ ID NO: 99 - RNA, 20 nt, synthetic construct
  source 1..20: mol_type = other RNA
ggctagactgaaatctataa

SEQ ID NO: 100 - RNA, 20 nt, synthetic construct
  source 1..20: mol_type = other RNA
gctagtttcatacttgagtg

SEQ ID NO: 101 - RNA, 20 nt, synthetic construct
  source 1..20: mol_type = other RNA
cacaatggaggttggaacag

SEQ ID NO: 102 - RNA, 20 nt, synthetic construct
  source 1..20: mol_type = other RNA
tccatcaattataattccac

SEQ ID NO: 103 - RNA, 20 nt, synthetic construct
  source 1..20: mol_type = other RNA
attagagacttggaaaatcc

SEQ ID NO: 104 - RNA, 20 nt, synthetic construct
  source 1..20: mol_type = other RNA
ggtccatgtctttagtcatc

SEQ ID NO: 105 - RNA, 20 nt, synthetic construct
  source 1..20: mol_type = other RNA
agacttgcaaactacggtgt

SEQ ID NO: 106 - RNA, 20 nt, synthetic construct
  source 1..20: mol_type = other RNA
ttttatcagtaaagggtccc

SEQ ID NO: 107 - RNA, 20 nt, synthetic construct
  source 1..20: mol_type = other RNA
agtctagagagaaatgtggg

SEQ ID NO: 108 - RNA, 20 nt, synthetic construct
  source 1..20: mol_type = other RNA
ggatagaattctcctactct

SEQ ID NO: 109 - RNA, 20 nt, synthetic construct
  source 1..20: mol_type = other RNA
caacaatcaacaattctagg

SEQ ID NO: 110 - RNA, 20 nt, synthetic construct
  source 1..20: mol_type = other RNA
ttccatagaaatgtcaacaa

SEQ ID NO: 111 - RNA, 20 nt, synthetic construct
  source 1..20: mol_type = other RNA
gactacaaagcatgatgaat

SEQ ID NO: 112 - RNA, 20 nt, synthetic construct
  source 1..20: mol_type = other RNA
catttatagcagttgactac

SEQ ID NO: 113 - RNA, 20 nt, synthetic construct
  source 1..20: mol_type = other RNA
gcttgaccaaggaaaagact

SEQ ID NO: 114 - RNA, 20 nt, synthetic construct
  source 1..20: mol_type = other RNA
gtggaagtactgaggagtcc

SEQ ID NO: 115 - RNA, 20 nt, synthetic construct
  source 1..20: mol_type = other RNA
taatcaaatatgacagtgga

SEQ ID NO: 116 - RNA, 20 nt, synthetic construct
  source 1..20: mol_type = other RNA
cctcccttcatcacttatgt

SEQ ID NO: 117 - RNA, 20 nt, synthetic construct
  source 1..20: mol_type = other RNA
actataatatatttcctccc

SEQ ID NO: 118 - RNA, 20 nt, synthetic construct
  source 1..20: mol_type = other RNA
ccctaaggtacccatgcaat

SEQ ID NO: 119 - RNA, 20 nt, synthetic construct
  source 1..20: mol_type = other RNA
gtcatagtcccattacccta

SEQ ID NO: 120 - RNA, 20 nt, synthetic construct
  source 1..20: mol_type = other RNA
ggaaactttttaggatgcaa

SEQ ID NO: 121 - RNA, 20 nt, synthetic construct
  source 1..20: mol_type = other RNA
cctcattatggaaacttttc

SEQ ID NO: 122 - RNA, 20 nt, synthetic construct
  source 1..20: mol_type = other RNA
cgatataaactcaatcctca

SEQ ID NO: 123 - RNA, 20 nt, synthetic construct
  source 1..20: mol_type = other RNA
gtagtgagggttgtctttcc

SEQ ID NO: 124 - RNA, 20 nt, synthetic construct
  source 1..20: mol_type = other RNA
ctcttgtctttttatgtgtg

SEQ ID NO: 125 - RNA, 20 nt, synthetic construct
  source 1..20: mol_type = other RNA
cgccttgatctcacctcttg

SEQ ID NO: 126 - RNA, 20 nt, synthetic construct
  source 1..20: mol_type = other RNA
gtccaatactagtttacgcc

SEQ ID NO: 127 - RNA, 20 nt, synthetic construct
  source 1..20: mol_type = other RNA
agtacttactggcttagtcc

SEQ ID NO: 128 - RNA, 20 nt, synthetic construct
  source 1..20: mol_type = other RNA
ctagagaactgaggactgag

SEQ ID NO: 129 - RNA, 20 nt, synthetic construct
  source 1..20: mol_type = other RNA
ggaaaactcccaacctagag

SEQ ID NO: 130 - RNA, 20 nt, synthetic construct
  source 1..20: mol_type = other RNA
aaatgaattggaggagaatg

SEQ ID NO: 131 - RNA, 20 nt, synthetic construct
  source 1..20: mol_type = other RNA
ggcgaaatttgtgttgaaga

SEQ ID NO: 132 - RNA, 20 nt, synthetic construct
  source 1..20: mol_type = other RNA
ttgaacaagaaataaaggcg

SEQ ID NO: 133 - RNA, 20 nt, synthetic construct
  source 1..20: mol_type = other RNA
gtattcataatagtcatgga

SEQ ID NO: 134 - RNA, 20 nt, synthetic construct
  source 1..20: mol_type = other RNA
tcagccttcatccataaata

SEQ ID NO: 135 - RNA, 20 nt, synthetic construct
  source 1..20: mol_type = other RNA
tatgccttaaaatgttcagc

SEQ ID NO: 136 - RNA, 20 nt, synthetic construct
  source 1..20: mol_type = other RNA
catgggagacttagtatgcc

SEQ ID NO: 137 - RNA, 20 nt, synthetic construct
  source 1..20: mol_type = other RNA
tgaaaaggtaccatatcatg

SEQ ID NO: 138 - RNA, 20 nt, synthetic construct
  source 1..20: mol_type = other RNA
gtatagaatgggaaaatgaa

SEQ ID NO: 139 - RNA, 20 nt, synthetic construct
  source 1..20: mol_type = other RNA
agcctagatgcagggtatag

SEQ ID NO: 140 - RNA, 20 nt, synthetic construct
  source 1..20: mol_type = other RNA
atgaagtcatgtggtatatt

SEQ ID NO: 141 - RNA, 20 nt, synthetic construct
  source 1..20: mol_type = other RNA
gccgtggcatgagcaatagc

SEQ ID NO: 142 - RNA, 20 nt, synthetic construct
  source 1..20: mol_type = other RNA
tgcaaattcaccaagtagat

SEQ ID NO: 143 - RNA, 20 nt, synthetic construct
  source 1..20: mol_type = other RNA
tgtgttatactgaaggatgc

SEQ ID NO: 144 - RNA, 20 nt, synthetic construct
  source 1..20: mol_type = other RNA
gcatagaattatgttgagct

SEQ ID NO: 145 - RNA, 20 nt, synthetic construct
  source 1..20: mol_type = other RNA
agatcaagttccttgtgcat

SEQ ID NO: 146 - RNA, 20 nt, synthetic construct
  source 1..20: mol_type = other RNA
ctggtgccatgaaagttgcc

SEQ ID NO: 147 - RNA, 20 nt, synthetic construct
  source 1..20: mol_type = other RNA
ggctcatactagtttataca

SEQ ID NO: 148 - RNA, 20 nt, synthetic construct
  source 1..20: mol_type = other RNA
aagctaaacaaaataggctc

SEQ ID NO: 149 - RNA, 20 nt, synthetic construct
  source 1..20: mol_type = other RNA
catggctcctggtgatagaa

SEQ ID NO: 150 - RNA, 20 nt, synthetic construct
  source 1..20: mol_type = other RNA
gatttcaaagtgcttcatgg

SEQ ID NO: 151 - RNA, 20 nt, synthetic construct
  source 1..20: mol_type = other RNA
agtcctcacgcttacttaat

SEQ ID NO: 152 - RNA, 20 nt, synthetic construct
  source 1..20: mol_type = other RNA
caggcacacacatcaggctg

SEQ ID NO: 153 - RNA, 20 nt, synthetic construct
  source 1..20: mol_type = other RNA
gcaggcacacacatcaggct

SEQ ID NO: 154 - RNA, 20 nt, synthetic construct
  source 1..20: mol_type = other RNA
atgcaggcacacacatcagg

SEQ ID NO: 155 - RNA, 20 nt, synthetic construct
  source 1..20: mol_type = other RNA
gaatgcaggcacacacatca

SEQ ID NO: 156 - RNA, 20 nt, synthetic construct
  source 1..20: mol_type = other RNA
tgcaggcacacacatcaggc

SEQ ID NO: 157 - RNA, 22 nt, synthetic construct
  source 1..22: mol_type = other RNA
tgcaggcacacacatcaggctg

SEQ ID NO: 158 - RNA, 20 nt, synthetic construct
  source 1..20: mol_type = other RNA
caggcacacacatcaggctg

SEQ ID NO: 159 - RNA, 20 nt, synthetic construct
  source 1..20: mol_type = other RNA
gcaggcacacacatcaggct

SEQ ID NO: 160 - RNA, 20 nt, synthetic construct
  source 1..20: mol_type = other RNA
atgcaggcacacacatcagg

SEQ ID NO: 161 - RNA, 20 nt, synthetic construct
  source 1..20: mol_type = other RNA
tgcaggcacacacatcaggc

SEQ ID NO: 162 - RNA, 19 nt, synthetic construct
  source 1..19: mol_type = other RNA
tgcaggcacacacatcagg

SEQ ID NO: 163 - RNA, 18 nt, synthetic construct
  source 1..18: mol_type = other RNA
tgcaggcacacacatcag

SEQ ID NO: 164 - RNA, 15 nt, synthetic construct
  source 1..15: mol_type = other RNA
tgcaggcacacacat

SEQ ID NO: 165 - skipped

SEQ ID NO: 166 - RNA, 20 nt, synthetic construct
  source 1..20: mol_type = other RNA
tgcaggcacacacatcaggc

SEQ ID NO: 167 - RNA, 20 nt, synthetic construct
  source 1..20: mol_type = other RNA
caggcatgaatgcaggcaca

SEQ ID NO: 168 - RNA, 20 nt, synthetic construct
  source 1..20: mol_type = other RNA
acaggcatgaatgcaggcac

SEQ ID NO: 169 - RNA, 20 nt, synthetic construct
  source 1..20: mol_type = other RNA
gtgacaggcatgaatgcagg

SEQ ID NO: 170 - RNA, 20 nt, synthetic construct
  source 1..20: mol_type = other RNA
agtgacaggcatgaatgcag

SEQ ID NO: 171 - RNA, 20 nt, synthetic construct
  source 1..20: mol_type = other RNA
gagtgacaggcatgaatgca

SEQ ID NO: 172 - RNA, 16 nt, synthetic construct
  source 1..16: mol_type = other RNA
aatgcaggcacacaca

SEQ ID NO: 173 - RNA, 16 nt, synthetic construct
  source 1..16: mol_type = other RNA
tgaatgcaggcacaca

SEQ ID NO: 174 - RNA, 16 nt, synthetic construct
  source 1..16: mol_type = other RNA
catgaatgcaggcaca

SEQ ID NO: 175 - RNA, 16 nt, synthetic construct
  source 1..16: mol_type = other RNA
tgcaggcacacacatc

SEQ ID NO: 176 - RNA, 16 nt, synthetic construct
  source 1..16: mol_type = other RNA
caggcacacacatcag

SEQ ID NO: 177 - RNA, 16 nt, synthetic construct
  source 1..16: mol_type = other RNA
ggcacacacatcaggc

SEQ ID NO: 178 - RNA, 16 nt, synthetic construct
  source 1..16: mol_type = other RNA
cacacacatcaggctg

SEQ ID NO: 179 - RNA, 16 nt, synthetic construct
  source 1..16: mol_type = other RNA
cacacatcaggctggg

SEQ ID NO: 180 - RNA, 30 nt, synthetic construct
  source 1..30: mol_type = other RNA
tgcaggcacacacatcaggctttttttttt

SEQ ID NO: 181 - RNA, 30 nt, synthetic construct
  source 1..30: mol_type = other RNA
tttttttttttgcaggcacacacatcaggc

SEQ ID NO: 182 - RNA, 20 nt, synthetic construct
  source 1..20: mol_type = other RNA
acattactttaaatgattac

SEQ ID NO: 183 - RNA, 20 nt, synthetic construct
  source 1..20: mol_type = other RNA
gatgttattacaaagttctt

SEQ ID NO: 184 - RNA, 20 nt, synthetic construct
  source 1..20: mol_type = other RNA
ggaggcaatattatgttaaa

SEQ ID NO: 185 - RNA, 20 nt, synthetic construct
  source 1..20: mol_type = other RNA
gcataatgctttttggaggc

SEQ ID NO: 186 - RNA, 20 nt, synthetic construct
  source 1..20: mol_type = other RNA
cttgcataatgctttttgga

SEQ ID NO: 187 - RNA, 20 nt, synthetic construct
  source 1..20: mol_type = other RNA
acttgcataatgctttttgg

SEQ ID NO: 188 - RNA, 20 nt, synthetic construct
  source 1..20: mol_type = other RNA
gacttgcataatgctttttg

SEQ ID NO: 189 - RNA, 20 nt, synthetic construct
  source 1..20: mol_type = other RNA
ccattgacttgcataatgct

SEQ ID NO: 190 - RNA, 20 nt, synthetic construct
  source 1..20: mol_type = other RNA
gccattgacttgcataatgc

SEQ ID NO: 191 - RNA, 20 nt, synthetic construct
  source 1..20: mol_type = other RNA
tctagcatttatgtcaatgc

SEQ ID NO: 192 - RNA, 20 nt, synthetic construct
  source 1..20: mol_type = other RNA
ctctgttaacactaaagtat

SEQ ID NO: 193 - RNA, 20 nt, synthetic construct
  source 1..20: mol_type = other RNA
tctctgttaacactaaagta

SEQ ID NO: 194 - RNA, 20 nt, synthetic construct
  source 1..20: mol_type = other RNA
atctctgttaacactaaagt

SEQ ID NO: 195 - RNA, 20 nt, synthetic construct
  source 1..20: mol_type = other RNA
gataataacccaccacacaa

SEQ ID NO: 196 - RNA, 20 nt, synthetic construct
  source 1..20: mol_type = other RNA
gaaatgtggaaatacagaat

SEQ ID NO: 197 - RNA, 20 nt, synthetic construct
  source 1..20: mol_type = other RNA
atgtcaagtactaggttgta

SEQ ID NO: 198 - RNA, 20 nt, synthetic construct
  source 1..20: mol_type = other RNA
ccttattgacatatctggta

SEQ ID NO: 199 - RNA, 20 nt, synthetic construct
  source 1..20: mol_type = other RNA
tcagggcactttattttatg

SEQ ID NO: 200 - RNA, 20 nt, synthetic construct
  source 1..20: mol_type = other RNA
gaaatccctcagggcacttt

SEQ ID NO: 201 - RNA, 20 nt, synthetic construct
  source 1..20: mol_type = other RNA
tgaaatccctcagggcactt

SEQ ID NO: 202 - RNA, 20 nt, synthetic construct
  source 1..20: mol_type = other RNA
gtgaaatccctcagggcact

SEQ ID NO: 203 - RNA, 20 nt, synthetic construct
  source 1..20: mol_type = other RNA
tgtgaaatccctcagggcac

SEQ ID NO: 204 - RNA, 20 nt, synthetic construct
  source 1..20: mol_type = other RNA
ctgtgaaatccctcagggca

SEQ ID NO: 205 - RNA, 20 nt, synthetic construct
  source 1..20: mol_type = other RNA
gcatttagcctgtgaaatcc

SEQ ID NO: 206 - RNA, 20 nt, synthetic construct
  source 1..20: mol_type = other RNA
tagaagcatttagcctgtga

SEQ ID NO: 207 - RNA, 20 nt, synthetic construct
  source 1..20: mol_type = other RNA
gtcttcttttctattaatac

SEQ ID NO: 208 - RNA, 20 nt, synthetic construct
  source 1..20: mol_type = other RNA
agtcactctctgataaaatg

SEQ ID NO: 209 - RNA, 20 nt, synthetic construct
  source 1..20: mol_type = other RNA
gtgagccaggtcatcaaagg

SEQ ID NO: 210 - RNA, 20 nt, synthetic construct
  source 1..20: mol_type = other RNA
tttgtgagccaggtcatcaa

SEQ ID NO: 211 - RNA, 20 nt, synthetic construct
  source 1..20: mol_type = other RNA
cattctttgtgagccaggtc

SEQ ID NO: 212 - RNA, 20 nt, synthetic construct
  source 1..20: mol_type = other RNA
ggaagtacattctttgtgag

SEQ ID NO: 213 - RNA, 20 nt, synthetic construct
  source 1..20: mol_type = other RNA
acttggaagtacattctttg

SEQ ID NO: 214 - RNA, 20 nt, synthetic construct
  source 1..20: mol_type = other RNA
gtgtacttggaagtacattc

SEQ ID NO: 215 - RNA, 20 nt, synthetic construct
  source 1..20: mol_type = other RNA
gaccctgtgtacttggaagt

SEQ ID NO: 216 - RNA, 20 nt, synthetic construct
  source 1..20: mol_type = other RNA
tggggaccctgtgtacttgg

SEQ ID NO: 217 - RNA, 20 nt, synthetic construct
  source 1..20: mol_type = other RNA
atcaggctggggaccctgtg

SEQ ID NO: 218 - RNA, 20 nt, synthetic construct
  source 1..20: mol_type = other RNA
acacatcaggctggggaccc

SEQ ID NO: 219 - RNA, 20 nt, synthetic construct
  source 1..20: mol_type = other RNA
ggcacacacatcaggctggg

SEQ ID NO: 220 - RNA, 20 nt, synthetic construct
  source 1..20: mol_type = other RNA
atgaatgcaggcacacacat

SEQ ID NO: 221 - RNA, 20 nt, synthetic construct
  source 1..20: mol_type = other RNA
aggcatgaatgcaggcacac

SEQ ID NO: 222 - RNA, 20 nt, synthetic construct
  source 1..20: mol_type = other RNA
tgacaggcatgaatgcaggc

SEQ ID NO: 223 - RNA, 20 nt, synthetic construct
  source 1..20: mol_type = other RNA
agagtgacaggcatgaatgc

SEQ ID NO: 224 - RNA, 20 nt, synthetic construct
  source 1..20: mol_type = other RNA
gagcagagtgacaggcatga

SEQ ID NO: 225 - RNA, 20 nt, synthetic construct
  source 1..20: mol_type = other RNA
cagctgagcagagtgacagg

SEQ ID NO: 226 - RNA, 20 nt, synthetic construct
  source 1..20: mol_type = other RNA
tgcacgcagctgagcagagt

SEQ ID NO: 227 - RNA, 20 nt, synthetic construct
  source 1..20: mol_type = other RNA
tgtctgcacgcagctgagca

SEQ ID NO: 228 - RNA, 20 nt, synthetic construct
  source 1..20: mol_type = other RNA
aagctgtctgcacgcagctg

SEQ ID NO: 229 - RNA, 20 nt, synthetic construct
  source 1..20: mol_type = other RNA
atataagctgtctgcacgca

SEQ ID NO: 230 - RNA, 20 nt, synthetic construct
  source 1..20: mol_type = other RNA
ccttcatataagctgtctgc

SEQ ID NO: 231 - RNA, 20 nt, synthetic construct
  source 1..20: mol_type = other RNA
gtcctccttcatataagctg

SEQ ID NO: 232 - RNA, 20 nt, synthetic construct
  source 1..20: mol_type = other RNA
ttcttctctgaccacatgta

SEQ ID NO: 233 - RNA, 20 nt, synthetic construct
  source 1..20: mol_type = other RNA
acattttaaaaaataagcat

SEQ ID NO: 234 - RNA, 20 nt, synthetic construct
  source 1..20: mol_type = other RNA
acacttttaggacagattat

SEQ ID NO: 235 - RNA, 20 nt, synthetic construct
  source 1..20: mol_type = other RNA
tgttacacttttaggacaga

SEQ ID NO: 236 - RNA, 20 nt, synthetic construct
  source 1..20: mol_type = other RNA
ggaaatgttacacttttagg

SEQ ID NO: 237 - RNA, 20 nt, synthetic construct
  source 1..20: mol_type = other RNA
gacatgaaaaaatttagtgg

SEQ ID NO: 238 - RNA, 20 nt, synthetic construct
  source 1..20: mol_type = other RNA
ggttggtagggaatgttaaa

SEQ ID NO: 239 - RNA, 20 nt, synthetic construct
  source 1..20: mol_type = other RNA
tggttggtagggaatgttaa

SEQ ID NO: 240 - RNA, 20 nt, synthetic construct
  source 1..20: mol_type = other RNA
ttggttggtagggaatgtta

SEQ ID NO: 241 - RNA, 20 nt, synthetic construct
  source 1..20: mol_type = other RNA
gttggttggtagggaatgtt

SEQ ID NO: 242 - RNA, 20 nt, synthetic construct
  source 1..20: mol_type = other RNA
agttggttggtagggaatgt

SEQ ID NO: 243 - RNA, 20 nt, synthetic construct
  source 1..20: mol_type = other RNA
gagttggttggtagggaatg

SEQ ID NO: 244 - RNA, 20 nt, synthetic construct
  source 1..20: mol_type = other RNA
agagttggttggtagggaat

SEQ ID NO: 245 - RNA, 20 nt, synthetic construct
  source 1..20: mol_type = other RNA
aagagttggttggtagggaa

SEQ ID NO: 246 - RNA, 20 nt, synthetic construct
  source 1..20: mol_type = other RNA
taagagttggttggtaggga

SEQ ID NO: 247 - RNA, 20 nt, synthetic construct
  source 1..20: mol_type = other RNA
ataagagttggttggtaggg

SEQ ID NO: 248 - RNA, 20 nt, synthetic construct
  source 1..20: mol_type = other RNA
ctataagagttggttggtag

SEQ ID NO: 249 - RNA, 20 nt, synthetic construct
  source 1..20: mol_type = other RNA
gagggagtttacattaggat

SEQ ID NO: 250 - RNA, 20 nt, synthetic construct
  source 1..20: mol_type = other RNA
tggaaaaaaatatattacac

SEQ ID NO: 251 - RNA, 20 nt, synthetic construct
  source 1..20: mol_type = other RNA
gctagactgaaatctataac

SEQ ID NO: 252 - RNA, 20 nt, synthetic construct
  source 1..20: mol_type = other RNA
gtgttcaggctagactgaaa

SEQ ID NO: 253 - RNA, 20 nt, synthetic construct
  source 1..20: mol_type = other RNA
agtgttcaggctagactgaa

SEQ ID NO: 254 - RNA, 20 nt, synthetic construct
  source 1..20: mol_type = other RNA
gagtgttcaggctagactga

SEQ ID NO: 255 - RNA, 20 nt, synthetic construct
  source 1..20: mol_type = other RNA
tgagtgttcaggctagactg

SEQ ID NO: 256 - RNA, 20 nt, synthetic construct
  source 1..20: mol_type = other RNA
ttgagtgttcaggctagact

SEQ ID NO: 257 - RNA, 20 nt, synthetic construct
  source 1..20: mol_type = other RNA
cttgagtgttcaggctagac

SEQ ID NO: 258 - RNA, 20 nt, synthetic construct
  source 1..20: mol_type = other RNA
acttgagtgttcaggctaga

SEQ ID NO: 259 - RNA, 20 nt, synthetic construct
  source 1..20: mol_type = other RNA
tacttgagtgttcaggctag

SEQ ID NO: 260 - RNA, 20 nt, synthetic construct
  source 1..20: mol_type = other RNA
atacttgagtgttcaggcta

SEQ ID NO: 261 - RNA, 20 nt, synthetic construct
  source 1..20: mol_type = other RNA
catacttgagtgttcaggct

SEQ ID NO: 262 - RNA, 20 nt, synthetic construct
  source 1..20: mol_type = other RNA
tcatacttgagtgttcaggc

SEQ ID NO: 263 - RNA, 20 nt, synthetic construct
  source 1..20: mol_type = other RNA
ttcatacttgagtgttcagg

SEQ ID NO: 264 - RNA, 20 nt, synthetic construct
  source 1..20: mol_type = other RNA
gtttcatacttgagtgttca

SEQ ID NO: 265 - RNA, 20 nt, synthetic construct
  source 1..20: mol_type = other RNA
ggctagtttcatacttgagt

SEQ ID NO: 266 - RNA, 20 nt, synthetic construct
  source 1..20: mol_type = other RNA
gtggctagtttcatacttga

SEQ ID NO: 267 - RNA, 20 nt, synthetic construct
  source 1..20: mol_type = other RNA
agtggctagtttcatacttg

SEQ ID NO: 268 - RNA, 20 nt, synthetic construct
  source 1..20: mol_type = other RNA
ggaggttggaacagccaata

SEQ ID NO: 269 - RNA, 20 nt, synthetic construct
  source 1..20: mol_type = other RNA
caatggaggttggaacagcc

SEQ ID NO: 270 - RNA, 20 nt, synthetic construct
  source 1..20: mol_type = other RNA
acaatggaggttggaacagc

SEQ ID NO: 271 - RNA, 20 nt, synthetic construct
  source 1..20: mol_type = other RNA
ccacaatggaggttggaaca

SEQ ID NO: 272 - RNA, 20 nt, synthetic construct
  source 1..20: mol_type = other RNA
tccacaatggaggttggaac

SEQ ID NO: 273 - RNA, 20 nt, synthetic construct
  source 1..20: mol_type = other RNA
ttccacaatggaggttggaa

SEQ ID NO: 274 - RNA, 20 nt, synthetic construct
  source 1..20: mol_type = other RNA
attccacaatggaggttgga

SEQ ID NO: 275 - RNA, 20 nt, synthetic construct
  source 1..20: mol_type = other RNA
aattccacaatggaggttgg

SEQ ID NO: 276 - RNA, 20 nt, synthetic construct
  source 1..20: mol_type = other RNA
taattccacaatggaggttg

SEQ ID NO: 277 - RNA, 20 nt, synthetic construct
  source 1..20: mol_type = other RNA
atccatcaattataattcca

SEQ ID NO: 278 - RNA, 20 nt, synthetic construct
  source 1..20: mol_type = other RNA
aatccatcaattataattcc

SEQ ID NO: 279 - RNA, 20 nt, synthetic construct
  source 1..20: mol_type = other RNA
aaatccatcaattataattc

SEQ ID NO: 280 - RNA, 20 nt, synthetic construct
  source 1..20: mol_type = other RNA
aaaatccatcaattataatt

SEQ ID NO: 281 - RNA, 20 nt, synthetic construct
  source 1..20: mol_type = other RNA
gaaaatccatcaattataat

SEQ ID NO: 282 - RNA, 20 nt, synthetic construct
  source 1..20: mol_type = other RNA
ggaaaatccatcaattataa

SEQ ID NO: 283 - RNA, 20 nt, synthetic construct
  source 1..20: mol_type = other RNA
tggaaaatccatcaattata

SEQ ID NO: 284 - RNA, 20 nt, synthetic construct
  source 1..20: mol_type = other RNA
ttggaaaatccatcaattat

SEQ ID NO: 285 - RNA, 20 nt, synthetic construct
  source 1..20: mol_type = other RNA
cttggaaaatccatcaatta

SEQ ID NO: 286 - RNA, 20 nt, synthetic construct
  source 1..20: mol_type = other RNA
acttggaaaatccatcaatt

SEQ ID NO: 287 - RNA, 20 nt, synthetic construct
  source 1..20: mol_type = other RNA
gacttggaaaatccatcaat

SEQ ID NO: 288 - RNA, 20 nt, synthetic construct
  source 1..20: mol_type = other RNA
agacttggaaaatccatcaa

SEQ ID NO: 289 - RNA, 20 nt, synthetic construct
  source 1..20: mol_type = other RNA
gagacttggaaaatccatca

SEQ ID NO: 290 - RNA, 20 nt, synthetic construct
  source 1..20: mol_type = other RNA
agagacttggaaaatccatc

SEQ ID NO: 291 - RNA, 20 nt, synthetic construct
  source 1..20: mol_type = other RNA
gaattagagacttggaaaat

SEQ ID NO: 292 - RNA, 20 nt, synthetic construct
  source 1..20: mol_type = other RNA
tgaattagagacttggaaaa

SEQ ID NO: 293 - RNA, 20 nt, synthetic construct
  source 1..20: mol_type = other RNA
ctgaattagagacttggaaa

SEQ ID NO: 294 - RNA, 20 nt, synthetic construct
  source 1..20: mol_type = other RNA
tctgaattagagacttggaa

SEQ ID NO: 295 - RNA, 20 nt, synthetic construct
  source 1..20: mol_type = other RNA
ttctgaattagagacttgga

SEQ ID NO: 296 - RNA, 20 nt, synthetic construct
  source 1..20: mol_type = other RNA
attctgaattagagacttgg

SEQ ID NO: 297 - RNA, 20 nt, synthetic construct
  source 1..20: mol_type = other RNA
tattctgaattagagacttg

SEQ ID NO: 298 - RNA, 20 nt, synthetic construct
  source 1..20: mol_type = other RNA
ggagaaggcagaaattttgg

SEQ ID NO: 299 - RNA, 20 nt, synthetic construct
  source 1..20: mol_type = other RNA
gtccatgtctttagtcatca

SEQ ID NO: 300 - RNA, 20 nt, synthetic construct
  source 1..20: mol_type = other RNA
acggtgtgtgtgtgtatata

SEQ ID NO: 301 - RNA, 20 nt, synthetic construct
  source 1..20: mol_type = other RNA
aactacggtgtgtgtgtgta

SEQ ID NO: 302 - RNA, 20 nt, synthetic construct
  source 1..20: mol_type = other RNA
tgcaaactacggtgtgtgtg

SEQ ID NO: 303 - RNA, 20 nt, synthetic construct
  source 1..20: mol_type = other RNA
gacttgcaaactacggtgtg

SEQ ID NO: 304 - RNA, 20 nt, synthetic construct
  source 1..20: mol_type = other RNA
atgcagacttgcaaactacg

SEQ ID NO: 305 - RNA, 20 nt, synthetic construct
  source 1..20: mol_type = other RNA
ccagatgcagacttgcaaac

SEQ ID NO: 306 - RNA, 20 nt, synthetic construct
  source 1..20: mol_type = other RNA
ggtcccagatgcagacttgc

SEQ ID NO: 307 - RNA, 20 nt, synthetic construct
  source 1..20: mol_type = other RNA
aaagggtcccagatgcagac

SEQ ID NO: 308 - RNA, 20 nt, synthetic construct
  source 1..20: mol_type = other RNA
cagtaaagggtcccagatgc

SEQ ID NO: 309 - RNA, 20 nt, synthetic construct
  source 1..20: mol_type = other RNA
tatcagtaaagggtcccaga

SEQ ID NO: 310 - RNA, 20 nt, synthetic construct
  source 1..20: mol_type = other RNA
ggcatgagagttttaatggg

SEQ ID NO: 311 - RNA, 20 nt, synthetic construct
  source 1..20: mol_type = other RNA
gatagaattctcctactcta

SEQ ID NO: 312 - RNA, 20 nt, synthetic construct
  source 1..20: mol_type = other RNA
ccatagaaatgtcaacaatc

SEQ ID NO: 313 - RNA, 20 nt, synthetic construct
  source 1..20: mol_type = other RNA
gcatgatgaattaaacatgt

SEQ ID NO: 314 - RNA, 20 nt, synthetic construct
  source 1..20: mol_type = other RNA
ccatttatagcagttgacta

SEQ ID NO: 315 - RNA, 20 nt, synthetic construct
  source 1..20: mol_type = other RNA
ttgaccaaggaaaagacttc

SEQ ID NO: 316 - RNA, 20 nt, synthetic construct
  source 1..20: mol_type = other RNA
agcttgaccaaggaaaagac

SEQ ID NO: 317 - RNA, 20 nt, synthetic construct
  source 1..20: mol_type = other RNA
agtccagagcttgaccaagg

SEQ ID NO: 318 - RNA, 20 nt, synthetic construct
  source 1..20: mol_type = other RNA
gaggagtccagagcttgacc

SEQ ID NO: 319 - RNA, 20 nt, synthetic construct
  source 1..20: mol_type = other RNA
agtactgaggagtccagagc

SEQ ID NO: 320 - RNA, 20 nt, synthetic construct
  source 1..20: mol_type = other RNA
agtggaagtactgaggagtc

SEQ ID NO: 321 - RNA, 20 nt, synthetic construct
  source 1..20: mol_type = other RNA
tgacagtggaagtactgagg

SEQ ID NO: 322 - RNA, 20 nt, synthetic construct
  source 1..20: mol_type = other RNA
tatgacagtggaagtactga

SEQ ID NO: 323 - RNA, 20 nt, synthetic construct
  source 1..20: mol_type = other RNA
tcctcccttcatcacttatg

SEQ ID NO: 324 - RNA, 20 nt, synthetic construct
  source 1..20: mol_type = other RNA
acccatgcaatgaatactat

SEQ ID NO: 325 - RNA, 20 nt, synthetic construct
  source 1..20: mol_type = other RNA
tacccatgcaatgaatacta

SEQ ID NO: 326 - RNA, 20 nt, synthetic construct
  source 1..20: mol_type = other RNA
gtacccatgcaatgaatact

SEQ ID NO: 327 - RNA, 20 nt, synthetic construct
  source 1..20: mol_type = other RNA
ggtacccatgcaatgaatac

SEQ ID NO: 328 - RNA, 20 nt, synthetic construct
  source 1..20: mol_type = other RNA
aggtacccatgcaatgaata

SEQ ID NO: 329 - RNA, 20 nt, synthetic construct
  source 1..20: mol_type = other RNA
aaggtacccatgcaatgaat

SEQ ID NO: 330 - RNA, 20 nt, synthetic construct
  source 1..20: mol_type = other RNA
taaggtacccatgcaatgaa

SEQ ID NO: 331 - RNA, 20 nt, synthetic construct
  source 1..20: mol_type = other RNA
cctaaggtacccatgcaatg

SEQ ID NO: 332 - RNA, 20 nt, synthetic construct
  source 1..20: mol_type = other RNA
tttaaatcagtcatagtccc

SEQ ID NO: 333 - RNA, 20 nt, synthetic construct
  source 1..20: mol_type = other RNA
gctgcacccactaatggaaa

SEQ ID NO: 334 - RNA, 20 nt, synthetic construct
  source 1..20: mol_type = other RNA
gtgctgcacccactaatgga

SEQ ID NO: 335 - RNA, 20 nt, synthetic construct
  source 1..20: mol_type = other RNA
ggaaacttttctttttaagt

SEQ ID NO: 336 - RNA, 20 nt, synthetic construct
  source 1..20: mol_type = other RNA
acgatataaactcaatcctc

SEQ ID NO: 337 - RNA, 20 nt, synthetic construct
  source 1..20: mol_type = other RNA
tagtgagggttgtctttcct

SEQ ID NO: 338 - RNA, 20 nt, synthetic construct
  source 1..20: mol_type = other RNA
tgtagtgagggttgtctttc

SEQ ID NO: 339 - RNA, 20 nt, synthetic construct
  source 1..20: mol_type = other RNA
gtgtagtgagggttgtcttt

SEQ ID NO: 340 - RNA, 20 nt, synthetic construct
  source 1..20: mol_type = other RNA
gtgtgtagtgagggttgtct

SEQ ID NO: 341 - RNA, 20 nt, synthetic construct
  source 1..20: mol_type = other RNA
tcttgtctttttatgtgtgt

SEQ ID NO: 342 - RNA, 20 nt, synthetic construct
  source 1..20: mol_type = other RNA
tactagtttacgccttgatc

SEQ ID NO: 343 - RNA, 20 nt, synthetic construct
  source 1..20: mol_type = other RNA
gcagtacttactggcttagt

SEQ ID NO: 344 - RNA, 20 nt, synthetic construct
  source 1..20: mol_type = other RNA
gaaaactcccaacctagaga

SEQ ID NO: 345 - RNA, 20 nt, synthetic construct
  source 1..20: mol_type = other RNA
ttggaggagaatgaaaatag

SEQ ID NO: 346 - RNA, 20 nt, synthetic construct
  source 1..20: mol_type = other RNA
cgaaatttgtgttgaagatc

SEQ ID NO: 347 - RNA, 20 nt, synthetic construct
  source 1..20: mol_type = other RNA
cataatagtcatggaagtat

SEQ ID NO: 348 - RNA, 20 nt, synthetic construct
  source 1..20: mol_type = other RNA
aatgttcagccttcatccat

SEQ ID NO: 349 - RNA, 20 nt, synthetic construct
  source 1..20: mol_type = other RNA
tcatgggagacttagtatgc

SEQ ID NO: 350 - RNA, 20 nt, synthetic construct
  source 1..20: mol_type = other RNA
ctagatgcagggtatagaat

SEQ ID NO: 351 - RNA, 20 nt, synthetic construct
  source 1..20: mol_type = other RNA
catgaagtcatgtggtatat

SEQ ID NO: 352 - RNA, 20 nt, synthetic construct
  source 1..20: mol_type = other RNA
ccgtggcatgagcaatagcc

SEQ ID NO: 353 - RNA, 20 nt, synthetic construct
  source 1..20: mol_type = other RNA
aagtagataaagaggggaaa

SEQ ID NO: 354 - RNA, 16 nt, synthetic construct
  source 1..16: mol_type = other RNA
ggcatgaatgcaggca

SEQ ID NO: 355 - RNA, 16 nt, synthetic construct
  source 1..16: mol_type = other RNA
caggcatgaatgcagg

SEQ ID NO: 356 - RNA, 16 nt, synthetic construct
  source 1..16: mol_type = other RNA
gacaggcatgaatgca

SEQ ID NO: 357 - RNA, 16 nt, synthetic construct
  source 1..16: mol_type = other RNA
gtgacaggcatgaatg

SEQ ID NO: 358 - RNA, 16 nt, synthetic construct
  source 1..16: mol_type = other RNA
cacatcaggctgggga

SEQ ID NO: 359 - RNA, 16 nt, synthetic construct
  source 1..16: mol_type = other RNA
catcaggctggggacc

SEQ ID NO: 360 - RNA, 16 nt, synthetic construct
  source 1..16: mol_type = other RNA
tcaggctggggaccct

SEQ ID NO: 361 - RNA, 16 nt, synthetic construct
  source 1..16: mol_type = other RNA
aggctggggaccctgt

SEQ ID NO: 362 - RNA, 16 nt, synthetic construct
  source 1..16: mol_type = other RNA
catgaatgcaggcaca

SEQ ID NO: 363 - RNA, 16 nt, synthetic construct
  source 1..16: mol_type = other RNA
tgaatgcaggcacaca

SEQ ID NO: 364 - RNA, 16 nt, synthetic construct
  source 1..16: mol_type = other RNA
aatgcaggcacacaca

SEQ ID NO: 365 - RNA, 16 nt, synthetic construct
  source 1..16: mol_type = other RNA
tgcaggcacacacatc

SEQ ID NO: 366 - RNA, 16 nt, synthetic construct
  source 1..16: mol_type = other RNA
caggcacacacatcag

SEQ ID NO: 367 - RNA, 16 nt, synthetic construct
  source 1..16: mol_type = other RNA
ggcacacacatcaggc

SEQ ID NO: 368 - RNA, 16 nt, synthetic construct
  source 1..16: mol_type = other RNA
cacacacatcaggctg

SEQ ID NO: 369 - RNA, 16 nt, synthetic construct
  source 1..16: mol_type = other RNA
cacacatcaggctggg

SEQ ID NO: 370 - RNA, 16 nt, synthetic construct
  source 1..16: mol_type = other RNA
ggcatgaatgcaggca

SEQ ID NO: 371 - RNA, 16 nt, synthetic construct
  source 1..16: mol_type = other RNA
caggcatgaatgcagg

SEQ ID NO: 372 - RNA, 16 nt, synthetic construct
  source 1..16: mol_type = other RNA
gacaggcatgaatgca

SEQ ID NO: 373 - RNA, 16 nt, synthetic construct
  source 1..16: mol_type = other RNA
gtgacaggcatgaatg

SEQ ID NO: 374 - RNA, 16 nt, synthetic construct
  source 1..16: mol_type = other RNA
cacatcaggctgggga

SEQ ID NO: 375 - RNA, 16 nt, synthetic construct
  source 1..16: mol_type = other RNA
catcaggctggggacc

SEQ ID NO: 376 - RNA, 16 nt, synthetic construct
  source 1..16: mol_type = other RNA
tcaggctggggaccct

SEQ ID NO: 377 - RNA, 16 nt, synthetic construct
  source 1..16: mol_type = other RNA
aggctggggaccctgt

SEQ ID NO: 378 - RNA, 18 nt, synthetic construct
  source 1..18: mol_type = other RNA
gcatgaatgcaggcacac

SEQ ID NO: 379 - RNA, 18 nt, synthetic construct
  source 1..18: mol_type = other RNA
gcacacacatcaggctgg

SEQ ID NO: 380 - RNA, 18 nt, synthetic construct
  source 1..18: mol_type = other RNA
acacacatcaggctgggg

SEQ ID NO: 381 - RNA, 18 nt, synthetic construct
  source 1..18: mol_type = other RNA
cacacacatcaggctggg

SEQ ID NO: 382 - RNA, 20 nt, synthetic construct
  source 1..20: mol_type = other RNA
ggcatgaatgcaggcacaca

SEQ ID NO: 383 - RNA, 20 nt, synthetic construct
  source 1..20: mol_type = other RNA
ggcatgaatgcaggcacaca

SEQ ID NO: 384 - RNA, 20 nt, synthetic construct
  source 1..20: mol_type = other RNA
ggcacacacatcaggctggg

SEQ ID NO: 385 - RNA, 20 nt, synthetic construct
  source 1..20: mol_type = other RNA
cacacacatcaggctgggga

SEQ ID NO: 386 - RNA, 20 nt, synthetic construct
  source 1..20: mol_type = other RNA
gcacacacatcaggctgggg

SEQ ID NO: 387 - RNA, 20 nt, synthetic construct
  source 1..20: mol_type = other RNA
tgcaggcacacacatcaggc

SEQ ID NO: 388 - RNA, 20 nt, synthetic construct
  source 1..20: mol_type = other RNA
atcaggctggggaccctgtg

SEQ ID NO: 389 - RNA, 20 nt, synthetic construct
  source 1..20: mol_type = other RNA
ggcacacacatcaggctggg

SEQ ID NO: 390 - RNA, 20 nt, synthetic construct
  source 1..20: mol_type = other RNA
atgaatgcaggcacacacat

SEQ ID NO: 391 - RNA, 20 nt, synthetic construct
  source 1..20: mol_type = other RNA
tgcaggcacacacatcaggc

SEQ ID NO: 392 - DNA, 20 bp, synthetic construct
  source 1..20: mol_type = other DNA
  modified_base order(1..5,7,8,10,11,13,14,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(6,9,12,15): mod_base = OTHER; note = Locked nucleic acid
tgcaggcacacacatcaggc

SEQ ID NO: 393 - DNA, 20 bp, synthetic construct
  source 1..20: mol_type = other DNA
  modified_base order(1..3,5,7,9,11,13,15,17,19,20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(4,6,8,10,12,14,16,18): mod_base = OTHER; note = Locked nucleic acid
tgcaggcacacacatcaggc

SEQ ID NO: 394 - DNA, 20 bp, synthetic construct
  source 1..20: mol_type = other DNA
  modified_base order(1,2,4,6,8,10,12,14,16,18..20): mod_base = OTHER; note = 2-prime-O-(2-Methoxyethyl) nucleotide
  modified_base order(3,5,7,9,11,13,15,17): mod_base = OTHER; note = Locked nucleic acid
  modified_base order(3,7,9,11,13,16,20): mod_base = OTHER; note = 5-methyl on the cytidine
tgcaggcacacacatcaggc

SEQ ID NO: 395 - DNA, 20 bp, synthetic construct
  source 1..20: mol_type = other DNA
  modified_base order(1,2,4,5,7,8,10,11,13,14,16,17,19,20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(3,6,9,12,15,18): mod_base = OTHER; note = Locked nucleic acid
tgcaggcacacacatcaggc

SEQ ID NO: 396 - DNA, 20 bp, synthetic construct
  source 1..20: mol_type = other DNA
  modified_base order(1..4,6..8,10..12,14..16,18..20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(5,9,13,17): mod_base = OTHER; note = Locked nucleic acid
tgcaggcacacacatcaggc

SEQ ID NO: 397 - DNA, 20 bp, synthetic construct
  source 1..20: mol_type = other DNA
  modified_base order(1..3,5..7,9..11,13..15,17..20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(4,8,12,16): mod_base = OTHER; note = Locked nucleic acid
tgcaggcacacacatcaggc

SEQ ID NO: 398 - DNA, 20 bp, synthetic construct
  source 1..20: mol_type = other DNA
  modified_base order(1..5,7..10,12..15,17..20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(6,11,16): mod_base = OTHER; note = Locked nucleic acid
tgcaggcacacacatcaggc

SEQ ID NO: 399 - DNA, 20 bp, synthetic construct
  source 1..20: mol_type = other DNA
  modified_base order(1..4,6..9,11..14,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(5,10,15): mod_base = OTHER; note = Locked nucleic acid
tgcaggcacacacatcaggc

SEQ ID NO: 400 - DNA, 20 bp, synthetic construct
  source 1..20: mol_type = other DNA
  modified_base order(2,4,17,19): mod_base = OTHER; note = 2-prime-O-(2-Methoxyethyl) nucleotide
  modified_base order(3,16,20): mod_base = OTHER; note = 5-methyl on the cytidine
  modified_base order(1,3,5,16,18,20): mod_base = OTHER; note = Locked nucleic acid
  misc_feature 6..15: note = DNA
  misc_feature order(1..5,16..20): note = RNA
tgcaggcacacacatcaggc

SEQ ID NO: 401 - DNA, 20 bp, synthetic construct
  source 1..20: mol_type = other DNA
  modified_base order(2,4,6..15,17,19): mod_base = OTHER; note = 2-prime-O-(2-Methoxyethyl) nucleotide
  modified_base order(3,7,9,13,16,20): mod_base = OTHER; note = 5-methyl on the cytidine
  modified_base order(1,3,5,16,18,20): mod_base = OTHER; note = Locked nucleic acid
tgcaggcacacacatcaggc

SEQ ID NO: 402 - DNA, 20 bp, synthetic construct
  source 1..20: mol_type = other DNA
  modified_base order(1..6,9,12,15..20): mod_base = OTHER; note = 2-prime-O-(2-Methoxyethyl) nucleotide
  modified_base order(3,9,16,20): mod_base = OTHER; note = 5-methyl on the cytidine
  misc_feature order(7,8,10,11,13,14): note = DNA
  misc_feature order(1..6,9,12,15..20): note = RNA
tgcaggcacacacatcaggc

SEQ ID NO: 403 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  misc_feature 6..15: note = DNA
  misc_feature order(1..5,16..20): note = RNA
  modified_base order(4^5,16^17): mod_base = OTHER; note = Phosphodiester linkage
tgcaggcacacacatcaggc

SEQ ID NO: 404 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  misc_feature 6..15: note = DNA
  misc_feature order(1..5,16..20): note = RNA
  modified_base order(3^4,4^5,16^17,17^18): mod_base = OTHER; note = Phosphodiester linkage
tgcaggcacacacatcaggc

SEQ ID NO: 405 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  misc_feature 6..15: note = DNA
  misc_feature order(1..5,16..20): note = RNA
  modified_base order(2^3,3^4,4^5,16^17,17^18,18^19): mod_base = OTHER; note = Phosphodiester linkage
tgcaggcacacacatcaggc

SEQ ID NO: 406 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  misc_feature 6..15: note = DNA
  misc_feature order(1..5,16..20): note = RNA
  modified_base order(1^2,2^3,3^4,4^5): mod_base = OTHER; note = Phosphodiester linkage
  modified_base order(16^17,17^18,18^19,19^20): mod_base = OTHER; note = Phosphodiester linkage
tgcaggcacacacatcaggc

SEQ ID NO: 407 - DNA, 22 bp, synthetic construct
  source 1..22: mol_type = other DNA
  modified_base order(1..6,17..22): mod_base = OTHER; note = 2-prime-O-(2-Methoxyethyl) nucleotide
  modified_base order(3,20): mod_base = OTHER; note = 5-methyl on the cytidine
  misc_feature 7..16: note = DNA
  misc_feature order(1..16,17..22): note = RNA
tgcaggcacacacatcaggctg

SEQ ID NO: 408 - DNA, 20 bp, synthetic construct
  source 1..20: mol_type = other DNA
  modified_base order(1,2,4,17,19,20): mod_base = OTHER; note = 2-prime-O-(2-Methoxyethyl) nucleotide
  modified_base order(3,16,20): mod_base = OTHER; note = 5-methyl on the cytidine
  modified_base order(3,5,16,18): mod_base = OTHER; note = Locked nucleic acid
  misc_feature order(1..5,16..20): note = RNA
  misc_feature 6..15: note = DNA
tgcaggcacacacatcaggc

SEQ ID NO: 409 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(3^4,4^5,16^17,17^18): mod_base = OTHER; note = Phosphodiester linkage
  misc_feature order(1..5,16..20): note = RNA
  misc_feature 6..15: note = DNA
tgcaggcacacacatcaggc

SEQ ID NO: 410 - DNA, 19 bp, synthetic construct
  source 1..19: mol_type = other DNA
  modified_base order(1..5,16..19): mod_base = OTHER; note = 2-prime-O-(2-Methoxyethyl) nucleotide
  modified_base order(3,16): mod_base = OTHER; note = 5-methyl on the cytidine
  misc_feature order(1..5,16..19): note = RNA
  misc_feature 6..15: note = DNA
  modified_base order(3^4,4^5,16^17,17^18): mod_base = OTHER; note = Phosphodiester linkage
tgcaggcacacacatcagg

SEQ ID NO: 411 - DNA, 18 bp, synthetic construct
  source 1..18: mol_type = other DNA
  modified_base order(1..5,16..18): mod_base = OTHER; note = 2-prime-O-(2-Methoxyethyl) nucleotide
  modified_base order(2,16): mod_base = OTHER; note = 5-methyl on the cytidine
  modified_base order(3^4,4^5,16^17,17^18): mod_base = OTHER; note = Phosphodiester linkage
  misc_feature order(1..5,16..18): note = RNA
  misc_feature 6..15: note = DNA
tgcaggcacacacatcag

SEQ ID NO: 412 - DNA, 15 bp, synthetic construct
  source 1..15: mol_type = other DNA
  modified_base 1..5: mod_base = OTHER; note = 2-prime-O-(2-Methoxyethyl) nucleotide
  modified_base 3: mod_base = OTHER; note = 5-methyl on the cytidine
  modified_base order(3^4,4^5): mod_base = OTHER; note = Phosphodiester linkage
  misc_feature 1..5: note = RNA
  misc_feature 6..15: note = DNA
tgcaggcacacacat

SEQ ID NO: 413 - skipped

SEQ ID NO: 414 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(3^4,4^5,16^17,17^18): mod_base = OTHER; note = Phosphodiester linkage
  misc_feature order(1..5,16..20): note = RNA
  misc_feature 6..15: note = DNA
tgcaggcacacacatcaggc

SEQ ID NO: 415 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(3^4,4^5,5^6,6^7,7^8,8^9,9^10,10^11,11^12,12^13,13^14,14^15,15^16,16^17,17^18): mod_base = OTHER; note = Phosphodiester linkage
  misc_feature order(1..5,16..20): note = RNA
  misc_feature 6..15: note = DNA
tgcaggcacacacatcaggc

SEQ ID NO: 416 - DNA, 20 bp, synthetic construct
  source 1..20: mol_type = other DNA
  modified_base order(3,7,9,11,13,16,20): mod_base = OTHER; note = 5-methyl on the cytidine
tgcaggcacacacatcaggc

SEQ ID NO: 417 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  misc_feature order(1..5,16..20): note = RNA
  misc_feature 6..15: note = DNA
tgcaggcacacacatcaggc

SEQ ID NO: 418 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(4^5,16^17): mod_base = OTHER; note = Phosphodiester linkage
  misc_feature order(1..5,16..20): note = RNA
  misc_feature 6..15: note = DNA
tgcaggcacacacatcaggc

SEQ ID NO: 419 - DNA, 20 bp, synthetic construct
  source 1..20: mol_type = other DNA
  modified_base order(1..5,16,20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(3^4,4^5,16^17,17^18): mod_base = OTHER; note = Phosphodiester linkage
  misc_feature order(1..5,16..20): note = RNA
  modified_base 6..15: mod_base = OTHER; note = DNA
tgcaggcacacacatcaggc

SEQ ID NO: 420 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(1^2,2^3,3^4,4^5,16^17,17^18,18^19,19^20): mod_base = OTHER; note = Phosphodiester linkage
  misc_feature order(1..5,16..20): note = RNA
  misc_feature 6..15: note = DNA
tgcaggcacacacatcaggc

SEQ ID NO: 421 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  misc_feature order(1..5,16..20): note = RNA
  misc_feature 6..15: note = DNA
tgcaggcacacacatcaggc

SEQ ID NO: 422 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  misc_feature order(1..5,16..20): note = RNA
  modified_base 6..15: mod_base = OTHER; note = DNA
  modified_base order(4^5,16^17): mod_base = OTHER; note = Phosphodiester linkage
tgcaggcacacacatcaggc

SEQ ID NO: 423 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(3^4,4^5,16^17,17^18): mod_base = OTHER; note = Phosphodiester linkage
  misc_feature order(1..5,16..20): note = RNA
  misc_feature 6..15: note = DNA
tgcaggcacacacatcaggc

SEQ ID NO: 424 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(1^2,2^3,3^4,4^5,16^17,17^18,18^19,19^20): mod_base = OTHER; note = Phosphodiester linkage
  misc_feature order(1..5,16..20): note = RNA
  misc_feature 6..15: note = DNA
tgcaggcacacacatcaggc

SEQ ID NO: 425 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(5,9,11,13,15): mod_base = OTHER; note = 5-methyl on the cytidine
  misc_feature order(1..3,14..16): note = RNA
  misc_feature 4..13: note = DNA
aatgcaggcacacaca

SEQ ID NO: 426 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(7,11,13,15): mod_base = OTHER; note = 5-methyl on the cytidine
  misc_feature 4..13: note = DNA
  misc_feature order(1..3,14..16): note = RNA
tgaatgcaggcacaca

SEQ ID NO: 427 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(1,9,13,15): mod_base = OTHER; note = 5-methyl on the cytidine
  misc_feature 4..13: note = DNA
  misc_feature order(1..3,14..16): note = RNA
catgaatgcaggcaca

SEQ ID NO: 428 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(3,7,9,11,13,16): mod_base = OTHER; note = 5-methyl on the cytidine
  misc_feature 4..13: note = DNA
  misc_feature order(1..3,14..16): note = RNA
tgcaggcacacacatc

SEQ ID NO: 429 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(1,5,7,9,11,14): mod_base = OTHER; note = 5-methyl on the cytidine
  misc_feature 4..13: note = DNA
  misc_feature order(1..3,14..16): note = RNA
caggcacacacatcag

SEQ ID NO: 430 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(3,5,7,9,12,16): mod_base = OTHER; note = 5-methyl on the cytidine
  misc_feature 4..13: note = DNA
  misc_feature order(1..3,14..16): note = RNA
ggcacacacatcaggc

SEQ ID NO: 431 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(1,3,5,7,10,14): mod_base = OTHER; note = 5-methyl on the cytidine
  misc_feature 4..13: note = DNA
  misc_feature order(1..3,14..16): note = RNA
cacacacatcaggctg

SEQ ID NO: 432 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(1,3,5,8,12): mod_base = OTHER; note = 5-methyl on the cytidine
  misc_feature 4..13: note = DNA
  misc_feature order(1..3,14..16): note = RNA
cacacatcaggctggg

SEQ ID NO: 433 - DNA, 30 bp, synthetic construct
  source 1..30: mol_type = other DNA
  modified_base order(1..5,16..20): mod_base = OTHER; note = 2-prime-O-(2-Methoxyethyl) nucleotide
  modified_base order(3,7,9,11,13,16,20): mod_base = OTHER; note = 5-methyl on the cytidine
  modified_base order(1^2,2^3,3^4,4^5,5^6,6^7,7^8,8^9,9^10,10^11,11^12,12^13,13^14,14^15): mod_base = OTHER; note = 5-methyl on the cytidine
  modified_base order(15^16,16^17,17^18,18^19,19^20,20^21): mod_base = OTHER; note = 5-methyl on the cytidine
  modified_base order(21^22,22^23,23^24,24^25,25^26,26^27,27^28,28^29,29^30): mod_base = OTHER; note = 5-methyl on the cytidine
  misc_feature order(1..5,14..18): note = RNA
  misc_feature order(6..15,21..30): note = DNA
tgcaggcacacacatcaggctttttttttt

SEQ ID NO: 434 - DNA, 30 bp, synthetic construct
  source 1..30: mol_type = other DNA
  modified_base order(11..15,26..30): mod_base = OTHER; note = 2-prime-O-(2-Methoxyethyl) nucleotide
  modified_base order(13,17,19,21,23,26,30): mod_base = OTHER; note = 5-methyl on the cytidine
  modified_base order(1^2,2^3,3^4,4^5,5^6,6^7,7^8,8^9,9^10,10^11,11^12,12^13,13^14,14^15,15^16,16^17,17^18,18^19,19^20,20^21,21^22,22^23,23^24,24^25,25^26,26^27,27^28,28^29,29^30): mod_base = OTHER; note = Phosphodiester linkage
  misc_feature order(1..10,16..25): note = DNA
  misc_feature order(11..15,26..30): note = RNA
tttttttttttgcaggcacacacatcaggc

SEQ ID NO: 435 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(3,11,15): mod_base = OTHER; note = 5-methyl on the cytidine
  misc_feature 4..13: note = DNA
  misc_feature order(1..3,14..16): note = RNA
ggcatgaatgcaggca

SEQ ID NO: 436 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(1,5,13): mod_base = OTHER; note = 5-methyl on the cytidine
  misc_feature 4..13: note = DNA
  misc_feature order(1..3,14..16): note = RNA
caggcatgaatgcagg

SEQ ID NO: 437 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(3,7,15): mod_base = OTHER; note = 5-methyl on the cytidine
  misc_feature 4..13: note = DNA
  misc_feature order(1..3,14..16): note = RNA
gacaggcatgaatgca

SEQ ID NO: 438 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(5,9): mod_base = OTHER; note = 5-methyl on the cytidine
  misc_feature order(1..3,14..16): note = RNA
  misc_feature 4..13: note = DNA
gtgacaggcatgaatg

SEQ ID NO: 439 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(1,3,6): mod_base = OTHER; note = 5-methyl on the cytidine
  misc_feature order(1..3,14..16): note = RNA
  misc_feature 4..13: note = DNA
cacatcaggctgggga

SEQ ID NO: 440 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(4,15,16): mod_base = OTHER; note = 5-methyl on the cytidine
  misc_feature 4..13: note = DNA
  misc_feature order(1..3,14..16): note = RNA
catcaggctggggacc

SEQ ID NO: 441 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  misc_feature order(1..3,14..16): note = RNA
  modified_base order(2,6,13..15): mod_base = OTHER; note = 5-methyl on the cytidine
  misc_feature 4..14: note = DNA
tcaggctggggaccct

SEQ ID NO: 442 - DNA, 16 bp, synthetic construct
  source 1..16: mol_type = other DNA
  modified_base order(1..3,14..16): mod_base = OTHER; note = Constrained ethyl 2-prime-4-prime-bridged nucleic acid
  modified_base order(4,11..13): mod_base = OTHER; note = 5-methyl on the cytidine
  misc_feature order(1..3,14..16): note = RNA
  misc_feature 4..13: note = DNA
aggctggggaccctgt

SEQ ID NO: 443 - DNA, 16 bp, synthetic construct
  source 1..16: mol_type = other DNA
  modified_base order(1..3,14..16): mod_base = OTHER; note = Locked nucleic acid
  modified_base order(1,9,13,15): mod_base = OTHER; note = 5-methyl on the cytidine
  misc_feature 4..13: note = DNA
  misc_feature order(1..3,14..16): note = RNA
catgaatgcaggcaca

SEQ ID NO: 444 - DNA, 16 bp, synthetic construct
  source 1..16: mol_type = other DNA
  modified_base order(1..3,14..16): mod_base = OTHER; note = Locked nucleic acid
  modified_base order(7,11,13,15): mod_base = OTHER; note = 5-methyl on the cytidine
  misc_feature order(1..3,14..16): note = RNA
  misc_feature 4..13: note = DNA
tgaatgcaggcacaca

SEQ ID NO: 445 - DNA, 16 bp, synthetic construct
  source 1..16: mol_type = other DNA
  modified_base order(1..3,14..16): mod_base = OTHER; note = Locked nucleic acid
  modified_base order(5,9,11,13,15): mod_base = OTHER; note = 5-methyl on the cytidine
  misc_feature order(1..3,14..16): note = RNA
  misc_feature 4..13: note = DNA
aatgcaggcacacaca

SEQ ID NO: 446 - DNA, 16 bp, synthetic construct
  source 1..16: mol_type = other DNA
  modified_base order(1..3,14..16): mod_base = OTHER; note = Locked nucleic acid
  modified_base order(3,7,9,11,13,16): mod_base = OTHER; note = 5-methyl on the cytidine
  misc_feature order(1..3,14..16): note = RNA
  misc_feature 4..13: note = DNA
tgcaggcacacacatc

SEQ ID NO: 447 - DNA, 16 bp, synthetic construct
  source 1..16: mol_type = other DNA
  modified_base order(1..3,14..16): mod_base = OTHER; note = Locked nucleic acid
  modified_base order(1,5,7,9,11,14): mod_base = OTHER; note = 5-methyl on the cytidine
  misc_feature order(1..3,14..16): note = RNA
  misc_feature 4..13: note = DNA
caggcacacacatcag

SEQ ID NO: 448 - DNA, 16 bp, synthetic construct
  source 1..16: mol_type = other DNA
  modified_base order(1..3,14..16): mod_base = OTHER; note = Locked nucleic acid
  modified_base order(3,5,7,9,12,16): mod_base = OTHER; note = 5-methyl on the cytidine
  misc_feature order(1..3,14..16): note = RNA
  misc_feature 4..13: note = DNA
ggcacacacatcaggc

SEQ ID NO: 449 - DNA, 16 bp, synthetic construct
  source 1..16: mol_type = other DNA
  modified_base order(1..3,14..16): mod_base = OTHER; note = Locked nucleic acid
  modified_base order(1,3,5,7,10,14): mod_base = OTHER; note = 5-methyl on the cytidine
  misc_feature order(1..3,14..16): note = RNA
  misc_feature 4..13: note = DNA
cacacacatcaggctg

SEQ ID NO: 450 - DNA, 16 bp, synthetic construct
  source 1..16: mol_type = other DNA
  modified_base order(1..3,14..16): mod_base = OTHER; note = Locked nucleic acid
  modified_base order(1,3,5,8,12): mod_base = OTHER; note = 5-methyl on the cytidine
  misc_feature order(1..3,14..16): note = RNA
  misc_feature 4..13: note = DNA
cacacatcaggctggg

SEQ ID NO: 451 - DNA, 16 bp, synthetic construct
  source 1..16: mol_type = other DNA
  modified_base order(1..3,14..16): mod_base = OTHER; note = Locked nucleic acid
  modified_base order(3,11,15): mod_base = OTHER; note = 5-methyl on the cytidine
  misc_feature order(1..3,14..16): note = RNA
  misc_feature 4..13: note = DNA
ggcatgaatgcaggca

SEQ ID NO: 452 - DNA, 16 bp, synthetic construct
  source 1..16: mol_type = other DNA
  modified_base order(1..3,14..16): mod_base = OTHER; note = Locked nucleic acid
  modified_base order(1,5,13): mod_base = OTHER; note = 5-methyl on the cytidine
  misc_feature order(1..3,14..16): note = RNA
  misc_feature 4..13: note = DNA
caggcatgaatgcagg

SEQ ID NO: 453 - DNA, 16 bp, synthetic construct
  source 1..16: mol_type = other DNA
  modified_base order(1..3,14..16): mod_base = OTHER; note = Locked nucleic acid
  modified_base order(3,7,15): mod_base = OTHER; note = Locked nucleic acid
  misc_feature order(1..3,14..16): note = RNA
  misc_feature 4..13: note = DNA
gacaggcatgaatgca

SEQ ID NO: 454 - DNA, 16 bp, synthetic construct
  source 1..16: mol_type = other DNA
  modified_base order(1..3,14..16): mod_base = OTHER; note = Locked nucleic acid
  modified_base order(5,9): mod_base = OTHER; note = 5-methyl on the cytidine
  misc_feature order(1..3,14..16): note = RNA
  misc_feature 4..13: note = DNA
gtgacaggcatgaatg

SEQ ID NO: 455 - DNA, 16 bp, synthetic construct
  source 1..16: mol_type = other DNA
  modified_base order(1..3,14..16): mod_base = OTHER; note = Locked nucleic acid
  modified_base order(1,3,6,10): mod_base = OTHER; note = 5-methyl on the cytidine
  misc_feature order(1..3,14..16): note = RNA
  misc_feature 4..13: note = DNA
cacatcaggctgggga

SEQ ID NO: 456 - DNA, 16 bp, synthetic construct
  source 1..16: mol_type = other DNA
  modified_base order(1..3,14..16): mod_base = OTHER; note = Locked nucleic acid
  modified_base order(1,4,8,15,16): mod_base = OTHER; note = 5-methyl on the cytidine
  misc_feature order(1..3,14..16): note = RNA
  misc_feature 4..13: note = DNA
catcaggctggggacc

SEQ ID NO: 457 - DNA, 16 bp, synthetic construct
  source 1..16: mol_type = other DNA
  modified_base order(1..3,14..16): mod_base = OTHER; note = Locked nucleic acid
  modified_base order(2,6,13,15): mod_base = OTHER; note = 5-methyl on the cytidine
  misc_feature order(1..3,14..16): note = RNA
  misc_feature 4..13: note = DNA
tcaggctggggaccct

SEQ ID NO: 458 - DNA, 16 bp, synthetic construct
  source 1..16: mol_type = other DNA
  modified_base order(1..3,14..16): mod_base = OTHER; note = Locked nucleic acid
  modified_base order(4,11..13): mod_base = OTHER; note = 5-methyl on the cytidine
  misc_feature order(1..3,14..16): note = RNA
  misc_feature 4..13: note = DNA
aggctggggaccctgt

SEQ ID NO: 459 - DNA, 18 bp, synthetic construct
  source 1..18: mol_type = other DNA
  modified_base order(1..4,15..18): mod_base = OTHER; note = 2-prime-O-(2-Methoxyethyl) nucleotide
  modified_base order(2,10,14,16,18): mod_base = OTHER; note = 5-methyl on the cytidine
  misc_feature 5..14: note = DNA
  misc_feature order(1..4,15..18): note = RNA
gcatgaatgcaggcacac

SEQ ID NO: 460 - DNA, 18 bp, synthetic construct
  source 1..18: mol_type = other DNA
  modified_base order(1,3,16,18): mod_base = OTHER; note = Locked nucleic acid
  modified_base order(2,4,15,17): mod_base = OTHER; note = 2-prime-O-(2-Methoxyethyl) nucleotide
  modified_base order(2,10,14,16,18): mod_base = OTHER; note = 5-methyl on the cytidine
  misc_feature order(1..4,15..18): note = RNA
  misc_feature 5..14: note = DNA
gcatgaatgcaggcacac

SEQ ID NO: 461 - DNA, 18 bp, synthetic construct
  source 1..18: mol_type = other DNA
  modified_base order(1..4,15..18): mod_base = OTHER; note = 2-prime-O-(2-Methoxyethyl) nucleotide
  modified_base order(2,4,6,8,11,15): mod_base = OTHER; note = 5-methyl on the cytidine
  misc_feature 5..14: note = DNA
  misc_feature order(1..4,15..18): note = RNA
gcacacacatcaggctgg

SEQ ID NO: 462 - DNA, 18 bp, synthetic construct
  source 1..18: mol_type = other DNA
  modified_base order(2,4,15,17): mod_base = OTHER; note = 2-prime-O-(2-Methoxyethyl) nucleotide
  modified_base order(1,3,16,18): mod_base = OTHER; note = Locked nucleic acid
  modified_base order(2,4,6,8,11,15): mod_base = OTHER; note = 5-methyl on the cytidine
  misc_feature order(1..4,15..18): note = RNA
  modified_base 5..14: mod_base = OTHER; note = 5-methyl on the cytidine
gcacacacatcaggctgg

SEQ ID NO: 463 - DNA, 18 bp, synthetic construct
  source 1..18: mol_type = other DNA
  modified_base order(1..4,15..18): mod_base = OTHER; note = 2-prime-O-(2-Methoxyethyl) nucleotide
  modified_base order(2,4,6,8,11,15): mod_base = OTHER; note = 5-methyl on the cytidine
  misc_feature order(1..4,15..18): note = RNA
  misc_feature 5..14: note = DNA
acacacatcaggctgggg

SEQ ID NO: 464 - DNA, 18 bp, synthetic construct
  source 1..18: mol_type = other DNA
  modified_base order(2,4,15,17): mod_base = OTHER; note = 2-prime-O-(2-Methoxyethyl) nucleotide
  modified_base order(1,3,16,18): mod_base = OTHER; note = Locked nucleic acid
  modified_base order(2,4,6,9,13): mod_base = OTHER; note = 5-methyl on the cytidine
  misc_feature order(1..4,15..18): note = RNA
  misc_feature 5..14: note = DNA
acacacatcaggctgggg

SEQ ID NO: 465 - DNA, 18 bp, synthetic construct
  source 1..18: mol_type = other DNA
  modified_base order(2..4,15..17): mod_base = OTHER; note = 2-prime-O-(2-Methoxyethyl) nucleotide
  modified_base order(1,18): mod_base = OTHER; note = Locked nucleic acid
  modified_base order(1,3,5,7,10,14): mod_base = OTHER; note = 5-methyl on the cytidine
  misc_feature 5..14: note = DNA
  misc_feature order(1..4,15..18): note = RNA
cacacacatcaggctggg

SEQ ID NO: 466 - DNA, 18 bp, synthetic construct
  source 1..18: mol_type = other DNA
  modified_base order(2,4,15,17): mod_base = OTHER; note = 2-prime-O-(2-Methoxyethyl) nucleotide
  modified_base order(1,3,16,18): mod_base = OTHER; note = Locked nucleic acid
  modified_base order(1,3,5,7,10,14): mod_base = OTHER; note = 5-methyl on the cytidine
  misc_feature 5..14: note = DNA
  misc_feature order(1..4,15..18): note = RNA
cacacacatcaggctggg

SEQ ID NO: 467 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,11,15,17,19): mod_base = OTHER; note = 5-methyl on the cytidine
  misc_feature order(1..5,16..20): note = RNA
  misc_feature 6..15: note = DNA
ggcatgaatgcaggcacaca

SEQ ID NO: 468 - DNA, 20 bp, synthetic construct
  source 1..20: mol_type = other DNA
  modified_base order(2,4,5,16,17,19): mod_base = OTHER; note = 2-prime-O-(2-Methoxyethyl) nucleotide
  modified_base order(1,3,18,20): mod_base = OTHER; note = Locked nucleic acid
  modified_base order(3,11,15,17,19): mod_base = OTHER; note = 5-methyl on the cytidine
  misc_feature order(1..5,16..20): note = RNA
  misc_feature 6..15: note = DNA
ggcatgaatgcaggcacaca

SEQ ID NO: 469 - DNA, 20 bp, synthetic construct
  source 1..20: mol_type = other DNA
  modified_base order(2,4,5,16,17,19): mod_base = OTHER; note = 2-prime-O-(2-Methoxyethyl) nucleotide
  modified_base order(1,3,9,12,18,20): mod_base = OTHER; note = Locked nucleic acid
  modified_base order(3,11,15,17,19): mod_base = OTHER; note = 5-methyl on the cytidine
  misc_feature order(1..5,16..20): note = RNA
  misc_feature 6..15: note = DNA
ggcatgaatgcaggcacaca

SEQ ID NO: 470 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,11,15,17,19): mod_base = OTHER; note = 5-methyl on the cytidine
  misc_feature order(1..5,16..20): note = RNA
  misc_feature 6..15: note = DNA
ggcatgaatgcaggcacaca

SEQ ID NO: 471 - DNA, 20 bp, synthetic construct
  source 1..20: mol_type = other DNA
  modified_base order(1,3,18,20): mod_base = OTHER; note = Locked nucleic acid
  modified_base order(2,4,5,16,17,19): mod_base = OTHER; note = 2-prime-O-(2-Methoxyethyl) nucleotide
  modified_base order(3,5,7,9,12,16): mod_base = OTHER; note = 5-methyl on the cytidine
  misc_feature order(1..5,16..20): note = RNA
  misc_feature 6..15: note = DNA
ggcacacacatcaggctggg

SEQ ID NO: 472 - DNA, 20 bp, synthetic construct
  source 1..20: mol_type = other DNA
  modified_base order(2,4,5,16,17,19): mod_base = OTHER; note = 2-prime-O-(2-Methoxyethyl) nucleotide
  modified_base order(1,3,9,12,18,20): mod_base = OTHER; note = Locked nucleic acid
  modified_base order(3,5,7,9,12,16): mod_base = OTHER; note = 5-methyl on the cytidine
  misc_feature order(1..5,9,12,16..20): note = RNA
  misc_feature 6..15: note = DNA
ggcacacacatcaggctggg

SEQ ID NO: 473 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,5,7,9,12,16): mod_base = OTHER; note = 5-methyl on the cytidine
  modified_base order(3^4,4^5,16^17,17^18): mod_base = OTHER; note = Phosphodiester linkage
  misc_feature order(1..5,16..20): note = RNA
  misc_feature 6..15: note = DNA
ggcacacacatcaggctggg

SEQ ID NO: 474 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(1,3,5,7,10,14): mod_base = OTHER; note = 5-methyl on the cytidine
  misc_feature order(1..5,16..20): note = RNA
  misc_feature 6..15: note = DNA
cacacacatcaggctgggga

SEQ ID NO: 475 - DNA, 20 bp, synthetic construct
  source 1..20: mol_type = other DNA
  modified_base order(2,4,5,16,17,19): mod_base = OTHER; note = 2-prime-O-(2-Methoxyethyl) nucleotide
  modified_base order(1,3,18,20): mod_base = OTHER; note = Locked nucleic acid
  modified_base order(1,3,5,7,10,14): mod_base = OTHER; note = 5-methyl on the cytidine
  misc_feature order(1..5,16..20): note = RNA
  misc_feature 6..15: note = DNA
cacacacatcaggctgggga

SEQ ID NO: 476 - DNA, 20 bp, synthetic construct
  source 1..20: mol_type = other DNA
  modified_base order(2,4,5,16,17,19): mod_base = OTHER; note = 2-prime-O-(2-Methoxyethyl) nucleotide
  modified_base order(1,3,9,12,18,20): mod_base = OTHER; note = Locked nucleic acid
  modified_base order(1,3,5,7,10,14): mod_base = OTHER; note = 5-methyl on the cytidine
  misc_feature order(1..5,9,12,16..20): note = RNA
  misc_feature order(6..8,10,11,13..15): note = DNA
cacacacatcaggctgggga

SEQ ID NO: 477 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(1,3,5,7,10,14): mod_base = OTHER; note = 5-methyl on the cytidine
  modified_base order(3^4,4^5,16^17,17^18): mod_base = OTHER; note = Phosphodiester linkage
  misc_feature order(1..5,16..20): note = RNA
  misc_feature 6..15: note = DNA
cacacacatcaggctgggga

SEQ ID NO: 478 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(2,4,6,8,11,15): mod_base = OTHER; note = 5-methyl on the cytidine
  misc_feature order(1..5,16..20): note = RNA
  misc_feature 6..15: note = DNA
gcacacacatcaggctgggg

SEQ ID NO: 479 - DNA, 20 bp, synthetic construct
  source 1..20: mol_type = other DNA
  modified_base order(2,4,5,16,17,19): mod_base = OTHER; note = 2-prime-O-(2-Methoxyethyl) nucleotide
  modified_base order(1,3,18,20): mod_base = OTHER; note = Locked nucleic acid
  modified_base order(2,4,6,8,11,15): mod_base = OTHER; note = 5-methyl on the cytidine
  misc_feature 6..15: note = DNA
  misc_feature order(1..5,16..20): note = RNA
gcacacacatcaggctgggg

SEQ ID NO: 480 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(2,4,6,8,11,15): mod_base = OTHER; note = 5-methyl on the cytidine
  modified_base order(3^4,4^5,16^17,17^18): mod_base = OTHER; note = Phosphodiester linkage
  misc_feature order(1..5,16..18): note = RNA
  modified_base 6..15: mod_base = OTHER; note = DNA
gcacacacatcaggctgggg

SEQ ID NO: 481 - DNA, 20 bp, synthetic construct
  source 1..20: mol_type = other DNA
  modified_base order(3,7,9,11,13,16,20): mod_base = OTHER; note = 5-methyl on the cytidine
tgcaggcacacacatcaggc

SEQ ID NO: 482 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,7,14,15,16): mod_base = OTHER; note = 5-methyl on the cytidine
  misc_feature 6..15: note = DNA
  misc_feature order(1..5,16..20): note = RNA
atcaggctggggaccctgtg

SEQ ID NO: 483 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,5,7,9,12,16): mod_base = OTHER; note = 5-methyl on the cytidine
  misc_feature 6..15: note = DNA
  misc_feature order(1..5,16..20): note = RNA
ggcacacacatcaggctggg

SEQ ID NO: 484 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(8,12,14,16,18): mod_base = OTHER; note = 5-methyl on the cytidine
  misc_feature order(1..5,16..20): note = RNA
  misc_feature 6..15: note = DNA
atgaatgcaggcacacacat

SEQ ID NO: 485 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  misc_feature order(1..5,16..20): note = RNA
  modified_base order(3,7,9,11,13,16,20): mod_base = OTHER; note = 5-methyl on the cytidine
  misc_feature 6..15: note = DNA
tgcaggcacacacatcaggc

SEQ ID NO: 486 - DNA, 20 bp, synthetic construct
  source 1..20: mol_type = other DNA
  modified_base order(1..5,16..20): mod_base = OTHER; note = 2-prime-O-(2-Methoxyethyl) nucleotide
  modified_base order(3,9,11,13,16,20): mod_base = OTHER; note = 5-methyl on the cytidine
  misc_feature order(1..5,7,16..20): note = RNA
  misc_feature order(6,8..15): note = DNA
  modified_base 7: mod_base = OTHER; note = Constrained ethyl 2-prime-4-prime-bridged nucleic acid
  modified_base order(3^4,4^5,16^17,17^18): mod_base = OTHER; note = Phosphodiester linkage
tgcaggcacacacatcaggc

SEQ ID NO: 487 - RNA, 20 nt, synthetic construct
  source 1..20: mol_type = other RNA
gcaatgacaaggtatttgat

SEQ ID NO: 488 - RNA, 20 nt, synthetic construct
  source 1..20: mol_type = other RNA
ggactacaagttgttgcaat

SEQ ID NO: 489 - RNA, 20 nt, synthetic construct
  source 1..20: mol_type = other RNA
gttagatcaaagagtcctta

SEQ ID NO: 490 - RNA, 20 nt, synthetic construct
  source 1..20: mol_type = other RNA
ttagtagagggttagatcaa

SEQ ID NO: 491 - RNA, 20 nt, synthetic construct
  source 1..20: mol_type = other RNA
gtagaaggacttagtagagg

SEQ ID NO: 492 - RNA, 20 nt, synthetic construct
  source 1..20: mol_type = other RNA
gtcacacatgaacaattctc

SEQ ID NO: 493 - RNA, 20 nt, synthetic construct
  source 1..20: mol_type = other RNA
gatgactgatttacttgaca

SEQ ID NO: 494 - RNA, 20 nt, synthetic construct
  source 1..20: mol_type = other RNA
tgtatagtgcaaatgatgac

SEQ ID NO: 495 - RNA, 20 nt, synthetic construct
  source 1..20: mol_type = other RNA
ttttgtcatctgtatagtgc

SEQ ID NO: 496 - RNA, 20 nt, synthetic construct
  source 1..20: mol_type = other RNA
tcacatattctctgaaccta

SEQ ID NO: 497 - RNA, 20 nt, synthetic construct
  source 1..20: mol_type = other RNA
ttcttggacaagtcacatat

SEQ ID NO: 498 - RNA, 20 nt, synthetic construct
  source 1..20: mol_type = other RNA
taatgccctgttcttggaca

SEQ ID NO: 499 - RNA, 20 nt, synthetic construct
  source 1..20: mol_type = other RNA
ctgttatgcatttcctaatg

SEQ ID NO: 500 - RNA, 20 nt, synthetic construct
  source 1..20: mol_type = other RNA
gaatctaacccctgttatgc

SEQ ID NO: 501 - RNA, 20 nt, synthetic construct
  source 1..20: mol_type = other RNA
ggcacaaaaagatctggaat

SEQ ID NO: 502 - RNA, 20 nt, synthetic construct
  source 1..20: mol_type = other RNA
gtaatagtgggatatactct

SEQ ID NO: 503 - RNA, 20 nt, synthetic construct
  source 1..20: mol_type = other RNA
aggctgtaacagtgtaatag

SEQ ID NO: 504 - RNA, 20 nt, synthetic construct
  source 1..20: mol_type = other RNA
ttattacattcagcaggctg

SEQ ID NO: 505 - RNA, 20 nt, synthetic construct
  source 1..20: mol_type = other RNA
gaaaatgttcagcactcatt

SEQ ID NO: 506 - RNA, 20 nt, synthetic construct
  source 1..20: mol_type = other RNA
ggtataaatgatcaacaacc

SEQ ID NO: 507 - RNA, 20 nt, synthetic construct
  source 1..20: mol_type = other RNA
gaaagttgatatcaagggta

SEQ ID NO: 508 - RNA, 20 nt, synthetic construct
  source 1..20: mol_type = other RNA
agaatttcaaggtgctctat

SEQ ID NO: 509 - RNA, 20 nt, synthetic construct
  source 1..20: mol_type = other RNA
ttttacttacaggaaagagg

SEQ ID NO: 510 - RNA, 20 nt, synthetic construct
  source 1..20: mol_type = other RNA
ccaatcggcacagttgtatt

SEQ ID NO: 511 - RNA, 20 nt, synthetic construct
  source 1..20: mol_type = other RNA
tagagtgctttgccaatcgg

SEQ ID NO: 512 - RNA, 20 nt, synthetic construct
  source 1..20: mol_type = other RNA
gcggctaatatttatagagt

SEQ ID NO: 513 - RNA, 20 nt, synthetic construct
  source 1..20: mol_type = other RNA
ggtatttgtcagcggctaat

SEQ ID NO: 514 - RNA, 20 nt, synthetic construct
  source 1..20: mol_type = other RNA
taattttatgtggcccttcc

SEQ ID NO: 515 - RNA, 20 nt, synthetic construct
  source 1..20: mol_type = other RNA
gcaatgacgcacttataatt

SEQ ID NO: 516 - RNA, 20 nt, synthetic construct
  source 1..20: mol_type = other RNA
gtttgacgacagcaatgacg

SEQ ID NO: 517 - RNA, 20 nt, synthetic construct
  source 1..20: mol_type = other RNA
gagacggtgggtttgacgac

SEQ ID NO: 518 - RNA, 20 nt, synthetic construct
  source 1..20: mol_type = other RNA
tttgatgacagagacggtgg

SEQ ID NO: 519 - RNA, 20 nt, synthetic construct
  source 1..20: mol_type = other RNA
ttcctctctctttgatgaca

SEQ ID NO: 520 - RNA, 20 nt, synthetic construct
  source 1..20: mol_type = other RNA
aggtttgcttttcctctctc

SEQ ID NO: 521 - RNA, 20 nt, synthetic construct
  source 1..20: mol_type = other RNA
cgacatatttagaggtttgc

SEQ ID NO: 522 - RNA, 20 nt, synthetic construct
  source 1..20: mol_type = other RNA
gggttgctttttcgacatat

SEQ ID NO: 523 - RNA, 20 nt, synthetic construct
  source 1..20: mol_type = other RNA
cctgtaaattgggttgcttt

SEQ ID NO: 524 - RNA, 20 nt, synthetic construct
  source 1..20: mol_type = other RNA
catctataatcattgccctg

SEQ ID NO: 525 - RNA, 20 nt, synthetic construct
  source 1..20: mol_type = other RNA
agacaccctgtagtcacatc

SEQ ID NO: 526 - RNA, 20 nt, synthetic construct
  source 1..20: mol_type = other RNA
tagatggtaaatggattggg

SEQ ID NO: 527 - RNA, 20 nt, synthetic construct
  source 1..20: mol_type = other RNA
tatgtccagttatagatggt

SEQ ID NO: 528 - RNA, 20 nt, synthetic construct
  source 1..20: mol_type = other RNA
aaggttagtagcattatgtc

SEQ ID NO: 529 - RNA, 20 nt, synthetic construct
  source 1..20: mol_type = other RNA
cagtcactttaaaggttagt

SEQ ID NO: 530 - RNA, 20 nt, synthetic construct
  source 1..20: mol_type = other RNA
gtagttacataatggtcagt

SEQ ID NO: 531 - RNA, 20 nt, synthetic construct
  source 1..20: mol_type = other RNA
atcattaggatgtagttaca

SEQ ID NO: 532 - RNA, 20 nt, synthetic construct
  source 1..20: mol_type = other RNA
gccttcactttaaaaactca

SEQ ID NO: 533 - RNA, 20 nt, synthetic construct
  source 1..20: mol_type = other RNA
acttttcttcctgccttcac

SEQ ID NO: 534 - RNA, 20 nt, synthetic construct
  source 1..20: mol_type = other RNA
tagtcctctatctacttttc

SEQ ID NO: 535 - RNA, 20 nt, synthetic construct
  source 1..20: mol_type = other RNA
gttattgtgaaatagtcctc

SEQ ID NO: 536 - RNA, 20 nt, synthetic construct
  source 1..20: mol_type = other RNA
ggacttgtgttattattcag

SEQ ID NO: 537 - RNA, 20 nt, synthetic construct
  source 1..20: mol_type = other RNA
attccatagaacagcccaag

SEQ ID NO: 538 - RNA, 20 nt, synthetic construct
  source 1..20: mol_type = other RNA
ggaggaatgccaagcacaaa

SEQ ID NO: 539 - RNA, 20 nt, synthetic construct
  source 1..20: mol_type = other RNA
ccagagttttggaggaatgc

SEQ ID NO: 540 - RNA, 20 nt, synthetic construct
  source 1..20: mol_type = other RNA
caatcattactggccagagt

SEQ ID NO: 541 - RNA, 20 nt, synthetic construct
  source 1..20: mol_type = other RNA
gccattactcacactcaatc

SEQ ID NO: 542 - RNA, 20 nt, synthetic construct
  source 1..20: mol_type = other RNA
tcatttcatggtgtaaaagc

SEQ ID NO: 543 - RNA, 20 nt, synthetic construct
  source 1..20: mol_type = other RNA
cactaagccatcatttcatg

SEQ ID NO: 544 - RNA, 20 nt, synthetic construct
  source 1..20: mol_type = other RNA
gacaaatttgtaggagcact

SEQ ID NO: 545 - RNA, 20 nt, synthetic construct
  source 1..20: mol_type = other RNA
acagtgcacagctaagaatg

SEQ ID NO: 546 - RNA, 20 nt, synthetic construct
  source 1..20: mol_type = other RNA
aagggtctgtcacagtgcac

SEQ ID NO: 547 - RNA, 20 nt, synthetic construct
  source 1..20: mol_type = other RNA
acagatctgtttcaagggtc

SEQ ID NO: 548 - RNA, 20 nt, synthetic construct
  source 1..20: mol_type = other RNA
cagttctagctcatggtgac

SEQ ID NO: 549 - RNA, 20 nt, synthetic construct
  source 1..20: mol_type = other RNA
ggatggattactgtgaaagc

SEQ ID NO: 550 - RNA, 20 nt, synthetic construct
  source 1..20: mol_type = other RNA
tggtctaattatgtggatgg

SEQ ID NO: 551 - RNA, 20 nt, synthetic construct
  source 1..20: mol_type = other RNA
ccacgacaaaatggtctaat

SEQ ID NO: 552 - RNA, 20 nt, synthetic construct
  source 1..20: mol_type = other RNA
tccatacaaccaccaccaca

SEQ ID NO: 553 - RNA, 20 nt, synthetic construct
  source 1..20: mol_type = other RNA
caataagtgctccatacaac

SEQ ID NO: 554 - RNA, 20 nt, synthetic construct
  source 1..20: mol_type = other RNA
gaatccaattctccaagatc

SEQ ID NO: 555 - RNA, 20 nt, synthetic construct
  source 1..20: mol_type = other RNA
ggcacagatatgattgaaat

SEQ ID NO: 556 - RNA, 20 nt, synthetic construct
  source 1..20: mol_type = other RNA
gagtacttacttaaaatggc

SEQ ID NO: 557 - RNA, 20 nt, synthetic construct
  source 1..20: mol_type = other RNA
accgcgaaattgagtactta

SEQ ID NO: 558 - RNA, 20 nt, synthetic construct
  source 1..20: mol_type = other RNA
agaccccagaaaccgcgaaa

SEQ ID NO: 559 - RNA, 20 nt, synthetic construct
  source 1..20: mol_type = other RNA
ggtgcatcttcatcagaccc

SEQ ID NO: 560 - RNA, 20 nt, synthetic construct
  source 1..20: mol_type = other RNA
gtaggtaaggataggtgcat

SEQ ID NO: 561 - RNA, 20 nt, synthetic construct
  source 1..20: mol_type = other RNA
cactctgtgagaaggtaggt

SEQ ID NO: 562 - RNA, 20 nt, synthetic construct
  source 1..20: mol_type = other RNA
gaatatcttcttctcactct

SEQ ID NO: 563 - RNA, 20 nt, synthetic construct
  source 1..20: mol_type = other RNA
gggtgacttggaatatcttc

SEQ ID NO: 564 - RNA, 20 nt, synthetic construct
  source 1..20: mol_type = other RNA
taatacttcaaagttgggtg

SEQ ID NO: 565 - RNA, 20 nt, synthetic construct
  source 1..20: mol_type = other RNA
tatgtgccatctaatacttc

SEQ ID NO: 566 - RNA, 20 nt, synthetic construct
  source 1..20: mol_type = other RNA
ccctatgattatatgtgcca

SEQ ID NO: 567 - RNA, 20 nt, synthetic construct
  source 1..20: mol_type = other RNA
gtaatgaaagttatccccta

SEQ ID NO: 568 - RNA, 20 nt, synthetic construct
  source 1..20: mol_type = other RNA
gtactcatgtatcagttcag

SEQ ID NO: 569 - RNA, 20 nt, synthetic construct
  source 1..20: mol_type = other RNA
ttgttagtaatacctcaagg

SEQ ID NO: 570 - RNA, 20 nt, synthetic construct
  source 1..20: mol_type = other RNA
actagatgtaaactatgaga

SEQ ID NO: 571 - RNA, 20 nt, synthetic construct
  source 1..20: mol_type = other RNA
agacattgtaactagatgta

SEQ ID NO: 572 - RNA, 20 nt, synthetic construct
  source 1..20: mol_type = other RNA
tagcaaaagaatgggaaagg

SEQ ID NO: 573 - RNA, 20 nt, synthetic construct
  source 1..20: mol_type = other RNA
aaggttagtggcattatgtc

SEQ ID NO: 574 - RNA, 20 nt, synthetic construct
  source 1..20: mol_type = other RNA
ggagcagtcactaaaggtta

SEQ ID NO: 575 - RNA, 20 nt, synthetic construct
  source 1..20: mol_type = other RNA
gcacgtatgttatatagcct

SEQ ID NO: 576 - RNA, 20 nt, synthetic construct
  source 1..20: mol_type = other RNA
gtaagccagaaagcacgtat

SEQ ID NO: 577 - RNA, 20 nt, synthetic construct
  source 1..20: mol_type = other RNA
cttagaaacatcaggtaagc

SEQ ID NO: 578 - RNA, 20 nt, synthetic construct
  source 1..20: mol_type = other RNA
aggcgaactaatagcttaga

SEQ ID NO: 579 - RNA, 20 nt, synthetic construct
  source 1..20: mol_type = other RNA
ggatgagtactgaggcgaac

SEQ ID NO: 580 - RNA, 20 nt, synthetic construct
  source 1..20: mol_type = other RNA
ttcagtgcttagttcagatt

SEQ ID NO: 581 - RNA, 20 nt, synthetic construct
  source 1..20: mol_type = other RNA
ggctttctacttttcagtgc

SEQ ID NO: 582 - RNA, 20 nt, synthetic construct
  source 1..20: mol_type = other RNA
tctggctttaggctttctac

SEQ ID NO: 583 - RNA, 20 nt, synthetic construct
  source 1..20: mol_type = other RNA
gacaaattgtgctgaaattc

SEQ ID NO: 584 - RNA, 20 nt, synthetic construct
  source 1..20: mol_type = other RNA
ccttgtaactgcagagaagt

SEQ ID NO: 585 - RNA, 20 nt, synthetic construct
  source 1..20: mol_type = other RNA
gaagtgttaggccttgtaac

SEQ ID NO: 586 - RNA, 20 nt, synthetic construct
  source 1..20: mol_type = other RNA
gctgttacagtgcttcagaa

SEQ ID NO: 587 - RNA, 20 nt, synthetic construct
  source 1..20: mol_type = other RNA
attgattactgctgttacag

SEQ ID NO: 588 - RNA, 20 nt, synthetic construct
  source 1..20: mol_type = other RNA
gtggtaaggcctaattgatt

SEQ ID NO: 589 - RNA, 20 nt, synthetic construct
  source 1..20: mol_type = other RNA
aactgagaatgtggtaaggc

SEQ ID NO: 590 - RNA, 20 nt, synthetic construct
  source 1..20: mol_type = other RNA
taaccaaagaccgatgactt

SEQ ID NO: 591 - RNA, 20 nt, synthetic construct
  source 1..20: mol_type = other RNA
ggcttatgtttaaccaaaga

SEQ ID NO: 592 - RNA, 20 nt, synthetic construct
  source 1..20: mol_type = other RNA
ccccttaacattggcttatg

SEQ ID NO: 593 - RNA, 20 nt, synthetic construct
  source 1..20: mol_type = other RNA
taaccttttgattttgtccc

SEQ ID NO: 594 - RNA, 20 nt, synthetic construct
  source 1..20: mol_type = other RNA
cctacactttatgaacatac

SEQ ID NO: 595 - RNA, 20 nt, synthetic construct
  source 1..20: mol_type = other RNA
gctcagtaaggcatattaat

SEQ ID NO: 596 - RNA, 20 nt, synthetic construct
  source 1..20: mol_type = other RNA
ctgaacaacttgccacatag

SEQ ID NO: 597 - RNA, 20 nt, synthetic construct
  source 1..20: mol_type = other RNA
ctccaggattctgaacaact

SEQ ID NO: 598 - RNA, 20 nt, synthetic construct
  source 1..20: mol_type = other RNA
ttatgatatgtaggttcctc

SEQ ID NO: 599 - RNA, 20 nt, synthetic construct
  source 1..20: mol_type = other RNA
atttatcaccaccatacagt

SEQ ID NO: 600 - RNA, 20 nt, synthetic construct
  source 1..20: mol_type = other RNA
gcagactgaaatataagacc

SEQ ID NO: 601 - RNA, 20 nt, synthetic construct
  source 1..20: mol_type = other RNA
cttctataatgcagactgaa

SEQ ID NO: 602 - RNA, 20 nt, synthetic construct
  source 1..20: mol_type = other RNA
ggttgctacattatgaaaga

SEQ ID NO: 603 - RNA, 20 nt, synthetic construct
  source 1..20: mol_type = other RNA
tgaatcaatagggttgctac

SEQ ID NO: 604 - RNA, 20 nt, synthetic construct
  source 1..20: mol_type = other RNA
ggtaactgactaatttgaag

SEQ ID NO: 605 - RNA, 20 nt, synthetic construct
  source 1..20: mol_type = other RNA
aaatttcatagggaggtaac

SEQ ID NO: 606 - RNA, 20 nt, synthetic construct
  source 1..20: mol_type = other RNA
ttcaaggctactctgtttgc

SEQ ID NO: 607 - RNA, 20 nt, synthetic construct
  source 1..20: mol_type = other RNA
gtgatctaaatgagttgaac

SEQ ID NO: 608 - RNA, 20 nt, synthetic construct
  source 1..20: mol_type = other RNA
gcttccatttgattttcttc

SEQ ID NO: 609 - RNA, 20 nt, synthetic construct
  source 1..20: mol_type = other RNA
ccctagggatctttagcttc

SEQ ID NO: 610 - RNA, 20 nt, synthetic construct
  source 1..20: mol_type = other RNA
ggtcaaagtttgtgttaggc

SEQ ID NO: 611 - RNA, 20 nt, synthetic construct
  source 1..20: mol_type = other RNA
gaggcccgaaaaaagctgcg

SEQ ID NO: 612 - RNA, 20 nt, synthetic construct
  source 1..20: mol_type = other RNA
atttgcaatgacggaggccc

SEQ ID NO: 613 - RNA, 20 nt, synthetic construct
  source 1..20: mol_type = other RNA
cttgatgggatttgtatttc

SEQ ID NO: 614 - RNA, 20 nt, synthetic construct
  source 1..20: mol_type = other RNA
tacgctgactgcccttgatg

SEQ ID NO: 615 - RNA, 20 nt, synthetic construct
  source 1..20: mol_type = other RNA
gaggagtttggtatgctacg

SEQ ID NO: 616 - RNA, 20 nt, synthetic construct
  source 1..20: mol_type = other RNA
tcacttcaagccttcaggag

SEQ ID NO: 617 - RNA, 20 nt, synthetic construct
  source 1..20: mol_type = other RNA
ggcatgctagaattcacttc

SEQ ID NO: 618 - RNA, 20 nt, synthetic construct
  source 1..20: mol_type = other RNA
tcagtatctcttaggcaatg

SEQ ID NO: 619 - RNA, 20 nt, synthetic construct
  source 1..20: mol_type = other RNA
gggtgctgtagaaagttttc

SEQ ID NO: 620 - RNA, 20 nt, synthetic construct
  source 1..20: mol_type = other RNA
catatttccaacctgttcca

SEQ ID NO: 621 - RNA, 20 nt, synthetic construct
  source 1..20: mol_type = other RNA
gcctttacatctgtattaga

SEQ ID NO: 622 - RNA, 20 nt, synthetic construct
  source 1..20: mol_type = other RNA
gactattcctctgcctttac

SEQ ID NO: 623 - RNA, 20 nt, synthetic construct
  source 1..20: mol_type = other RNA
tgctatggaaatggagacta

SEQ ID NO: 624 - RNA, 20 nt, synthetic construct
  source 1..20: mol_type = other RNA
taggtcttattgctatggaa

SEQ ID NO: 625 - RNA, 20 nt, synthetic construct
  source 1..20: mol_type = other RNA
cgatctagaggtgacattag

SEQ ID NO: 626 - RNA, 20 nt, synthetic construct
  source 1..20: mol_type = other RNA
gactttctgacgatctagag

SEQ ID NO: 627 - RNA, 20 nt, synthetic construct
  source 1..20: mol_type = other RNA
atttgctcatgtctagccag

SEQ ID NO: 628 - RNA, 20 nt, synthetic construct
  source 1..20: mol_type = other RNA
ccctcattccaatttgctca

SEQ ID NO: 629 - RNA, 20 nt, synthetic construct
  source 1..20: mol_type = other RNA
gtgctttcctctccctcatt

SEQ ID NO: 630 - RNA, 20 nt, synthetic construct
  source 1..20: mol_type = other RNA
taatcttgacctgtgctttc

SEQ ID NO: 631 - RNA, 20 nt, synthetic construct
  source 1..20: mol_type = other RNA
ggactagtactaatcttgac

SEQ ID NO: 632 - RNA, 20 nt, synthetic construct
  source 1..20: mol_type = other RNA
actgcccaagggactagtac

SEQ ID NO: 633 - RNA, 20 nt, synthetic construct
  source 1..20: mol_type = other RNA
agcattctggctgtagaact

SEQ ID NO: 634 - RNA, 20 nt, synthetic construct
  source 1..20: mol_type = other RNA
ttgggtcagaagctaagcat

SEQ ID NO: 635 - RNA, 20 nt, synthetic construct
  source 1..20: mol_type = other RNA
cagtaccctagtcccagctt

SEQ ID NO: 636 - RNA, 20 nt, synthetic construct
  source 1..20: mol_type = other RNA
gcacatcactcagtacccta

SEQ ID NO: 637 - RNA, 20 nt, synthetic construct
  source 1..20: mol_type = other RNA
ctttgagagttaggccctaa

SEQ ID NO: 638 - RNA, 20 nt, synthetic construct
  source 1..20: mol_type = other RNA
tagtgtcatggtgtatctct

SEQ ID NO: 639 - RNA, 20 nt, synthetic construct
  source 1..20: mol_type = other RNA
aggaggctgtcactgttagt

SEQ ID NO: 640 - RNA, 20 nt, synthetic construct
  source 1..20: mol_type = other RNA
gagtgaggtacctgagatgc

SEQ ID NO: 641 - RNA, 20 nt, synthetic construct
  source 1..20: mol_type = other RNA
ctaggttaagtgagtgaggt

SEQ ID NO: 642 - RNA, 20 nt, synthetic construct
  source 1..20: mol_type = other RNA
gtttaaatgtcctagactag

SEQ ID NO: 643 - RNA, 20 nt, synthetic construct
  source 1..20: mol_type = other RNA
gagcttcttgacataaaact

SEQ ID NO: 644 - RNA, 20 nt, synthetic construct
  source 1..20: mol_type = other RNA
caacgccttgacattatgag

SEQ ID NO: 645 - RNA, 20 nt, synthetic construct
  source 1..20: mol_type = other RNA
tagtcaccaacaacgccttg

SEQ ID NO: 646 - RNA, 20 nt, synthetic construct
  source 1..20: mol_type = other RNA
agatggcctttcttttgaac

SEQ ID NO: 647 - RNA, 20 nt, synthetic construct
  source 1..20: mol_type = other RNA
tctaattgggagatggcctt

SEQ ID NO: 648 - RNA, 20 nt, synthetic construct
  source 1..20: mol_type = other RNA
gcaatacttctctaattggg

SEQ ID NO: 649 - RNA, 20 nt, synthetic construct
  source 1..20: mol_type = other RNA
tacttttggcatgcaatact

SEQ ID NO: 650 - RNA, 20 nt, synthetic construct
  source 1..20: mol_type = other RNA
gatggacaactttacatact

SEQ ID NO: 651 - RNA, 20 nt, synthetic construct
  source 1..20: mol_type = other RNA
aattagtttgcctgatggac

SEQ ID NO: 652 - RNA, 20 nt, synthetic construct
  source 1..20: mol_type = other RNA
gagctgttgcagaattagtt

SEQ ID NO: 653 - RNA, 20 nt, synthetic construct
  source 1..20: mol_type = other RNA
agtcccaaatgagctgttgc

SEQ ID NO: 654 - RNA, 20 nt, synthetic construct
  source 1..20: mol_type = other RNA
ttaattgtgttcagtcccaa

SEQ ID NO: 655 - RNA, 20 nt, synthetic construct
  source 1..20: mol_type = other RNA
catctatggcaaactacatt

SEQ ID NO: 656 - RNA, 20 nt, synthetic construct
  source 1..20: mol_type = other RNA
ggacggatttcatctatggc

SEQ ID NO: 657 - RNA, 20 nt, synthetic construct
  source 1..20: mol_type = other RNA
tatagacacattttaggacg

SEQ ID NO: 658 - RNA, 20 nt, synthetic construct
  source 1..20: mol_type = other RNA
ggttatcacacacttttaag

SEQ ID NO: 659 - RNA, 20 nt, synthetic construct
  source 1..20: mol_type = other RNA
gtacacgttagatgtaagag

SEQ ID NO: 660 - RNA, 20 nt, synthetic construct
  source 1..20: mol_type = other RNA
tattggagcctctgtacacg

SEQ ID NO: 661 - RNA, 20 nt, synthetic construct
  source 1..20: mol_type = other RNA
ctcattatcatattggagcc

SEQ ID NO: 662 - RNA, 20 nt, synthetic construct
  source 1..20: mol_type = other RNA
gtcacaggaaatataactca